(19) United States
(12) Patent Application Publication
(10) Pub. No.: US 2026/0272652 A1
(43) Pub. Date: Sep. 17, 2026

(54) HEART VALVE REPAIR DEVICES AND DELIVERY DEVICES THEREFOR

(71) Applicant: Edwards Lifesciences Corporation, Irvine, CA (US)

(72) Inventors: Sergio Delgado, Irvine, CA (US); Timothy Allen Dalton, Garden Grove, CA (US); Samuel Jacob Kipperman, Boulder, CO (US); Bingquan Su, Irvine, CA (US); Yevgeniy Davidovich Kaufman, Denver, CO (US)

(21) Appl. No.: 19/677,263

(22) Filed: May 14, 2026

Related U.S. Application Data
(63) Continuation of application No. PCT/US2024/054911, filed on Nov. 7, 2024.
(60) Provisional application No. 63/600,023, filed on Nov. 16, 2023.
(60) Provisional application No. 63/650,167, filed on May 21, 2024.

(51) Int. Cl. A61F 2/24

(52) U.S. Cl. CPC A61F 2/246; A61F 2/2466; A61F 2220/0008; A61F 2250/0006

(57) ABSTRACT

Devices are configured to be positioned within a native heart valve to impede regurgitation through the native heart valve. The devices can have a small size that allows the device to be inserted through a steerable catheter. The steerable catheter extend through a guide sheath. The devices can include adjustable spring closing force. The devices can include biasing members with variable loading and unloading. The devices can include gripping members or clasp members with hinge portions having reduced sizes.

### RELATED APPLICATIONS

The present application is a continuation of Patent Cooperation Treaty Application No. PCT/US2024/054911, filed on Nov. 7, 2024, which claims the benefit of U.S. Provisional Application No. 63/600,023, filed on Nov. 16, 2023 and 63/650,167, filed on May 21, 2024, which are all incorporated herein by reference in their entireties for all purposes.

### BACKGROUND

The native heart valves (i.e., the aortic, pulmonary, tricuspid, and mitral valves) serve critical functions in assuring the forward flow of an adequate supply of blood through the cardiovascular system. These heart valves may be damaged, and thus rendered less effective, for example, by congenital malformations, inflammatory processes, infectious conditions, disease, etc. Such damage to the valves may result in serious cardiovascular compromise or death. Damaged valves can be surgically repaired or replaced during open heart surgery. However, open heart surgeries are highly invasive, and complications may occur. Transvascular techniques can be used to introduce and implant devices to treat a heart in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to implant a device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus may form a “D”-shaped, oval, or otherwise out-of-round cross-sectional shape having major and minor axes. The anterior leaflet may be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation may have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation may be similar, but on the right side of the heart.

### SUMMARY

This summary is meant to provide some examples and is not intended to limit of the scope of the disclosed subject matter in any way. For example, any feature included in an example of this summary is not required by the claims, unless the claims explicitly recite the feature. Also, the features, components, steps, concepts, etc. described in examples in this summary and elsewhere in this disclosure can be combined in a variety of ways. Various features and steps as described elsewhere in this disclosure can be included in the examples summarized here.

Devices for repairing and/or treating a native valve of a patient are disclosed. The devices can be valve repair devices, implantable devices, valve treatment devices, implants, etc. While the devices may be described and/or depicted as implantable devices in some examples herein, similar configurations can be used on other devices, e.g., valve repair devices, treatment devices, etc., that are not necessarily implanted and may be removed after treatment.

In some implementations, there is provided a device (e.g., a treatment device, a repair device, an implantable device, an implant, etc.) that is configured to be positioned within a native heart valve to allow the native heart valve to form a more effective seal.

In some implementations, a device (e.g., an implantable device, implant, treatment device, etc.) includes an anchor portion. In some implementations, the anchor portion includes one or more anchors. In some implementations, each anchor includes one or more paddles that are each movable between an open position and a closed position.

In some implementations, a device (e.g., a valve treatment device, an implantable device, a treatment device, etc.) includes a base. In some implementations, the device includes an actuation assembly. In some implementations, the valve treatment device includes one or more paddles. In some implementations, the actuation assembly is coupled to the base. In some implementations, the one or more paddles are pivotally coupled to the base. In some implementations, the actuation assembly is configured to convert a rotational input motion to pivoting movement of the one or more paddles relative to the base.

In some implementations the base comprises a frame with an open interior.

In some implementations, the actuation assembly is disposed in the open interior of the base.

In some implementations, the actuation assembly comprises one or more of a rack and pinion assemblies, a worm gear assembly, and a planetary gear assembly.

In some implementations, the actuation assembly comprises a threaded drive member, a threaded driven member, and a gear rack.

In some implementations, each of the one or more paddles comprises a pinion gear.

In some implementations, the actuation assembly comprises a rack and each of the one or more paddles comprises a pinion gear that is driven by the rack.

In some implementations, the device further comprises a gripping member configured to grasp a native valve leaflet.

In some implementations, the gripping member is configured to grasp the native valve leaflet with one of the one or more paddles.

In some implementations, each of the one or more paddles extends from a pivotable portion that is pivotably connected to the base to a free end.

In some implementations, the device further comprises a paddle frame connected to each of the one or more paddles, wherein the paddle frame is configured to coapt two native valve leaflets together.

In some implementations, each paddle of the one or more paddles can form an angle with the base that is greater than 175 degrees.

In some implementations, the gripping member can form an angle of 180 degrees with one of the one or more paddles.

In some implementations, a valve treatment system comprises a catheter coupled to the valve repair device.

In some implementations, a treatment and/or repair system comprises one or more (e.g., one, some, or all) of a guide sheath, a steerable catheter, a device or implant catheter, and a treatment and/or repair device. In some implementations, the steerable catheter can be configured to extend through the guide sheath. In some implementations, the device/implant catheter can be configured to extend through the steerable catheter and/or the guide sheath.

In some implementations, the treatment and/or repair device is coupled to the device/implant catheter. In some implementations, the treatment and/or repair device includes one or more paddles that are movable between an open position and a closed position.

In some implementations, the one or more paddles are each configured to be secured to a native valve leaflet by moving the paddle from the open position to the closed position.

In some implementations, a passage of the steerable catheter and a size of the treatment and/or repair device are configured to allow the treatment and/or repair device to slide through the steerable catheter.

In some implementations, the valve repair device is configured to be secured to leaflets of a native mitral valve having a mitral valve area (MVA) in cm² between 2.2 and 6.

In some implementations, the valve repair device is configured to be secured to leaflets of a native mitral valve when a transeptal puncture height in cm is between 2.0 and 4.5.

In some implementations, the valve repair device is configured to be implanted where a mitral valve area is between 2.2 and 6 cm² and a transeptal puncture height is between 2.0 and 4.5 cm.

In some implementations, a medial-lateral width of the valve repair device is between 2 mm and 4 mm.

In some implementations, an anterior-posterior width of the valve repair device is between 2 mm and 9 mm.

In some implementations, a leaflet capture height is between 5 mm and 10 mm.

In some implementations, a valve treatment device for attaching to a native valve of a patient is provided. In some implementations, the valve treatment device can include a base, one or more paddles, an actuation assembly, and a biasing member.

In some implementations, the one or more paddles can be pivotably coupled to the base such that the paddles are movable between an open position and a closed position.

In some implementations, the actuation assembly is coupled to the base and configured to convert a rotational input motion to pivoting movement of the one or more paddles relative to the base such that the one or more paddles move between the open and closed positions.

In some implementations, ta proximal end of the actuation assembly is positioned proximate such that the actuation assembly can linearly move within the gap.

In some implementations, the biasing member engages the actuation assembly to maintain the actuation assembly in a desired position relative to the gap.

In some implementations, when the one or more paddles are in the closed position and attached to the native valve, movement of the leaflets during the diastolic phase causes the actuation assembly to linearly move within the gap.

In some implementations, the base comprises a frame with an open interior.

In some implementations, the actuation assembly is disposed in the open interior of the base.

In some implementations, the actuation assembly comprises one or more of a rack and pinion assembly, a worm gear assembly, and a planetary gear assembly.

In some implementations, the actuation assembly comprises a threaded drive member, a threaded driven member, and a gear rack.

In some implementations, the threaded drive member comprises a head that moves linearly within the gap due to movement of the leaflets during the diastolic phase.

In some implementations, each of the one or more paddles comprises one or more pinion gears.

In some implementations, the actuation assembly comprises a rack and each of the one or more paddles comprises one or more pinion gears that are driven by the rack.

In some implementations, the device further comprises a gripping member configured to grasp a native valve leaflet.

In some implementations, the gripping member is configured to grasp the native valve leaflet with one of the one or more paddles.

In some implementations, the device further comprises a second actuation assembly coupled to the base.

In some implementations, the second actuation assembly is configured to move the gripping member between an open position and a closed position.

In some implementations, the second actuation assembly comprises one or more (e.g., one, some, or all) a threaded drive member and a threaded driven member. In some implementations, the gripping member is connected to the threaded driven member such that movement of the threaded driven member by the threaded drive member causes the gripping member to move between the open and closed positions.

In some implementations, each of the one or more paddles extends from a pivotable portion that is pivotably connected to the base to a free end.

In some implementations, the device further comprises a paddle frame connected to each of the one or more paddles.

In some implementations, the paddle frame is configured to coapt two native valve leaflets together.

In some implementations, the second actuation assembly is configured to move the paddle frame between a narrowed configuration and an expanded configuration.

In some implementations, the second actuation assembly comprises a threaded drive member and a threaded driven member.

In some implementations, the paddle frame is connected to the threaded driven member such that movement of the threaded driven member by the threaded drive member causes the paddle frame to move between the narrowed and expanded configurations.

In some implementations, the device further comprises frame actuation elements that are pivotably coupled to the one or more paddles.

In some implementations, the frame actuation elements are connected to the threaded driven member and the paddle frame such that movement of the threaded driven member causes the paddle frame to move between the narrowed and expanded configurations.

In some implementations, the second actuation assembly includes a threaded drive member and a threaded driven member.

In some implementations, the second actuation assembly is configured to actuate one or more of the base, a gripping member, and a paddle frame of the device.

In some implementations, each paddle of the one or more paddles can form an angle with the base that is greater than 175 degrees.

In some implementations, the biasing member is positioned within the gap and engages a threaded drive member of the actuation assembly to maintain the actuation assembly in a desired position relative to the gap.

In some implementations, the biasing member is positioned outside of the gap and engages a threaded driven member of the actuation assembly to maintain the actuation assembly in a desired position relative to the gap.

In some implementations, the device further comprises a coupling, and the gap is disposed within the coupling.

In some implementations, the biasing member comprises a spring made of Nitinol.

In some implementations, the valve treatment device can be included in a valve treatment system that includes a catheter, where the valve treatment device is coupled to the catheter.

In some implementations, a valve treatment device for attaching to a native valve of a patient is provided. In some implementations, the valve treatment device can include a base, one or more paddles, a first actuation assembly, and a second actuation assembly.

In some implementations, the one or more paddles can be pivotably coupled to the base such that the paddles are movable between an open position and a closed position.

In some implementations, the first actuation assembly can be coupled to the base and configured to convert a rotational input motion to pivoting movement of the one or more paddles relative to the base such that the one or more paddles move between the closed position and the open position.

In some implementations, the second actuation assembly can be coupled to the base and configured to convert rotational input motion to movement of one or more components of the valve treatment device relative to the base.

In some implementations, the second actuation assembly can include a threaded drive member and a threaded driven member that is operatively attached to the threaded drive member such that rotation of the threaded drive member causes the threaded drive member to move linearly relative to the threaded drive member.

In some implementations, the one or more components of the valve treatment device comprises a gripping member that is configured to grasp a native valve leaflet.

In some implementations, linear movement of the threaded driven member relative to the threaded drive member causes the gripping member to move between an open position and a closed position.

In some implementations, the gripping member is configured to grasp the native valve leaflet with one of the one or more paddles.

In some implementations, the gripping member is attached to the driven member by an actuation line.

In some implementations, the one or more components of the valve treatment device comprises a paddle frame connected to each of the one or more paddles.

In some implementations, the paddle frame comprises a body portion, a first arm pivotable relative to the body portion, and a second arm pivotable relative to the body portion.

In some implementations, linear movement of the threaded driven member relative to the threaded drive member causes the first and second arms of the paddle frame to pivot relative to the body portion to move the paddle frame between a narrowed configuration and an expanded configuration.

In some implementations, the valve treatment device further comprises a first frame actuation element and a second frame actuation element.

In some implementations, the first and second frame actuation elements are pivotable relative to a paddle of the one or more paddles between an open position and a closed position.

In some implementations, the first arm of the paddle frame is attached to the first frame actuation element and the second arm of the paddle frame is attached to the second frame actuation element.

In some implementations, the first and second frame actuation elements are attached to the threaded driven member by one or more actuation lines.

In some implementations, the one or more components of the valve treatment device comprises a link assembly of the base, where the link assembly comprises a lower link that is pivotably attached to the base and an upper link that is pivotably attached to the threaded driven member.

In some implementations, linear movement of the threaded driven member relative to the threaded drive member causes the upper and lower links to pivot relative to each other and move the base between a narrowed configuration and an expanded configuration.

In some implementations, the one or more components of the valve treatment device comprises a movable member of the one or more paddles, where each of the one or more paddles comprises a fixed member and the movable member that is movable relative to the fixed member.

In some implementations, linear movement of the threaded driven member relative to the threaded drive member causes the movable member to move relative to the fixed member such that the one or more paddles move between a shortened configuration and a lengthened configuration.

In some implementations, the movable member is attached to the threaded driven member by one or more actuation lines.

In some implementations, the valve treatment device can be included in a valve treatment system that includes a catheter, where the valve treatment device is coupled to the catheter.

In some implementations, a valve treatment system is provided that includes a delivery device and a valve treatment device.

In some implementations, the delivery device includes an actuation element, which comprises a catheter, a drive end connected to the catheter, and a securing element.

In some implementations, the drive end has a passage that is in communication with the catheter and a slot at a distal end that is in communication with the passage.

In some implementations, the securing element is configured to be moved through the catheter and the passage such that the securing element can be inserted into the slot at the distal end of the drive end.

In some implementations, the valve treatment device comprises a base and an actuation assembly.

In some implementations, the actuation assembly is coupled to the base and includes a drive member that has a head.

In some implementations, the head has a bar that is configured to be inserted into the slot of the drive end of the actuation element of the delivery device. In some implementations, the head of the actuation assembly of the valve treatment device is secured to the actuation element of the delivery device when the bar of the head of the drive member is inserted into the slot of the drive end of the actuation element and the securing element of the actuation element is positioned within the slot.

In some implementations, rotation of the drive end of the actuation element causes the drive element of the actuation assembly of the valve treatment device to rotate when the head of the actuation assembly of the valve treatment device is secured to the actuation element.

In some implementations, the delivery device further comprises an outer catheter for housing the actuation element.

In some implementations, the outer catheter comprises one or more slots that are configured to receive one or more projections of the head of the drive member of the valve treatment device such that the outer catheter can be secured to the valve head of the drive member by inserting the projections of the head into the slots of the outer catheter.

In some implementations, the passage of the drive end has a proximal opening that is sized to align with the catheter of the actuation element and one or more sloped walls such that the passage narrows from the proximal opening to a distal opening.

In some implementations, this disclosure relates to a valve treatment device for attaching to a native valve of a patient. In some implementations, the valve treatment device can include a base, one or more paddles, and an actuation assembly.

In some implementations, the one or more paddles pivotably are coupled to the base such that the one or more paddles are movable between an open position and a closed position.

In some implementations, the actuation assembly is coupled to the base, such that the actuation assembly is configured to pivot the one or more paddles relative to the base between the open position and the closed position responsive to the actuation assembly receiving an input. In some implementations, a proximal end of the actuation assembly is positioned proximate a gap such that the actuation assembly can linearly move within the gap.

In some implementations, when the one or more paddles are in the closed position and attached to the native valve of the patient, movement of the leaflets during the diastolic phase causes the actuation assembly to linearly move within the gap.

In some implementations, the device further comprises a biasing member that engages the actuation assembly to maintain the actuation assembly in a desired position relative to the gap. In some implementations, the desired position of the actuation assembly relative to the gap maintains the one or more paddles biased towards the closed position.

In some implementations, the biasing member is positioned within the gap and engages a threaded drive member of the actuation assembly to maintain the actuation assembly in a desired position relative to the gap.

In some implementations, the biasing member is positioned outside of the gap and engages a threaded driven member of the actuation assembly to maintain the actuation assembly in a desired position relative to the gap.

In some implementations, the device further comprises a coupling attached to the base.

In some implementations, the gap is disposed within the coupling.

In some implementations, the biasing member comprises a spring made of Nitinol.

In some implementations, the base comprises a frame. In some implementations, the actuation assembly is disposed in an open interior of the frame.

In some implementations, the actuation assembly comprises one or more of a rack and pinion assembly, a worm gear assembly, and a planetary gear assembly.

In some implementations, the actuation assembly comprises a threaded drive member, a threaded driven member, and a gear rack.

In some implementations, the threaded drive member comprises a head that moves linearly within the gap due to movement of the leaflets during the diastolic phase.

In some implementations, each of the one or more paddles comprises one or more pinion gears.

In some implementations, the actuation assembly comprises a rack and each of the one or more paddles comprises one or more pinion gears that are driven by the rack.

In some implementations, the device further comprises a gripping member (e.g., a gripper or a clasp) configured to grasp a native valve leaflet.

In some implementations, the device further comprises a second actuation assembly coupled to the base. In some implementations, the second actuation assembly is configured to move the gripping member between an open position and a closed position. In some implementations, the second actuation assembly comprises a threaded drive member and a threaded driven member. In some implementations, the gripping member is connected to the threaded driven member such that movement of the threaded driven member by the threaded drive member causes the gripping member to move between the open and closed position.

In some implementations, the gripping member is configured to grasp the native valve leaflet with one of the one or more paddles.

In some implementations, each of the one or more paddles extends from a pivotable portion, that is pivotably connected to the base, to a free end.

In some implementations, the device further comprises a paddle frame connected to each of the one or more paddles, wherein the paddle frame is configured to coapt two native valve leaflets together.

In some implementations, the device further comprises a second actuation assembly coupled to the base. In some implementations, the second actuation assembly is configured to move the paddle frame between a narrowed configuration and an expanded configuration.

In some implementations, the second actuation assembly comprises a threaded drive member and a threaded driven member. In some implementations, the paddle frame is connected to the threaded driven member such that movement of the threaded driven member by the threaded drive member causes the paddle frame to move between the narrowed and expanded configurations.

In some implementations, the device further comprises frame actuation elements that are pivotably coupled to the one or more paddles. In some implementations, the frame actuation elements are connected to the threaded driven member such that movement of the threaded driven member causes the paddle frame to move between the narrowed and expanded configurations.

In some implementations, each paddle of the one or more paddles can form an angle with the base that is greater than 175 degrees.

In some implementations, the device further comprises a second actuation assembly that includes a threaded drive member and a threaded driven member. In some implementations, the second actuation assembly is configured to actuate one or more of the base, a gripping member, and a paddle frame of the device.

In some implementations, apparatuses, systems, and/or methods described herein relate to a valve treatment device for attaching to a native valve of a patient. In some implementations, the valve treatment device includes a base. In some implementations, the valve treatment device includes one or more paddles pivotably coupled to the base such that the paddles are movable between an open position and a closed position. In some implementations, the valve treatment device includes an actuation assembly coupled to the base.

In some implementations, the actuation assembly is configured to move of the one or more paddles relative to the base such that the one or more paddles move between the closed position and the open position. In some implementations, a proximal end of the actuation assembly is positioned proximate a gap. In some implementations, the actuation assembly can linearly move within the gap.

In some implementations, a biasing member that engages the actuation assembly to maintain the actuation assembly in a desired position relative to the gap. In some implementations, a bias adjustment assembly is configured to position the bias member in any one or more bias positions.

In some implementations, when the one or more paddles are in the closed position and attached to the native valve of the patient, movement of the leaflets during the diastolic phase causes the actuation assembly to linearly move within the gap.

In some implementations, apparatuses, systems, and/or methods described herein relate to a device wherein the bias adjustment assembly includes a bias member space configured to receive at least a portion of the bias member.

In some implementations, apparatuses, systems, and/or methods described herein relate to a device wherein the bias adjustment assembly includes a positioner space configured to receive at least a portion of a bias member positioner.

In some implementations, apparatuses, systems, and/or methods described herein relate to a device wherein the bias adjustment assembly includes a positioner for changing the position of the bias member.

In some implementations, apparatuses, systems, and/or methods described herein relate to a device wherein the bias adjustment assembly includes a threaded positioner.

In some implementations, apparatuses, systems, and/or methods described herein relate to a device wherein the bias adjustment assembly includes a threaded positioner having a central opening.

In some implementations, apparatuses, systems, and/or methods described herein relate to a device wherein the bias adjustment assembly includes a threaded positioner space configured to receive at least a portion of the threaded positioner.

In some implementations, apparatuses, systems, and/or methods described herein relate to a device wherein the bias adjustment assembly includes at least first and second states. In some implementations, the first state includes a first bias position and the second state includes a second bias position different form the first bias position.

In some implementations, apparatuses, systems, and/or methods described herein relate to a device wherein the bias adjustment assembly includes at least first and second states. In some implementations, the first state includes a first bias position providing a first bias force on the actuation assembly. In some implementations, the second state includes a second bias position providing a second bias force on the actuation assembly and wherein the second bias force is different from the first bias force.

In some implementations, apparatuses, systems, and/or methods described herein relate to a device wherein the bias adjustment assembly. In some implementations, the bias adjustment assembly includes at least first and second states. In some implementations, the first state includes a first bias position providing a first range of bias forces on the actuation assembly and the second state includes a second bias position providing a second range of bias forces on the actuation assembly. In some implementations, the second range of bias forces is different from the first range of bias forces.

In some implementations, apparatuses, systems, and/or methods described herein relate to a device wherein the bias member includes a first spring coefficient on loading of the bias member and a second spring coefficient on unloading of the bias member.

In some implementations, apparatuses, systems, and/or methods described herein relate to a device wherein the bias member includes a first spring coefficient on loading of the bias member and second and third spring coefficients on unloading of the bias member.

In some implementations, apparatuses, systems, and/or methods described herein relate to a device wherein the bias member includes a first force displacement curve on loading of the bias member and second force displacement curve on unloading of the bias member.

In some implementations, apparatuses, systems, and/or methods described herein relate to a device wherein the bias member includes a first force displacement curve on loading of the bias member and second and third force displacement curves on unloading of the bias member.

In some implementations, apparatuses, systems, and/or methods described herein relate to a device wherein the bias member includes a top and bottom loading surfaces and a plurality of struts connected to the top and bottom surfaces and wherein the struts each include a curved shape.

In some implementations, apparatuses, systems, and/or methods described herein relate to a device wherein the bias member includes top and bottom loading surfaces. In some implementations, the bias member includes a plurality of struts connected to the top and bottom surfaces. In some implementations, the struts each include a concave portion, a convex portion, and an inflexion portion between the concave and convex portions.

In some implementations, apparatuses, systems, and/or methods described herein relate to a device wherein the bias member includes top and bottom loading surfaces. In some implementations, the bias member includes a plurality of struts connected to the top and bottom surfaces. In some implementations, the struts each include a first curved portion, a second curved portion, and an inflexion portion between the first and second curved portion.

In some implementations, apparatuses, systems, and/or methods described herein relate to a device wherein the bias member includes a compound spring member having a plurality of stacked spring portions.

In some implementations, apparatuses, systems, and/or methods described herein relate to a device wherein the bias member includes a spring member having a plurality of arcuate sections with concave and convex portions.

In some implementations, apparatuses, systems, and/or methods described herein relate to a device wherein the bias member includes a plurality of stacked spring members having a plurality of arcuate sections with concave and convex portions.

In some implementations, apparatuses, systems, and/or methods described herein relate to a device wherein the bias member includes a rectangular spring member having a plurality of sections with concave and convex portions.

In some implementations, apparatuses, systems, and/or methods described herein relate to a device wherein the bias member includes a plurality of stacked rectangular spring members having a plurality of sections with concave and convex portions.

In some implementations, apparatuses, systems, and/or methods described herein relate to a device wherein the bias member includes a spring member having a plurality of protruding portions and each protruding portion includes a plateaued linear surface.

In some implementations, apparatuses, systems, and/or methods described herein relate to a device wherein the bias member includes a plurality of spring members having a plurality of protruding portions and each protruding portion includes a plateaued linear surface.

In some implementations, apparatuses, systems, and/or methods described herein relate to a device wherein the bias member includes curved body having first and second distal end portions and a gap between the distal end portions.

In some implementations, apparatuses, systems, and/or methods described herein relate to a device wherein the bias member includes curved body having first and second distal end portions. In some implementations, a gap between the distal end portions and wherein the first and second distal end portions are offset from each other.

In some implementations, apparatuses, systems, and/or methods described herein relate to a device wherein the bias member includes curved body having first and second distal end portions. In some implementations, a gap between the distal end portions and wherein the first and second distal end portions include convex and concave portions.

In some implementations, apparatuses, systems, and/or methods described herein relate to a device wherein the bias member includes cylindrical side wall having first and second openings. In some implementations, the first and second openings having a common center line.

In some implementations, apparatuses, systems, and/or methods described herein relate to a device wherein the bias member includes rectangular side wall having first and second openings. In some implementations, the first and second openings having a common center line.

In some implementations, apparatuses, systems, and/or methods described herein relate to a clasp for a valve treatment device for attaching to a native valve of a patient. In some implementations, in some implementations, the clasp includes a base portion. In some implementations, the clasp has a movable arm portion. In some implementations, the clasp has a joint portion connected to the base portion and the movable arm portion. In some implementations, the joint portion includes two or more joint elements and one or more gaps between the joint elements. In some implementations, the one or more gaps include at least one tapered space.

In some implementations, apparatuses, systems, and/or methods described herein relate to a clasp wherein the at least one tapered space includes a space that gradually decreases in size.

In some implementations, apparatuses, systems, and/or methods described herein relate to a clasp wherein the at least one tapered space includes a space having a linear taper.

In some implementations, apparatuses, systems, and/or methods described herein relate to a clasp wherein the at least one tapered space includes a space having a curvilinear taper.

In some implementations, apparatuses, systems, and/or methods described herein relate to a clasp wherein the at least one tapered space includes first and second side wall. In some implementations, the first side wall is a side wall of a first joint element and the second side wall is a side wall of a second joint element. In some implementations, the first and second side walls are non-parallel with respect to each other.

In some implementations, apparatuses, systems, and/or methods described herein relate to a clasp wherein the at least one tapered space includes first and second end portions. In some implementations, the first and second end portions includes different sizes.

In some implementations, apparatuses, systems, and/or methods described herein relate to a clasp wherein the at least one tapered space includes a first end portion having a first opening and a second end portions having a second opening. In some implementations, the first opening is larger than the second opening.

In some implementations, apparatuses, systems, and/or methods described herein relate to a clasp wherein the joint portion includes outer side portions and the at least one tapered space is disposed on the outer side portions.

In some implementations, apparatuses, systems, and/or methods described herein relate to a clasp wherein the at least one tapered space includes a plurality of tapered spaces having a plurality of different tapers.

In some implementations, apparatuses, systems, and/or methods described herein relate to a clasp wherein the at least one tapered space includes a taper angle in the range of 5 to 45 degrees.

In some implementations, apparatuses, systems, and/or methods described herein relate to a valve treatment device for attaching to a native valve of a patient. In some implementations, the valve treatment device includes a base. In some implementations, the valve treatment device includes one or more paddles pivotably coupled to the base such that the one or more paddles are movable between an open position and a closed position. In some implementations, the valve treatment device includes an actuation assembly coupled to the base. In some implementations, the actuation assembly is configured to pivot the one or more paddles relative to the base between the open position and the closed position responsive to the actuation assembly receiving an input.

In some implementations, one or more clasps are coupled to the one or more paddles. In some implementations, a base portion is connected to the one or more paddles. In some implementations, the one or more clasps have a movable arm portion. In some implementations, the one or more clasps have a joint portion connected to the base portion and the movable arm portion. In some implementations, the joint portion including one or more joint elements and one or more gaps between the joint elements. In some implementations, the one or more gaps include at least one tapered space.

In some implementations, apparatuses, systems, and/or methods described herein relate to a device wherein the at least one tapered space includes a space that gradually decreases in size.

In some implementations, apparatuses, systems, and/or methods described herein relate to a device wherein the at least one tapered space includes a space having a linear taper.

In some implementations, apparatuses, systems, and/or methods described herein relate to a device wherein the at least one tapered space includes a space having a curvilinear taper.

In some implementations, apparatuses, systems, and/or methods described herein relate to a device wherein the at least one tapered space includes first and second side wall. In some implementations, the first side wall is a side wall of a first joint element and the second side wall is a side wall of a second joint element. In some implementations, the first and second side walls are non-parallel with respect to each other.

In some implementations, apparatuses, systems, and/or methods described herein relate to a device wherein the at least one tapered space includes first and second end portions. In some implementations, the first and second end portions includes different sizes.

In some implementations, apparatuses, systems, and/or methods described herein relate to a device wherein the at least one tapered space includes a first end portion having a first opening and a second end portions having a second opening. In some implementations, the first opening is larger than the second opening.

In some implementations, apparatuses, systems, and/or methods described herein relate to a device wherein the joint portion includes outer side portions and the at least one tapered space is disposed on the outer side portions.

In some implementations, apparatuses, systems, and/or methods described herein relate to a device wherein the at least one tapered space includes a plurality of tapered spaces having a plurality of different tapers.

In some implementations, apparatuses, systems, and/or methods described herein relate to a device wherein the at least one tapered space includes a taper angle in the range of 5 to 45 degrees.

In some implementations, apparatuses, systems, and/or methods described herein relate to a clasp for a valve treatment device for attaching to a native valve of a patient. In some implementations, the clasp has a base portion connected to the one or more paddles. In some implementations, the clasp has a movable arm portion. In some implementations, the clasp has a joint portion connected to the base portion and the movable arm portion. In some implementations, the movable arm portion includes an arm thickness and wherein the joint portion includes a joint thickness and wherein the arm thickness is greater than the joint thickness.

In some implementations, apparatuses, systems, and/or methods described herein relate to a clasp wherein base portion includes a base thickness and the arm thickness is greater than the base thickness.

In some implementations, apparatuses, systems, and/or methods described herein relate to a clasp wherein the movable arm portion include a barbed portion having a barb thickness and wherein the base thickness is the same as the arm thickness.

In some implementations, apparatuses, systems, and/or methods described herein relate to a clasp that includes a transition portion between the movable arm portion and the joint portion. In some implementations, the transition portion includes a step transition from the movable arm portion to the joint portion.

In some implementations, apparatuses, systems, and/or methods described herein relate to a clasp that includes a transition portion between the movable arm portion. In some implementations, the joint portion and the transition portion includes a linear slope transition from the movable arm portion to the joint portion.

In some implementations, apparatuses, systems, and/or methods described herein relate to a clasp that further includes a transition portion between the movable arm portion and the joint portion and the transition portion includes a curved slope transition from the movable arm portion to the joint portion.

In some implementations, apparatuses, systems, and/or methods described herein relate to a clasp that includes a single piece of material having the base, movable arm and joint portions.

In some implementations, apparatuses, systems, and/or methods described herein relate to a clasp wherein the movable arm includes an arm stiffness and the joint portion includes a joint stiffness and wherein the arm stiffness is greater than the joint stiffness.

In some implementations, apparatuses, systems, and/or methods described herein relate to a valve treatment device for attaching to a native valve of a patient. In some implementations, the valve treatment device includes a base. In some implementations, the valve treatment device includes one or more paddles pivotably coupled to the base such that the one or more paddles are movable between an open position and a closed position. In some implementations, the valve treatment device includes an actuation assembly coupled to the base. In some implementations, the actuation assembly is configured to pivot the one or more paddles relative to the base between the open position and the closed position responsive to the actuation assembly receiving an input.

In some implementations, one or more clasps coupled to the one or more paddles. In some implementations, the one or more clasps have a base portion connected to the one or more paddles. In some implementations, the one or more clasps have a movable arm portion. In some implementations, the one or more clasps have a joint portion connected to the base portion and the movable arm portion. In some implementations, the movable arm portion includes an arm thickness and wherein the joint portion includes a joint thickness and wherein the arm thickness is greater than the joint thickness.

In some implementations, apparatuses, systems, and/or methods described herein relate to a device wherein base portion includes a base thickness and the arm thickness is greater than the base thickness.

In some implementations, apparatuses, systems, and/or methods described herein relate to a device wherein the movable arm portion include a barbed portion having a barb thickness and wherein the base thickness is the same as the arm thickness.

In some implementations, apparatuses, systems, and/or methods described herein relate to a device wherein the one or more clasps further include a transition portion between the movable arm portion and the joint portion. In some implementations, the transition portion includes a step transition from the movable arm portion to the joint portion.

In some implementations, apparatuses, systems, and/or methods described herein relate to a device wherein the one or more clasps include a transition portion between the movable arm portion. In some implementations, the joint portion and the transition portion includes a linear slope transition from the movable arm portion to the joint portion.

In some implementations, apparatuses, systems, and/or methods described herein relate to a device wherein the one or more clasps further include a transition portion between the movable arm portion and the joint portion and the transition portion includes a curved slope transition from the movable arm portion to the joint portion.

In some implementations, apparatuses, systems, and/or methods described herein relate to a device wherein the each of the one or more clasps include a single piece of material having the base, movable arm and joint portions.

In some implementations, apparatuses, systems, and/or methods described herein relate to a device wherein the movable arm includes an arm stiffness and the joint portion includes a joint stiffness and wherein the arm stiffness is greater than the joint stiffness.

In some implementations, apparatuses, systems, and/or methods described herein relate to a valve treatment device for attaching to a native valve of a patient, the valve treatment device includes a base. In some implementations, the valve treatment device includes one or more paddles pivotably coupled to the base such that the one or more paddles are movable between an open position and a closed position. In some implementations, the valve treatment device includes an actuation assembly coupled to the base. In some implementations, the actuation assembly is configured to pivot the one or more paddles relative to the base between the open position and the closed position responsive to the actuation assembly receiving an input.

In some implementations, one or more clasps are coupled to the one or more paddles. In some implementations, the one or more clasps have a base portion connected to the one or more paddles. In some implementations, the one or more clasps have a movable arm portion. In some implementations, the one or more clasps have a joint portion connected to the base portion and the movable arm portion. In some implementations, the joint portion includes an opening for receiving at least a portion of the one or more paddles.

In some implementations, apparatuses, systems, and/or methods described herein relate to a device wherein the portion of the one or more paddles received in the opening includes a gear portion.

In some implementations, apparatuses, systems, and/or methods described herein relate to a device wherein the portion of the one or more paddles received in the opening includes a portion of a paddle arm.

In some implementations, apparatuses, systems, and/or methods described herein relate to a device wherein the opening includes a rectangular opening.

In some implementations, apparatuses, systems, and/or methods described herein relate to a device wherein the opening includes a curved opening.

In some implementations, apparatuses, systems, and/or methods described herein relate to a device wherein the opening extends into a portion of the base portion.

In some implementations, apparatuses, systems, and/or methods described herein relate to a device wherein the base portion includes first and second base portion separated by a cut.

In some implementations, apparatuses, systems, and/or methods described herein relate to a device wherein the one or more paddle include a pivot axis and the joint portion of the one or more clasps includes a central axis. In some implementations, the central axis is offset from the pivot axis.

In some implementations, apparatuses, systems, and/or methods described herein relate to a device wherein the one or more paddle include a pivot axis and the joint portion of the one or more clasps includes a central axis. In some implementations, the central axis is coaxial with the pivot axis.

In some implementations, apparatuses, systems, and/or methods described herein relate to a clasp for a valve treatment device for attaching to a native valve of a patient. In some implementations, the clasp includes a base portion. In some implementations, the clasp includes a movable arm portion. In some implementations, the clasp includes a joint portion connected to the base portion and the movable arm portion. In some implementations, the joint portion includes at least one coil spring portion.

In some implementations, apparatuses, systems, and/or methods described herein relate to a clasp wherein the base portion, movable arm portion, joint portion, and coil spring portion are made from a single piece of material.

In some implementations, apparatuses, systems, and/or methods described herein relate to a clasp wherein the base portion is made from a first piece of material, the movable arm portion is made from a second piece of material, and the coil spring portion is made from a third piece of material. In some implementations, the third piece of material is joined to the first and second pieces of material.

In some implementations, apparatuses, systems, and/or methods described herein relate to a clasp wherein the coil spring portion includes a bias force urging the base portion and the movable arm portion together.

In some implementations, apparatuses, systems, and/or methods described herein relate to a clasp wherein the coil spring portion includes a coil axis that is offset from a paddle pivot axis.

In some implementations, apparatuses, systems, and/or methods described herein relate to a valve treatment device for attaching to a native valve of a patient. In some implementations, the valve treatment device includes a base. In some implementations, the valve treatment device includes one or more paddles pivotably coupled to the base such that the one or more paddles are movable between an open position and a closed position. In some implementations, an actuation assembly is coupled to the base. In some implementations, the actuation assembly is configured to pivot the one or more paddles relative to the base between the open position and the closed position responsive to the actuation assembly receiving an input.

In some implementations, one or more clasps are coupled to the one or more paddles. In some implementations, the one or more clasps include a base portion connected to the one or more paddles. In some implementations, the one or more clasps include a movable arm portion. In some implementations, the one or more clasps include a joint portion connected to the base portion and the movable arm portion. In some implementations, the joint portion includes at least one coil spring portion.

In some implementations, apparatuses, systems, and/or methods described herein relate to a device wherein the base portion, movable arm portion, joint portion, and coil spring portion are made from a single piece of material.

In some implementations, apparatuses, systems, and/or methods described herein relate to a device wherein the base portion is made from a first piece of material, the movable arm portion is made from a second piece of material, and the coil spring portion is made from a third piece of material. In some implementations, the third piece of material is joined to the first and second pieces of material.

In some implementations, apparatuses, systems, and/or methods described herein relate to a device wherein the coil spring portion includes a bias force urging the base portion and the movable arm portion together.

In some implementations, apparatuses, systems, and/or methods described herein relate to a device wherein the coil spring portion includes a coil axis that is offset from a paddle pivot axis.

In some implementations, a valve repair device comprises a pair of paddles. In some implementations, the pair of paddles are adjustable to a plurality of different implantation positions. In some implementations, the pair of paddles are movable in response to movement of the leaflets of the native valve leaflets during the diastolic phase toward an open position from a set implantation position.

In some implementations, the paddles are allowed to move from the implantation position with the movement of the native valve leaflets by allowing movement of a drive member.

In some implementations, a degree or distance that the paddles can move from the implantation position is settable or controllable.

In some implementations, the degree or distance that the paddles can move from the implantation position is settable or controllable by adjusting a space or gap that receives a drive member.

In some implementations, the degree or distance that the paddles can move from the implantation position is settable or controllable by adjusting a stop that engages a drive member.

In some implementations, the degree or distance that the paddles can move from the implantation position is settable or controllable by adjusting an amount of compression of a spring.

In some implementations, the spring is configured to bottom out or fully compress to limit movement of the drive member.

In some implementations, an amount of force and/or a force profile required to move the paddles from the implantation position is settable or controllable by adjusting one or more springs.

In some implementations, an amount of force and/or a force profile required to move the paddles from the implantation position is settable or controllable compressing or relaxing one or more springs.

Any of the above method(s) and any methods of using the systems, assemblies, apparatuses, devices, etc. herein can be performed on a living subject (e.g., human or other animal) or on a simulation (e.g., a cadaver, cadaver heart, imaginary person, simulator, etc.). With a simulation, the body parts can optionally be referred to as “simulated” (e.g., simulated heart, simulated tissue, etc.) and can optionally comprise computerized and/or physical representations.

Any of the above systems, assemblies, devices, apparatuses, components, etc. can be sterilized (e.g., with heat, radiation, ethylene oxide, hydrogen peroxide, etc.) to ensure they are safe usable and/or for use with patients, and the methods herein can comprise (or additional methods comprise or consist of) sterilization of one or more systems, devices, apparatuses, components, etc. herein (e.g., with heat, radiation, ethylene oxide, hydrogen peroxide, etc.).

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of examples in the present disclosure, a more particular description of certain examples and implementations will be made by reference to various aspects of the appended drawings. These drawings depict only example implementations of the present disclosure and are therefore not to be considered limiting of the scope of the disclosure. Moreover, while the FIGS. can be drawn to scale for some examples, the FIGS. are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a systolic phase showing valve regurgitation;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIGS. 8-14 show an example device or implant, in various stages of deployment;

FIG. 15 shows an example device that is similar to the device illustrated by FIGS. 8-14, but where the paddles are independently controllable;

FIGS. 16-21 show the example device of FIGS. 8-14 being delivered and deployed within a native valve;

FIG. 22 shows a perspective view of an example device in a closed position;

FIG. 23 shows a perspective view of an example device in a closed position;

FIG. 24 illustrates an example device with paddles in an open position;

FIG. 25A illustrates an example device with paddles in a closed position;

FIG. 25B illustrates a top view of an example device;

FIG. 26 illustrates a perspective view of an example device having paddles of adjustable widths;

FIG. 27 is a cross-section of the example device of FIG. 26 in which the device is bisected;

FIG. 28 is a cross-section of the example device of FIG. 26 in which the device is bisected along a plane perpendicular to the plane illustrated in FIG. 28;

FIG. 29 is a schematic illustration of an example catheter assembly coupled to an example device in which an actuation element is coupled to a paddle actuation control and to a driver head of the device;

FIG. 30 is an illustration of the assembly of FIG. 29 with the example device rotated 90 degrees to show the paddle width adjustment element coupled to an inner end of the connector of the device and coupled to a paddle width control;

FIG. 31 illustrates a perspective view of an example treatment and/or repair device including aesthetic features thereof;

FIG. 32 is a side view of the example device illustrated by FIG. 31 including aesthetic features thereof;

FIG. 33 is a front view of the example device illustrated by FIG. 31 including aesthetic features thereof;

FIG. 34 is a top view of the example device illustrated by FIG. 31 including aesthetic features thereof;

FIG. 35 is a top view of the example device illustrated by FIG. 31 including aesthetic features thereof;

FIG. 36 is an exploded perspective view of the example device illustrated by FIG. 31 including aesthetic features thereof;

FIG. 37 is a cross-sectional view of the example device illustrated by FIG. 31 including aesthetic features thereof;

FIG. 38 is a cross-sectional view of the example device illustrated by FIG. 31 including aesthetic features thereof;

FIG. 39 is a perspective cross-sectional view of the example device illustrated by FIG. 31 including aesthetic features thereof;

FIG. 40 is a perspective view of the example device illustrated by FIG. 31, including aesthetic features thereof, in a partially open position with gripping members closed;

FIG. 41 is a perspective view of the example device illustrated by FIG. 31, including aesthetic features thereof, in an open position with gripping members closed;

FIG. 42 is a perspective view of the example device illustrated by FIG. 31, including aesthetic features thereof, in an open position with gripping members open;

FIG. 43 is a perspective view of is a perspective view of the example device illustrated by FIG. 31, including aesthetic features thereof, in an extended position with gripping members closed;

FIG. 44 is a perspective view of and actuation assembly for the treatment and/or repair device illustrated by FIGS. 31-43;

FIG. 45 is an exploded perspective view of components of the actuation assembly illustrated by FIG. 44;

FIGS. 46 and 47 illustrate an interface between the example device illustrated by FIGS. 31-43 and the actuation assembly illustrated by FIGS. 44-45;

FIG. 48 is a side view of the example treatment and/or repair device illustrated by FIG. 31 including aesthetic features thereof;

FIG. 49 is a front view of the example device illustrated by FIG. 31 including aesthetic features thereof;

FIG. 50 illustrates a perspective view of an example treatment and/or repair device including aesthetic features thereof;

FIG. 51 is a side view of the example device illustrated by FIG. 50 including aesthetic features thereof;

FIG. 52 is a front view of the example device illustrated by FIG. 50 including aesthetic features thereof;

FIG. 53 illustrates a distal end of an example system or assembly including a delivery system and an implantable device including aesthetic features thereof;

FIG. 54 illustrates a perspective view of an example treatment and/or repair device including aesthetic features thereof;

FIG. 55 is a perspective cross-sectional view of the example device illustrated by FIG. 54 including aesthetic features thereof;

FIG. 56 is a cross-sectional view of the example device illustrated by FIG. 54;

FIGS. 56A and 56B illustrate an example of a drive member and spring configuration including aesthetic features thereof that can be used in the example device illustrated by FIG. 54;

FIG. 56C illustrates an example of a treatment and/or repair device including aesthetic features thereof similar to that shown in FIG. 54 and including a bias adjustment assembly.

FIG. 57 is a perspective view of an example biasing element including aesthetic features thereof for the example device illustrated by FIG. 54;

FIG. 58 illustrates an example sheet of material including aesthetic features thereof for making the example biasing element illustrated by FIG. 57;

FIG. 59 is a perspective view of an example biasing element including aesthetic features thereof for the example device illustrated by FIG. 54;

FIG. 60 illustrates an example sheet of material including aesthetic features thereof for making the example biasing element illustrated by FIG. 59;

FIG. 61 illustrates a perspective view of an example treatment and/or repair device including aesthetic features thereof;

FIG. 62 is a cross-sectional view of the example device illustrated by FIG. 61 including aesthetic features thereof;

FIG. 63 illustrates a perspective view of an example treatment and/or repair device including aesthetic features thereof, where the device is shown with paddles in an open position;

FIG. 64 illustrates a perspective view of the example device including aesthetic features thereof illustrated by FIG. 63, where the device is shown with paddles in a closed position;

FIG. 65 illustrates a perspective view of the example device including aesthetic features thereof illustrated by FIG. 63, where the device includes gripping members;

FIG. 66 is a cross-sectional view of the example device including aesthetic features thereof illustrated by FIG. 63;

FIG. 67 is a cross-sectional view of the example device including aesthetic features thereof illustrated by FIG. 63;

FIG. 68 illustrates a perspective view of an example treatment and/or repair device including aesthetic features thereof;

FIG. 69 illustrates a perspective view of an example paddle frame including aesthetic features thereof for the device illustrated by FIG. 68;

FIG. 70 illustrates a side view of the example device including aesthetic features thereof illustrated by FIG. 68, where the paddle frame is attached to a driven member of the device in a first configuration;

FIG. 71 illustrates a side view of the example device including aesthetic features thereof illustrated by FIG. 68, where the paddle frame is attached to a driven member of the device in a second configuration;

FIG. 72 illustrates a perspective view of the example device including aesthetic features thereof illustrated by FIG. 68, where the paddle frames are shown in a narrowed position;

FIG. 73 illustrates a side view of the example device including aesthetic features thereof illustrated by FIG. 68, where the paddle frames are shown in a narrowed position;

FIG. 74 illustrates a perspective view of an example treatment and/or repair device including aesthetic features thereof, where the device is shown with paddle frames in an expanded position;

FIG. 75 illustrates a side view of the example device including aesthetic features thereof illustrated by FIG. 74, where the device is shown with paddles in an expanded position;

FIG. 76 illustrates a perspective view of the example device including aesthetic features thereof illustrated by FIG. 74, where the device is shown with paddle frames in a narrowed position;

FIG. 77 illustrates a side view of the example device including aesthetic features thereof illustrated by FIG. 74, where the device is shown with paddles in a narrowed position;

FIG. 78 illustrates a perspective view of an example treatment and/or repair device including aesthetic features thereof;

FIG. 79 illustrates a perspective view of an example treatment and/or repair device including aesthetic features thereof;

FIG. 80 illustrates a perspective view of the device including aesthetic features thereof illustrated by FIG. 79, where a body of the device is shown in an expanded position;

FIG. 81 illustrates a side view of the device including aesthetic features thereof illustrated by FIG. 79, where a body of the device is shown in an expanded position;

FIG. 82 illustrates a perspective view of the device including aesthetic features thereof illustrated by FIG. 79, where a body of the device is shown in a narrowed position;

FIG. 83 illustrates a side view of the device including aesthetic features thereof illustrated by FIG. 79, where a body of the device is shown in a narrowed position;

FIG. 84 illustrates a perspective view of an example treatment and/or repair device including aesthetic features thereof, where paddles of the device are shown in a shortened position;

FIG. 85 illustrates a perspective view of the device including aesthetic features thereof illustrated by FIG. 84, where paddles of the device are shown in an extended position;

FIG. 86 illustrates a perspective view of an example head of a drive member of an actuation assembly including aesthetic features thereof for a treatment and/or repair device and an example actuation element of an implant catheter for actuating the actuation assembly of the treatment and/or repair device;

FIG. 87 illustrates a top view of the example head including aesthetic features thereof illustrated by FIG. 86;

FIG. 88 illustrates a cross-sectional view of the example head including aesthetic features thereof illustrated by FIG. 86;

FIG. 89 illustrates a perspective view of an interface including aesthetic features thereof between the example head and example actuation element of FIG. 86, where the head and actuation element are in a disconnected configuration;

FIG. 90 illustrates a cross-sectional view of an interface including aesthetic features thereof between the example head and example actuation element of FIG. 86, where the head and actuation element are in a disconnected configuration;

FIG. 91 illustrates a cross-sectional view of an interface including aesthetic features thereof between the example head and example actuation element of FIG. 86, where the head and actuation element are in an engaged configuration;

FIG. 92 illustrates a cross-sectional view of an interface including aesthetic features thereof between the example head and example actuation element of FIG. 86, where the head and actuation element are in a connected configuration;

FIG. 93 illustrates a perspective view of an interface including aesthetic features thereof between the example head and example actuation element of FIG. 86, where the head and actuation element are in an optional further connected configuration; and

FIG. 94 illustrates a cross-sectional view of an interface including aesthetic features thereof between the example head and example actuation element of FIG. 86, where the head and actuation element are in an optional further connected configuration.

FIG. 95 illustrates a partial perspective view of one example of a bias member or element including aesthetic features thereof.

FIG. 96 illustrates a force displacement diagram of one example of a bias member or element including aesthetic features thereof.

FIGS. 97A and 97B illustrate views of an example of a bias member or element including aesthetic features thereof.

FIGS. 98A and 98B illustrate views of an example of a bias member or element including aesthetic features thereof.

FIGS. 99A and 99B illustrate views of an example of a bias member or element including aesthetic features thereof.

FIGS. 100A and 100B illustrate views of an example of a bias member or element including aesthetic features thereof.

FIGS. 101A and 101B illustrate views of an example of a bias member or element including aesthetic features thereof.

FIGS. 102A and 102B illustrate additional views of the example of bias member or element shown in FIGS. 101A and 101B including aesthetic features thereof.

FIG. 103 illustrates a perspective view of an example of a bias member or element including aesthetic features thereof.

FIGS. 104A, 104B, 104C, and 104D illustrate views of an example of a bias member or element including aesthetic features thereof.

FIGS. 105A, 105B, and 105C illustrate views of an example of a bias member or element including aesthetic features thereof.

FIGS. 106A, 106B, and 106C illustrate views of an example of a bias member or element including aesthetic features thereof.

FIGS. 107A, 107B, 107C, and 107D illustrate views of a gripping member or clasp including aesthetic features thereof having a joint portion.

FIGS. 108A and 108B illustrate views of bending or flexing positions of gripping members or clasps including aesthetic features thereof.

FIGS. 109A, 109B, and 109C illustrate views of a gripping member or clasp including aesthetic features thereof having portions of different configurations and/or thicknesses.

FIGS. 110A and 110B illustrate views of an example of a gripping or clasp member including aesthetic features thereof.

FIGS. 111A, 111B, 111C and 111D illustrate views of an example of a gripping or clasp member including aesthetic features thereof.

FIGS. 112A and 112B illustrate views of an example of a gripping or clasp member including aesthetic features thereof.

FIG. 113 illustrates one example of a heart valve repair device in a delivery configuration and having a spacer or coaptation element including aesthetic features thereof.

### DETAILED DESCRIPTION

The following description refers to the accompanying drawings, which illustrate example implementations of the present disclosure. Other implementations having different structures and operation do not depart from the scope of the present disclosure.

Some implementations of the present disclosure are directed to systems, devices, methods, etc. for repairing a defective heart valve. For example, some implementations of devices, treatment device, repair devices, valve treatment devices, valve repair devices, implantable devices, implants, and systems (including systems for delivery thereof) are disclosed herein, and any combination of these options can be made unless specifically excluded. In other words, individual components of the disclosed devices and systems can be combined unless mutually exclusive or otherwise physically impossible.

The techniques, methods, processes, operations, steps, etc. described or suggested herein or in the references incorporated herein, and any methods of using the systems, assemblies, apparatuses, devices, etc. herein, can be performed on a living subject (e.g., human, other animal, etc.) or on a simulation (e.g., a cadaver, cadaver heart, simulator, imaginary person, etc.). When performed on a simulation, the body parts, e.g., heart, tissue, valve, etc., can be assumed to be simulated or can optionally be referred to as “simulated” (e.g., simulated heart, simulated tissue, simulated valve, etc.) and can optionally comprise computerized and/or physical representations of body parts, tissue, etc. The term “simulation” covers use on a cadaver, computer simulator, imaginary person (e.g., if they are just demonstrating in the air on an imaginary heart), etc.”

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of). The terms “clasp” and “clasp arm” are often used herein with respect to specific examples, but the terms “gripping member” and/or “gripper arm” can be used in place of and function in the same or similar ways, even if not configured in the same way as a typical clasp.

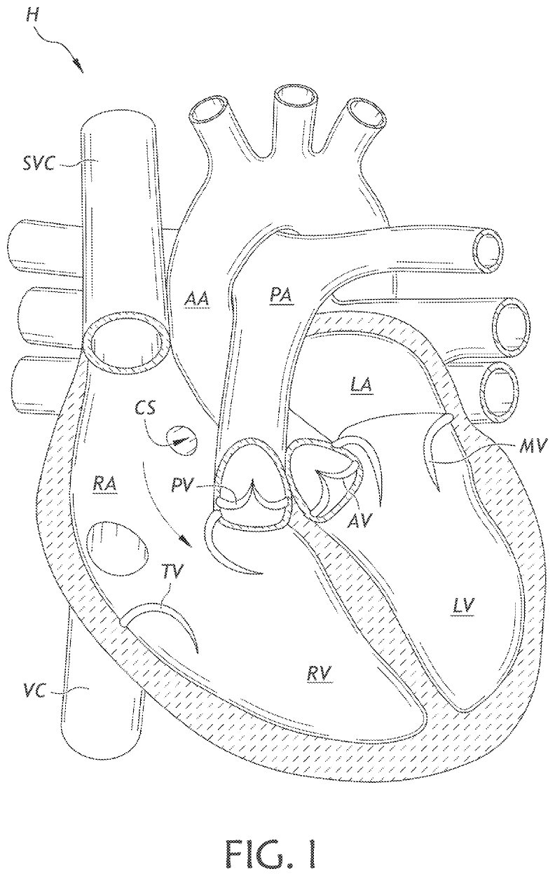

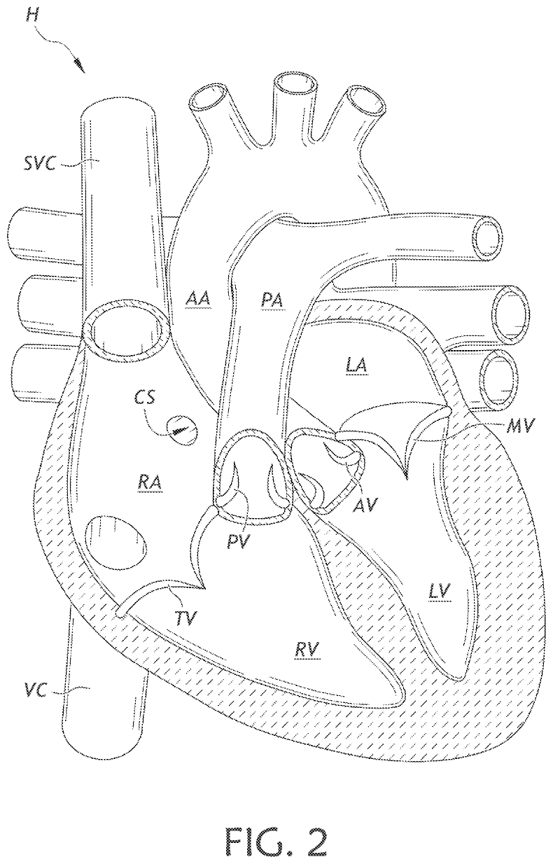

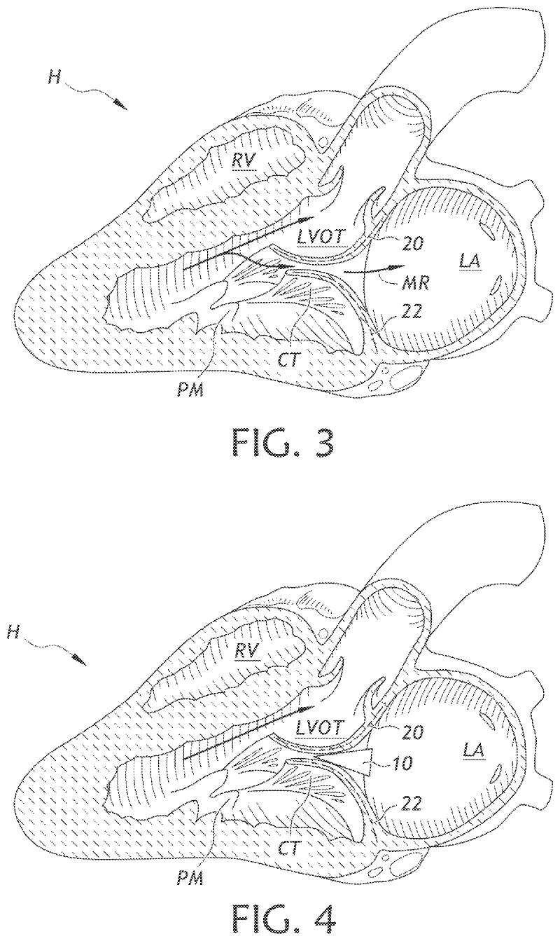

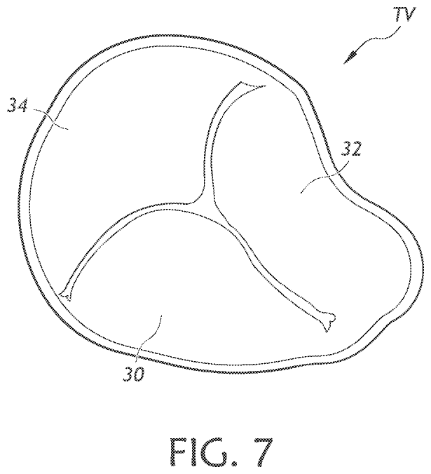

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-6 and leaflets 30, 32, 34 shown in FIG. 7) extending inward across the respective orifices that come together or “coapt” in the flow stream to form the one-way, fluid-occluding surfaces. The native valve repair and/or treatment systems of the present disclosure are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein. In some implementations, the devices described by the present disclosure are used to repair the function of a defective mitral valve MV. That is, the devices are configured to help close the leaflets of the mitral valve to prevent, inhibit or reduce blood from regurgitating from the left ventricle LV and back into the left atrium LA. Many of the devices described in the present disclosure are designed to easily grasp and secure the native leaflets around a coaptation element or spacer that beneficially acts as a filler in the regurgitant orifice to prevent or inhibit back flow or regurgitation during systole, though this is not necessary.

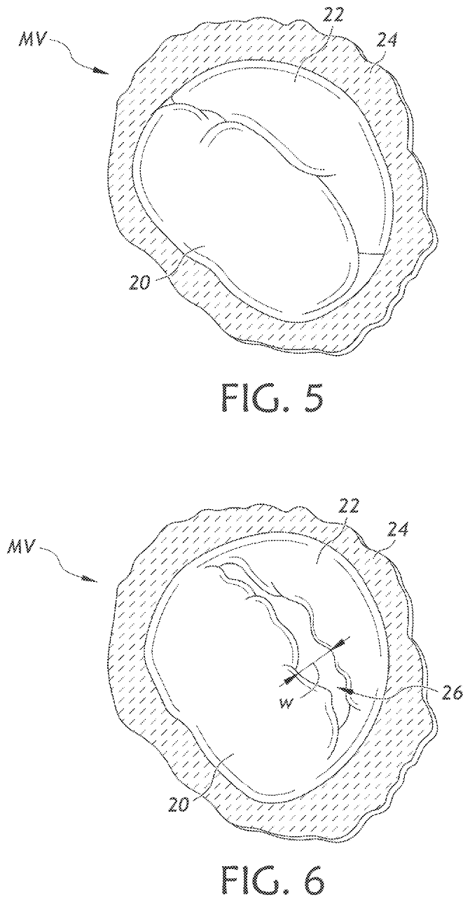

Referring now to FIGS. 1-7, the mitral valve MV includes two leaflets, the anterior leaflet 20 and the posterior leaflet 22. The mitral valve MV also includes an annulus 24 (see FIG. 5), which is a variably dense fibrous ring of tissues that encircles the leaflets 20, 22. Referring to FIGS. 3 and 4, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes can impair proper function of one or more of the native valves of the heart H. These disease processes include degenerative processes (e.g., Barlow's Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) may distort a native valve's geometry, which may cause the native valve to dysfunction. However, the majority of patients undergoing valve surgery, such as surgery to the mitral valve MV, suffer from a degenerative disease that causes a malfunction in a leaflet (e.g., leaflets 20, 22) of a native valve (e.g., the mitral valve, tricuspid valve, aortic valve, and pulmonic valve), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Valve stenosis can result from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant—or incompetent—which include Carpentier's type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction may be caused by rheumatic disease or dilation of a ventricle.

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 3. Referring to FIG. 6, the gap 26 may have a width W between about 2.5 mm and about 17.5 mm, between about 5 mm and about 15 mm, between about 7.5 mm and about 12.5 mm, or about 10 mm. In some situations, the gap 26 may have a width W greater than 15 mm or even 17.5 mm. As set forth above, there are several different ways that a leaflet (e.g., leaflets 20, 22 of mitral valve MV) may malfunction which may thereby lead to valvular regurgitation.

In any of the above-mentioned situations, a device (e.g., an implantable device, implant, treatment device, etc.) is desired that is capable of engaging the anterior leaflet 20 and the posterior leaflet 22 to close the gap 26 and prevent or inhibit regurgitation of blood through the mitral valve MV. As can be seen in FIG. 4, an abstract representation of a repair or treatment device 10 (e.g., a valve treatment device, a valve repair device, an implantable device, an implant, etc.) is shown implanted between the leaflets 20, 22 such that regurgitation does not occur during systole (compare FIG. 3 with FIG. 4). In some implementations, the coaptation element (e.g., spacer, coaption element, gap filler, membrane, sheet, plug, wedge, balloon, etc.) of the device 10 has a generally tapered or triangular shape that naturally adapts to the native valve geometry and to its expanding leaflet nature (toward the annulus). In this disclosure, the terms spacer, coaption element, coaptation element, gap filler, plug, etc. are used interchangeably and refer to an element that fills a portion of the space between native valve leaflets and/or that is configured such that the native valve leaflets engage or “coapt” against (e.g., such that the native leaflets coapt against the coaption element, coaptation element, spacer, etc. instead of only against one another).

Although stenosis or regurgitation may affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) are primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves can either be repaired or replaced. Repair can involve the preservation and correction of the patient's native valve. Replacement can involve replacing the patient's native valve with a biological or mechanical substitute. The aortic valve AV and pulmonary valve PV can be more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, may prevent the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation may occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

The devices and procedures disclosed herein often make reference to repairing the structure of a mitral valve. However, it should be understood that the devices and concepts provided herein can be used to repair any native valve, as well as any component of a native valve. Such devices can be used between the leaflets 20, 22 of the mitral valve MV to prevent or inhibit regurgitation of blood from the left ventricle into the left atrium. With respect to the tricuspid valve TV (FIG. 7), any of the devices and concepts herein can be used between any two of the anterior leaflet 30, septal leaflet 32, and posterior leaflet 34 to prevent or inhibit regurgitation of blood from the right ventricle into the right atrium. In addition, any of the devices and concepts provided herein can be used on all three of the leaflets 30, 32, 34 together to prevent or inhibit regurgitation of blood from the right ventricle to the right atrium. That is, the treatment device, repair devices, implants, etc. provided herein can be centrally located between the three leaflets 30, 32, 34.

An example device (e.g., valve repair device, valve treatment device, implantable device, implant, etc.) can optionally have a coaptation element (e.g., spacer, coaption element, gap filler, membrane, sheet, plug, wedge, balloon, etc.) and at least one anchor (e.g., one, two, three, or more). In some implementations, a device (e.g., a valve repair device, a valve treatment device, an implantable device, an implant, etc.) can have any combination or sub-combination of the features disclosed herein without a coaptation element. When included, the coaptation element (e.g., spacer, coaption element, gap filler, membrane, sheet, plug, wedge, balloon, etc.) is configured to be positioned within the native heart valve orifice to help fill the space between the leaflets and form a more effective seal, thereby reducing or preventing or inhibiting regurgitation described above. The coaptation element can have a structure that is impervious to blood (or that resists blood flow therethrough) and that allows the native leaflets to close around the coaptation element during ventricular systole to block blood from flowing from the left or right ventricle back into the left or right atrium, respectively. The device can be configured to seal against two or three native valve leaflets; that is, the device can be used in the native mitral (bicuspid) and tricuspid valves. The coaptation element is sometimes referred to herein as a spacer because the coaptation element can fill a space between improperly functioning native leaflets (e.g., mitral leaflets 20, 22 or tricuspid leaflets 30, 32, 34) that do not close completely.

The optional coaptation element (e.g., spacer, coaptation element, gap filler, membrane, sheet, plug, wedge, balloon, etc.) can have various shapes. In some implementations, the coaptation element can have an elongated cylindrical shape having a round cross-sectional shape. In some implementations, the coaptation element can have an oval cross-sectional shape, an ovoid cross-sectional shape, a crescent cross-sectional shape, a rectangular cross-sectional shape, or various other non-cylindrical shapes. In some implementations, the coaptation element can have an atrial portion positioned in or adjacent to the atrium, a ventricular or lower portion positioned in or adjacent to the ventricle, and a side surface that extends between the native leaflets. In some implementations, configured to be used and/or for use in the tricuspid valve, the atrial or upper portion is positioned in or adjacent to the right atrium, and the ventricular or lower portion is positioned in or adjacent to the right ventricle, and the side surfaces extend between the native tricuspid leaflets.

In some implementations, the anchor can be configured to secure the device to one or both of the native leaflets such that the coaptation element is positioned between the two native leaflets.

In some implementations, the anchor can be configured to be used and/or for use in the tricuspid valve, the anchor is configured to secure the device to one, two, or three of the tricuspid leaflets such that the coaptation element is positioned between the three native leaflets.

In some implementations, the anchor can attach to the coaptation element at a location adjacent the ventricular portion of the coaptation element. In some implementations, the anchor can attach to an actuation element (e.g., an actuation shaft, actuation tube, actuation wire, etc.) to which the coaptation element is also attached. In some implementations, the anchor and the coaptation element can be positioned independently with respect to each other by separately moving each of the anchor and the coaptation element along the longitudinal axis of the actuation element (e.g., actuation shaft, actuation rod, actuation tube, actuation wire, etc.). In some implementations, the anchor and the coaptation element can be positioned simultaneously by moving the anchor and the coaptation element together along the longitudinal axis of the actuation element (e.g., shaft, actuation wire, etc.). The anchor can be configured to be positioned behind a native leaflet when deployed such that the leaflet is grasped by the anchor.

The device can be configured to be deployed and/or implanted via a delivery system or other means for delivery. The delivery system can comprise one or more of a guide/delivery sheath, a delivery catheter, a steerable catheter, an implant catheter, tube, combinations of these, etc. The coaptation element and the anchor can be compressible to a radially compressed state and can be self-expandable to a radially expanded state when compressive pressure is released. The device can be configured for the anchor to be expanded radially away from the still compressed coaptation element initially in order to create a gap between the coaptation element and the anchor. A native leaflet can then be positioned in the gap. The coaptation element can be expanded radially, closing the gap between the coaptation element and the anchor and capturing the leaflet between the coaptation element and the anchor. In some implementations, the anchor and coaptation element are optionally configured to self-expand. The implantation and/or deployment methods for some implementations can be different and are more fully discussed below with respect to each implementation. Additional information regarding these and other delivery methods that can be used with the concepts herein can be found in U.S. Pat. No. 8,449,599 and U.S. Patent Application Publication Nos. 2014/0222136, 2014/0067052, 2016/0331523, PCT patent application publication Nos. WO2020/076898, WO2023/278663, WO2023/004098, WO2023/091520, WO2023/107296, WO2023/086340, WO2023/003755, and WO2022/231889 each of which is incorporated herein by reference in its entirety for all purposes. These method(s) can be performed on a living animal or on a simulation, such as on a cadaver, cadaver heart, simulator (e.g., with the body parts, heart, tissue, etc. being simulated), etc. mutatis mutandis.

The disclosed devices or implants can be configured such that the anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

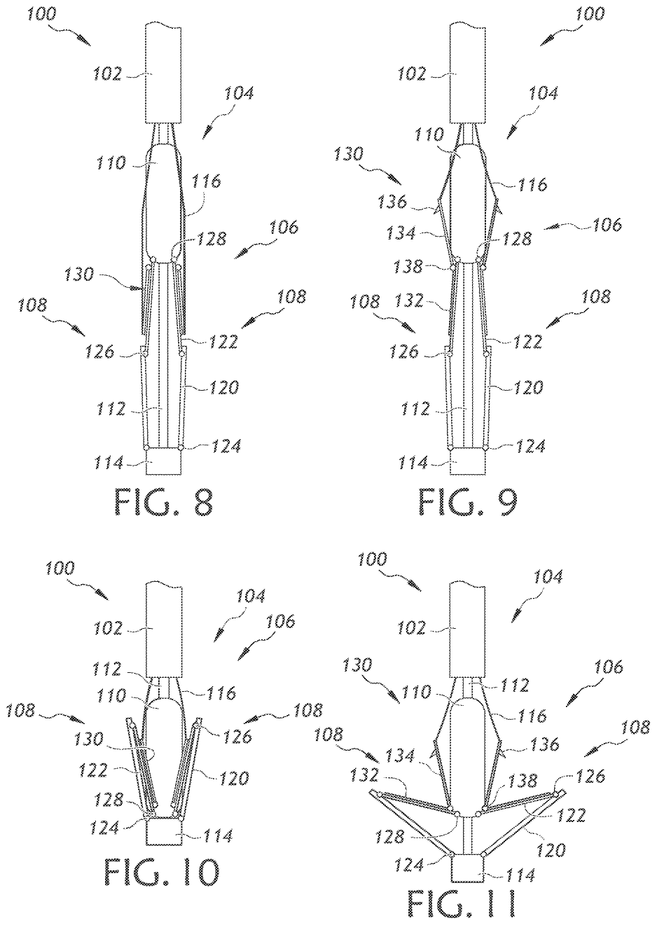

Referring now to FIGS. 8-15, a schematically illustrated device 100 (e.g., a prosthetic device, a valve repair device, valve treatment device, implantable device, implant, etc.) is shown in various stages of deployment. The device 100 and other similar devices and/or implants are described in more detail in PCT patent application publication Nos. WO2018/195215, WO2020/076898, WO2019/139904, WO2023278663, WO2023/004098, WO2023/091520, WO2023/107296, WO2023/086340, WO2023/003755, and WO2022/231889, which are incorporated herein by reference in their entirety for all purposes. The devices herein can include any other features for another device or implant discussed in the present disclosure or the applications cited above, and the devices herein can be positioned to engage valve tissue (e.g., leaflets 20, 22, 30, 32, 34) as part of any suitable treatment and/or repair system (e.g., any treatment and/or repair system described in the present disclosure, or the applications cited herein).

The device 100 is deployed from a delivery system 102. The delivery system 102 can comprise one or more of a catheter, a sheath, a guide catheter/sheath, a delivery catheter/sheath, a steerable catheter, an implant catheter, a tube, a channel, a pathway, combinations of these, etc. The device 100 includes a coaptation portion 104 and an anchor portion 106.

In some implementations, the coaptation portion 104 of the device 100 includes a coaptation element 110 that is adapted to be deployed and/or implanted between leaflets of a native valve (e.g., a native mitral valve, native tricuspid valve, etc.) and is slidably attached to an actuation element 112 (e.g., actuation wire, shaft, tube, hypotube, line, suture, braid, etc.). The anchor portion 106 includes one or more anchors 108 that are actuatable between open and closed conditions and can take a wide variety of forms, such as, for example, paddles, gripping elements, or the like. Actuation of the actuation element 112 opens and closes the anchor portion 106 of the device 100 to grasp the native valve leaflets during deployment and/or implantation. The actuation element 112 (as well as other actuation elements disclosed herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 106 relative to the coaptation portion 104. Or, the actuation element can be unthreaded, such that pushing or pulling the actuation element 112 moves the anchor portion 106 relative to the coaptation portion 104.

The anchor portion 106 and/or anchors of the device 100 include outer paddles 120 and inner paddles 122 that are, in some implementations, connected between a cap 114 and a coaptation element 110 by portions 124, 126, 128. The portions 124, 126, 128 can be jointed and/or flexible to move between all of the positions described below. The interconnection of the outer paddles 120, the inner paddles 122, the coaptation element 110, and the cap 114 by the portions 124, 126, and 128 can constrain the device to the positions and movements illustrated herein.

In some implementations, the delivery system 102 includes a steerable catheter, implant catheter, and the actuation element 112 (e.g., actuation wire, shaft, tube, hypotube, line, suture, braid, etc.). These can be configured to extend through a guide catheter/sheath (e.g., a transseptal sheath, etc.). In some implementations, the actuation element 112 extends through a delivery catheter and the coaptation element 110 to the distal end (e.g., a cap 114 or other attachment portion at the distal connection of the anchor portion 106). Extending and retracting the actuation element 112 increases and decreases the spacing between the coaptation element 110 and the distal end of the device (e.g., the cap 114 or other attachment portion), respectively. In some implementations, a collar or other attachment element (e.g., clamp, clip, lock, sutures, friction fit, buckle, snap fit, lasso, etc.) removably attaches the coaptation element 110 to the delivery system 102, either directly or indirectly, so that the actuation element 112 slides through the collar or other attachment element and, in some implementations, through a coaptation element 110 during actuation to open and close the paddles 120, 122 of the anchor portion 106 and/or anchors 108.

In some implementations, the anchor portion 106 and/or anchors 108 can include attachment portions or gripping members (e.g., gripping arms, clasp arms, etc.). The illustrated gripping members can comprise clasps 130 that include a base or fixed arm 132, a movable arm 134, optional friction-enhancing elements, other securing structures 136 (e.g., barbs, protrusions, ridges, grooves, textured surfaces, adhesive, etc.), and a joint portion 138. The fixed arms 132 are attached to the inner paddles 122. In some implementations, the fixed arms 132 are attached to the inner paddles 122 with the joint portion 138 disposed proximate the coaptation element 110. The joint portion 138 provides a spring force between the fixed and movable arms 132, 134 of the clasp 130. The joint portion 138 can be any suitable joint, such as a flexible joint, a spring joint, a pivot joint, or the like. In some implementations, the joint portion 138 is a flexible piece of material integrally formed with the fixed and movable arms 132, 134. The fixed arms 132 are attached to the inner paddles 122 and remain stationary or substantially stationary relative to the inner paddles 122 when the movable arms 134 are opened to open the clasps 130 and expose the optional barbs or other friction-enhancing elements 136.

In some implementations, the clasps 130 are opened by applying tension to actuation lines 116 attached to the movable arms 134, thereby causing the movable arms 134 to articulate, flex, or pivot on the joint portions 138. The actuation lines 116 extend through the delivery system 102 (e.g., through a steerable catheter, an implant catheter, etc.). Other actuation mechanisms are also possible.

The actuation line 116 can take a wide variety of forms, such as, for example, a line, a suture, a wire, a rod, a catheter, or the like. The clasps 130 can be spring loaded so that in the closed position the clasps 130 continue to provide a pinching force on the grasped native leaflet. Optional barbs or other friction-enhancing elements 136 of the clasps 130 can grab, pinch, and/or pierce the native leaflets to further secure the native leaflets.

During deployment and/or implantation, the paddles 120, 122 can be opened and closed, for example, to grasp the native leaflets (e.g., native mitral valve leaflets, tricuspid valve, etc.) between the paddles 120, 122 and/or between the paddles 120, 122 and a coaptation element 110 (e.g., a spacer, plug, membrane, etc.).

The clasps 130 can be used to grasp and/or further secure the native leaflets by engaging the leaflets with optional barbs or other friction-enhancing elements 136 and pinching the leaflets between the movable and fixed arms 134, 132. The optional barbs or other friction-enhancing elements 136 (e.g., protrusions, ridges, grooves, textured surfaces, adhesive, etc.) of the clasps 130 increase friction with the leaflets or can partially or completely puncture the leaflets.

In some implementations, the actuation lines 116 can be actuated separately (or both separately and simultaneously) so that each clasp 130 can be opened and closed separately. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires.

Referring now to FIG. 8, the device 100 is shown in an elongated or fully open condition for deployment from a delivery catheter of the delivery system 102. The device 100 is disposed at the end of the catheter of the delivery system 102 in the fully open position. In the elongated condition the cap 114 is spaced apart from the coaptation element 110 such that the paddles 120, 122 are fully extended. In some implementations, an angle formed between the interior of the outer and inner paddles 120, 122 is approximately 180 degrees. The clasps 130 can be kept in a closed condition during deployment through the delivery system. The actuation lines 116 can extend and attach to the movable arms 134.

Referring now to FIG. 9, the device 100 is shown in an elongated condition, similar to FIG. 8, but with the clasps 130 in a fully open position, ranging from about 140 degrees to about 200 degrees, from about 170 degrees to about 190 degrees, or about 180 degrees between fixed and movable arms 132, 134 of the clasps 130.

Referring now to FIG. 10, the device 100 is shown in a shortened or fully closed condition. To move the device 100 from the elongated condition to the shortened condition, the actuation element 112 is retracted to pull the cap 114 towards the coaptation element 110. The connection portion(s) 126 (e.g., joint(s), flexible connection(s), etc.) between the outer paddle 120 and inner paddle 122 are constrained in movement such that compression forces acting on the outer paddle 120 from the cap 114 being retracted towards the coaptation element 110 cause the paddles or gripping elements to move radially outward. During movement from the open position to the closed position, the outer paddles 120 maintain an acute angle with the actuation element 112. The outer paddles 120 can optionally be biased toward a closed position. The inner paddles 122 during the same motion move through a considerably larger angle as they are oriented away from the coaptation element 110 in the open condition and collapse along the sides of the coaptation element 110 in the closed condition.

Referring now to FIGS. 11-13, the device 100 is shown in a partially open, grasp-ready condition. To transition from the fully closed to the partially open condition, the actuation element (e.g., actuation wire, shaft, tube, hypotube, line, suture, braid, etc.) is extended to push the cap 114 away from the coaptation element 110, thereby pulling on the outer paddles 120, which in turn pull on the inner paddles 122, causing the anchors or anchor portion 106 to partially unfold. The actuation lines 116 are also retracted to open the clasps 130 so that the leaflets can be grasped. In some implementations, the pair of inner and outer paddles 122, 120 are moved in unison, rather than independently, by a single actuation element 112. Also, the positions of the clasps 130 are dependent on the positions of the paddles 122, 120. For example, referring to FIG. 10 closing the paddles 122, 120 also closes the clasps. In some implementations, the paddles 120, 122 can be independently controllable. In the example illustrated by FIG. 15, the device 100 can have two actuation elements 111, 113 and two independent caps 115, 117 (or other attachment portions), such that one independent actuation element (e.g., actuation wire, shaft, tube, hypotube, line, suture, braid, etc.) and cap (or other attachment portion) are used to control one paddle, and the other independent actuation element and cap (or other attachment portion) are used to control the other paddle.

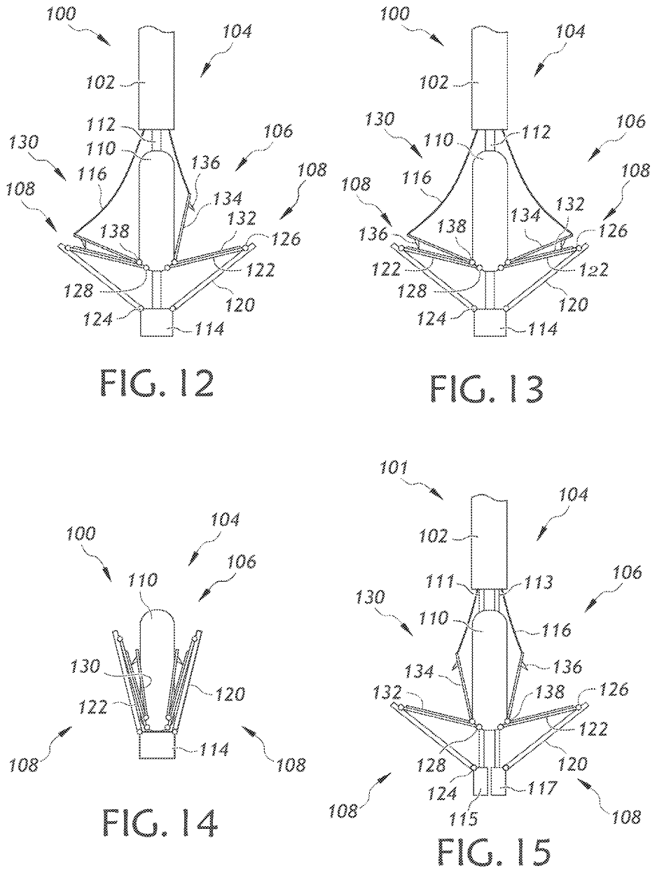

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 can be repeatedly actuated to repeatedly open and close the clasps 130.

Referring now to FIG. 14, the device 100 is shown in a fully closed and deployed condition. The delivery system 102 and actuation element 112 are retracted and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the connection portions 124, 126, 128, the joint portions 138, and/or the inner and outer paddles 122, and/or an additional biasing component (not shown) can be formed of metals such as steel or shape-memory alloy, such as Nitinol-produced in a wire, sheet, tubing, or laser sintered powder- and are biased to hold the outer paddles 120 closed around the coaptation element 110 and the clasps 130 pinched around native leaflets. Similarly, the fixed and movable arms 132, 134 of the clasps 130 are biased to pinch the leaflets. In some implementations, the attachment or connection portions 124, 126, 128, joint portions 138, and/or the inner and outer paddles 122, and/or an additional biasing component (not shown) can be formed of any other suitably elastic material, such as a metal or polymer material, to maintain the device 100 in the closed condition after deployment and/or implantation.

FIG. 15 illustrates an example where the paddles 120, 122 are independently controllable. The device 101 illustrated by FIG. 15 is similar to the device illustrated by FIG. 11, except the device 100 of FIG. 15 includes an actuation element that is configured as two independent actuation elements 111, 113 that are coupled to two independent caps 115, 117. To transition a first inner paddle 122 and a first outer paddle 120 from the fully closed to the partially open condition, the actuation element 111 is extended to push the cap 115 away from the coaptation element 110, thereby pulling on the outer paddle 120, which in turn pulls on the inner paddle 122, causing the first anchor 108 to partially unfold. To transition a second inner paddle 122 and a second outer paddle 120 from the fully closed to the partially open condition, the actuation element 113 is extended to push the cap 115 away from the spacer or coaptation element 110, thereby pulling on the outer paddle 120, which in turn pulls on the inner paddle 122, causing the second anchor 108 to partially unfold. The independent paddle control illustrated by FIG. 15 can be implemented on any of the devices disclosed by the present disclosure. For comparison, in the example illustrated by FIG. 11, the pair of inner and outer paddles 122, 120 are moved in unison, rather than independently, by a single actuation element 112.

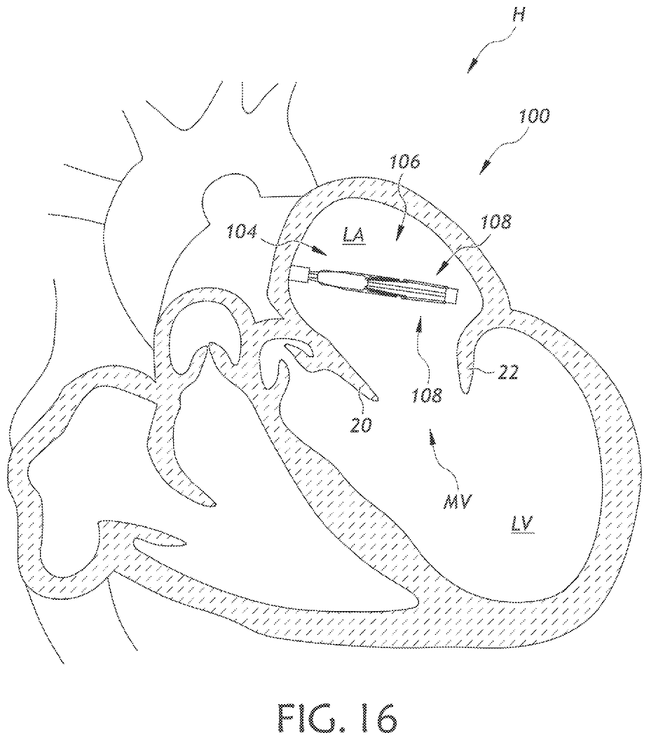

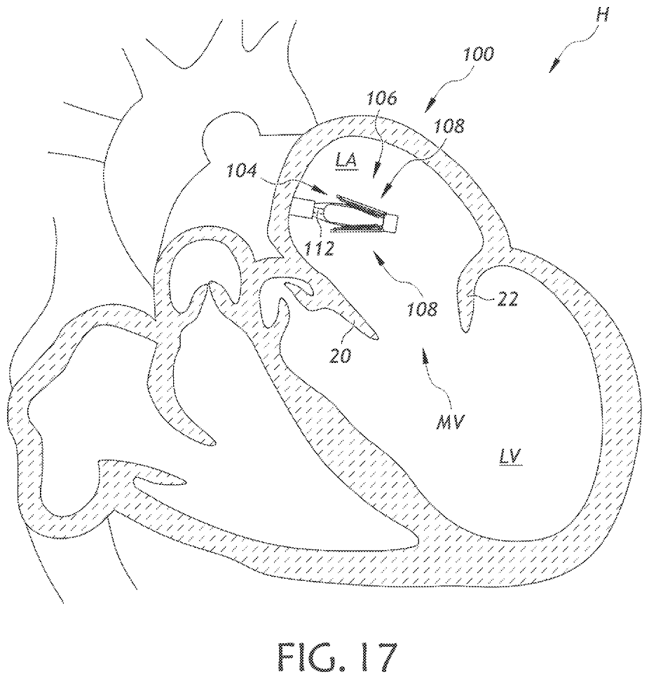

Referring now to FIGS. 16-21, the device 100 of FIGS. 8-14 is shown being delivered and deployed within the native mitral valve MV of the heart H. Referring to FIG. 16, a delivery sheath/catheter is inserted into the left atrium LA through the septum and the implant/device 100 is deployed from the delivery catheter/sheath in the fully open condition as illustrated in FIG. 16. The actuation element 112 is then retracted to move the implant/device into the fully closed condition shown in FIG. 17.

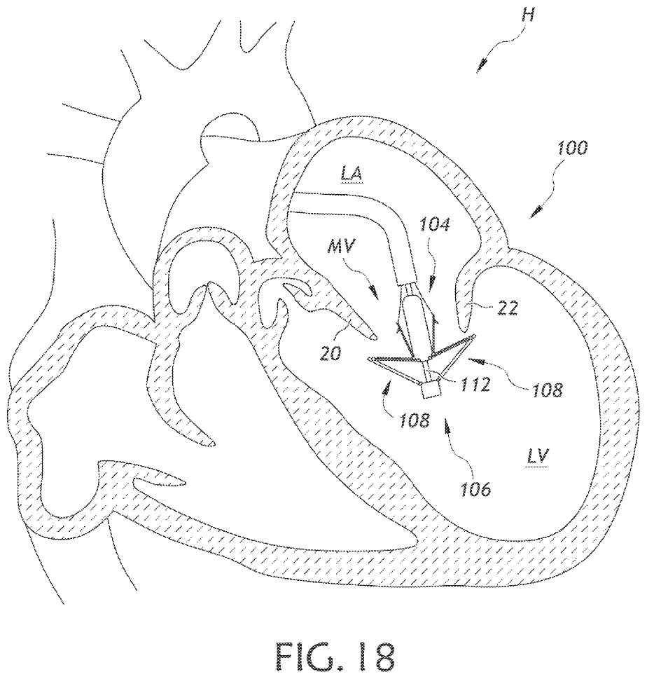

As can be seen in FIG. 18, the implant/device is moved into position within the mitral valve MV into the ventricle LV and partially opened so that the leaflets 20, 22 can be grasped. For example, a steerable catheter can be advanced and steered or flexed to position the steerable catheter as illustrated by FIG. 18. The device or implant catheter connected to the implant/device can be advanced from inside the steerable catheter to position the implant as illustrated by FIG. 18.

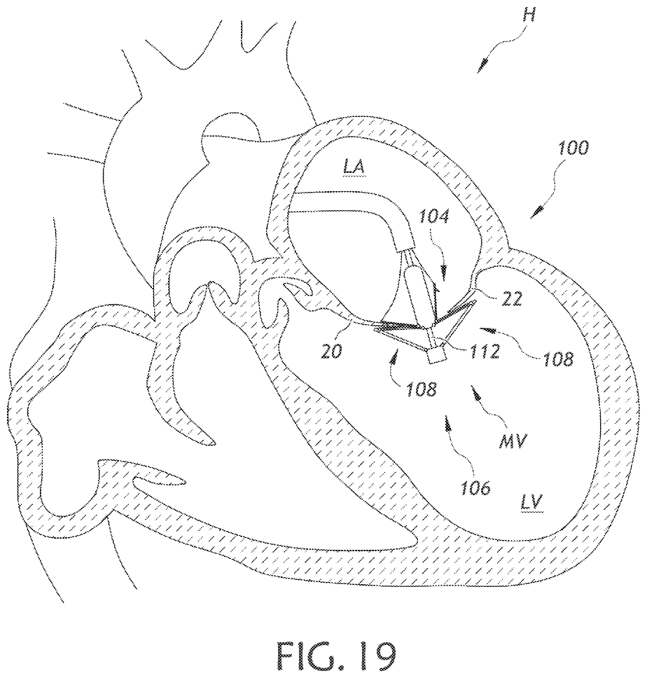

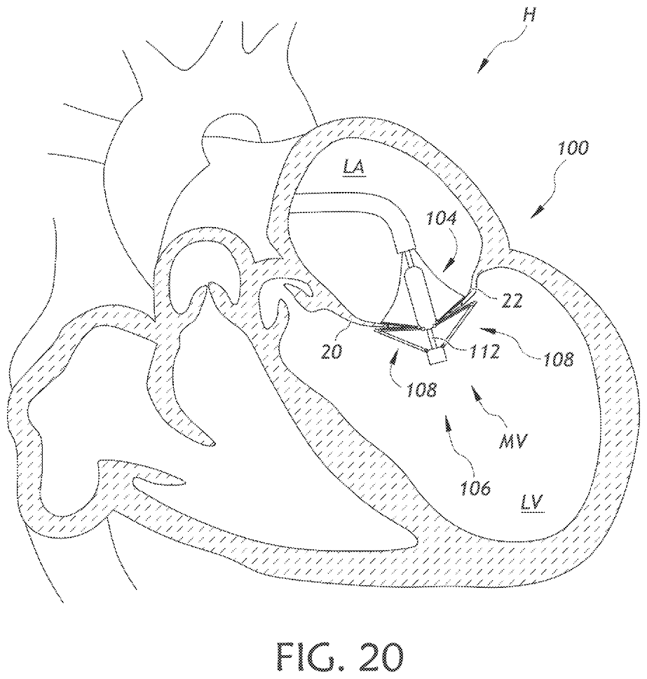

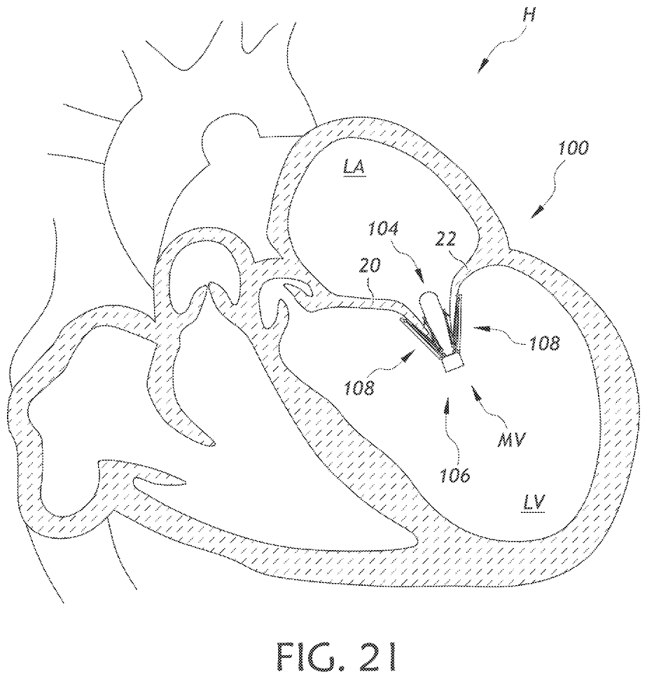

Referring now to FIG. 19, the device catheter can be retracted into the steerable catheter to position the mitral valve leaflets 20, 22 in the clasps 130. An actuation line 116 is extended to close one of the clasps 130, capturing a leaflet 20. FIG. 20 shows the other actuation line 116 being then extended to close the other clasp 130, capturing the remaining leaflet 22. Lastly, as can be seen in FIG. 21, the delivery system 102 (e.g., steerable catheter, implant catheter, etc.), actuation element 112 and actuation lines 116 are then retracted and the device 100 is fully closed and deployed in the native mitral valve MV.

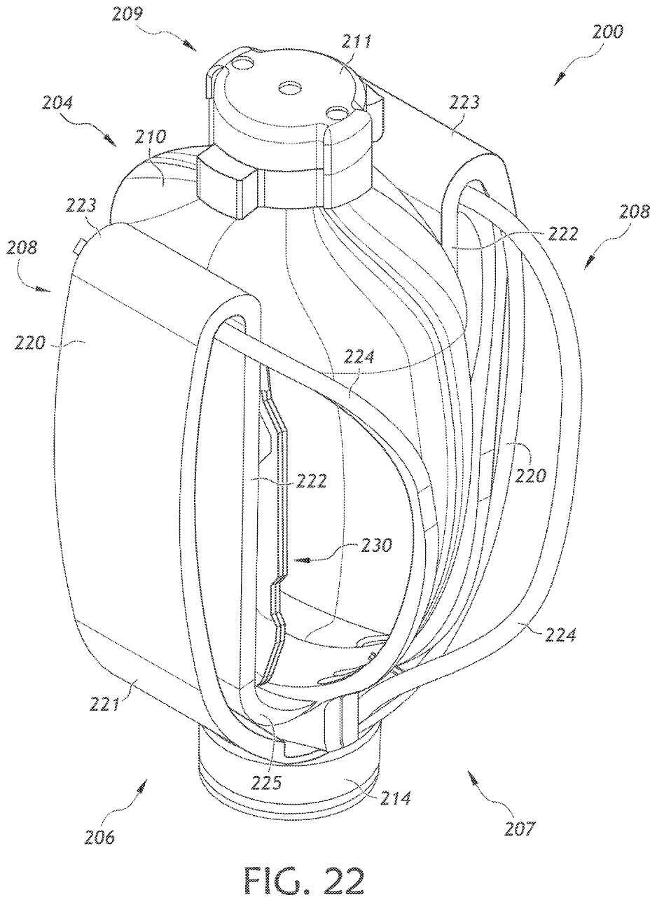

Any of the features described by the present disclosure can be used in a wide variety of different treatment devices and/or repair devices. FIGS. 22-24 illustrate examples of valve treatment and/or repair devices that can be modified to include any of the features described by the present disclosure. Any combination or sub-combination of the features described by the present disclosure can be combined with, substituted for, and/or added to any combination or sub-combination of the features of the devices illustrated by FIGS. 8-24.

Referring now to FIG. 22, an example of a device 200 (e.g., treatment device, repair device, implantable device, implant, etc.) is shown. The device 200 can be configured as an implantable device or implant or other valve treatment device (e.g., one that does not necessarily remain implanted). The device 200 is one of the many different configurations that the device 100 that is schematically illustrated in FIGS. 8-14 can take. The device 200 can include any other features for a device or implant discussed in the present disclosure, and the device 200 can be positioned to engage valve tissue 20, 22 as part of any suitable treatment and/or repair system (e.g., any treatment and/or repair system described in the present disclosure, or the applications cited herein). The device/implant 200 can be a prosthetic spacer device, valve repair device, treatment device, or another type of implant that attaches to leaflets of a native valve.

In some implementations, the device 200 includes a coaptation portion 204, a proximal or attachment portion 209, an anchor portion 206, and a distal portion 207. In some implementations, the coaptation portion 204 of the device optionally includes a coaptation element 210 (e.g., a spacer, coaption element, plug, membrane, sheet, gap filler, plug, wedge, balloon, etc.) for deployment and/or implantation between leaflets of a native valve. In some implementations, the anchor portion 206 includes a plurality of anchors 208. The anchors can be configured in a variety of ways. In some implementations, each anchor 208 includes outer paddles 220, inner paddles 222, paddle extension members or paddle frames 224, and clasps 230. In some implementations, the attachment portion 209 includes a first or proximal collar 211 (or other attachment element) for engaging with a capture mechanism of a delivery system. A delivery system for the device 200 can be the same as or similar to delivery system 102 described above and can comprise one or more of a catheter, a sheath, a guide catheter/sheath, a delivery catheter/sheath, a steerable catheter, an implant catheter, a tube, a channel, a pathway, combinations of these, etc. The capture mechanism can be configured in a variety of ways and, in some implementations, can comprise one or more of a clamp, clip, pin, suture, line, lasso, noose, snare, buckle, lock, latch, etc.

In some implementations, the coaptation element 210 and paddles 220, 222 are formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for deployment and/or implantation in the human body.

An actuation element (e.g., actuation wire, shaft, tube, hypotube, line, suture, braid, etc.) can extend from a delivery system (not shown) to engage and enable actuation of the device or implant 200. In some implementations, the actuation element extends through the proximal collar 211, and spacer or coaptation element 210 to engage a cap 214 of the distal portion 207. The actuation element can be configured to removably engage the cap 214 with a threaded connection, or the like, so that the actuation element can be disengaged and removed from the device 200 after implantation.

The coaptation element 210 extends from the proximal collar 211 (or other attachment element) to the inner paddles 222. In some implementations, the coaptation element 210 has a generally elongated and round shape, though other shapes and configurations are possible. In some implementations, the coaptation element 210 has an elliptical shape or cross-section when viewed from above and has a tapered shape or cross-section when seen from a front view and a round shape or cross-section when seen from a side view. A blend of these three geometries can result in the three-dimensional shape of the illustrated coaptation element 210 that achieves the benefits described herein. The round shape of the coaptation element 210 can also be seen, when viewed from above, to substantially follow or be close to the shape of the paddle frames 224.

The size and/or shape of the coaptation element 210 can be selected to minimize the number of implants that a single patient will require (preferably one), while at the same time maintaining low transvalvular gradients. In some implementations, the anterior-posterior distance at the top of the coaptation element is about 5 mm, and the medial-lateral distance of the coaptation element at its widest is about 10 mm. In some implementations, the overall geometry of the device 200 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 220 are jointably attached to the cap 214 of the distal portion 207 by connection portions 221 and to the inner paddles 222 by connection portions 223. The inner paddles 222 are jointably attached to the coaptation element by connection portions 225. In this manner, the anchors 208 are configured similar to legs in that the inner paddles 222 are like upper portions of the legs, the outer paddles 220 are like lower portions of the legs, and the connection portions 223 are like knee portions of the legs.

In some implementations, the inner paddles 222 are stiff, relatively stiff, rigid, have rigid portions and/or are stiffened by a stiffening member or a fixed portion of the clasps 230. The inner paddle 222, the outer paddle 220, and the coaptation element can all be interconnected as described herein.

In some implementations, the paddle frames 224 are attached to the cap 214 at the distal portion 207 and extend to the connection portions 223 between the inner and outer paddles 222, 220. In some implementations, the paddle frames 224 are formed of a material that is more rigid and stiff than the material forming the paddles 222, 220 so that the paddle frames 224 provide support for the paddles 222, 220.

The paddle frames 224 can provide additional pinching force between the inner paddles 222 and the coaptation element 210 and assist in wrapping the leaflets around the sides of the coaptation element 210. That is, the paddle frames 224 can be configured with a round three-dimensional shape extending from the cap 214 to the connection portions 223 of the anchors 208. The connections between the paddle frames 224, the outer and inner paddles 220, 222, the cap 214, and the coaptation element 210 can constrain each of these parts to the movements and positions described herein. In particular the connection portion 223 is constrained by its connection between the outer and inner paddles 220, 222 and by its connection to the paddle frame 224. Similarly, the paddle frame 224 is constrained by its attachment to the connection portion 223 (and thus the inner and outer paddles 222, 220) and to the cap 214.

The wide configuration of the paddle frames 224 provides increased surface area compared to the inner paddles 222 alone. The increased surface area can distribute the clamping force of the paddles 220 and paddle frames 224 against the native leaflets over a relatively larger surface of the native leaflets in order to further protect the native leaflet tissue.

Additional features of the device 200, modified versions of the device, delivery systems for the device, and methods for using the device and delivery system are disclosed by Patent Cooperation Treaty International Application No. PCT/US2018/028189 (International Publication No. WO 2018/195215) and the other applications incorporated herein. Any combination or sub-combination of the features described by the present disclosure can be combined with any combination or sub-combination of the features disclosed by Patent Cooperation Treaty International Application No. PCT/US2018/028189 (International Publication No. WO 2018/195215) and/or the other applications incorporated herein. Patent Cooperation Treaty International Application No. PCT/US2018/028189 (International Publication No. WO 2018/195215) is incorporated herein by reference in its entirety.

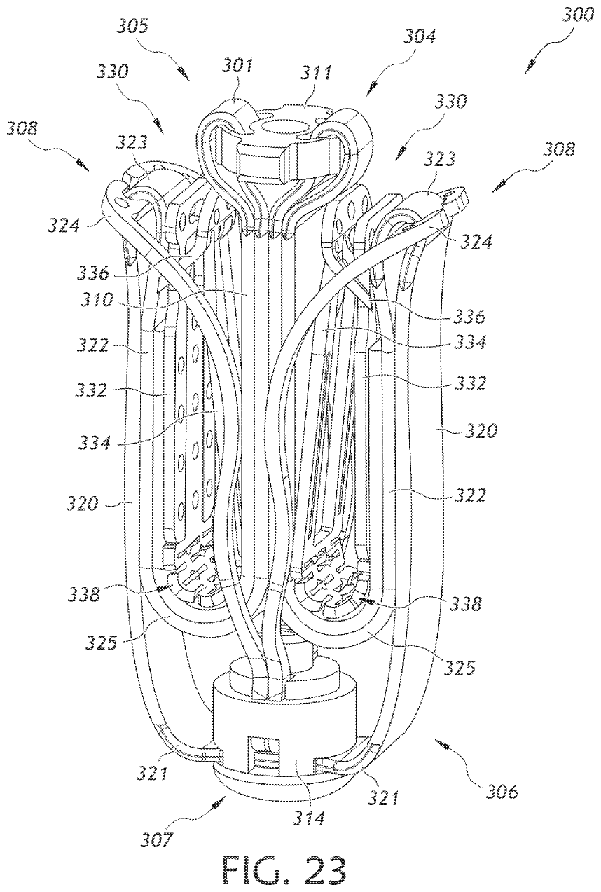

Referring now to FIG. 23, an example of a device 300 (e.g., a valve repair device, a valve treatment device, an implantable device, an implant, etc.) is shown. The device 300 is one of the many different configurations that the device 100 that is schematically illustrated in FIGS. 8-14 can take. The device 300 can include any other features for a device or implant discussed in the present disclosure, and the device 300 can be positioned to engage valve tissue 20, 22 as part of any suitable treatment and/or repair system (e.g., any treatment and/or repair system described in the present disclosure, or the applications cited herein).

The device or implant 300 includes a proximal or attachment portion 305, an anchor portion 306, and a distal portion 307. In some implementations, the device/implant 300 includes a coaptation portion 304, and the coaptation portion 304 can optionally include a coaptation element 310 (e.g., spacer, plug, membrane, sheet, etc.) for deployment and/or implantation between the leaflets 20, 22 of the native valve. In some implementations, the anchor portion 306 includes a plurality of anchors 308. In some implementations, each anchor 308 can include one or more paddles, e.g., outer paddles 320, inner paddles 322, paddle extension members or paddle frames 324. The anchors can also include and/or be coupled to clasps 330. In some implementations, the attachment portion 305 includes a first or proximal collar 311 (or other attachment element) for engaging with a capture mechanism of a delivery system.

The anchors 308 can be attached to the other portions of the device and/or to each other in a variety of different ways (e.g., directly, indirectly, welding, sutures, adhesive, links, latches, integrally formed, a combination of some or all of these, etc.). In some implementations, the anchors 308 are attached to a coaptation element 310 by connection portions 325 and to a cap 314 by connection portions 321.

The anchors 308 can comprise first portions or outer paddles 320 and second portions or inner paddles 322 separated by connection portions 323. The connection portions 323 can be attached to paddle frames 324 that are hingeably attached to a cap 314 or other attachment portion. In this manner, the anchors 308 are configured similar to legs in that the inner paddles 322 are like upper portions of the legs, the outer paddles 320 are like lower portions of the legs, and the connection portions 323 are like knee portions of the legs.

In some implementations, with a coaptation element 310, the coaptation element 310 and the anchors 308 can be coupled together in various ways. As shown in the illustrated example, the coaptation element 310 and the anchors 308 can be coupled together by integrally forming the coaptation element 310 and the anchors 308 as a single, unitary component. This can be accomplished, for example, by forming the coaptation element 310 and the anchors 308 from a continuous strip 301 of a braided or woven material, such as braided or woven nitinol wire. In the illustrated example, the coaptation element 310, the outer paddle portions 320, the inner paddle portions 322, and the connection portions 321, 323, 325 are formed from a continuous strip 301.

Like the anchors 208 of the device 200 described above, the anchors 308 can be configured to move between various configurations by axially moving the distal end of the device (e.g., cap 314, etc.) relative to the proximal end of the device (e.g., proximal collar 311 or other attachment element, etc.). This movement can be along a longitudinal axis extending between the distal end (e.g., cap 314, etc.) and the proximal end (e.g., collar 311 or other attachment element, etc.) of the device.

In some implementations, in the straight configuration, the paddle portions 320, 322 are aligned or straight in the direction of the longitudinal axis of the device. In some implementations, the connection portions 323 of the anchors 308 are adjacent the longitudinal axis of the spacer or coaptation element 310. From the straight configuration, the anchors 308 can be moved to a fully folded configuration (e.g., FIG. 23), e.g., by moving the proximal end and distal end toward each other and/or toward a midpoint or center of the device.

In some implementations, the clasps comprise a movable arm coupled to an anchor. In some implementations, the clasps 330 include a base or fixed arm 332, a movable arm 334, optional barbs/friction-enhancing elements 336, and a joint portion 338. The fixed arms 332 are attached to the inner paddles 322, with the joint portion 338 disposed proximate the coaptation element 310. The joint portion 338 is spring-loaded so that the fixed and movable arms 332, 334 are biased toward each other when the clasp 330 is in a closed condition.

The fixed arms 332 are attached to the inner paddles 322 through holes or slots with sutures. The fixed arms 332 can be attached to the inner paddles 322 with any suitable means, such as screws or other fasteners, crimped sleeves, mechanical latches or snaps, welding, adhesive, or the like. The fixed arms 332 remain substantially stationary relative to the inner paddles 322 when the movable arms 334 are opened to open the clasps 330 and expose the optional barbs 336. The clasps 330 are opened by applying tension to actuation lines attached to the movable arms 334, thereby causing the movable arms 334 to articulate, pivot, and/or flex on the joint portions 338.

In short, the device 300 is similar in configuration and operation to the device 200 described above, except that the coaptation element 310, outer paddles 320, inner paddles 322, and connection portions 321, 323, 325 are formed from the single strip of material 301. In some implementations, the strip of material 301 is attached to the proximal collar 311, cap 314, and paddle frames 324 by being woven or inserted through openings in the proximal collar 311, cap 314, and paddle frames 324 that are configured to receive the continuous strip of material 301. The continuous strip 301 can be a single layer of material or can include two or more layers. In some implementations, portions of the device 300 have a single layer of the strip of material 301 and other portions are formed from multiple overlapping or overlying layers of the strip of material 301.

For example, FIG. 23 shows a coaptation element 310 and inner paddles 322 formed from multiple overlapping layers of the strip of material 301. The single continuous strip of material 301 can start and end in various locations of the device 300. The ends of the strip of material 301 can be in the same location or different locations of the device 300. For example, in the illustrated example of FIG. 23, the strip of material 301 begins and ends in the location of the inner paddles 322.

As with the device 200 described above, the size of the coaptation element 310 can be selected to minimize the number of implants that a single patient will require (preferably one), while at the same time maintaining low transvalvular gradients. In particular, forming many components of the device 300 from the strip of material 301 allows the device 300 to be made smaller than the device 200. For example, in some implementations, the anterior-posterior distance at the top of the coaptation element 310 is less than 2 mm, and the medial-lateral distance of the device 300 (i.e., the width of the paddle frames 324 which are wider than the coaptation element 310) at its widest is about 5 mm.

Additional features of the device 300, modified versions of the device, delivery systems for the device, and methods for using the device and delivery system are disclosed by Patent Cooperation Treaty International Application No. PCT/US2019/055320 (International Publication No. WO 2020/076898) and/or any other applications incorporated herein. Any combination or sub-combination of the features described by the present disclosure can be combined with any combination or sub-combination of the features disclosed by Patent Cooperation Treaty International Application No. PCT/US2019/055320 (International Publication No. WO 2020/076898) and/or any other applications incorporated herein. Patent Cooperation Treaty International Application No. PCT/US2019/055320 (International Publication No. WO 2020/076898) is incorporated herein by reference in its entirety.

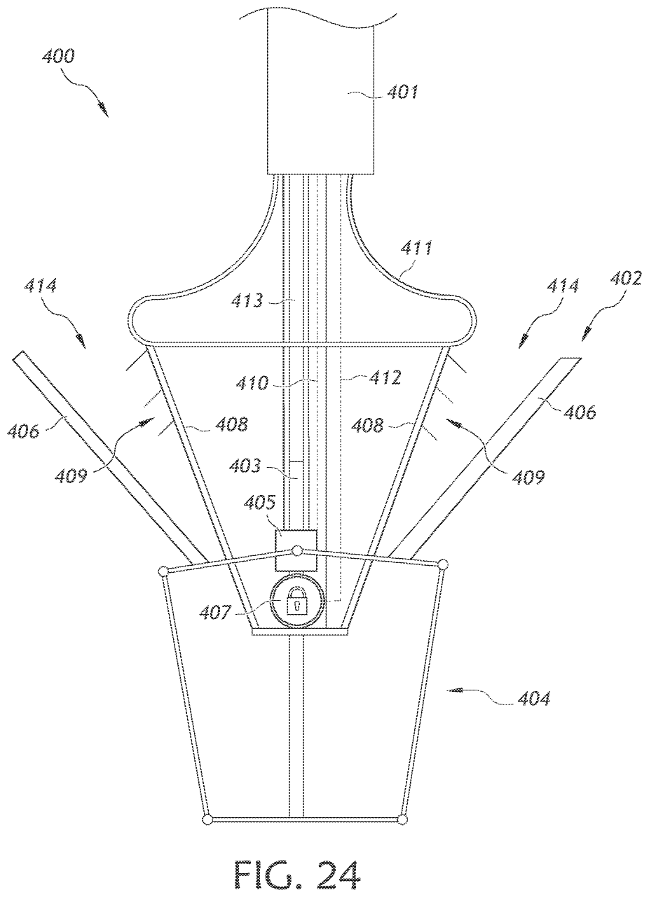

FIG. 24 illustrates an example of one of the many treatment and/or repair systems 400 for treating and/or repairing a native valve of a patient that the concepts of the present disclosure can be applied to. The treatment and/or repair system 400 includes a delivery device 401 and a treatment and/or repair device 402.

In some implementations, the treatment device or repair device 402 includes a base assembly 404, a pair of paddles 406, and a pair of gripping members 408 (e.g., clasps, clasp arms, grippers, gripping arms, latches, etc.). In one example, the paddles 406 can be integrally formed with the base assembly. For example, the paddles 406 can be formed as extensions of links of the base assembly. In the illustrated example, the base assembly 404 of the device 402 has a shaft 403, a coupler 405 configured to move along the shaft, and a lock 407 configured to lock the coupler in a stationary position on the shaft. The coupler 405 is mechanically connected to the paddles 406, such that movement of the coupler 405 along the shaft 403 causes the paddles to move between an open position and a closed position. In this way, the coupler 405 serves as a means for mechanically coupling the paddles 406 to the shaft 403 and, when moving along the shaft 403, for causing the paddles 406 to move between their open and closed positions.

In some implementations, the gripping members 408 are pivotally connected to the base assembly 404 (e.g., the gripping members 408 can be pivotally connected to the shaft 403, or any other suitable member of the base assembly), such that the gripping members can be moved to adjust the width of the opening 414 between the paddles 406 and the gripping members 408. The gripping member 408 can include an optional barbed portion 409 for attaching the gripping members to valve tissue when the device 402 is attached to the valve tissue. When the paddles 406 are in the closed position, the paddles engage the gripping members 408, such that, when valve tissue is attached to the barbed portion 409 of the gripping members, the paddles secure the device 402 to the valve tissue. In some implementations, the gripping members 408 are configured to engage the paddles 406 such that the barbed portion 409 engages the valve tissue member and the paddles 406 to secure the device 402 to the valve tissue member. For example, in certain situations, it can be advantageous to have the paddles 406 maintain an open position and have the gripping members 408 move outward toward the paddles 406 to engage valve tissue and the paddles 406.

While the example shown in FIG. 24 illustrates a pair of paddles 406 and a pair of gripping members 408, it should be understood that the device 402 can include any suitable number of paddles and gripping members.

In some implementations, the system 400 includes a placement shaft 413 that is removably attached to the shaft 403 of the base assembly 404 of the device 402. In some implementations, after the device 402 is secured to valve tissue, the placement shaft 413 can be removed from the shaft 403 to remove the device 402 from the remainder of the treatment and/or repair system 400, such that the device 402 can remain attached to the valve tissue, and the delivery device 401 can be removed from a patient's body.

The treatment and/or repair system 400 can also include a paddle control mechanism 410, a gripper control mechanism 411, and a lock control mechanism 412. The paddle control mechanism 410 is mechanically attached to the coupler 405 to move the coupler along the shaft, which causes the paddles 406 to move between the open and closed positions. The paddle control mechanism 410 can take any suitable form, and can comprise, for example, a shaft, wire, tube, hypotube, rod, suture, line, etc. For example, the paddle control mechanism can comprise a hollow shaft, a catheter tube or a sleeve that fits over the placement shaft 413 and the shaft 403 and is connected to the coupler 405.

The gripper control mechanism 411 is configured to move the gripping members 408 such that the width of the opening 414 between the gripping members and the paddles 406 can be altered. The gripper control mechanism 411 can take any suitable form, such as, for example, a line, a suture or wire, a rod, a catheter, a tube, a hypotube, etc.

The lock control mechanism 412 is configured to lock and unlock the lock. The lock 407 locks the coupler 405 in a stationary position with respect to the shaft 403 and can take a wide variety of different forms and the type of lock control mechanism 412 can be dictated by the type of lock used. In examples in which the lock 407 includes a pivotable plate, the lock control mechanism 412 is configured to engage the pivotable plate to move the plate between the tilted and substantially non-tilted positions. The lock control mechanism 412 can be, for example, a rod, a suture, a wire, or any other member that is capable of moving a pivotable plate of the lock 407 between a tilted and substantially non-tilted position.

The device 402 is movable from an open position to a closed position. The base assembly 404 includes links that are moved by the coupler 405. The coupler 405 is movably attached to the shaft 403. In order to move the device from the open position to the closed position, the coupler 405 is moved along the shaft 403, which moves the links.

The gripper control mechanism 411 is moves the gripping members 408 to provide a wider or a narrower gap at the opening 414 between the gripping members and the paddles 406. In the illustrated example, the gripper control mechanism 411 includes a line, such as a suture, a wire, etc. that is connected to an opening in an end of the gripping members 408. When the line(s) is pulled, the gripping members 408 move inward, which causes the opening 414 between the gripping members and the paddles 406 to become wider.

In order to move the device 402 from the open position to the closed position, the lock 407 is moved to an unlocked condition by the lock control mechanism 412. Once the lock 407 is in the unlocked condition, the coupler 405 can be moved along the shaft 403 by the paddle control mechanism 410.

After the paddles 406 are moved to the closed position, the lock 407 is moved to the locked condition by the lock control mechanism 412 to maintain the device 402 in the closed position. After the device 402 is maintained in the locked condition by the lock 407, the device 402 is removed from the delivery device 401 by disconnecting the shaft 403 from the placement shaft 413. In addition, the device 402 is disengaged from the paddle control mechanism 410, the gripper control mechanism 411, and the lock control mechanism 412.

Additional features of the device 402, modified versions of the device, delivery systems for the device, and methods for using the device and delivery system are disclosed by Patent Cooperation Treaty International Application No. PCT/US2019/012707 (International Publication No. WO 2019139904) and/or any other applications incorporated herein. Any combination or sub-combination of the features described by the present disclosure can be combined with any combination or sub-combination of the features disclosed by Patent Cooperation Treaty International Application No. PCT/US2019/012707 (International Publication No. WO 2019139904) and/or any other applications incorporated herein. Patent Cooperation Treaty International Application No. PCT/US2019/012707 (International Publication No. WO 2019139904) is incorporated herein by reference in its entirety.

Clasps or leaflet gripping devices disclosed herein can take a wide variety of different forms. Examples of clasps are disclosed by Patent Cooperation Treaty International Application No. PCT/US2018/028171 (International Publication No. WO 2018195201). Any combination or sub-combination of the features described by the present disclosure can be combined with any combination or sub-combination of the features disclosed by Patent Cooperation Treaty International Application No. PCT/US2018/028171 (International Publication No. WO 2018195201). Patent Cooperation Treaty International Application No. PCT/US2018/028171 (International Publication No. WO 2018195201) is incorporated herein by reference in its entirety.

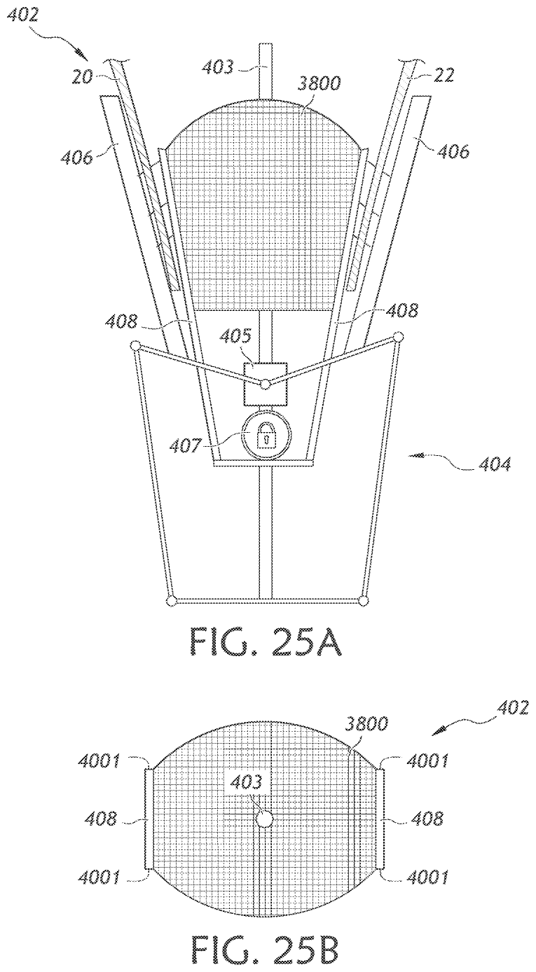

Referring to FIGS. 25A-25B, an example of a treatment and/or repair device 402 has a coaptation element 3800 is shown. The device 402 can have the same configuration as the device illustrated by FIG. 24 with the addition of the coaptation element. The coaptation element 3800 can take a wide variety of different forms. The coaptation element 3800 can be compressible and/or expandable. For example, the coaptation element can be compressed to fit inside one or more catheters of a delivery system, can expand when moved out of the one or more catheters, and/or can be compressed by the paddles 406 to adjust the size of the coaptation element. In the example illustrated by FIGS. 25A and 25B, the size of the coaptation element 3800 can be reduced by squeezing the coaptation element with the paddles 406 and can be increased by moving the paddles 406 away from one another. The coaptation element 3800 can extend past outer edges 4001 of the gripping members or clasps 408 as illustrated for providing additional surface area for closing the gap of a mitral valve.

The coaptation element 3800 can be coupled to the device 402 in a variety of different ways. For example, the coaptation element 3800 can be fixed to the shaft 403, can be slidably disposed around the shaft, can be connected to the coupler 405, can be connected to the lock 407, and/or can be connected to a central portion of the clasps or gripping members 408. In some implementations, the coupler 405 can take the form of the coaptation element 3800. That is, a single element can be used as the coupler 405 that causes the paddles 406 to move between the open and closed positions and the coaptation element 3800 that closes the gap between the leaflets 20, 22 when the device 402 is attached to the leaflets.

The coaptation element 3800 can be disposed around one or more of the shafts or other control elements of the system 400. For example, the coaptation element 3800 can be disposed around the shaft 403, the shaft 413, the paddle control mechanism 410, and/or the lock control mechanism 412.

The device 402 can include any other features for a device, treatment device, repair device, implant, etc. discussed in the present disclosure, and the device 402 can be positioned to engage valve tissue as part of any suitable treatment and/or repair system (e.g., any treatment and/or repair system described in the present disclosure, or the applications cited herein). Additional features of the device 402, modified versions of the device, delivery systems for the device, and methods for using the device and delivery system are disclosed by Patent Cooperation Treaty International Application No. PCT/US2019/012707 (International Publication No. WO 2019139904). Any combination or sub-combination of the features described by the present disclosure can be combined with any combination or sub-combination of the features disclosed by Patent Cooperation Treaty International Application No. PCT/US2019/012707 (International Publication No. WO 2019139904).

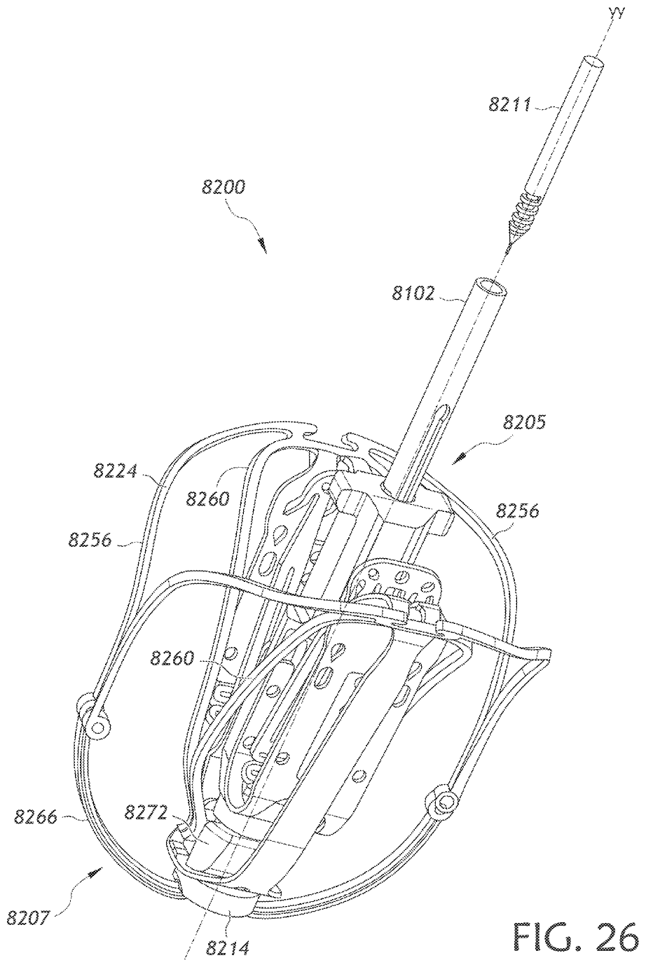

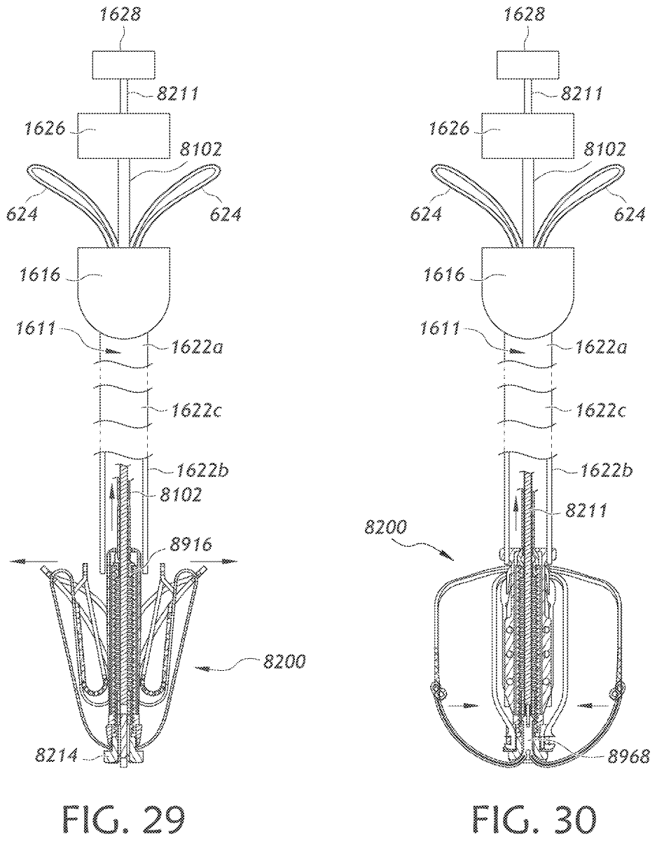

FIGS. 26-30 illustrate an example of one of the many systems for treating and/or repairing a native valve of a patient that the concepts of the present disclosure can be applied to. Referring to FIGS. 29 and 30, the system includes a catheter assembly 1611 (e.g., a device catheter assembly, an implant catheter assembly, treatment catheter assembly, etc.) and a treatment and/or repair device 8200. Referring to FIGS. 26-28, the device 8200 includes a proximal or attachment portion 8205, paddle frames 8224, and a distal portion 8207. The attachment portion 8205, the distal portion 8207, and the paddle frames 8224 can be configured in a variety of ways.

In the example illustrated in FIG. 26, the paddle frames 8224 can be symmetric along longitudinal axis YY. However, in some implementations, the paddle frames 8224 are not symmetric about the axis YY. Moreover, referring to FIG. 26, the paddle frames 8224 include outer frame portions 8256 and inner frame portions 8260.

In some implementations, the connector 8266 (e.g., shaped metal component, shaped plastic component, tether, wire, strut, line, cord, suture, etc.) attaches to the outer frame portions 8256 at outer ends of the connector 8266 and to a coupler 8972 at an inner end 8968 of the connector 8266 (see FIG. 28). Between the connector 8266 and the attachment portion 8205, the outer frame portions 8256 form a curved shape. For example, in the illustrated example, the shape of the outer frame portions 8256 resembles an apple shape in which the outer frame portions 8256 are wider toward the attachment portion 8205 and narrower toward the distal portion 8207. In some implementations, however, the outer frame portions 8256 can be otherwise shaped.

The inner frame portions 8260 extend from the attachment portion 8205 toward the distal portion 8207. The inner frame portions 8260 then extend inward to form retaining portions 8272 that are attached to the actuation cap 8214. The retaining portions 8272 and the actuation cap 8214 can be configured to attach in any suitable manner.

In some implementations, the inner frame portions 8260 are rigid frame portions, while the outer frame portions 8256 are flexible frame portions. The proximal end of the outer frame portions 8256 connect to the proximal end of the inner frame portions 8260, as illustrated in FIG. 26.

The width adjustment element 8211 (e.g., width adjustment wire, width adjustment shaft, width adjustment tube, width adjustment line, width adjustment cord, width adjustment suture, width adjustment screw or bolt, etc.) is configured to move the outer frame portions 8256 from the expanded position to the narrowed position by pulling the inner end 8968 (FIG. 28) and portions of the connector 8266 into the actuation cap 8214. The actuation element 8102 is configured to move the inner frame portions 8260 to open and close the paddles in accordance with some implementations disclosed herein.

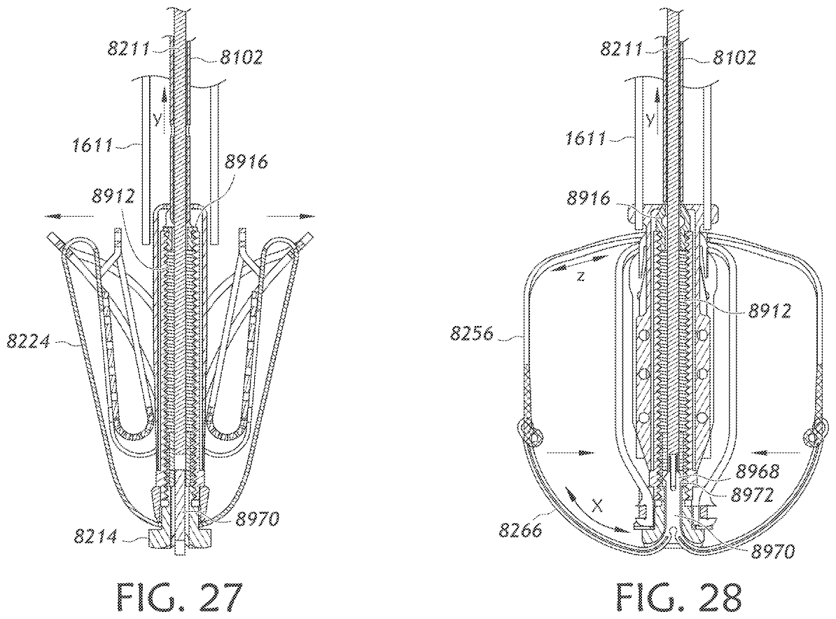

As shown in FIGS. 27 and 28, the connector 8266 has an inner end 8968 that engages with the width adjustment element 8211 such that a user can move the inner end 8968 inside the receiver 8912 (e.g., an internally threaded element, a column, a conduit, a hollow member, a notched receiving portion, a tube, a shaft, a sleeve, a post, a housing, a cylinder, tracks, etc.) to move the outer frame portions 8256 between a narrowed position and an expanded position. In the illustrated example, the inner end 8968 includes a post 8970 that attaches to the outer frame portions 8256 and a coupler 8972 that extends from the post 8970. The coupler 8972 is configured to attach and detach from both the width adjustment element 8211 and the receiver 8912. The coupler 8972 can take a wide variety of different forms. For example, the coupler 8972 can include one or more of a threaded connection, features that mate with threads, detent connections, such as outwardly biased arms, walls or other portions. When the coupler 8972 is attached to the width adjustment element 8211, the coupler is released from the receiver 8912. When the coupler 8972 is detached from the width adjustment element 8211, the coupler is secured to the receiver. The inner end 8968 of the connector can, however, be configured in a variety of ways. Any configuration that can suitably attach the outer frame portions 8256 to the coupler to allow the width adjustment element 8211 to move the outer frame portions 8256 between the narrowed position and the expanded position can be used. The coupler can be configured in a variety of ways as well and can be a separate component or be integral with another portion of the device, e.g., of the connector or inner end of the connector.

The width adjustment element 8211 allows a user to expand or contract the outer frame portions 8256 of the device 8200. In the example illustrated in FIGS. 27 and 28, the width adjustment element 8211 includes an externally threaded end that is threaded into the coupler 8972. The width adjustment element 8211 moves the coupler in the receiver 8912 to adjust the width of the outer frame portions 8256. When the width adjustment element 8211 is unscrewed from the coupler 8972, the coupler engages the inner surface of the receiver 8912 to set the width of the outer frame portions 8256.

In some implementations, the receiver 8912 can be integrally formed with a distal cap 8214. Moving the cap 8214 relative to a body of the attachment portion 8205 opens and closes the paddles. In the illustrated example, the receiver 8912 slides inside the body of the attachment portion. When the coupler 8972 is detached from the width adjustment element 8211, the width of the outer frame portions 8256 is fixed while the actuation element 8102 moves the receiver 8912 and cap 8214 relative to a body of the attachment portion 8205. Movement of the cap can open and close the device in the same manner as some of the examples disclosed above.

In the illustrated example, a driver head 8916 is disposed at a proximal end of the actuation element 8102. The driver head 8916 releasably couples the actuation element 8102 to the receiver 8912. In the illustrated example, the width adjustment element 8211 extends through the actuation element 8102. The actuation element is axially advanced in the direction opposite to direction Y to move the distal cap 8214. Movement of the distal cap 8214 relative to the attachment portion 8205 is effective to open and close the paddles, as indicated by the arrows in FIG. 27. That is, movement of the distal cap 8214 in the direction Y closes the device and movement of the distal cap in the direction opposite to direction Y opens the device.

Also illustrated in FIGS. 27 and 28, the width adjustment element 8211 extends through the actuation element 8102, the driver head 8916, and the receiver 8912 to engage the coupler 8972 attached to the inner end 8968. The movement of the outer frame portions 8256 to the narrowed position can allow the device or implant 8200 to maneuver more easily into position for deployment and/or implantation in the heart by reducing the contact and/or friction between the native structures of the heart—e.g., chordae—and the device 8200. The movement of the outer frame portions 8256 to the expanded position provides the anchor portion of the device 8200 with a larger surface area to engage and capture leaflet(s) of a native heart valve.

Referring to FIGS. 29 and 30, an example of a catheter assembly 1611 (e.g., a device catheter assembly, an implant catheter assembly, treatment catheter assembly, etc.) in which clasp actuation lines 624 extend through a handle 1616, the actuation element 8102 is coupled to a paddle actuation control 1626, and the width adjustment element 8211 is coupled to a paddle width control 1628. A proximal end portion 1622a of the shaft or catheter of the catheter assembly 1611 can be coupled to the handle 1616, and a distal end portion 1622b of the shaft or catheter can be coupled to the device 8200. The actuation element 8102 can extend distally from the paddle actuation control 1626, through the handle 1616, through the delivery shaft or catheter of the catheter assembly 1611, and through the proximal end of the device 8200, where it couples with the driver head 8916. The actuation element 8102 can be axially movable relative to the outer shaft of the catheter assembly 1611 and the handle 1616 to open and close the device.

The width adjustment element 8211 can extend distally from the paddle width control 1628, through the paddle actuation control 1626 and through the actuation element 8102 (and, consequently, through the handle 1616, the outer shaft of the implant catheter assembly 1611, and through the device 8200), where it couples with the movable coupler 8972. The width adjustment element 8211 can be axially movable relative to the actuation element 8102, the outer shaft of the implant catheter assembly 1611, and the handle 1616. The clasp actuation lines 624 can extend through and be axially movable relative to the handle 1616 and the outer shaft of the implant catheter assembly 1611. The clasp actuation lines 624 can also be axially movable relative to the actuation element 8102.

Referring to FIGS. 29 and 30, the width adjustment element 8211 can be releasably coupled to the coupler 8972 of the device 8200. Advancing and retracting the width adjustment element 8211 with the paddle width control 1628 widens and narrows the paddles. Advancing and retracting the actuation element 8102 with the paddle actuation control 1626 opens and closes the paddles of the device.

In the examples of FIGS. 29 and 30, the catheter or shaft of the catheter assembly 1611 is an elongate shaft extending axially between the proximal end portion 1622a, which is coupled to the handle 1616, and the distal end portion 1622b, which is coupled to the device 8200. The outer shaft of the catheter assembly 1611 can also include an intermediate portion 1622c disposed between the proximal and distal end portions 1622a, 1622b.

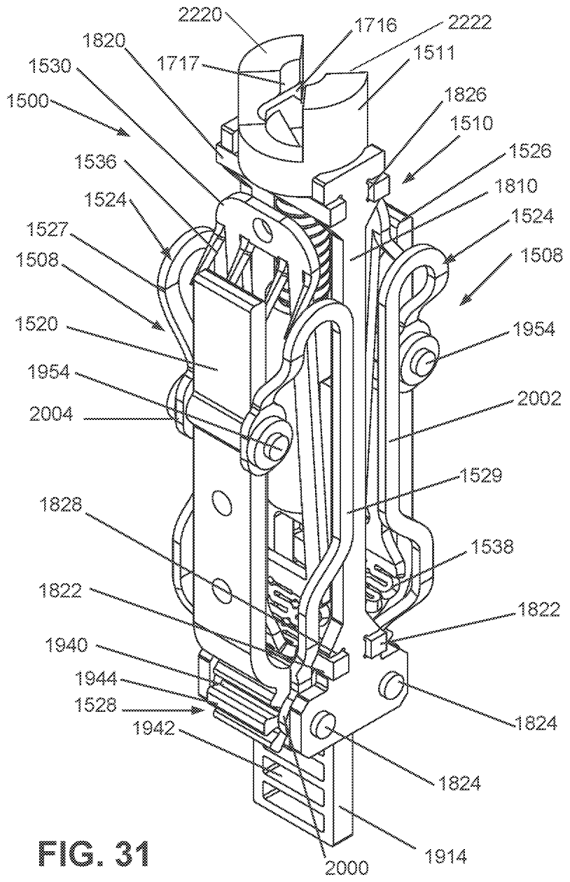

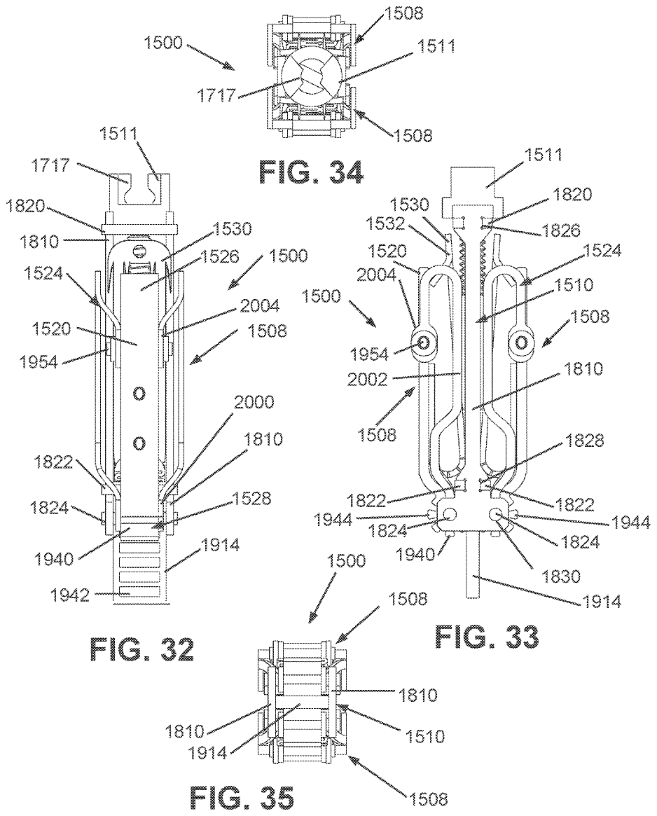

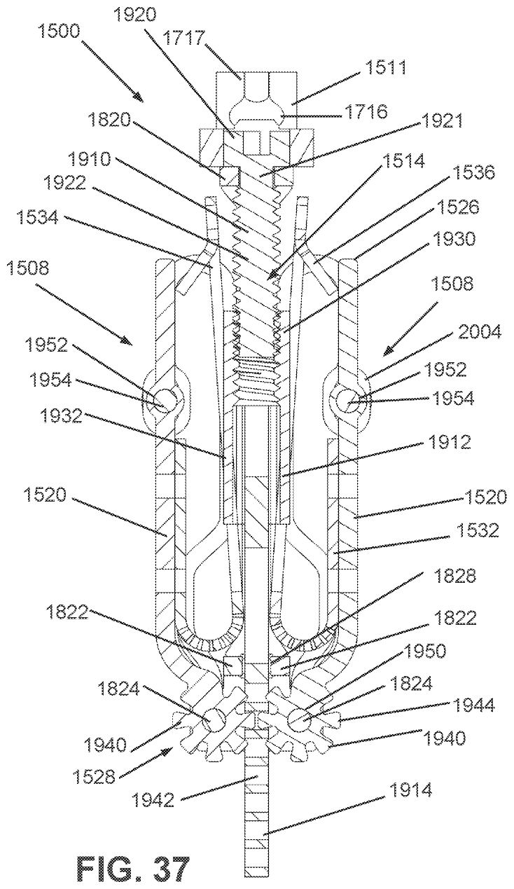

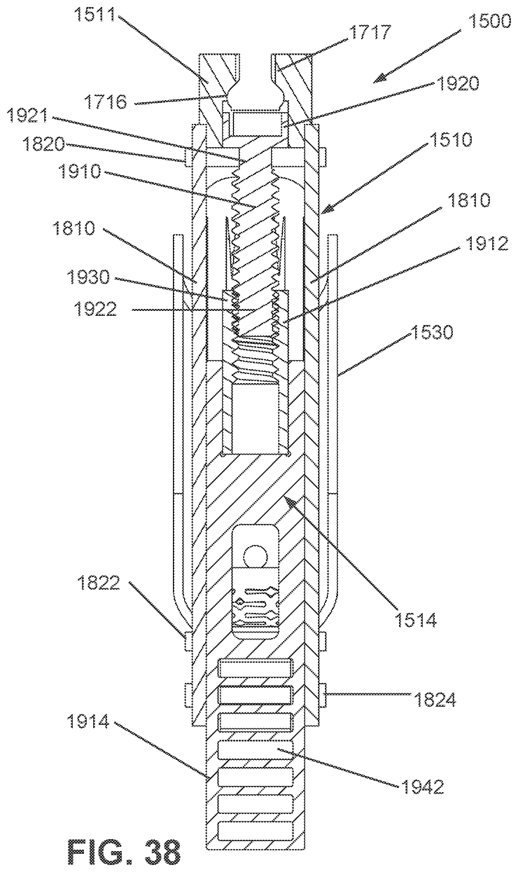

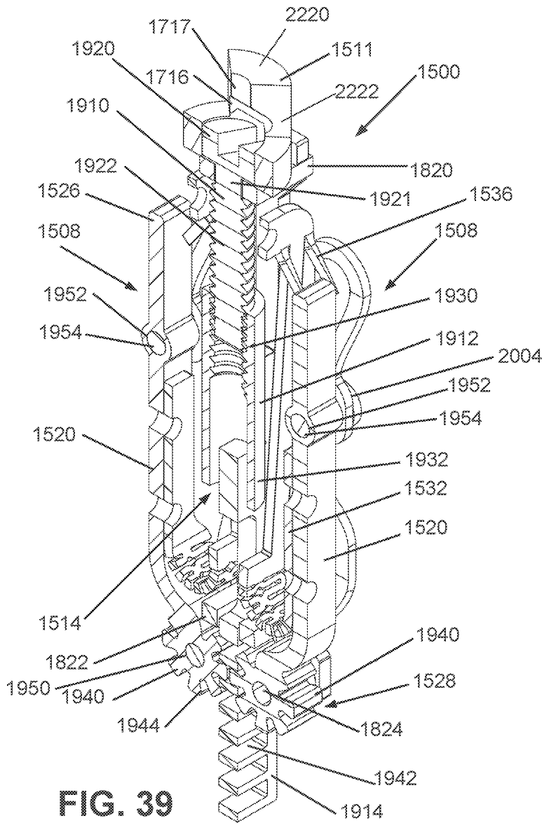

Referring now to FIGS. 31-43, an example of a device 1500 (e.g., a treatment device, a repair device, a prosthetic device, a valve repair device, valve treatment device, implantable device, implant, etc.) is shown. The device 1500 can include any other features for a device discussed in the present disclosure, and the device 1500 can be positioned to engage valve tissue 20, 22, 30, 32, 34 as part of any suitable treatment and/or repair system (e.g., any treatment and/or repair system described in the present disclosure).

The device 1500 can comprise an optional coupling 1511, a base 1510, an actuation assembly 1514, and one or more anchors 1508. In some implementations, the coupling 1511 is connected to a proximal end of the base 1510. In some implementations, the actuation assembly 1514 is housed at least partially within the base 1510. In some implementations, the one or more anchors can comprise one anchor 1508 (e.g., a device configured to attach to only a single native valve leaflet), two anchors 1508 (e.g., a device configured to attach to two leaflet, such as the leaflets of the mitral valve or two leaflets of the tricuspid valve), or three anchors 1508 (e.g., a device configured to attach to the three leaflets of the tricuspid valve).

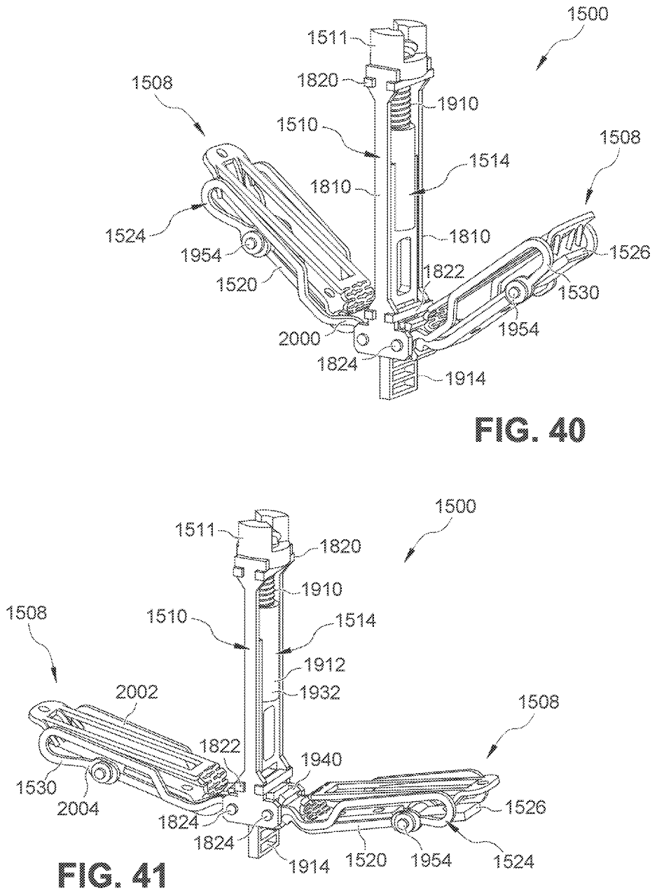

In some implementations, the one or more anchors 1508 are coupled to the actuation assembly 1514, such that operation of the actuation assembly can move the anchors between two or more of a closed position (see FIG. 31), a partially open or capture ready position (see FIG. 40), a fully open position (see FIG. 41), and an extended position (see FIG. 43).

The coupling 1511 can take a variety of different forms. For example, the coupling 1511 can have any of the features of any of the couplings or proximal collars described in the present disclosure and/or can have any of the features of any of the couplings or proximal collars disclosed in PCT application No. PCT/US2018/028189, published as PCT Publication WO2018/195215.

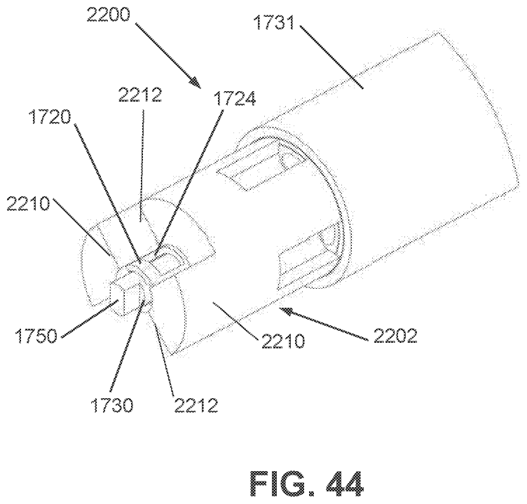

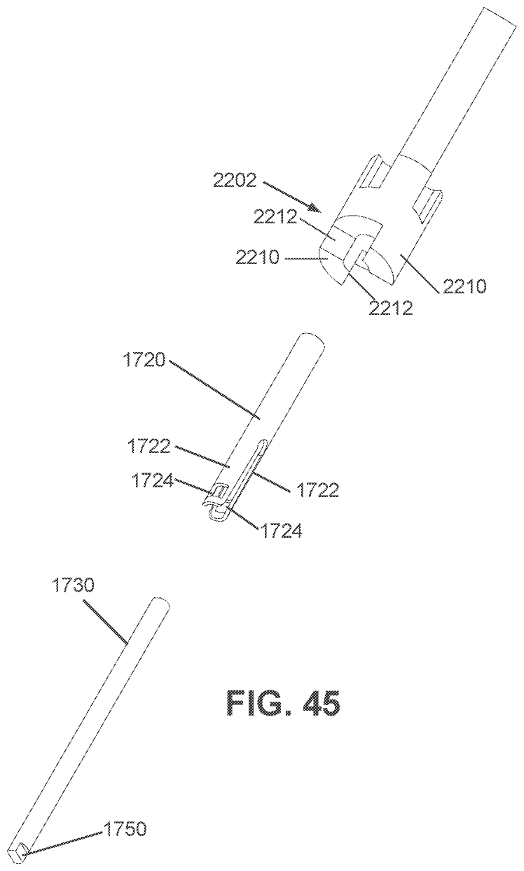

In some implementations, an implantable device 1500 has a coupling 1511 that accepts an actuation element 1730, such as a shaft, tube, rod, wire, etc. The actuation element 1730 can extend through an implant catheter 1731 (see FIGS. 44 and 47) and through a coupler 1720 (see FIGS. 44 and 47) that extends from or is extendable from a distal end of the implant catheter 1731. The actuation element 1730 (e.g., shaft, rod, wire, etc.) can be coupled to the device 1500 to open and close the device.

In some implementations, the coupling 1511 has radially disposed recesses 1716 or undercuts that form corresponding radially disposed, inwardly extending, projections 1717 or slots. In some implementations, the coupler 1720 has movable arms 1722 or fingers that can be moved between open and closed positions. In some implementations, the movable arms 1722 include openings 1724 or windows configured to engage or surround the projections 1717 of the coupling 1511 of the device 1500. In some implementations, the movable arms 1722 are biased inward so that moving the actuation element 1730 in a distal direction Y (see FIG. 47) through the coupler 1720 and between the movable arms 1722 spreads the movable arms 1722 outwards so that the openings 1724 surround and/or engage the projections 1717. In some implementations, moving the actuation element 1730 in a retraction direction X allows the movable arms 1722 to move inward so that the openings 1724 disengage the projections 1717. In this way the device 1500 can be released and coupled to the coupler 1720.

The base 1510 can take a variety of different forms. For example, the base 1510 can be any of the coaption elements described in the present disclosure. The base can be formed from a single component or can be assembled from a plurality of components. In some implementations, the base 1510 comprises a frame with an open interior for other components of the device, such as a metal frame.

In some implementations, the base 1510 comprises a pair of longitudinally extending members 1810 (e.g., plates, bars, walls, sheets, etc.), a pair of laterally extending proximal members 1820 (e.g., bars, shafts, struts, etc.), an optional pair of laterally extending distal members 1822 (e.g., bars, shafts, struts, etc.), and a pair of pivot members 1824 (e.g., cylindrical shafts, tubes, rods, etc.).

In some implementations, the pair of laterally extending proximal members 1820 extend between and connect proximal end portions of the pair of longitudinally extending members 1810. In some implementations, the pair of laterally extending proximal members 1820 are disposed in cutouts 1826, such as slots in the proximal end portions of the pair of longitudinally extending members 1810. In some implementations, the pair of laterally extending distal members 1822 extend between and connect distal end portions of the pair of longitudinally extending members 1810. In some implementations, the pair of laterally extending distal members 1822 are disposed in cutouts 1828, such as slots in the distal end portions of the pair of longitudinally extending members 1810.

In some implementations, the pair of pivot members 1824 extend between and can connect distal end portions of the pair of longitudinally extending members 1810. In some implementations, the pair of pivot members 1824 are disposed in holes 1830, such as blind holes, through holes, slots, etc., in the distal end portions of the pair of longitudinally extending members 1810.

The actuation assembly 1514 can take a variety of different forms. For example, the actuation assembly 1514 can be any of the actuation elements, actuation assemblies, and/or actuation linkages described in the present disclosure. In some implementations, the actuation assembly 1514 can be comprised of longitudinally movable components, such as one or more shafts, rods, tubes and/or can comprise one or more assemblies that convert rotational movement to translational movement, such as gear assemblies (e.g., rack and pinion assemblies, worm gear assemblies etc.) and/or that convert rotational movement about an axis in first plane to rotational movement about an axis in a second plane, that can be orthogonal to the first plane (e.g., worm gear assemblies, planetary gear assemblies, etc.) The actuation assembly 1514 can take any form where a longitudinal movement and/or a rotational movement is used to open and/or close one or more components of the anchors 1508.

In some implementations, the actuation assembly 1514 comprises a drive member 1910, a driven member 1912, and a rack 1914. The drive member 1910 can take a variety of different forms. In some implementations, the drive member 1910 comprises a head 1920, a neck 1921, and a threaded portion 1922.

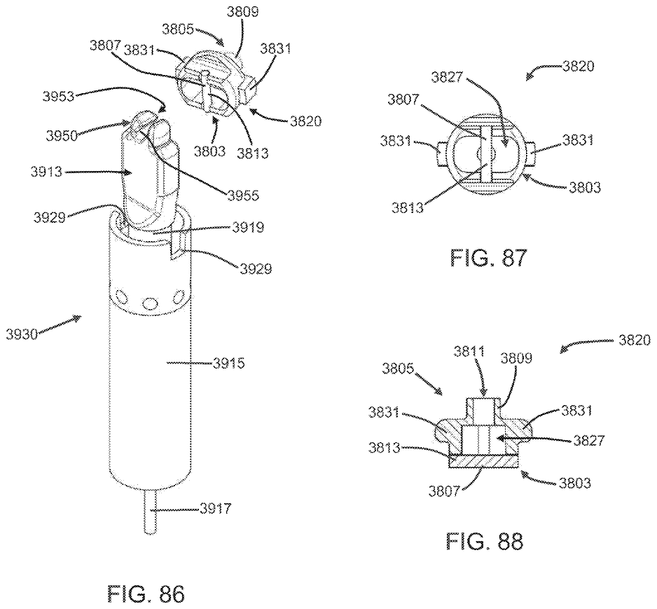

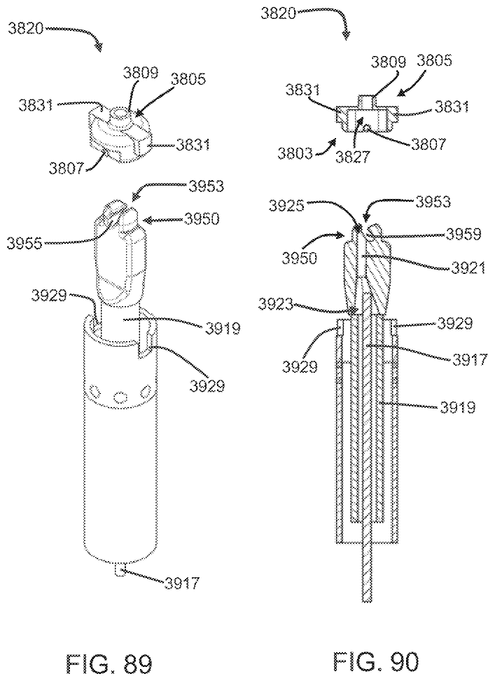

In some implementations, the head 1920 can be configured to be engaged by a drive end 1750 of the actuation element 1730. For example, the head 1920 and the drive end 1750 can have a corresponding mating feature 1753, such as slotted, Phillips, hex, star, or any other mating shapes. In some implementations, the head 1920 can take the form of the head 3820 shown in FIGS. 86-94, and the actuation element 1730 can take the form of the actuation element 3930 shown in FIGS. 86 and 89-94. Any configuration that allows the actuation element 1730 to drive the head 1920 can be used.

In some implementations, the neck 1921 is configured to be constrained between the pair of laterally extending proximal members 1820 of the base 1510, such that the drive member 1910 is rotatable relative to the base 1510 but cannot move longitudinally relative to the base.

The driven member 1912 can take a variety of different forms. In some implementations, the driven member 1912 can have a threaded portion 1930 and a coupling end portion 1932. In some implementations, the threaded portion 1930 can be configured to be mate with the threaded portion 1922 of the drive member 1910. For example, the rotation of the drive member 1910 can longitudinally move the driven member 1912. The threaded portion 1922 can be a male threaded portion that engages with a female threaded portion 1930 or the threaded portion 1922 can be a female threaded portion that engages with a male threaded portion 1930.

The coupling end portion 1932 can take a variety of different forms. Any configuration that can couple the driven member 1912 to the rack 1914 can be used. In some implementations, the coupling end portion 1932 can include a slot 1934 that is configured to accept a portion of the rack 1914. In some implementations, the rack 1914 can be configured to prevent or inhibit rotation of the driven member 1912. In some implementations, the rack 1914 fits between the pair of laterally extending distal members 1822, such that the laterally extending distal members 1822 are prevented or inhibited from rotating. In some implementations, interaction between the slot 1934 in the coupling end portion 1932 and the rack 1914 inhibits relative rotation between the coupling end portion 1932 and the rack 1914. As a result, rotation of the drive member 1910 is converted to translational movement of the driven member 1912 and the rack 1914.

The rack 1914 can take a variety of different forms. In some implementations, the rack 1914 is a gear rack that is configured to drive one or two pinion gears 1940. A rack 1914 can be configured to engage two pinion gears 1940 in a variety of different ways. For example, teeth can be provided on both sides of the rack. In some implementations, the rack 1914 includes spaced apart openings 1942 that are sized and spaced to accept teeth 1944 of two pinion gears 1940 on opposite sides of the rack 1914.

The one or more anchors 1508 can take a variety of different forms. For example, the one or more anchors 1508 can include one or more of the features of any of the anchors described in the present disclosure. In some implementations, each of the anchors include a paddle 1520, an optional paddle frame 1524, and an optional gripping member 1530, such as a clasp. The paddle 1520 can take a variety of different forms.

In some implementations, the paddle 1520 can extend from a pivotable portion 1528 to a free end 1526. In some implementations, the pivotable portion 1528 can include the pinion gear 1940. In some implementations, the rack 1914 drives or rotates the pinion gear 1940 to move the paddle 1520 between two or more of the closed position, the partially open position, the open position, and the extended position.

In some implementations, the pivotable portion 1528 can include a passage 1950 that accepts the pivot member 1824 of the base 1510 to allow the paddle 1520 to pivot relative to the base 1510. In some implementations, the paddle 1520 includes a passage 1952 that accepts a paddle frame attachment member 1954 (e.g., rod, shaft, bar, threaded fastener, pin, bar, etc.)

The paddle frame 1524 can take a variety of different forms. For example, the one or more paddle frames 1524 can include one or more of the features of any of the paddle frames described in the present disclosure. In some implementations, the paddle frames 1524 are configured to press, coapt, bring together, or bring closer together two native valve leaflets (e.g., leaflets 20, 22 or two of leaflets 30, 32, 34). In some implementations, the paddle frames 1524 comprise one, two, or more separate components that are attached to or coupled to the paddles 1520. In some implementations, the paddle frames 1524 can be omitted. In some implementations, the paddles 1520 can be shaped to press, coapt, bring together, or bring closer together two native valve leaflets (e.g., leaflets 20, 22 or two of leaflets 30, 32, 34), without having an additional paddle frame component.

In some implementations, each paddle frame 1524 comprises a first paddle frame member 1527 and a second paddle frame member 1529. In some implementations, the first paddle frame member 1527 and the second paddle frame member are mirror images of one another. In some implementations, the first paddle frame member 1527 and the second paddle frame member are not mirror images of one another.

In some implementations, the first paddle frame member 1527 and the second paddle frame member 1529 can each include a pivot connection portion 2000, a leaflet engagement portion 2002, and a paddle connection portion 2004. In some implementations, the pivot connection portion 2000 includes an opening 2010 that accepts the pivot member 1824 to allow the paddle frame member 1527 or 1529 to pivot relative to the base 1510. In some implementations, the paddle connection portion 2004 includes an opening 2020 that accepts the paddle frame attachment member 1954 to couple or connect the paddle frame member 1527 or 1529 to the paddle 1520. In some implementations, the paddle 1520 and the paddle frame 1524 pivot together relative to the base 1510 about the pivot member 1824.

The paddle frame member 1527 or 1529 can be made from a variety of different materials and can be shaped in a variety of different ways. In some implementations, the paddle frame member 1527 or 1529 can be formed (e.g., cut and bent) from sheet material, can be molded, can be cast, 3-D printed, etc.).

In some implementations, the pivot connection portion 2000 and the paddle connection portion 2004 are connected to the leaflet engagement portion 2002. In some implementations, the pivot connection portion 2000 and the paddle connection portion 2004 are shaped to position the leaflet engagement portion 2002 to press, coapt, bring together, or bring closer together two native valve leaflets (e.g., leaflets 20, 22 or two of leaflets 30, 32, 34) and/or to wrap the native valve leaflets around the base 1510 connected to the leaflet engagement portion 2002.

The optional gripping member 1530 can take a variety of different forms. For example, the gripping member 1530 can have any of the features of any of the gripping members or clasps described in the present disclosure. In some implementations, the gripping members 1530 comprise a base or fixed arm 1532, a movable arm 1534, optional barbs 1536, and a joint portion 1538.

In some implementations, the fixed arms 1532 can be attached to the paddles 1520, with the joint portion 1538 disposed proximate the base 1510. In some implementations, the fixed arms 1532 can be attached to the paddles 1520 with any suitable means, such as sutures, screws or other fasteners, crimped sleeves, mechanical latches or snaps, welding, adhesive, or the like. In some implementations, the fixed arms 1532 remain substantially stationary relative to the paddles 1520 when the movable arms 1534 are opened to open the gripping members 1530.

In some implementations, the gripping members 1530 are opened by applying tension to actuation lines 116 (e.g., sutures, wires, etc.) attached to the movable arms 1534, thereby causing the movable arms 1534 to pivot on the joint portions 1538.

In some implementations, during treatment and/or implantation, the anchors 1508 are opened and closed to grasp the native mitral valve leaflets between the paddles 1520 and the base 1510. The optional gripping members 1530 can further secure the native leaflets by pinching the leaflets between the movable and fixed arms 1534, 1532. The optional barbs 1536 can increase friction with the leaflets or can partially or completely puncture the leaflets.

In some implementations, the actuation lines can be actuated separately so that each clasp 1530 can be opened and closed separately. Separate operation allows one leaflet to be grasped at a time, or for the repositioning of a gripping element 1530 on a leaflet, without altering a successful grasp on the other leaflet. In some implementations, the gripping members 1530 can open and close when the paddle 1520 is not closed, thereby allowing leaflets to be grasped in a variety of positions as the particular situation requires.

An implant catheter assembly 2200 for positioning and operating the device 1500 can take a variety of different forms. In some implementations, the implant catheter assembly 2200 can include a catheter 1731, a coupler 1720, an actuation element 1730, and an optional coupler support 2202. In some implementations, the optional coupler support 2202 is configured to mate with the coupling 1511. In some implementations, the optional coupler support 2202 can be configured to mate with the coupling 1511 in a variety of different ways. In some implementations, the coupler support 2202 and the coupling 1511 have mating surfaces that prevent or inhibit the coupler support 2202 and the coupling 1511 from rotating relative to one another, for example, when torque is applied by the actuation element 1730 to the device 1500.

In some implementations, the coupler support 2202 has one or more projections 2210 and one or more cutouts or recesses 2212. In some implementations, the coupling 1511 has one or more projections 2220 and one or more cutouts or recesses 2222. In some implementations, the projections 2210 can be placed in the recesses 2222 and projections 2220 can be placed in the recesses 2212 to prevent or inhibit the coupler support 2202 and the coupling 1511 from rotating relative to one another, for example, when torque is applied by the actuation element 1730 to the device 1500.

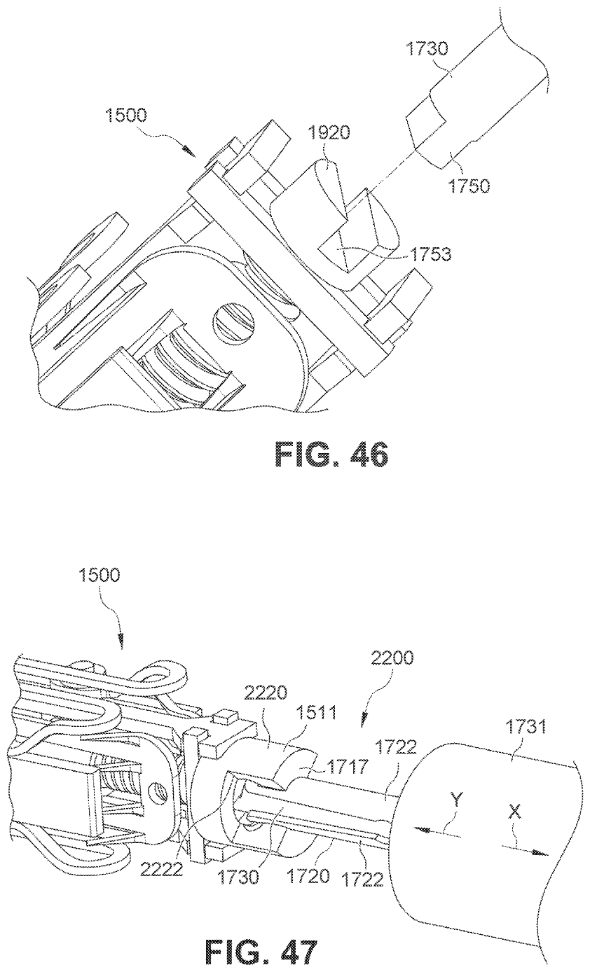

Referring to FIGS. 46 and 47, in some implementations the implant catheter assembly 2200 is coupled to the treatment and/or repair device 1500 by placing the coupler 1720 in the in the coupling 1511 without the actuation element 1730 in the movable arms 1722 and optionally placing the projections 2210 in the recesses 2222 and the projections 2220 in the recesses 2212.

Then, in some implementations, the actuation element is advanced into the movable arms 1722 to force the movable arms 1722 apart to secure the coupler 1720 to the coupling 1511. The drive end 1750 can be rotated into alignment with the mating feature 1753 (e.g., slot, Phillips recess, hex recess, star recess, or any other mating shapes) and then advanced to couple the drive end 1750 with the mating feature.

In some implementations, the drive end 1750 can be a male feature with the mating feature 1753 being a female feature, the drive end 1750 can be a female feature with the mating feature 1753 being a male feature, or the drive end 1750 and the mating feature 1753 can each comprise male and female features.

In some implementations, the above steps are reversed to uncouple the implant catheter assembly 2200 from the treatment and/or repair device 1500.

In some implementations, when the implant catheter assembly 2200 is coupled to the device 1500, the device 1500 can be moved between at least the configurations illustrated by FIGS. 40-43 and positions between the configurations illustrated by FIGS. 40-43. In some implementations, the paddles 1520 and paddle frames 1527, 1529 can be moved to the positions illustrated by FIGS. 40-43 by rotating the actuation element 1730. In some implementations, the actuation element 1730 rotates the driver 1910 to advance and retract the rack 1914. In some implementations, the rack 1914 drives the pinion gears 1940 to open and close the paddles 1520 and the optional paddle frames 1524.

In some implementations, the device 1500 can be deployed from a delivery sheath (see delivery sheath 2606). In some implementations, the base 1510 of the device 1500 can be implanted between the leaflets of the native mitral valve. The anchors 1508 are actuatable between open and closed conditions.

In some implementations, during implantation, the paddles 1520 are opened and closed to grasp the native mitral valve leaflets between the paddles 1520, 122 and the base 1510. In some implementations, the gripping members 1530 can be opened separately by pulling on an attached actuation line 116 that extends through the delivery sheath to the gripping member 1530.

The actuation line 116 can take a wide variety of forms, such as, for example, a line, a suture, a wire, a rod, a catheter, or the like. In some implementations, the gripping members 1530 can be spring loaded so that in the closed position the gripping members 1530 continue to provide a pinching force on the grasped native leaflet. In some implementations, the optional barbs 1536 can pierce the native leaflets.

Referring now to FIG. 43, the device 1500 is shown in a substantially elongated or substantially fully open condition. In some implementations, the device 1500 can optionally be loaded in the delivery sheath in the fully open position. In some implementations, the paddles 1520, 122 can form an angle with the base 1510 that is approximately 180 degrees or 180 degrees. In some implementations, the gripping elements 1530 can be kept in a closed condition during deployment through the delivery sheath 2606. In some implementations, the gripping members 1530 can be in a fully open position, ranging from about 140 degrees to about 200 degrees, to about 170 degrees to about 190 degrees, or about 180 degrees with the paddles 1520 in the substantially fully open position or in the fully open position.

Referring now to FIG. 31, the device 1500 can be moved to a fully closed condition. The compact size and/or configuration of the device 1500 in the fully closed condition allows for easier maneuvering and placement within the heart. In some implementations, to move the device 1500 from the elongated condition to the shortened condition, the actuation element 1730 is rotated to advance the rack 1914 distally. In some implementations, this distal movement of the rack 1914 rotates the pinion gears 1940 to close the paddles 1520 and the optional paddle frames 1527, 1529. In some implementations, the threads of the drive member 1910 and the driven member 1912 can be configured to maintains or lock the paddles 1520 and the optional paddle frames 1527, 1529 in any of the positions when the actuation element 1730 is disengaged from the head 1920.

Referring now to FIG. 40, the device 1500 is shown in a partially open configuration. In some implementations, the lines 116 can be pulled to open the gripping members 1530 and place the device 1500 in a leaflet capture ready condition. In some implementations, to transition from the fully closed to the partially open condition, the actuation element 1730 is rotated to move the paddles 1520. Also, the positions of the gripping members can be dependent on the positions of the paddles 1520. For example, closing the paddles 1520 can also close the clasps.

In some implementations, the paddles 1520 can be independently controllable. For example, the device 1500 can have two independent drive assemblies 1514 with two independent racks 1914. In some implementations, the gripping elements 1530 are independently controllable. One of the actuation lines 116 is extended to allow one of the gripping elements 1530 to close. The other actuation line 116 ca be extended to allow the other gripping element to close. Either or both of the actuation lines 116 can be repeatedly actuated to repeatedly open and close the gripping elements 1530.

In some implementations, the fixed and movable arms 1532, 1534 of the gripping elements 1530 can be biased to pinch the leaflets. In some implementations, the joint portions 1538 can be formed of any suitably elastic material, such as a metal or polymer material, to maintain the gripping elements 1530 in the closed condition after implantation.

In some implementations, the implantable device 1500 can be delivered and implanted within the native mitral valve MV or the native tricuspid valve TV of the heart H. For example, when implanted in the tricuspid valve TV the delivery sheath can be inserted into the left atrium LA through the septum and the device 1500 is deployed from the delivery sheath. The device 100 can be moved into position within the mitral valve MV into the ventricle LV and partially opened so that the leaflets 20, 22 can be grasped.

In some implementations, an actuation line 116 can be extended to close one of the gripping members 1530, capturing a leaflet 20. In some implementations, the other actuation line 116 can be extended to close the other gripping member 1530, capturing the remaining leaflet 22. In some implementations, the paddles 1520 can be closed or partially closed such that the leaflets 20, 22 are brought together or coapted between the paddle frames 1527, 1529.

In some implementations, the device 1500 can then be decoupled from implant catheter assembly 2200. In some implementations, the sheath 2606 and actuation lines 116 are then retracted leaving the device 1500 deployed in the native mitral valve MV (or the tricuspid valve).

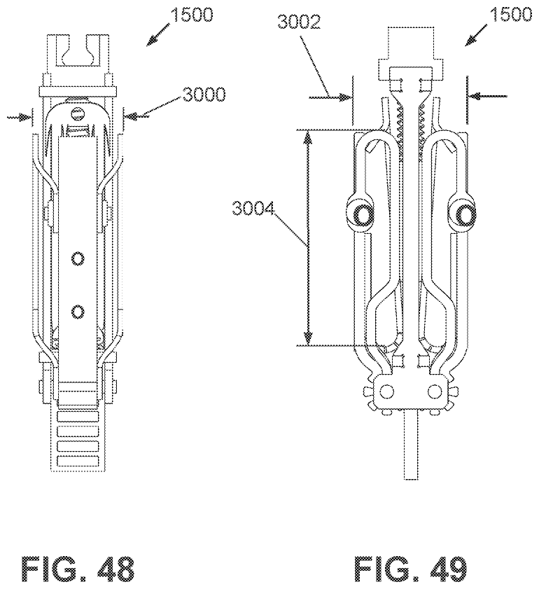

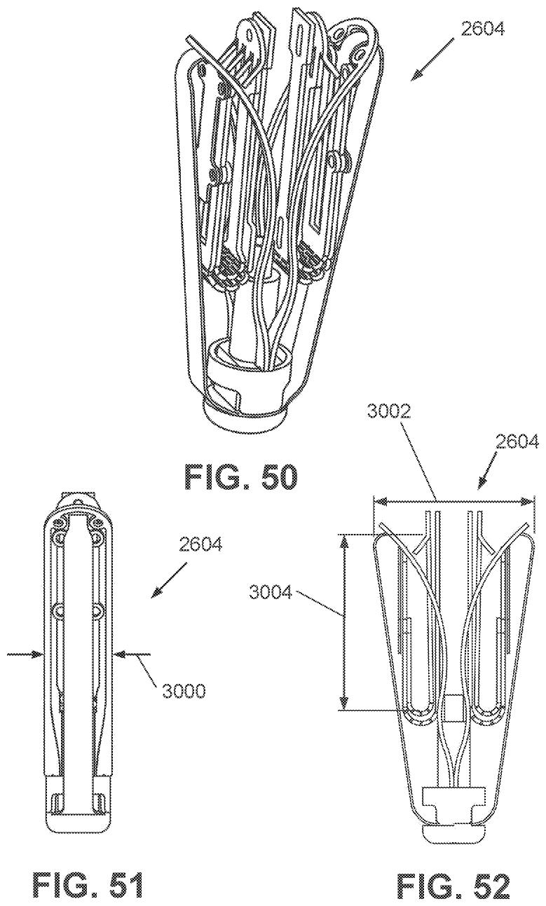

In some implementations, treatment and/or repair devices, such as any of the treatment and/or repair devices disclosed in this application or any known treatment and/or repair device, can be made with a small size. FIGS. 48-52 illustrate example devices that can be made with a small size. The device 1500 illustrated by FIGS. 48 and 49 can be the same device 1500 illustrated by FIGS. 31-43 and described above.

In some implementations, the treatment and/or repair device 2604 can have the same or similar components as the treatment and/or repair devices 200, 300, and/or 8200 described above and can operate in the same or similar way to the treatment and/or repair devices 200, 300, and/or 8200 described above. The small size can allow two or more devices to be implanted, can allow one or more devices to be implanted in patients having smaller anatomies, and/or can allow the device to be implanted in anatomical locations where current devices are considered oversized. When more than one device needs to be implanted, the small device can inhibit or reduce elevation of mitral gradients or tricuspid gradients.

In some implementations, devices, such as any of the treatment and/or repair devices disclosed herein, can be configured to be implanted in mitral valves having a mitral valve area (MVA) in cm² between 2.2 and 6, such as between 2.6 and 5, such as between 2.8 and 4.5, such as between 2.8 and 4.2 or any subrange of any of these ranges.

In some implementations, devices, such as any of the treatment and/or repair devices disclosed herein, can be configured to be implanted with a transeptal puncture height (the height from the mitral valve annulus to the puncture through the septum, TSP) in cm between 2.0 and 4.5, such as between 2.5 and 4.5, such as between 2.6 and 4.2, such as between 2.7 and 4.0 or any subrange of any of these ranges.

In some implementations, devices, such as any of the treatment and/or repair devices disclosed herein, can be configured to be implanted where MVA is between 2.2 and 6 and TSP is between 2.0 and 4.5, such as MVA between 2.6 and 5 and TSP between 2.5 and 4.5, such as MVA between 2.8 and 4.5 and TSP between 2.6 and 4.2, such as MVA between 2.8 and 4.2 and TSP between 2.7 and 4.0 or any subrange of any of these ranges.

In some implementations, a medial-lateral width 3000 of a treatment and/or repair device, such as any of the treatment and/or repair devices disclosed herein is between 2 mm and 4 mm, such as between 2 mm and 3.8 mm, such as between 2 mm and 3.6 mm, such as between 2 mm and 3.4 mm, such as between 2 mm and 3.2 mm, or any subrange of these ranges.

In some implementations, an anterior-posterior width 3002 of a treatment and/or repair device, such as any of the treatment and/or repair devices disclosed herein is between 2 mm and 9 mm, such as between 2 mm and 6 mm, such as between 2 mm and 5 mm, such as between 2 mm and 4.8 mm, such as between 3 mm and 4.8 mm, or any subrange of these ranges.

In some implementations, a treatment and/or repair device, such as any of the treatment and/or repair devices disclosed herein has a medial-lateral width between 2 mm and 4 mm and an anterior-posterior width between 2 mm and 9 mm, such as a medial-lateral width between 2 mm and 3.8 mm and an anterior-posterior width between 2 mm and 6 mm, such as a medial-lateral width between 2 mm and 3.6 mm and an anterior-posterior width between 2 mm and 5 mm, such as a medial-lateral width between 2 mm and 3.4 mm and an anterior-posterior width between 2 mm and 4.8 mm, such as a medial-lateral width between 2 mm and 3.2 mm and an anterior-posterior width between 3 mm and 4.8 mm, or any subrange of these ranges.

In some implementations, a leaflet capture height 3004 (e.g., an insertion length of a native valve leaflet in a clasp of the treatment and/or repair device or a length of engagement of a native valve leaflet in the treatment and/or repair device is between 5 mm and 10 mm, such as between 6 mm and 9 mm, such as between 7 mm and 9 mm or any subrange of these ranges.

In some implementations, a device, such as any of the treatment and/or repair devices disclosed herein has a medial-lateral width between 2 mm and 4 mm, an anterior-posterior width between 2 mm and 9 mm and a leaflet capture height between 5 mm and 10 mm, such as a medial-lateral width between 2 mm and 3.8 mm, an anterior-posterior width between 2 mm and 6 mm, and a leaflet capture height between 6 mm and 9 mm, such as a medial-lateral width between 2 mm and 3.6 mm, an anterior-posterior width between 2 mm and 5 mm, and a leaflet capture height between 7 mm and 9 mm, or any subrange of these ranges.

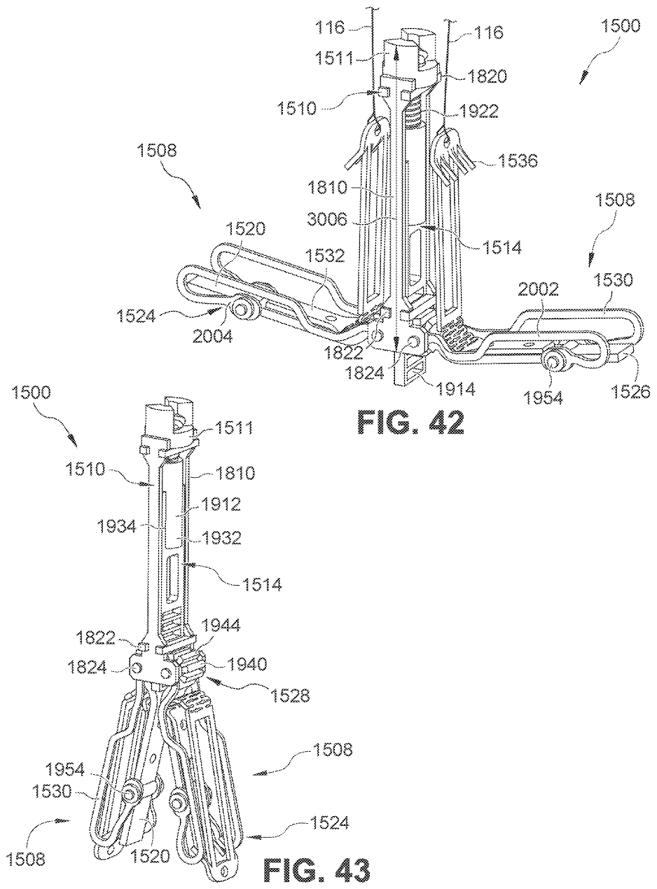

In some implementations, the treatment and/or repair device can have a height 3006 in an open position (see FIG. 42) between 10 and 22 mm, such as between 12 and 20 mm, such as between 14 and 18 mm, such as between 14 and 16 mm.

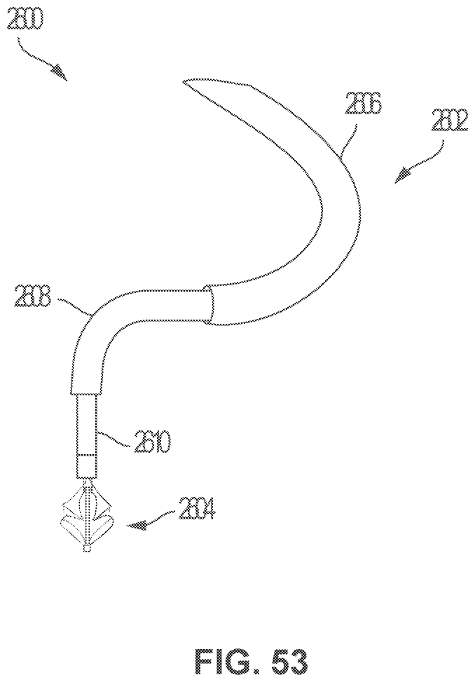

Referring to FIG. 53, in some implementations a system or assembly 2600 (e.g., a valve treatment system or assembly, valve repair system or assembly, valve replacement system or assembly, etc.) can comprise a delivery assembly or delivery system 2602 and an implantable device or implant 2604, such as any of the treatment and/or repair devices disclosed herein. In some implementations, the delivery system 2602 can comprise a plurality of catheter assemblies. In some implementations, the delivery system 2602 can also comprise one or more optional catheter stabilizers or stabilizing systems/devices (not shown in FIG. 53).

In some implementations, the delivery system 2602 includes a first catheter 2606, a second catheter 2608, and a third catheter 2610. Though, in some implementation, the delivery system 2602 can include fewer or more catheters than shown. In some implementations, the first catheter 2606 is configured as a guide sheath that is configured to be introduced through the femoral artery, through the inferior vena cava, into the right atrium, through the atrial septum, and into the left atrium, above the mitral valve MV.

In some implementations, the first catheter 2606 is optionally configured as a steerable catheter. In some implementations, the second catheter 2608 is optionally configured as a steerable catheter. In some implementations, the second catheter 2608 can be routed through the first catheter 2606 and positioned in or near the mitral valve annulus to position the treatment and/or repair device 2604 for implantation. In some implementations, the third catheter 2610 is an implant catheter that is optionally not steerable, but is used to control the treatment and/or repair device 2604. In some implementations, the third catheter 2610 can extend through the second catheter 2608 to position the treatment and/or repair device 2604 for implantation.

In some implementations, the second catheter 2608 extends coaxially through the first catheter 2606, and the third catheter or implant catheter 2610 extends coaxially through the second catheter 2608 and the first catheter 2606. In some implementations, the implantable device 2604 can be releasably coupled to a distal portion of the third catheter 2610. The implantable device 2604 can be the same as or similar to any device described herein.

In some implementations, the delivery catheter 2606 and the steerable catheter 2608 can be used, for example, to access an implantation location (e.g., a native mitral valve region of a heart or a native tricuspid valve region of the heart) and/or to position the implant catheter 2610 at the implantation location. Accordingly, in some implementations, the delivery catheter 2606 and the steerable catheter 2608 are configured to be steerable. The catheter assemblies or features of the catheter assemblies disclosed by U.S. Pat. Nos. 10,653,862 and 10,646,342 can be used as or in the catheters 2606, 2608, 2610. U.S. Pat. Nos. 10,653,862 and 10,646,342 are hereby incorporated by reference in their entireties.

In some implementations, the device 2604, such as any of the treatment and/or repair devices disclosed herein can be sized and/or an interior passage of the steerable catheter 2608 can be sized to allow the device to slide through the interior passage of the steerable catheter 2608.

In some implementations, after a first treatment and/or repair device 2604 is implanted and released from the implant catheter 2610, the implant catheter 2610 is pulled proximally and out of the steerable catheter 2608, leaving the steerable catheter 2608 in the guide sheath. If additional implants 2604 are needed, the additional implant 2604 can be routed through the steerable catheter 2608 with another implant catheter and the additional implant can be implanted on the native valve. This process can be repeated for any number of devices 2604.

In some implementations, the devices 2604 that are sequentially implanted can be the same size and/or type of device or the devices 2604 can be different sizes and/or types of treatment and/or repair devices. For example, a first treatment and/or repair device 2604 that is implanted can be larger than a second treatment and/or repair device that is routed through the steerable catheter 2608 and implanted. In some implementations, the first treatment and/or repair device 2604 that is implanted is too large to fit through the steerable catheter 2608, but is small enough to fit inside the guide sheath 2608, and the second treatment and/or repair device 2604 is small enough to be routed through the steerable catheter 2608.

In some implementations, a device 2604, such as any of the treatment and/or repair devices disclosed herein can be sized and/or an interior passage of the steerable catheter 2608 can be sized to allow the first treatment and/or repair device to slide through the interior passage of the steerable catheter 2608. In some implementations, after a first treatment and/or repair device 2604 is implanted and released from the implant catheter 2610, the implant catheter 2610 is pulled proximally and out of the steerable catheter 2608, leaving the steerable catheter 2608 in the guide sheath. If additional implants 2604 are needed, the additional implant 2604 can also be routed through the steerable catheter 2608 with another implant catheter and the additional implant can be implanted on the native valve. This process can be repeated for any number of treatment and/or repair devices 2604.

In some implementations, treatment and/or repair devices 2604 that are sequentially implanted through one steerable catheter 2608 can be the same size and/or type of treatment and/or repair device or the treatment and/or repair devices 2604 can be different sizes and/or types of treatment and/or repair devices. For example, a first treatment and/or repair device 2604 that is routed through the steerable catheter 2608 and implanted can be larger than a second treatment and/or repair device that is routed through the steerable catheter 2608 and implanted.

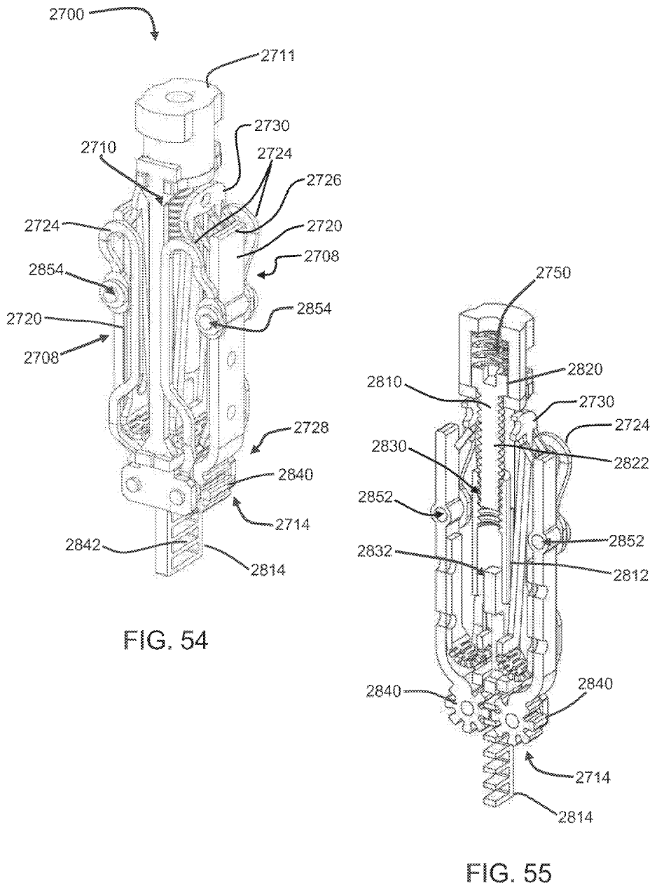

Referring now to FIGS. 54-60, an example of a device 2700 (e.g., a treatment device, a repair device, a prosthetic device, a valve repair device, valve treatment device, implantable device, implant, etc.) is shown. The device 2700 can include any other features for a device discussed in the present disclosure, and the device 2700 can be positioned to engage valve tissue 20, 22, 30, 32, 34 as part of any suitable treatment and/or repair system (e.g., any treatment and/or repair system described in the present disclosure).

In some implementations, the device 2700 can comprise an optional coupling 2711, a base 2710, an actuation assembly 2714, and one or more anchors 2708. In some implementations, the coupling 2711 is connected to a proximal end of the base 2710. In some implementations, the actuation assembly 2714 is housed at least partially within the base 2710. In some implementations, the one or more anchors can comprise one anchor 2708 (e.g., a device configured to attach to only a single native valve leaflet), two anchors 2708 (e.g., a device configured to attach to two leaflet, such as the leaflets of the mitral valve or two leaflets of the tricuspid valve), or three anchors 2708 (e.g., a device configured to attach to the three leaflets of the tricuspid valve).

In some implementations, the one or more anchors 2708 are coupled to the actuation assembly 2714, such that operation of the actuation assembly can move the anchors between two or more of a closed position (see FIG. 54), a partially open or capture ready position (e.g., similar to the position of the device 1500 shown in FIG. 40), a fully open position (e.g., similar to the position of the device 1500 shown in FIG. 41), and an extended position (e.g., similar to the position of the device 1500 shown in FIG. 43).

In some implementations, the coupling 2711 can take a variety of different forms. For example, the coupling 2711 can have any of the features of any of the couplings or proximal collars described in the present disclosure and/or can have any of the features of any of the couplings or proximal collars disclosed in PCT Application No. PCT/US2018/028189, published as PCT Publication WO2018/195215.

In some implementations, an implantable device 2700 has a coupling 2711 that accepts an actuation element (e.g., any actuation element described in the present disclosure). The actuation element can extend through an implant catheter (e.g., any implant catheter described in the present disclosure) and through a coupler (e.g., any coupler described in the present disclosure) that extends from or is extendable from a distal end of the implant catheter. The actuation element can be coupled to the device 2700 to open and close the anchor(s) 2708 of the device 2700.

The base 2710 can take a variety of different forms, such as, for example, any form described for the base 1510 shown in FIGS. 31-43. In some implementations, the base 2710 can be any of the coaption elements described in the present disclosure. The base can be formed from a single component or can be assembled from a plurality of components. In some implementations, the base 2710 comprises a frame with an open interior for other components of the device, such as a metal frame.

The actuation assembly 2714 can take a variety of different forms, such as, for example, any form described for actuation assembly 1514 shown in FIGS. 31-43. For example, the actuation assembly 2714 can be any of the actuation elements, actuation assemblies, and/or actuation linkages described in the present disclosure. In some implementations, the actuation assembly 2714 can be comprised of longitudinally movable components, such as one or more shafts, rods, tubes and/or can comprise one or more assemblies that convert rotational movement to translational movement, such as gear assemblies (e.g., rack and pinion assemblies, worm gear assemblies etc.) and/or that convert rotational movement about an axis in first plane to rotational movement about an axis in a second plane, that can be orthogonal to the first plane (e.g., worm gear assemblies, planetary gear assemblies, etc.) The actuation assembly 2714 can take any form where a longitudinal movement and/or a rotational movement is used to open and/or close one or more components of the anchors 2708.

In some implementations, the actuation assembly 2714 comprises a drive member 2810, a driven member 2812, and a rack 2814. The drive member 2810 can take a variety of different forms, such as any form described for the drive member 1910 shown in FIGS. 31-43. In some implementations, the drive member 2810 comprises a head 2811, a neck (not shown), and a threaded portion 2822.

In some implementations, the head 2811 can be configured to be engaged by a drive end of the actuation element. For example, the head 2811 and the drive end can have a corresponding mating feature, such as slotted, Phillips, hex, star, or any other mating shapes. In some implementations, the head 2811 can take the form of the head 3820 shown in FIGS. 86-94, and the actuation element can take the form of the actuation element 3930 shown in FIGS. 86 and 89-94. Any configuration that allows the actuation element to drive the head 2811 can be used.

In some implementations, the drive member 2810 is configured to be constrained by the base 2710 such that the drive member 2810 is rotatable relative to the base 2710 but cannot move longitudinally relative to the base. For example, the drive member 2810 can be constrained by the base 2710 in a similar manner in which drive member 1910 is constrained by the base 1510 described in the present disclosure.

The driven member 2812 can take a variety of different forms, such as any form described for the driven member 1912 shown in FIGS. 31-43. In some implementations, the driven member 2812 can have a threaded portion 2830 and a coupling end portion 2832. In some implementations, the threaded portion 2830 can be configured to be mate with the threaded portion 2822 of the drive member 2810. For example, the rotation of the drive member 2810 can longitudinally move the driven member 2812.

In some implementations, the threaded portion 2822 can be a male threaded portion that engages with a female threaded portion 2830 or the threaded portion 2822 can be a female threaded portion that engages with a male threaded portion 2830. In some implementations, the coupling end portion 2832 can take a variety of different forms, such as any form described for the drive member 1932 of the driven member 1912 shown in FIGS. 31-43.

The rack 2814 can take a variety of different forms, such as any form described for the rack 1914 shown in FIGS. 31-43. In some implementations, the rack 2814 is a gear rack that is configured to drive one or two pinion gears 2840. A rack 2814 can be configured to engage two pinion gears 2840 in a variety of different ways. For example, teeth can be provided on both sides of the rack. In some implementations, the rack 2814 includes spaced apart openings 2842 that are sized and spaced to accept teeth of two pinion gears 2840 on opposite sides of the rack 2814.

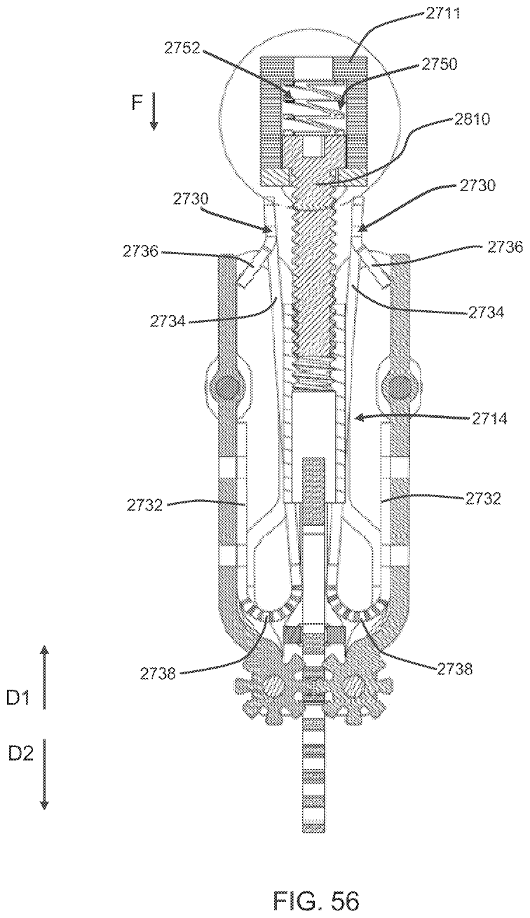

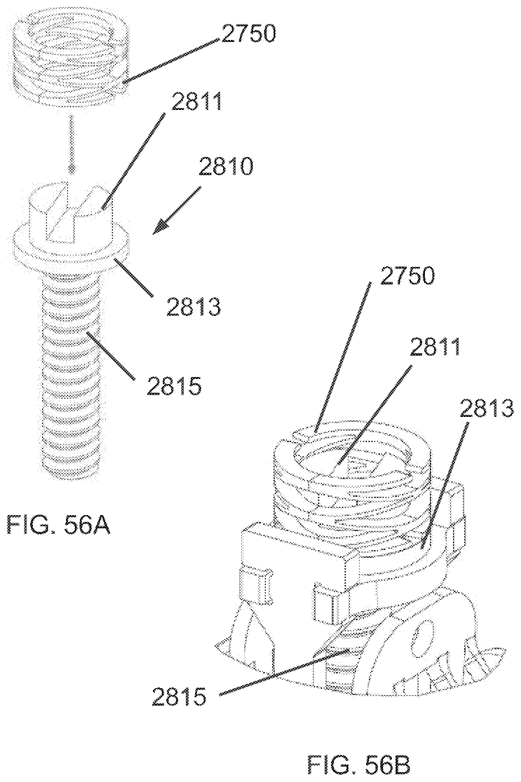

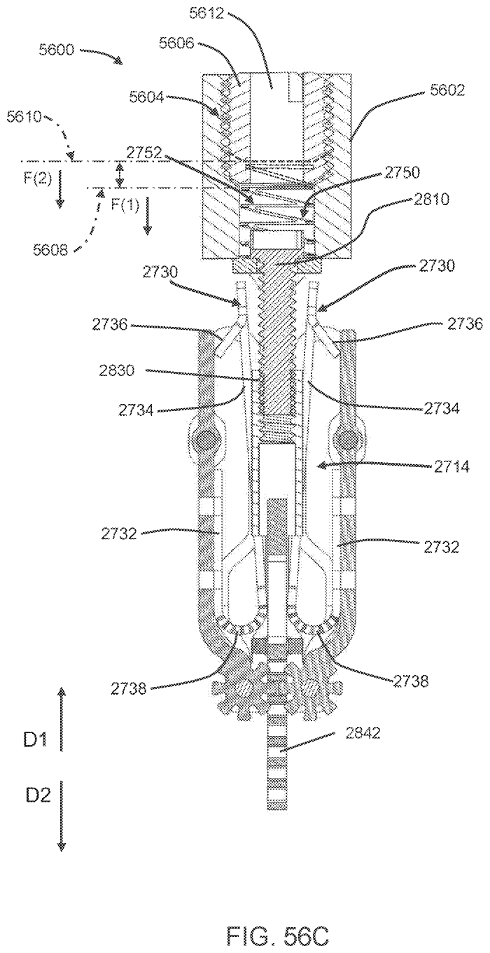

The engagement and interaction between the drive member 2810 and the driven member 2812 can take any suitable form that allows for the drive member 2810 to drive the driven member 2812, such as, for example, any form described with reference to the drive member 1910 and the driven member 1912 shown in FIGS. 31-43. The engagement and interaction between the driven member 2812 and the rack 2814 can take any suitable form that allows for the rack to be moved in directions D1, D2 (FIG. 56) such that the rack engages the pinion gears 2840 of the paddles 2720 of the anchors 2708 to move the paddles 2720 between two or more of the closed position, the partially open position, the open position, and the extended position.

The one or more anchors 2708 can take a variety of different forms, such as, for example, any form described for the anchors 1508 shown in FIGS. 31-43. The one or more anchors 2708 can include one or more of the features of any of the other anchors described in the present disclosure. In some implementations, each of the anchors 2708 include a paddle 2720, an optional paddle frame 2724, and an optional gripping member 2730, such as a clasp. The paddle 2720 can take a variety of different forms.

In some implementations, the paddle 2720 can extend from a pivotable portion 2728 to a free end 2726. In some implementations, the pivotable portion 2728 can include the pinion gear 2840. In some implementations, the rack 2814 drives or rotates the pinion gear 2840 to move the paddle 2720 between two or more of the closed position, the partially open position, the open position, and the extended position.

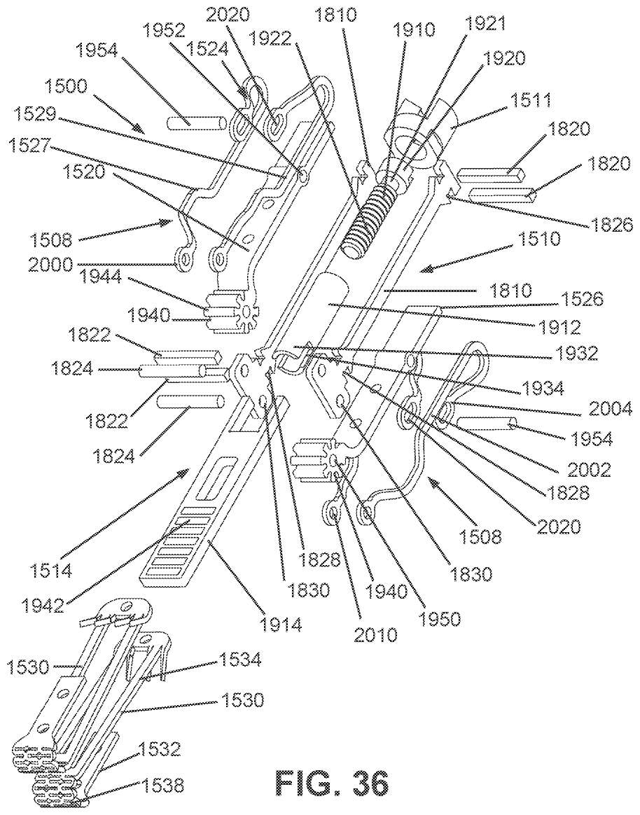

In some implementations, the pivotable portion 2728 can include a passage (e.g., similar to passage 1950 for the paddle 1520 shown in FIG. 36) that accepts the pivot member of the base 2710 (e.g., similar to the pivot member 1824 of the base 1510 for the device 1500 shown in FIGS. 31-43) to allow the paddle 2720 to pivot relative to the base 2710. In some implementations, the paddle 2720 includes a passage 2852 that accepts a paddle frame attachment member 2854 (e.g., rod, shaft, bar, threaded fastener, pin, bar, etc.)

The paddle frame 2724 can take a variety of different forms, such as, for example, any form described for the paddle frame 1524 shown in FIGS. 31-43, or any other features of any of the paddle frames described in the present disclosure. The paddle frame 2724 can be connected to the paddle 2720 in any suitable manner, such as, for example, any connection described between the paddle frame 1524 and the paddle 1520 shown in FIGS. 31-43.

In some implementations, the paddle frames 2724 are configured to press, coapt, bring together, or bring closer together two native valve leaflets (e.g., leaflets 20, 22 or two of leaflets 30, 32, 34). In some implementations, the paddle frames 2724 comprise one, two, or more separate components that are attached to or coupled to the paddles 2720. In some implementations, the paddle frames 2724 can be omitted. In some implementations, the paddles 2720 can be shaped to press, coapt, bring together, or bring closer together two native valve leaflets (e.g., leaflets 20, 22 or two of leaflets 30, 32, 34), without having an additional paddle frame component. The paddle frame 2724 can be made from a variety of different materials and can be shaped in a variety of different ways. In some implementations, one or more components of the paddle frame 2724 can be formed (e.g., cut and bent) from sheet material, can be molded, can be cast, 3-D printed, etc.).

The optional gripping member 2730 can take a variety of different forms. For example, the gripping member 2730 can have any of the features of any of the gripping members or clasps described in the present disclosure. In some implementations, the gripping members 2730 comprise a base or fixed arm 2732, a movable arm 2734, optional barbs 2736, and a joint portion 2738.

In some implementations, the fixed arms 2732 can be attached to the paddles 2720, with the joint portion 2738 disposed proximate the base 2710. The fixed arms 2732 can be attached to the paddles 2720 with any suitable means, such as sutures, screws or other fasteners, crimped sleeves, mechanical latches or snaps, welding, adhesive, or the like. In some implementations, the fixed arms 2732 remain substantially stationary relative to the paddles 2720 when the movable arms 2734 are opened to open the gripping members 2730.

In some implementations, the gripping members 2730 are opened by applying tension to actuation lines (e.g., sutures, wires, etc.) attached to the movable arms 2734, thereby causing the movable arms 2734 to pivot on the joint portions 2738.

In some implementations, during treatment and/or implantation, the anchors 2708 are opened and closed to grasp the native mitral valve leaflets (or tricuspid valve leaflets) between the paddles 2720 and the base 2710. The optional gripping members 2730 can further secure the native leaflets by pinching the leaflets between the movable and fixed arms 2734, 2732. The optional barbs 2736 can increase friction with the leaflets or can partially or completely puncture the leaflets.

In some implementations, the actuation lines can be actuated separately so that each clasp 2730 can be opened and closed separately. Separate operation allows one leaflet to be grasped at a time, or for the repositioning of a gripping element 2730 on a leaflet, without altering a successful grasp on the other leaflet. In some implementations, the gripping members 2730 can open and close when the paddle 2720 is not closed, thereby allowing leaflets to be grasped in a variety of positions as the particular situation requires.

In some implementations, the device 2700 can be configured to be removably connected to an implant catheter assembly (not shown), where the implant catheter assembly can include an actuation element for engaging the actuation assembly 2714 of the device 2700. The implant catheter assembly can take any suitable form that is capable of removably attaching to the coupling 2711 of the device 2700, such as, for example, any form of an implant catheter assembly described in the present disclosure. The actuation element of the implant catheter assembly can take any suitable form that is capable of engaging the actuation assembly 2714 of the device 2700 to move the paddles 2720 between two or more of the closed position, the partially open position, the open position, and the extended position.

In some implementations, the paddles 2720 can be moved by rotating the actuation element to cause the drive member 2810 to rotate, which consequently causes the driven member 2812 to engage the rack 2814 and cause the rack 2814 to move in a proximal direction D1 (FIG. 56) or a distal direction D2 (FIG. 56). In some implementations, movement of the rack 2814 in the proximal direction D1 causes the paddles 2720 to move toward the closed position (as shown in FIG. 54), and movement of the rack 2814 in the distal direction D2 causes the paddle 2720 to move toward at least one of the partially open, the open, and the extended positions.

In some implementations, movement of the rack 2814 in the distal direction D2 causes the paddles 2720 to move toward the closed position (as shown in FIG. 54), and movement of the rack 2814 in the proximal direction D1 causes the paddle 2720 to move toward at least one of the partially open, the open, and the extended positions. In some implementations, the engagement between the rack 2814 and the pinion gears 2840 allows for the movement of the paddles 2720 between two or more of the closed position, the partially open position, the open position, and the extended position. The optional paddle frames 2724 can be moved with the paddles 2720 due to a connection between the paddles 2720 and the paddle frames 2724.

In some implementations, the device 2700 can be deployed from a delivery sheath (see delivery sheath 2606). In some implementations, the base 2710 of the device 2700 can be implanted between the leaflets of the native mitral valve or the tricuspid valve. The anchors 2708 are actuatable between open and closed conditions. In some implementations, during implantation, the paddles 2720 are opened and closed to grasp the native mitral valve leaflets between the paddles 2720 and the base 2710.

In some implementations, the device 2700 can be configured such that the paddles 2720 are able to move between various positions (e.g., the closed position, the partially open position, the open position, etc.) in response to movement of the leaflets of the native valve (e.g., movement of the leaflets to the open position during the diastolic phase) when the device is attached to the leaflets. The movability of the paddles 2720 is advantageous because it reduces the stress on the leaflets resulting from the attachment between the device 2700 and the leaflets.

In some implementations, the device 2700 can be configured such that the paddles are movable between various positions (e.g., the closed position, the partially open position, the open position, the bailout position, etc.) to an implantation position in response to adjustment of the drive member 2810 by a tool and are also able to move in response to movement of the leaflets of the native valve (e.g., opening of the leaflets during the diastolic phase moves the paddles toward the open position from the position set by the tool). This combination of features (i.e., being able to set the position of the paddles of the implanted device and allowing the paddles to move from the implanted position with the beating of the heart) can optionally be implemented on any of the devices disclosed herein.

The device 2700 can be configured to set the paddles 2720 to a selected implanted position and/or to allow the paddles to move to a degree with the movement of the native valve leaflets in a variety of different ways. In some implementations, the paddles 2720 can be adjusted to an implantation position by any of the paddle control or adjustment mechanisms disclosed herein. For example, the paddles can be adjusted to an implanted position by a shaft that moves a coupler that is lockable at a range of positions, a rack and pinion mechanism, a threaded mechanism, etc. In some implementations, the paddles can be allowed to move from the implanted position with the movement of the native valve leaflets by allowing movement of the drive member 2810, allowing movement of the paddles 2720 without movement of the drive member, and/or allowing movement of one or more members that couple the drive member 2810 and the paddles 2720.

In some implementations, the device 2700 can be configured such that the paddles are movable between various positions (e.g., the closed position, the partially open position, the open position, the bailout position, etc.) to an implantation position in response to adjustment of the drive member 2810 by a tool, a degree or distance that paddles can move from the position set by the tool by movement of the native valve can be controlled and/or an amount of force or a force profile required to move the paddles from the position set by the tool can be controlled. This combination of features (i.e., being able to set the position of the paddles of the implanted device, setting an amount or degree that the paddles can move from the implantation position, and/or setting an amount of force (or a force curve or profile) can optionally be implemented on any of the devices disclosed herein.

The degree or distance that paddles can move from the position set by the tool by movement of the native valve can be controlled in a variety of different ways. For example, a space or gap can be provided to allow movement of the drive member after the position of the drive member is set by the tool, a stop can be provided that limits movement of the drive member, and/or a spring can be configured to allow movement of the drive member until the spring fully compresses or bottoms out.

The amount of force or a force profile required to move the paddles from the position set by the tool can be controlled in a variety of different ways. For example, one or more springs can be provided that resist movement of the drive member 2810 from the position set by the tool. In some implementations, the force that the spring or other biasing member applies to the drive member can be adjusted. For example, the spring can optionally be compressed or relaxed to adjust the force or force profile required to move the paddles from the position set by the tool. In some implementations, an adjustment member is movable to adjust an amount of compression of a spring that sets the force or force profile needed to move the paddles and/or to adjust the degree or distance that the paddles can move from the position set by the tool.

In the illustrated example, the device 2700 has a gap 2752 positioned within the coupling 2711 proximate the drive member 2810 of the actuation assembly 2714, which allows for the drive member 2810 to move in the proximal direction D1 and/or distal direction D2 within the gap 2752. The gap 2752 allows for the actuation assembly 2714 to be engaged in a reverse direction with movement of the paddles 2720 causing movement of the actuation assembly 2714, as compared to normal movement of the device in which the actuation assembly causes movement of the paddles 2720.

In some implementations, the height of the gap 2752 controls the distance at which the drive member 2810 is able to move in the proximal direction D1 and/or distal direction D2 and, consequently, controls the amplitude at which the paddles 2720 are able to flex as a result of the moving of the leaflets of the native valve. In some implementations, the height of the gap 2752 can be between about 0.2 mm about 4 mm, such as between 0.3 mm and 3 mm, such as between 0.4 mm and 2.5 mm, such as between 0.5 mm and 2 mm, or any sub range of these ranges. The gap 2752 is shown being positioned within the coupling 2711 of the device 2700, it should be understood that the gap 2752 can be positioned at any other suitable location that allows for the actuation assembly 2714 to move in response to the paddles 2720 moving to an open position due to the attachment between the device 2700 and the leaflets. In some implementations, the force required to open the paddles 2720 optionally increases as the gap is reduced.

In some implementations, the amplitude at which the paddles 2720 are able to flex as a result of the moving of the leaflets of the native valve the gap 2752 is adjustable. This amplitude can be adjustable in a variety of different ways.

In some implementations, the gap is adjustable. By adjusting the gap 2752 the amount or magnitude that the paddles 2720 can open can be adjusted. For example, the gap can be adjusted between a position that allows a maximum amount of paddle opening and a position that prevents any opening of the paddles 2720. The gap 2752 can be adjusted in a variety of different ways.

In some implementations, the gap 2752 can be adjusted by rotating the drive member 2810 beyond a position where the paddles are fully closed. This causes threads of the drive member 2810 and driven member 2812 to push the drive member proximal and reduce the gap. The gap can be at a maximum where the drive member 2810 moves the paddles 2720 to the closed position, but no further rotation of the drive member 2810 occurs after the paddles are fully closed. The paddles can move a maximum amount with the native leaflets when the gap is at a maximum. The gap can be at a minimum when the drive member 2810 is continued to be rotated as far as possible after the paddles are fully closed. When the gap is at a minimum, the paddles 2720 can be rigid or cannot move as the native valve leaflets move.

In some implementations, in which movement of the rack 2814 in the distal direction D2 (FIG. 56) causes the paddles 2720 to move to the closed position, the opening of the leaflets (e.g., during the diastolic phase) can cause the paddles 2720 to move to the partially open or open position, which causes the rack 2814 to move in the proximal direction D1 (via the connection between the rack 2814 and the pinion gears 2840), which consequently causes the driven member 2812 and the drive member 2810 to move in the proximal direction D1. The gap 2752 allows for such movement of the drive member 2810 in the proximal direction D1, thus allowing the paddle 2720 to move to the partially open or open position.

In some implementations, the device 2700 can include a biasing member 2750 that is configured to engage the actuation assembly 2714 to maintain, e.g., bias, the paddles 2720 in the closed position when the device 2700 is attached to the leaflets of the native valve, while allowing for the paddles to move to the open position in response to the opening of the leaflets (e.g., during the diastolic phase). In the illustrated example, the biasing member 2750 is positioned within the gap 2752 in the coupling 2711 such that the biasing member 2750 engages the drive member 2810 of the actuation assembly 2714 and the coupling 2711 to maintain a compliance or bias force F on the drive member 2810. In some implementations, the biasing member engages a proximal end of the drive member 2810 as illustrated by FIG. 56.

In some implementations, the drive member 2810 includes a head 2811, that is attached to a collar 2813, that is attached to a shaft or shank 2815 as is illustrated by FIGS. 56A and 56B. The biasing member 2750 is disposed around the head 2811 and engages the collar 2813. Placing the biasing member 2750 around the head 2811 increases the stroke or effective area of the gap that the drive member can move in. This configuration increases the distance or magnitude that the paddles can be opened and/or the precision of the paddle opening.

In some implementations in which movement of the rack 2814 in the distal direction D2 causes the paddles 2720 to move to the closed position, the biasing member 2750 can be configured to engage the drive member 2810 to provide a force F (FIG. 56) on the drive member 2810 to bias the paddles 2720 toward the closed position. When the device 2700 is attached to the leaflets of the native valve (e.g., via the connection between the paddles 2720 and the gripping members 2730), and the leaflets move to the open position (e.g., during the diastolic phase), the paddles 2720 can move to cause the rack 2814, the driven member 2812, and the drive member 2810 to move and engage the biasing member 2750 and cause a force on the biasing member 2750 that allows for the paddles to move to the partially open or open position.

The biasing member 2750 can take any suitable form that is capable of engaging the actuation assembly 2714 such that the paddles are maintained in a closed position but are capable of moving to a partially open or open position when a force is applied to the paddles (e.g., the force applied to the paddles as the leaflets to which the paddles are attached move to an open position during the diastolic phase). For example, the biasing member 2750 can be a spring that is manufactured by cutting (e.g., laser cutting) a flat metal strip or sheet with a desired pattern and then rolling the patterned metal strip or sheet into a tube or other shaped that is capable of acting as a spring. In some implementations, the spring can be cut from a preexisting tube. The biasing member 2750 can be made of, for example, Nitinol or any other suitable material.

Referring now to FIG. 56C, another example of a heart valve device 5600 similar to that shown in FIG. 56 is illustrated. Heart valve device 5600 includes a bias adjustment arrangement or assembly (e.g., clamp, set screw, etc.) that allows for adjustment or modification of the compliance or bias force (including the force range as well) provided by biasing member 2750 (e.g., spring, resilient plug, etc.) and/or that allows for adjustment of the stroke of the drive member 2810. Compliance or bias force adjustment refers to changing the amount of force applied by biasing member 2750 to drive member 2810. This change can include one or more applied bias force values including, for example, the range of force(s) provided or applied by biasing member 2750. The assembly is arranged to position biasing member 2750 in any one or more bias positions with each bias position changing the forces or range of forces provided by biasing member 2750. Biasing member 2750 can thus be arranged to be softer (e.g., more compliant) or stiffer (e.g., more rigid) by the bias adjustment assembly.

The bias adjustment assembly includes a body or base 5602 having a space 5604 configured to receive at least a portion of a positioner element 5606 (e.g., set screw, clamp, threaded member, etc.) Positioner element 5606 is configured to change position in space 5604. This can be accomplished by any suitable means including a threaded arrangement whereby threads are included in space 5604 and on positioner element 5606. With a threaded arrangement, positioner element 5606 can be rotated and thus moved within space 5604. Movement of positioner element 5606 allows biasing member 2750 to either further expand or contract into space or gap 2752. Or, alternatively, gap 2752 can be made large or smaller as positioner element 5606 moves within space 5604. Positioner element 5606 can also include a central passageway 5612, which can be keyed or otherwise formed to allow a turning device to engage with positioner element 5606 to allow for rotational movement along the provided threads to move positioner element 5606.

In operation, positioner element 5606 is placed at a first bias position, location, or state 5608 with space 5604. This places or anchors one end or portion of biasing member 2750 against one end of positioner element 5606 at position 5608. With this bias position or location, biasing member 2750 can provide first compliance or bias force or force range F (1). Positioner element 5606 can be placed at a second bias position, location, or state 5610 (e.g., by an unscrewing or screwing action). This moves or changes the placement or anchor position of biasing member 2750 to position, location, or state 5610. With this bias position, location or state, biasing member 2750 can provide a second compliance or bias force or force range F (2). In some implementations, the first bias force F (1) is different from the second bias force F (2) at these positions or locations. In some implementations, the first bias force range F (1) is less than the second bias force range F (2). In some implementations, the first bias force range F (1) is greater than the second bias force range F (2).

Other arrangements than a threaded arrangement can also be used to allow positioner element 5606 to change positions. This includes, for example, gear and tooth arrangements, snap/step arrangements, cams/cam followers, etc.

Hence, by adjusting the bias position of, for example, positioner element 5606, the force or force range of biasing member 2750 can be changed, modified or adjusted. In some implementations, positioner element 5606 can have other structural shapes and geometries so long as it allows biasing member 2750 to provide an adjustable range of compliance of bias force(s) or force range(s). The bias adjustment assembly can be used or incorporated into any of the devices described herein.

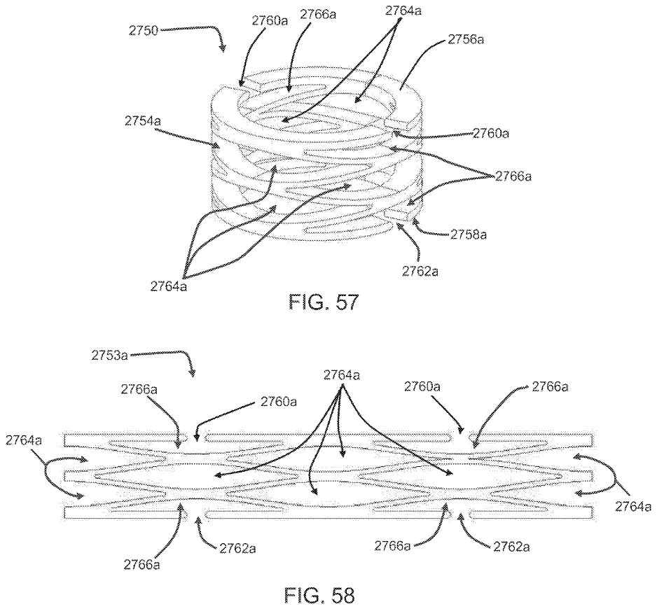

Referring to FIG. 57, in some implementations, the biasing member 2750 can include a body 2754a that has a top surface 2756a and bottom surface 2758a. The top surface 2756a includes one or more slits 2760a, and the bottom surface 2758a can include one or more slits 2762a. While the illustrated example shows the body 2754a having two slits 2760a on the top surface 2756a and two slits 2762a on the bottom surface 2758a, it should be understood that the top and bottom surfaces 2756a, 2758a can have any suitable number of slits.

In some implementations, the body 2754a can include a plurality of first cutouts or openings 2764a and a plurality of second cutouts or openings 2766a. The first openings 2764a can be positioned within a central portion of the body 2754a between the top and bottom surfaces 2756a, 2758a. In the illustrated example, the first openings 2764a have a diamond shape, but it should be understood that the first openings 2764a can have any other suitable shape. Each of the first openings 2764a can have the same shape, or one or more of the first openings 2764a can have different shapes.

In some implementations, the second openings 2766a can be positioned proximate the top and bottom surfaces 2756a, 2758a. In some implementations, one or more of the second openings 2766a can be positioned proximate the one or more slits 2760a, 2762a such that the slits 2760a, 2762a extend into the second openings 2766a. In the illustrated example, the second openings 2766a have a triangular shape, but it should be understood that the second openings 2766a can have any other suitable shape. Each of the second openings 2766a can have the same shape, or one or more of the second openings 2766a can have different shapes. FIG. 58 illustrates an example laser cut sheet 2753a for making the body 2754a shown in FIG. 57, where the sheet 2753a includes the slits 2760a, 2762a, the first openings 2764a, and the second openings 2766a.

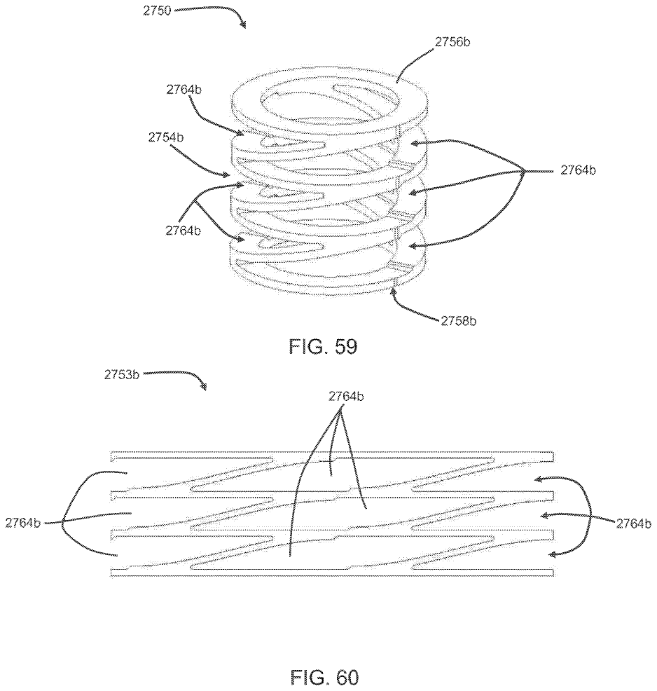

Referring to FIG. 59, in some implementations, the biasing member 2750 can include a body 2754b that has a top surface 2756b and bottom surface 2758b. The body 2754b can include a plurality of cutouts or openings 2764b between the top and bottom surfaces 2756b, 2758b. The openings 2764b can have any suitable shape. Each of the first openings 2764a can have the same shape, or one or more of the openings 2764b can have different shapes. FIG. 60 illustrates an example laser cut sheet 2753b for making the body 2754b shown in FIG. 59, where the sheet 2753b includes the openings 2764b.

In some implementations, the paddles 2720 can be independently controllable. For example, the device 2700 can have two independent drive assemblies 2714 with two independent racks 2814. In some implementations, each independent drive assembly 2714 can include a gap 2752 and biasing member 2750 that allows the paddles 2720 to independently move between various positions (e.g., the closed position, the partially open position, the open position, etc.) in response to movement of the leaflets of the native valve (e.g., movement of the leaflets to the open position during the diastolic phase) when the device is attached to the leaflets, thus reducing the stress on the leaflets due to the connection between the device 2700 and the leaflets. In some implementations, the gripping elements 2730 can be independently controllable, such as, for example, any means described for independently controlling gripping elements described in the present disclosure.

In some implementations, the implantable device 2700 can be delivered and implanted within the native mitral valve MV or the native tricuspid valve TV of the heart H. For example, when implanted in the tricuspid valve TV the delivery sheath can be inserted into the left atrium LA through the septum and the device 2700 is deployed from the delivery sheath. The device 2700 can be moved into position within the mitral valve MV into the ventricle LV and partially opened so that the leaflets 20, 22 can be grasped.

In some implementations, the device 2700 can then be decoupled from the implant catheter assembly. In some implementations, the sheath and actuation lines are then retracted leaving the device 2700 deployed in the native mitral valve MV.

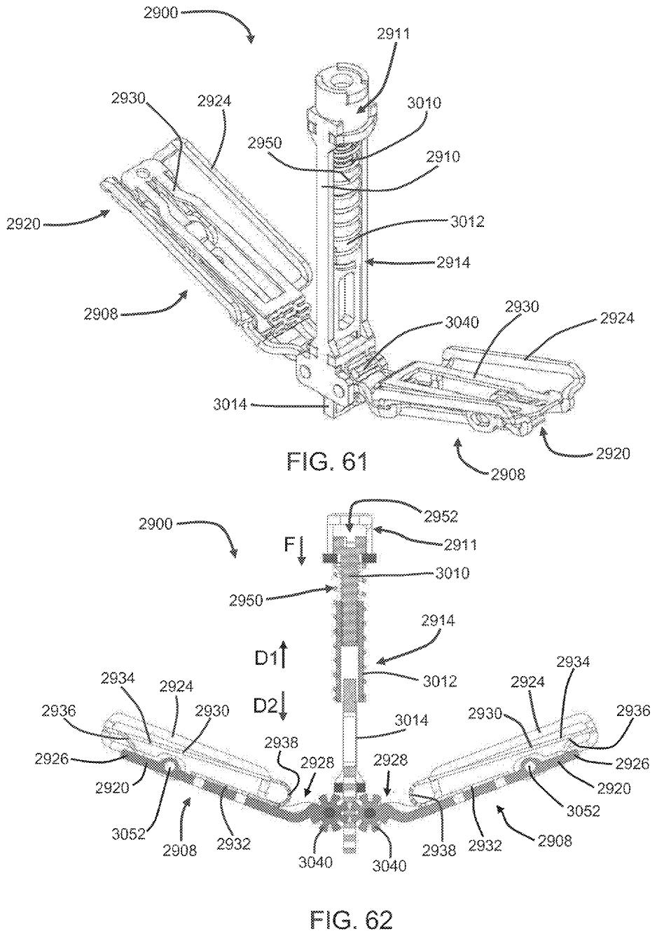

Referring now to FIGS. 61-62, an example of a device 2900 (e.g., a treatment device, a repair device, a prosthetic device, a valve repair device, valve treatment device, implantable device, implant, etc.) is shown. The device 2900 can include any features for a device discussed in the present disclosure, and the device 2900 can be positioned to engage valve tissue 20, 22, 30, 32, 34 as part of any suitable treatment and/or repair system (e.g., any treatment and/or repair system described in the present disclosure).

In some implementations, the device 2700 can comprise an optional coupling 2911, a base 2910, an actuation assembly 2914, and one or more anchors 2908. In some implementations, the coupling 2911 is connected to a proximal end of the base 2910. In some implementations, the actuation assembly 2914 is housed at least partially within the base 2910. In some implementations, the one or more anchors can comprise one anchor 2908 (e.g., a device configured to attach to only a single native valve leaflet), two anchors 2908 (e.g., a device configured to attach to two leaflet, such as the leaflets of the mitral valve or two leaflets of the tricuspid valve), or three anchors 2908 (e.g., a device configured to attach to the three leaflets of the tricuspid valve).

In some implementations, the one or more anchors 2908 are coupled to the actuation assembly 2914 such that operation of the actuation assembly 2914 can move the anchors between two or more of a closed position (e.g., similar to the position of the device 2700 shown in FIG. 54), a partially open or capture ready position (e.g., similar to the position of the device 1500 shown in FIG. 40), a fully open position (as shown in FIGS. 61 and 62), and an extended position (e.g., similar to the position of the device 1500 shown in FIG. 43).

The coupling 2911 can take a variety of different forms. For example, the coupling 2911 can have any of the features of any of the couplings or proximal collars described in the present disclosure and/or can have any of the features of any of the couplings or proximal collars disclosed in PCT Application No. PCT/US2018/028189, published as PCT Publication WO2018/195215.

In some implementations, an implantable device 2900 has a coupling 2911 that accepts an actuation element (e.g., any actuation element described in the present disclosure). The actuation element can extend through an implant catheter (e.g., any implant catheter described in the present disclosure) and through a coupler (e.g., any coupler described in the present disclosure) that extends from or is extendable from a distal end of the implant catheter. The actuation element can be coupled to the device 2900 to open and close the anchor(s) 2908 of the device 2900.

The base 2910 can take a variety of different forms, such as, for example, any form described for the base 1510 shown in FIGS. 31-43. In some implementations, the base 2910 can be any of the coaption elements described in the present disclosure. The base can be formed from a single component or can be assembled from a plurality of components. In some implementations, the base 2910 comprises a frame with an open interior for other components of the device, such as a metal frame.

The actuation assembly 2914 can take a variety of different forms, such as, for example, any form described for actuation assembly 1514 shown in FIGS. 31-43. For example, the actuation assembly 2914 can be any of the actuation elements, actuation assemblies, and/or actuation linkages described in the present disclosure. In some implementations, the actuation assembly 2914 can be comprised of longitudinally movable components, such as one or more shafts, rods, tubes and/or can comprise one or more assemblies that convert rotational movement to translational movement, such as gear assemblies (e.g., rack and pinion assemblies, worm gear assemblies etc.) and/or that convert rotational movement about an axis in first plane to rotational movement about an axis in a second plane, that can be orthogonal to the first plane (e.g., worm gear assemblies, planetary gear assemblies, etc.) The actuation assembly 2914 can take any form where a longitudinal movement and/or a rotational movement is used to open and/or close one or more components of the anchors 2908.

In some implementations, the actuation assembly 2914 comprises a drive member 3010, a driven member 3012, and a rack 3014. The drive member 3010 can take a variety of different forms, such as any form described for the drive member 1910 shown in FIGS. 31-43. In some implementations, the drive member 3010 comprises a head, a neck, and a threaded portion. In some implementations, the head can be configured to be engaged by a drive end of the actuation element. For example, the head and the drive end can have a corresponding mating feature, such as slotted, Phillips, hex, star, or any other mating shapes. Any configuration that allows the actuation element to drive the head can be used.

In some implementations, the drive member 3010 is configured to be constrained by the base 2910 such that the drive member 3010 is rotatable relative to the base 2910 but cannot move longitudinally relative to the base. For example, the drive member 3010 can be constrained by the base 2910 in a similar manner in which drive member 1910 is constrained by the base 1510 described in the present disclosure.

The driven member 3012 can take a variety of different forms, such as any form described for the driven member 1912 shown in FIGS. 31-43. In some implementations, the driven member 3012 can have a threaded portion and a coupling end portion. In some implementations, the threaded portion can be configured to be mated with the threaded portion of the drive member 2810. For example, the rotation of the drive member 3010 can longitudinally move the driven member 3012. The threaded portion can be a male threaded portion that engages with a female threaded portion, or the threaded portion can be a female threaded portion that engages with a male threaded portion. The coupling end portion can take a variety of different forms, such as any form described for the drive member 1932 of the driven member 1912 shown in FIGS. 31-43.

The rack 3014 can take a variety of different forms, such as any form described for the rack 1914 shown in FIGS. 31-43. In some implementations, the rack 3014 is a gear rack that is configured to drive one or two pinion gears 3040. A rack 3014 can be configured to engage two pinion gears 3040 in a variety of different ways. For example, teeth can be provided on both sides of the rack. In some implementations, the rack 3014 includes spaced apart openings that are sized and spaced to accept teeth of two pinion gears 3040 on opposite sides of the rack 3014.

The engagement and interaction between the drive member 3010 and the driven member 3012 can take any suitable form that allows for the drive member 3010 to drive the driven member 3012, such as, for example, any form described with reference to the drive member 1910 and the driven member 1912 shown in FIGS. 31-43. The engagement and interaction between the driven member 3012 and the rack 3014 can take any suitable form that allows for the rack to be moved in directions D1, D2 (FIG. 62) such that the rack engages the pinion gears 3040 of the paddles 2920 of the anchors 2908 to move the paddles 2920 between two or more of the closed position, the partially open position, the open position, and the extended position.

The one or more anchors 2908 can take a variety of different forms, such as, for example, any form described for the anchors 1508 shown in FIGS. 31-43. The one or more anchors 2908 can include one or more of the features of any of the anchors described in the present disclosure. In some implementations, each of the anchors 2908 include a paddle 2920, an optional paddle frame 2924, and an optional gripping member 2930, such as a clasp. The paddle 2920 can take a variety of different forms.

In some implementations, the paddle 2920 can extend from a pivotable portion 2928 to a free end 2926. In some implementations, the pivotable portion 2928 can include the pinion gear 3040. In some implementations, the rack 3014 drives or rotates the pinion gear 3040 to move the paddle 2920 between two or more of the closed position, the partially open position, the open position, and the extended position.

In some implementations, the pivotable portion 2928 can include a passage (e.g., similar to passage 1950 for the paddle 1520 shown in FIG. 36) that accepts the pivot member of the base 2910 (e.g., similar to the pivot member 1824 of the base 1510 for the device 1500 shown in FIGS. 31-43) to allow the paddle 2920 to pivot relative to the base 2910. In some implementations, the paddle 2920 includes a passage 3052 that accepts a paddle frame attachment member (e.g., rod, shaft, bar, threaded fastener, pin, bar, etc.)

The paddle frame 2924 can take a variety of different forms, such as, for example, any form described for the paddle frame 1524 shown in FIGS. 31-43, or any features of any of the paddle frames described in the present disclosure. The paddle frame 2924 can be connected to the paddle 2920 in any suitable manner, such as, for example, any connection described between the paddle frame 1524 and the paddle 1520 shown in FIGS. 31-43.

In some implementations, the paddle frames 2924 are configured to press, coapt, bring together, or bring closer together two native valve leaflets (e.g., leaflets 20, 22 or two of leaflets 30, 32, 34). In some implementations, the paddle frames 2924 comprise one, two, or more separate components that are attached to or coupled to the paddles 2920. In some implementations, the paddle frames 2924 can be omitted.

In some implementations, the paddles 2920 can be shaped to press, coapt, bring together, or bring closer together two native valve leaflets (e.g., leaflets 20, 22 or two of leaflets 30, 32, 34), without having an additional paddle frame component. The paddle frame 2924 can be made from a variety of different materials and can be shaped in a variety of different ways. In some implementations, one or more components of the paddle frame 2924 can be formed (e.g., cut and bent) from sheet material, can be molded, can be cast, 3-D printed, etc.).

The optional gripping member 2930 can take a variety of different forms. For example, the gripping member 2930 can have any of the features of any of the gripping members or clasps described in the present disclosure. In some implementations, the gripping members 2930 comprise a base or fixed arm 2732, a movable arm 2734, optional barbs 2736, and a joint portion 2738.

In some implementations, the fixed arms 2932 can be attached to the paddles 2920, with the joint portion 2938 disposed proximate the base 2910. The fixed arms 2932 can be attached to the paddles 2920 with any suitable means, such as sutures, screws or other fasteners, crimped sleeves, mechanical latches or snaps, welding, adhesive, or the like. In some implementations, the fixed arms 2932 remain substantially stationary relative to the paddles 2920 when the movable arms 2934 are opened to open the gripping members 2930.

In some implementations, the gripping members 2930 are opened by applying tension to actuation lines (e.g., sutures, wires, etc.) attached to the movable arms 2934, thereby causing the movable arms 2934 to pivot on the joint portions 2938.

In some implementations, during treatment and/or implantation, the anchors 2908 are opened and closed to grasp the native mitral valve leaflets between the paddles 2920 and the base 2910. The optional gripping members 2930 can further secure the native leaflets by pinching the leaflets between the movable and fixed arms 2934, 2932. The optional barbs 2936 can increase friction with the leaflets or can partially or completely puncture the leaflets.

In some implementations, the actuation lines can be actuated separately so that each clasp 2930 can be opened and closed separately. Separate operation allows one leaflet to be grasped at a time, or for the repositioning of a gripping element 2930 on a leaflet, without altering a successful grasp on the other leaflet. In some implementations, the gripping members 2930 can open and close when the paddle 2920 is not closed, thereby allowing leaflets to be grasped in a variety of positions as the particular situation requires.

The device 2900 can be configured to be removably connected to an implant catheter assembly (not shown), where the implant catheter assembly can include an actuation element for engaging the actuation assembly 2914 of the device 2900. The implant catheter assembly can take any suitable form that is capable of removably attaching to the coupling 2911 of the device 2900, such as, for example, any form of an implant catheter assembly described in the present disclosure. The actuation element of the implant catheter assembly can take any suitable form that is capable of engaging the actuation assembly 2914 of the device 2900 to move the paddles 2720 between two or more of the closed position, the partially open position, the open position, and the extended position.

In some implementations, the paddles 2920 can be moved by rotating the actuation element to cause the drive member 3010 to rotate, which consequently causes the driven member 3012 to engage the rack 3014 and cause the rack 3014 to move in a proximal direction D1 or a distal direction D2 (FIG. 62). In some implementations, movement of the rack 3014 in the distal direction D2 causes the paddles 2920 to move toward the closed position, and movement of the rack 3014 in the proximal direction D1 causes the paddle 2920 to move toward at least one of the partially open, the open, and the extended positions. In some implementations, the engagement between the rack 3014 and the pinion gears 3040 allows for the movement of the paddles 2920 between two or more of the closed position, the partially open position, the open position, and the extended position. The optional paddle frames 2924 can be moved with the paddles 2920 due to a connection between the paddles 2920 and the paddle frames 2924.

In some implementations, the device 2900 can be deployed from a delivery sheath (see, e.g., delivery sheath 2606). In some implementations, the base 2910 of the device 2900 can be implanted between the leaflets of the native mitral valve or tricuspid valve. The anchors 2908 are actuatable between open and closed conditions. In some implementations, during implantation, the paddles 2920 are opened and closed to grasp the native mitral valve leaflets between the paddles 2920 and the base 2910.

The device 2900 can be configured such that the paddles 2920 are able to move between various positions (e.g., the closed position, the partially open position, the open position, etc.) in response to movement of the leaflets of the native valve (e.g., movement of the leaflets to the open position during the diastolic phase) when the device is attached to the leaflets. The movability of the paddles 2920 is advantageous because it reduces the stress on the leaflets resulting from the attachment between the device 2900 and the leaflets.

In the illustrated example, the device 2900 has a gap 2952 positioned within the coupling 2911 proximate the drive member 3010 of the actuation assembly 2914, which allows for the drive member 3010 to move in the proximal direction D1 and/or distal direction D2 within the gap 2952. The gap 2952 allows for the actuation assembly 2914 to be engaged in a reverse direction with movement of the paddles 2920 causing movement of the actuation assembly 2914, as compared to normal movement of the device in which the actuation assembly causes movement of the paddles 2920.

In some implementations, the height of the gap 2952 controls the distance at which the drive member 3010 is able to move in the proximal direction D1 and/or distal direction D2 and, consequently, controls the amplitude at which the paddles 2920 are able to flex as a result of the moving of the leaflets of the native valve. In some implementations, the height of the gap 2952 can be between about 0.2 mm and about 4 mm, such as between 0.3 mm and 3 mm, such as between 0.4 mm and 2.5 mm, such as between 0.5 mm and 2 mm, or any sub range of these ranges. While the gap 2952 is shown being positioned within the coupling 2911 of the device 2900, it should be understood that the gap 2952 can be positioned at any suitable location that allows for the actuation assembly 2914 to move in response to the paddles 2920 moving to an open position due to the attachment between the device 2900 and the leaflets.

In some implementations, in which movement of the rack 3014 in the distal direction D2 (FIG. 62) causes the paddles 2920 to move to the closed position, the opening of the leaflets (e.g., during the diastolic phase) can cause the paddles 2920 to move to the partially open or open position, which causes the rack 3014 to move in the proximal direction D1 (via the connection between the rack 3014 and the pinion gears 3040), which consequently causes the driven member 3012 and the drive member 3010 to move in the proximal direction D1. The gap 2952 allows for such movement of the drive member 3010 in the proximal direction D1, thus allowing the paddle 2920 to move to the partial open or open position.

In some implementations, the device 2900 can include a biasing member 2950 that is configured to engage the actuation assembly 2914 to maintain, e.g., bias the paddles 2920 toward the closed position when the device 2900 is attached to the leaflets of the native valve, while allowing for the paddles to move to the open position in response to the opening of the leaflets (e.g., during the diastolic phase).

In some implementations, as illustrated in FIG. 62, the biasing member 2950 is attached to the driven member 3012 and the coupler 2911 such that the biasing member 2950 maintains a force F on the driven member 3012 to bias the paddles 2920 toward the closed position. In some implementations, the biasing member 2950 can be attached to the driven member 3012 and any other portion of the device 2900 (e.g., the base 2910) that causes the force F on the driven member 3012 that biases the paddles 2920 toward the closed position. In some implementations, rather than the biasing member 2950 causing a force F on the drive member 3020 to bias the paddles 2920 toward the closed position, the biasing member 2950 can be attached to other portions of the actuation assembly 2914 that causes the force F on the actuation assembly 2914 that biases the paddles 2920 toward the closed position.

In some implementations, the amplitude at which the paddles 2920 are able to flex as a result of the moving of the leaflets of the native valve the gap 2952 is adjustable. This amplitude can be adjustable in a variety of different ways. In some implementations, the gap is adjustable. By adjusting the gap 2952 the amount or magnitude that the paddles 2920 can open can be adjusted. For example, the gap can be adjusted between a position that allows a maximum amount of paddle opening and a position that prevents any opening of the paddles 2920.

The gap 2952 can be adjusted in a variety of different ways. In some implementations, the gap 2952 can be adjusted by rotating the drive member 3010 beyond a position where the paddles are fully closed. This causes threads of the drive member 3010 and driven member 3012 to push the drive member proximal and reduce the gap. The gap can be at a maximum where the drive member 3010 moves the paddles 2920 to the closed position, but no further rotation of the drive member 3010 occurs after the paddles are fully closed. In some implementations, the paddles can move a maximum amount with the native leaflets when the gap is at a maximum. The gap can be at a minimum when the drive member 3010 is continued to be rotated as far as possible after the paddles are fully closed. When the gap is at a minimum, the paddles 2920 can be rigid or cannot move as the native valve leaflets move.

In some implementations, in which movement of the rack 3014 in the distal direction D2 causes the paddles 2920 to move to the closed position, the biasing member 2950 can be configured to engage the drive member 3010 to provide a force F (FIG. 62) on the drive member 3010 to bias the paddles 2920 toward the closed position. When the device 2900 is attached to the leaflets of the native valve (e.g., via the connection between the paddles 2920 and the gripping members 2930), and the leaflets move to the open position (e.g., during the diastolic phase), the paddles 2920 can move to cause the rack 3014, the driven member 3012, and the drive member 3010 to move and engage the biasing member 2950 and cause a force on the biasing member 2950 that allows for the paddles to move to the partially open or open position.

The biasing member 2950 can take any suitable form that is capable of engaging the actuation assembly 2914 such that the paddles are maintained in a closed position but capable of moving to a partially open or open position when a force is applied to the paddles (e.g., the force applied to the paddles as the leaflets to which the paddles are attached move to an open position during the diastolic phase). For example, the biasing member 2950 can be a spring that takes the form of any spring described in the present disclosure. In the illustrated, example, the biasing member 2950 is a coiled wire spring.

In some implementations, the paddles 2920 can be independently controllable. For example, the device 2900 can have two independent drive assemblies 2914 with two independent racks 3014. In some implementations, each independent drive assembly 2914 can include a gap 2952 and biasing member 2950 that allows the paddles 2920 to independently move between various positions (e.g., the closed position, the partially open position, the open position, etc.) in response to movement of the leaflets of the native valve (e.g., movement of the leaflets to the open position during the diastolic phase) when the device is attached to the leaflets, thus reducing the stress on the leaflets due to the connection between the device 2900 and the leaflets. In some implementations, the gripping elements 2930 can be independently controllable, such as, for example, any means described for independently controlling gripping elements described in the present disclosure.

In some implementations, the implantable device 2900 can be delivered and implanted within the native mitral valve MV or the native tricuspid valve TV of the heart H. For example, when implanted in the tricuspid valve TV the delivery sheath can be inserted into the left atrium LA through the septum and the device 2900 is deployed from the delivery sheath. The device 2900 can be moved into position within the mitral valve MV into the ventricle LV and partially opened so that the leaflets 20, 22 can be grasped.

In some implementations, the device 2900 can then be decoupled from the implant catheter assembly. In some implementations, the sheath and actuation lines are then retracted leaving the device 2900 deployed in the native mitral valve MV.

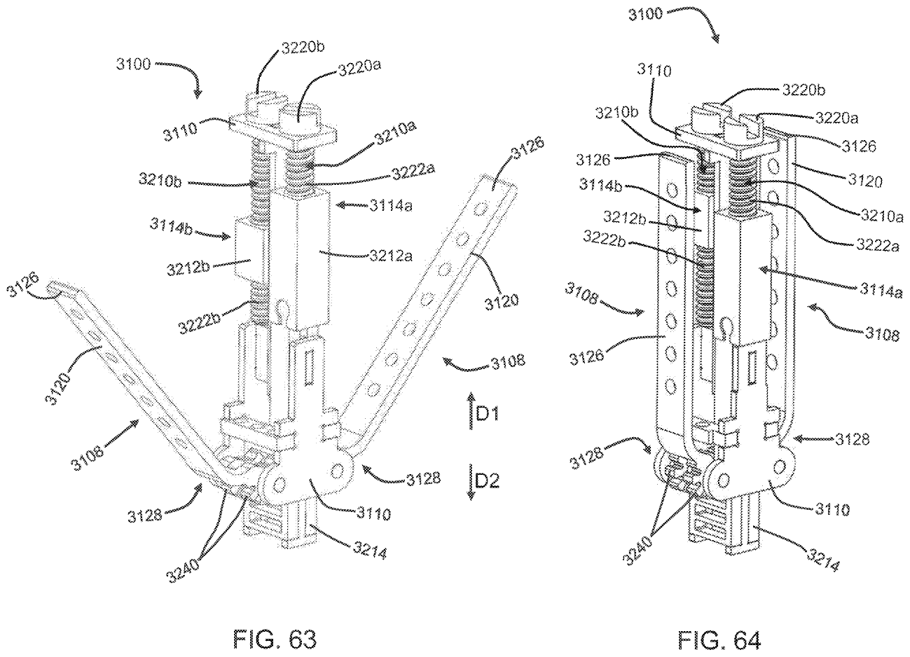

Referring now to FIGS. 63-67, an example of a device 3100 (e.g., a treatment device, a repair device, a prosthetic device, a valve repair device, valve treatment device, implantable device, implant, etc.) is shown. The device 3100 can include any features for a device discussed in the present disclosure, and the device 3100 can be positioned to engage valve tissue 20, 22, 30, 32, 34 as part of any suitable treatment and/or repair system (e.g., any treatment and/or repair system described in the present disclosure).

The device 3100 can comprise an optional coupling (not shown), a base 3110, a first actuation assembly 3114a, a second actuation assembly 3114b, and one or more anchors 3108. In some implementations, the coupling is connected to a proximal end of the base 3110. In some implementations, one or both of the first actuation assembly 3114a and the second actuation assembly 3114b are housed at least partially within the base 3110. In some implementations, the one or more anchors can comprise one anchor 3108 (e.g., a device configured to attach to only a single native valve leaflet), two anchors 3108 (e.g., a device configured to attach to two leaflets, such as the leaflets of the mitral valve or two leaflets of the tricuspid valve), or three anchors 3108 (e.g., a device configured to attach to the three leaflets of the tricuspid valve).

In some implementations, the one or more anchors 3108 are coupled to the first actuation assembly 3114a, such that operation of the first actuation assembly 3114a can move the anchors between two or more of a closed position (see FIG. 64), a partially open or capture ready position (e.g., similar to the position of the device 1500 shown in FIG. 40), a fully open position (e.g., similar to the position of the device 1500 shown in FIG. 41), and an extended position (e.g., similar to the position of the device 1500 shown in FIG. 43).

The coupling can take a variety of different forms. For example, the coupling can have any of the features of any of the couplings or proximal collars described in the present disclosure and/or can have any of the features of any of the couplings or proximal collars disclosed in PCT application No. PCT/US2018/028189, published as PCT Publication WO2018/195215.

In some implementations, an implantable device 2700 has a coupling that accepts an actuation element (e.g., any actuation element described in the present disclosure). The actuation element can extend through an implant catheter (e.g., any implant catheter described in the present disclosure) and through a coupler (e.g., any coupler described in the present disclosure) that extends from or is extendable from a distal end of the implant catheter. The actuation element can be coupled to the device 2700 to open and close the anchor(s) 3108 of the device 3100.

The base 3110 can take a variety of different forms, such as, for example, any form described for the base 1510 shown in FIGS. 31-43. In some implementations, the base 3110 can be any of the coaption elements described in the present disclosure. The base can be formed from a single component or can be assembled from a plurality of components. In some implementations, the base 3110 comprises a frame with an open interior for other components of the device, such as a metal frame.

The first actuation assembly 3114a can take a variety of different forms, such as, for example, any form described for actuation assembly 1514 shown in FIGS. 31-43. For example, the first actuation assembly 3114a can be any of the actuation elements, actuation assemblies, and/or actuation linkages described in the present disclosure. In some implementations, the first actuation assembly 3114a can be comprised of longitudinally movable components, such as one or more shafts, rods, tubes and/or can comprise one or more assemblies that convert rotational movement to translational movement, such as gear assemblies (e.g., rack and pinion assemblies, worm gear assemblies etc.) and/or that convert rotational movement about an axis in a first plane to rotational movement about an axis in a second plane, that can be orthogonal to the first plane (e.g., worm gear assemblies, planetary gear assemblies, etc.) The first actuation assembly 3114a can take any form where a longitudinal movement and/or a rotational movement is used to open and/or close one or more components of the anchors 3108.

In some implementations, the first actuation assembly 3114a comprises a drive member 3210a, a driven member 3212a, and a rack 3214. The drive member 3210a can take a variety of different forms, such as any form described for the drive member 1910 shown in FIGS. 31-43. In some implementations, the drive member 3210a comprises a head 3220a, a neck (not shown), and a threaded portion 3222a.

In some implementations, the head 3220a can be configured to be engaged by a drive end of the actuation element. For example, the head 3220a and the drive end can have a corresponding mating feature, such as slotted, Phillips, hex, star, or any other mating shapes. Any configuration that allows the actuation element to drive the head 3220a can be used.

In some implementations, the drive member 3210a is configured to be constrained by the base 3110 such that the drive member 3210a is rotatable relative to the base 3110 but cannot move longitudinally relative to the base. For example, the drive member 3210a can be constrained by the base 3110 in a similar manner in which drive member 1910 is constrained by the base 1510 described in the present disclosure.

The driven member 3212a can take a variety of different forms, such as any form described for the driven member 1912 shown in FIGS. 31-43. In some implementations, the driven member 3212a can have a threaded portion 3230 (FIG. 67) and a coupling end portion 3232 (FIG. 67). In some implementations, the threaded portion 3230 can be configured to be mated with the threaded portion 3222a of the drive member 3210a. For example, the rotation of the drive member 3210a can longitudinally move the driven member 3212a. The threaded portion 3222a can be a male threaded portion that engages with a female threaded portion 3230 or the threaded portion 3222a can be a female threaded portion that engages with a male threaded portion 3230. The coupling end portion 3232 can take a variety of different forms, such as any form described for the drive member 1932 of the driven member 1912 shown in FIGS. 31-43.

The rack 3214 can take a variety of different forms, such as any form described for the rack 1914 shown in FIGS. 31-43. In some implementations, the rack 3214 is a gear rack that is configured to drive one or more pinion gears 3240. A rack 2814 can be configured to engage pinion gears 3240 in a variety of different ways. For example, teeth can be provided on both sides of the rack. In some implementations, the rack 3214 includes spaced apart openings that are sized and spaced to accept teeth of pinion gears 3240 on opposite sides of the rack 3214.

The engagement and interaction between the drive member 3210a and the driven member 3212a can take any suitable form that allows for the drive member 3210a to drive the driven member 3212a, such as, for example, any form described with reference to the drive member 1910 and the driven member 1912 shown in FIGS. 31-43. The engagement and interaction between the driven member 3212a and the rack 3214 can take any suitable form that allows for the rack to be moved in directions D1, D2 (FIG. 63) such that the rack engages the pinion gears 3240 of the paddles 3120 of the anchors 3108 to move the paddles 3120 between two or more of the closed position, the partially open position, the open position, and the extended position.

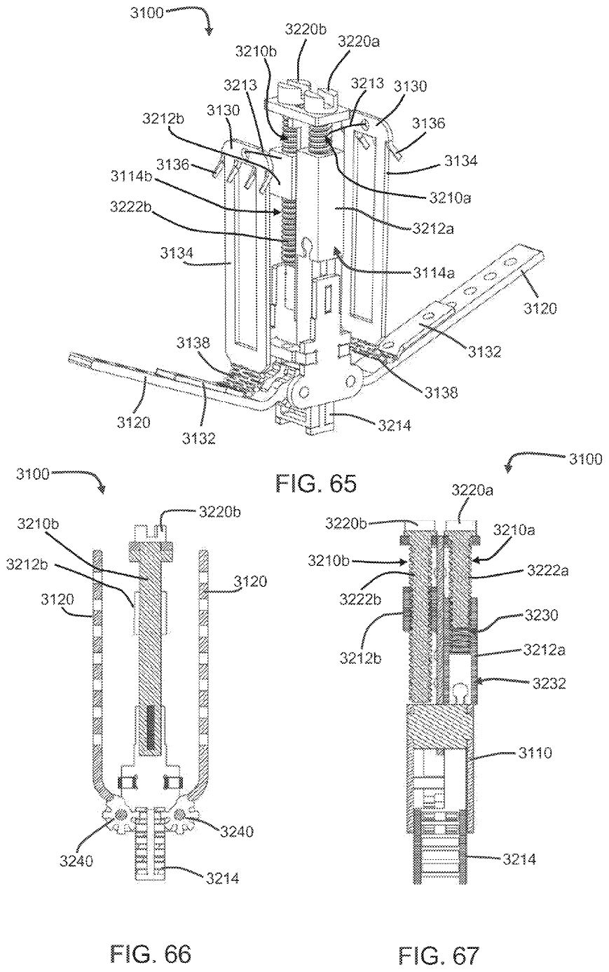

The one or more anchors 3108 can take a variety of different forms, such as, for example, any form described for the anchors 1508 shown in FIGS. 31-43. The one or more anchors 3108 can include one or more of the features of any of the anchors described in the present disclosure. In some implementations, each of the anchors 3108 include a paddle 3120, an optional paddle frame (not shown), and an optional gripping member 3130 (FIG. 65), such as a clasp.

The paddle 3120 can take a variety of different forms. In some implementations, the paddle 3120 can extend from a pivotable portion 3128 to a free end 3126. In some implementations, the pivotable portion 3128 can include one or more pinion gear 3240. In the illustrated example, each paddle 3120 includes two pinion gears 3240. In some implementations, the rack 3214 drives or rotates the pinion gear(s) 3240 to move the paddle 3120 between two or more of the closed position, the partially open position, the open position, and the extended position.

In some implementations, the pivotable portion 3128 can include a passage (e.g., similar to passage 1950 for the paddle 1520 shown in FIG. 36) that accepts the pivot member of the base 3110 (e.g., similar to the pivot member 1824 of the base 1510 for the device 1500 shown in FIGS. 31-43) to allow the paddle 3120 to pivot relative to the base 3110. In some implementations, the paddle 3120 includes a passage (not shown) that accepts a paddle frame attachment member (e.g., rod, shaft, bar, threaded fastener, pin, bar, etc.).

The optional paddle frame can take a variety of different forms, such as, for example, any form described for the paddle frame 1524 shown in FIGS. 31-43, or any features of any of the paddle frames described in the present disclosure. The paddle frame can be connected to the paddle 3120 in any suitable manner, such as, for example, any connection described between the paddle frame 1524 and the paddle 1520 shown in FIGS. 31-43.

In some implementations, the paddle frames can be configured to press, coapt, bring together, or bring closer together two native valve leaflets (e.g., leaflets 20, 22 or two of leaflets 30, 32, 34). In some implementations, the paddle frames comprise one, two, or more separate components that are attached to or coupled to the paddles 3120. In some implementations, one or more components of the paddle frame can be formed (e.g., cut and bent) from sheet material, can be molded, can be cast, 3-D printed, etc.).

The optional gripping member 3130 can take a variety of different forms. For example, the gripping member 3130 can have any of the features of any of the gripping members or clasps described in the present disclosure. Referring to FIG. 65, in some implementations, the gripping members 3130 comprise a base or fixed arm 3132, a movable arm 3134, optional barbs 3136, and a joint portion 3138.

In some implementations, the fixed arms 3132 can be attached to the paddles 3120, with the joint portion 3138 disposed proximate the base 3110. The fixed arms 3132 can be attached to the paddles 3120 with any suitable means, such as sutures, screws or other fasteners, crimped sleeves, mechanical latches or snaps, welding, adhesive, or the like. In some implementations, the fixed arms 3132 remain substantially stationary relative to the paddles 3120 when the movable arms 3134 are opened to open the gripping members 3130.

The second actuation assembly 3114b can take a variety of different forms. In some implementations, the second actuation assembly 3114b can include one or more features of actuation assembly 1514 shown in FIGS. 31-43 or any of the actuation elements, actuation assemblies, and/or actuation linkages described in the present disclosure. In some implementations, the second actuation assembly 3114b can be comprised of longitudinally movable components, such as one or more shafts, rods, tubes and/or can comprise one or more assemblies that convert rotational movement to translational movement, such as gear assemblies (e.g., rack and pinion assemblies, worm gear assemblies etc.) and/or that convert rotational movement about an axis in first plane to rotational movement about an axis in a second plane, that can be orthogonal to the first plane (e.g., worm gear assemblies, planetary gear assemblies, etc.) The second actuation assembly 3114b can take any form where a longitudinal movement and/or a rotational movement is used to control one or more components (e.g., gripping members, paddle frame element(s), component(s) of the base, etc.) of the device 3100.

In some implementations, the second actuation assembly 3114b comprises a drive member 3210b and a driven member 3212b. The drive member 3210b can take a variety of different forms, such as any form described for the drive member 1910 shown in FIGS. 31-43. In some implementations, the drive member 3210b comprises a head 3220b, a neck (not shown), and a threaded portion 3222b.

In some implementations, the head 3220b can be configured to be engaged by a drive end of the actuation element. For example, the head 3220b and the drive end can have a corresponding mating feature, such as slotted, Phillips, hex, star, or any other mating shapes. Any configuration that allows the actuation element to drive the head 3220b can be used.

In some implementations, the drive member 3210b is configured to be constrained by the base 3110 such that the drive member 3210b is rotatable relative to the base 3110 but cannot move longitudinally relative to the base. For example, the drive member 3210b can be constrained by the base 3110 in a similar manner in which drive member 3210a of the first actuation assembly 3114a is constrained by the base 3110.

The driven member 3212b can take a variety of different forms. In some implementations, the driven member 3212b can be threaded (as shown in FIG. 67) such that the threaded driven member 3212b can be configured to mate with the threaded portion 3222b of the drive member 3210b. For example, the rotation of the drive member 3210b can longitudinally move the driven member 3212b. The threaded driven member 3212b can include male threads that are configured to engage with female threads of the threaded portion 3222b, or the threaded driven member 3212b can include female threads that are configured to engage with male threads of the threaded portion 3222b.

The engagement and interaction between the drive member 3210b and the driven member 3212b can take any suitable form that allows for the drive member 3210b to drive the driven member 3212b, such as, for example, any form described with reference to the drive member 1910 and the driven member 1912 shown in FIGS. 31-43. The driven member 3212b can be operatively connected to one or more components (e.g., gripping members, paddle frame element(s), component(s) of the base, etc.) of the device 3100 such that movement of the driven member 3212b in the directions D1, D2 (FIG. 63) actuates the connected components.

Referring to FIG. 65, in some implementations, the gripping members 3130 are operatively connected to the second actuation assembly 3114b such that the second actuation assembly 3114b can be engaged to move the gripping members 3130 between open and closed positions. For example, the movable arm 3134 of the gripping member 3130 can be connected to the driven member 3212b by one or more actuation lines 3213 (e.g., suture(s), wire(s), etc.) such that movement of the driven member 3212b in the directions D1, D2 (FIG. 63) causes the actuation lines 3213 to engage the movable arm 3134 and cause the movable arm 3134 to pivot at the joint portion 3138 to move the gripping member 3130 between the open and closed positions.

In some implementations, movement of the driven member 3212b in the proximal direction D1 causes the gripping member 3130 to move to the open position, and movement of the driven member 3212b in the distal direction D2 causes the gripping member 3130 to move to the closed position. In some implementations, movement of the driven member 3212b in the distal direction D2 causes the gripping member 3130 to move to the open position, and movement of the driven member 3212b in the proximal direction D1 causes the gripping member 3130 to move to the closed position.

In some implementations, each gripping member 3130 is attached to the same driven member 3212b such that the gripping members 3130 are opened simultaneously by movement of the driven member 3212b. In some implementations, the device 3100 can include one or more additional actuation assemblies that are similar to the actuation assembly 3114b, and each gripping member 3130 is attached to a different actuation assembly such that the gripping members 3130 can be opened independently from each other.

In some implementations, the gripping members 3130 are not operatively connected to the actuation assembly 3114b, but one or more other components of the device can be operatively connected to the actuation assembly 3114b. In these implementations, one or more actuation lines (e.g., sutures, wires, etc.) can be attached to the gripping members 3130 such that a user can engaged the actuation lines to move the gripping members 3130 between the open and closed positions. For example, the gripping members 3130 can be opened by applying tension to actuation lines (e.g., sutures, wires, etc.) that are attached to the movable arms 3134, thereby causing the movable arms 3134 to pivot on the joint portions 3138.

In some implementations, the actuation lines can be actuated separately so that each clasp 3130 can be opened and closed separately. Separate operation allows one leaflet to be grasped at a time, or for the repositioning of a gripping element 3130 on a leaflet, without altering a successful grasp on the other leaflet. In some implementations, the gripping members 3130 can open and close when the paddle 3120 is not closed, thereby allowing leaflets to be grasped in a variety of positions as the particular situation requires.

In some implementations, during treatment and/or implantation, the anchors 3108 are opened and closed to grasp the native mitral valve leaflets (or tricuspid valve leaflets) between the paddles 3120 and the base 3110. The optional gripping members 3130 can further secure the native leaflets by pinching the leaflets between the movable and fixed arms 3134, 3132. The optional barbs 3136 can increase friction with the leaflets or can partially or completely puncture the leaflets.

The device 3100 can be configured to be removably connected to an implant catheter assembly (not shown), where the implant catheter assembly can include one or more actuation elements for engaging the first and second actuation assemblies 3114a, 3114b of the device 3100. The implant catheter assembly can take any suitable form that is capable of removably attaching to the coupling of the device 3100, such as, for example, any form of an implant catheter assembly described in the present disclosure. The actuation element(s) of the implant catheter assembly can take any suitable form that is capable of engaging the first and second actuation assemblies 3114a, 3114b of the device 3100 to actuate one or more components (e.g., paddles, gripping members, paddle frame element(s), component(s) of the base, etc.) of the device 3100.

In some implementations, the device 3100 can be deployed from a delivery sheath (see delivery sheath 2606). In some implementations, the base 3110 of the device 3100 can be implanted between the leaflets of the native mitral valve or tricuspid valve. The anchors 3108 are actuatable between open and closed conditions. In some implementations, during implantation, the paddles 3120 are opened and closed to grasp the native mitral valve leaflets between the paddles 3120 and the base 3110.

In some implementations, the device 3100 can be configured such that the paddles 3120 are able to move between various positions (e.g., the closed position, the partially open position, the open position, etc.) in response to movement of the leaflets of the native valve (e.g., movement of the leaflets to the open position during the diastolic phase) when the device is attached to the leaflets. For example, the device 3100 can include one or more features that allow for the paddles 3120 to move between closed and partially open or open positions as a result of the leaflet(s) moving between closed and open positions, such as, for example, the biasing member and/or gap features of the device 2700 shown in FIGS. 54-60 and/or the device shown in FIGS. 61-62. The movability of the paddles 3120 is advantageous because it reduces the stress on the leaflets resulting from the attachment between the device 3100 and the leaflets.

In some implementations, the paddles 3120 can be independently controllable. For example, the device 3100 can have two independent drive assemblies (e.g., each being similar to the first actuation assembly 3114a) with each paddle 3120 being controlled by a separate drive assembly. In some implementations, the gripping elements 3130 can be independently controllable, such as, for example, any means described for independently controlling gripping elements described in the present disclosure.

In some implementations, the implantable device 3100 can be delivered and implanted within the native mitral valve MV or the native tricuspid valve TV of the heart H. For example, when implanted in the tricuspid valve TV the delivery sheath can be inserted into the left atrium LA through the septum and the device 3100 is deployed from the delivery sheath. The device 3100 can be moved into position within the mitral valve MV into the ventricle LV and partially opened so that the leaflets 20, 22 can be grasped.

In some implementations, the device 3100 can then be decoupled from the implant catheter assembly. In some implementations, the sheath and actuation lines are then retracted leaving the device 3100 deployed in the native mitral valve MV.

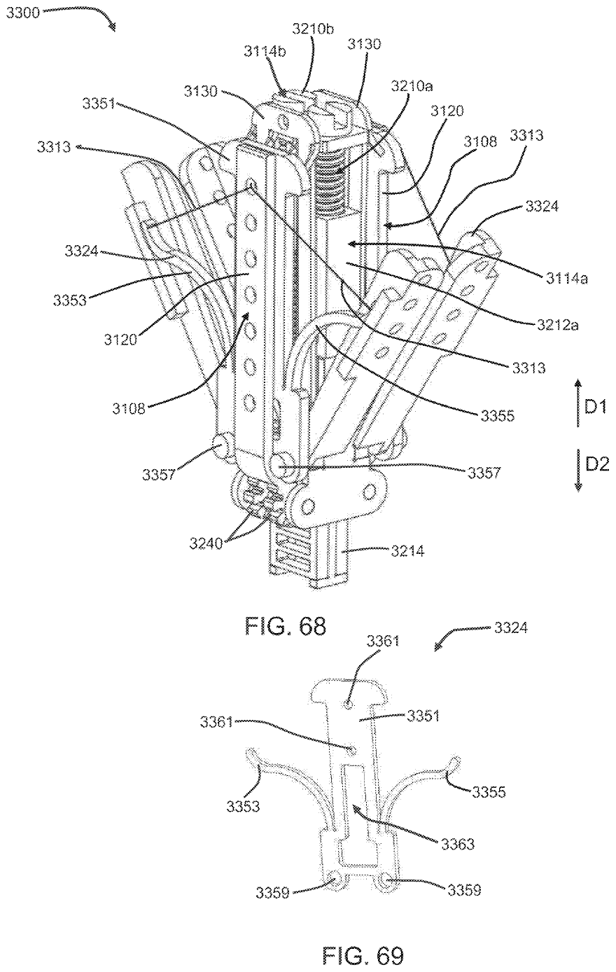

Referring now to FIGS. 68-73, an example of a device 3300 (e.g., a treatment device, a repair device, a prosthetic device, a valve repair device, valve treatment device, implantable device, implant, etc.) is shown. The device 3300 can include any features for a device discussed in the present disclosure, and the device 3300 can be positioned to engage valve tissue 20, 22, 30, 32, 34 as part of any suitable treatment and/or repair system (e.g., any treatment and/or repair system described in the present disclosure).

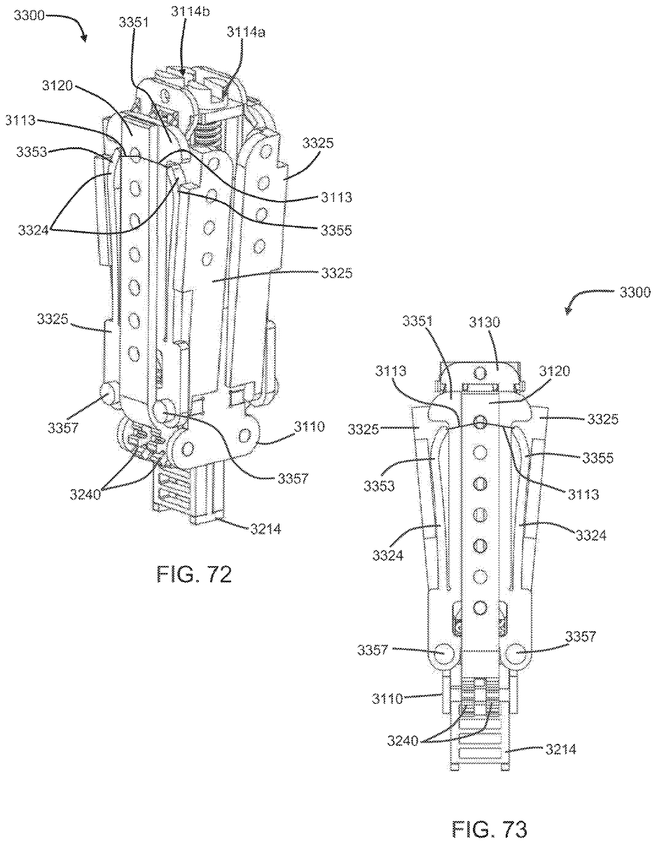

In the illustrated example, the device 3300 takes a similar form to the device 3100 shown in FIGS. 63-67, except that the device 3300 further includes a paddle frame 3324 that is movable between a narrowed configuration (as shown in FIGS. 72-73) and an expanded configuration (as shown in FIGS. 68-71).

In some implementations, the device 3300 can include the optional coupling (not shown), base 3110, first actuation assembly 3114a, second actuation assembly 3114b, and one or more anchors 3108 (including paddles 3120 and optional gripping members 3130) of the device 3100. The coupling, base 3110, first actuation assembly 3114a, second actuation assembly 3114b, and the one or more anchors 3108 can take any suitable form, such as, for example, any form described with reference to FIGS. 63-67.

In some implementations, the first actuation assembly 3114a can be configured to move the paddles 3120 of the anchors 3108 between two or more of the closed position, the partially open position, the open position, and the extended position. For example, the first actuation assembly 3114a can include a drive member 3210a, a driven member 3212a, and a rack 3214, each of which can take any suitable form, such as, for example, any form described with reference to the actuation assembly 3114a shown in FIGS. 63-67.

In some implementations, rotation of the drive member 3210a can cause the driven member 3212a to move in directions D1, D2 (FIG. 68), which causes the rack 3214 to move in the directions D1, D2. The engagement and interaction between the first actuation assembly and the one or more paddles 3120 can take any suitable form. For example, movement of the rack 3214 of the first actuation assembly 3114a in the directions D1, D2 can cause the rack 3214 to engage pinion gears 3240 of the paddles 3120 to move the paddles 3120 between two or more of the closed position, the partially open position, the open position, and the extended position.

The second actuation assembly 3114b can take a variety of different forms, such as, for example, any form described with reference to FIGS. 63-67. For example, the second actuation assembly 3114b can include a drive member 3210b and a driven member 3212b (see FIGS. 63-67), each of which can take any suitable form, such as, for example, any form described with reference to the actuation assembly 3114b shown in FIGS. 63-67. Rotation of the drive member 3210b can cause the driven member 3212b to move in directions D1, D2 (FIG. 68).

The driven member 3212b of the second actuation assembly 3114b can be operatively connected to one or more components (e.g., gripping members, paddle frame element(s), component(s) of the base, etc.) of the device 3300 such that movement of the driven member 3212b in the directions D1, D2 actuates the connected components. In the illustrated example, the driven member 3212b is operatively connected to the paddle frame(s) 3324 such that movement of the driven member 3212b in the directions D1, D2 move the paddle frames 3324 between the narrowed and expanded positions.

The paddle frame(s) 3324 can take a variety of different forms. In the illustrated example, each of the paddle frames 3324 include a body portion 3351, a first arm 3353 that is attached to a first side of the body portion 3351, and a second arm 3355 that is attached to a second side of the body portion 3351. The first and second arms 3353, 3355 are pivotable relative to the body portion 3351 such that the paddle frames 3324 can be moved between the narrowed configuration (as shown in FIGS. 72-73) and an expanded configuration (as shown in FIGS. 68-71).

In some implementations, the paddle frames 3324 can be fixedly attached to a corresponding paddle 3120 such that the paddle frames 3324 move with the paddles 3120 as the paddle 3120 moves between the open and closed positions. In the illustrated example, the body portion 3351 is attached to the paddle 3120 by inserting a fastener (e.g., any suitable type of fastener) through openings 3361 of the paddle frame 3324 and corresponding openings of the paddle 3120. However, it should be understood that the paddle frame 3324 can be attached to the paddle 3120 by any suitable means. In some examples, the paddle frame includes an opening 3363 (FIG. 69) for receiving a portion of the gripping member 3130 (e.g., a fixed arm of the gripping member 3130) such that the gripping member 3130 can be attached to the paddle 3120.

In some implementations, each arm 3353, 3355 of the paddle frames 3324 can be attached to a corresponding frame actuation element 3325, and the frame actuation elements 3325 are movable between an open position (as shown in FIGS. 68 and 70-71) and a closed position (as shown in FIGS. 72-73) to cause the arms 3353, 3355 to pivot relative to the body 3351 and move the paddle frame 3324 between the narrowed and expanded configurations. The arms 3353, 3355 can be attached to the actuation element 3325 by any suitable means that allows for the arms 3353, 3355 to pivot relative to the body portion 3351 of the paddle frame 3324 when the frame actuation elements 3325 move between the open and closed positions.

In some implementations, the paddle frames 3324 are movable between the narrowed and expanded configurations via a connection with the second actuation assembly 3114b. For example, the frame actuation elements 3325 can be attached to the driven member 3212b of the second actuation assembly 3114b by one or more actuation lines 3313 (e.g., suture(s), wire(s), etc.) such that movement of the driven member 3212b in the directions D1, D2 causes the frame actuation elements 3325 to move between an open position (as shown in FIGS. 68 and 70-71) and a closed position (as shown in FIGS. 72-73).

In some implementations, the paddle frame 3324 can be in an expanded configuration when the frame actuation element 3325 is in the open position, and the paddle frame can be in the narrowed configuration when the frame actuation element 3325 is in the closed position. While the paddle frame(s) 3324 are described as being attached to the second actuation assembly 3114b by the frame actuation element(s) 3325 and the actuation line(s) 3313, it should be understood that the paddle frame(s) 3324 can be attached to the second actuation assembly 3114b by any suitable means.

While the paddle frame(s) 3324 are described as being moved between the narrowed and expanded configurations by the second actuation assembly 3114b, it should be understood that the paddle frame(s) 3324 can be moved between the narrowed and expanded configurations by any suitable means.

In the illustrated example (FIGS. 68-73), the frame actuation elements 3325 are pivotably connected to a corresponding paddle 3120 by a pivot member 3357 such that the frame actuation elements 3325 can pivot relative to the paddle 3120. For example, the pivot members 3357 can attach the frame actuation elements 3325 to the paddle 3120 by openings 3359 of the paddle 3120. In some implementations, the frame actuation elements 3325 are attached to a distal portion of the paddles 3120 by pivot members 3357. In some implementations, the frame actuation elements 3325 can have an upward facing opening when in the open position. In some examples, rather than pivotally attaching to the paddles 3120, the frame actuation elements 3325 can pivotably attach to the base 3110 by one or more pivot members.

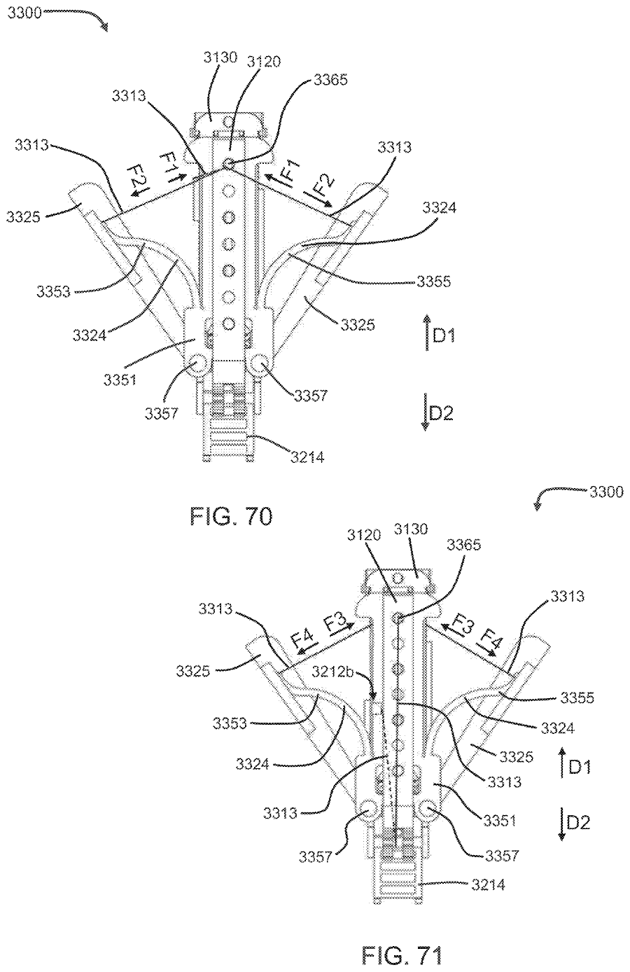

Referring to FIG. 70, in some implementations, movement of the driven member 3212b of the second actuation element 3114b in the distal direction D2 causes the frame actuation elements 3325 to pivot inward to the closed position and, consequently, causes the paddle frame 3324 to be in the narrowed configuration, and movement of the driven member 3212b in the proximal direction D1 causes the frame actuation elements 3325 to pivot outward to the open position and, consequently, causes the paddle frame 3324 to be in the expanded configuration.

As shown in FIG. 70, the arms 3353, 3355 of the paddle frame 3324 and/or the frame actuation elements 3325 are attached to the driven member 3212b by actuation line(s) 3313, where the actuation lines extend through an opening 3365 of the paddle 3120 and attach to the driven member 3212b from an upper portion of the device 3300. In some implementations, movement of the driven member 3212b in the distal direction D2 can cause a force F1 on the actuation lines 3313 that causes the frame actuation elements 3325 to move to the closed position. In some implementations, movement of the driven member 3212b in the proximal direction D1 can cause a force F2 on the actuation lines 3313 that causes the frame actuation elements 3325 to move to the open position.

In some implementations, the paddle frame 3324 can be biased in the expanded configuration such that movement of the driven member 3212b in the proximal direction D1 removes the force F1 being applied to the actuation lines 3313 and allows the arms 3353, 3355 of the paddle frame 3324 to move back to the normally expanded configuration.

Referring to FIG. 71, in some implementations, movement of the driven member 3212b of the second actuation element 3114b in the proximal direction D1 causes the frame actuation elements 3325 to pivot outward to the closed position and, consequently, causes the paddle frame 3324 to be in the narrowed configuration, and movement of the driven member 3212b in the distal direction D2 causes the frame actuation elements 3325 to pivot outward to the open position and, consequently, causes the paddle frame 3324 to be in the expanded configuration.

As shown in FIG. 71, the arms 3353, 3355 of the paddle frame 3324 and/or the frame actuation elements 3325 are attached to the driven member 3212b by actuation line(s) 3313, where the actuation lines extend through an opening 3365 of the paddle 3120, extend distally along the paddle 3120, and attach to the driven member 3212b from a lower portion of the device 3300. In some implementations, movement of the driven member 3212b in the proximal direction D1 can cause a force F3 on the actuation lines 3313 that causes the frame actuation elements 3325 to move to the closed position. In some implementations, movement of the driven member 3212b in the distal direction D2 can cause a force F4 on the actuation lines 3313 that causes the frame actuation elements 3325 to move to the open position.

In some implementations, the paddle frame 3324 can be biased in the expanded configuration such that movement of the driven member 3212b in the distal direction D2 removes the force F3 being applied to the actuation lines 3313 and allows the arms 3353, 3355 of the paddle frame 3324 to move back to the normally expanded configuration.

The paddle frame(s) 3324 can include one or more of the features of any of the paddle frames described in the present disclosure. In some implementations, the paddle frames 3324 are configured to press, coapt, bring together, or bring closer together two native valve leaflets (e.g., leaflets 20, 22 or two of leaflets 30, 32, 34). While the illustrated example shows each of the paddle frames 3324 being made of a single component, it should be understood that the paddle frame can be made of two or more separate components that are attached (e.g., two or more components that are attached via a connection with the corresponding paddle 3120).

The paddle frame 3324 can be made from a variety of different materials and can be shaped in a variety of different ways. For example, the paddle frame 3324 can be made of any material described for a paddle frame in the present disclosure. In some implementations, the paddle frame 3324 can be formed (e.g., cut and/or bent) from sheet material, can be molded, can be cast, 3-D printed, etc.).

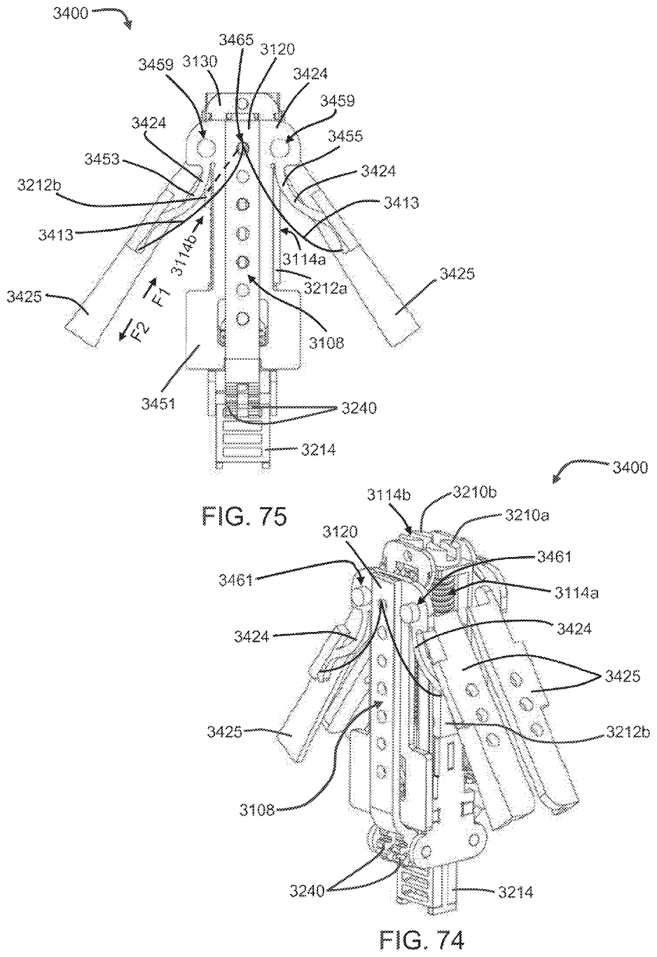

Referring now to FIGS. 74-77, an example of a device 3400 (e.g., a treatment device, a repair device, a prosthetic device, a valve repair device, valve treatment device, implantable device, implant, etc.) is shown. The device 3400 can include any features for a device discussed in the present disclosure, and the device 3400 can be positioned to engage valve tissue 20, 22, 30, 32, 34 as part of any suitable treatment and/or repair system (e.g., any treatment and/or repair system described in the present disclosure).

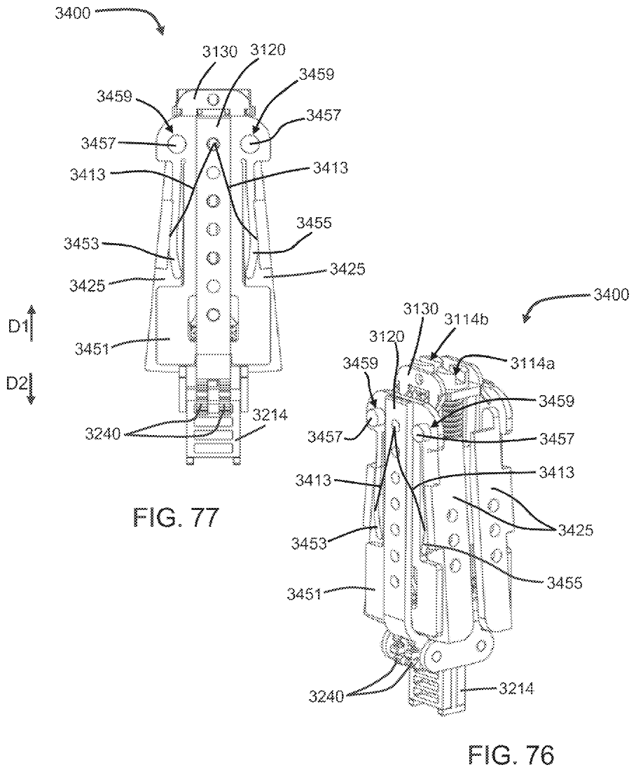

In the illustrated example, the device 3400 is similar in form to the device 3100 shown in FIGS. 63-67, except that the device 3400 further includes a paddle frame 3424 that is movable between a narrowed configuration (as shown in FIGS. 76-77) and an expanded configuration (as shown in FIGS. 74-75). In some implementations, the device 3400 can include the optional coupling (not shown), base 3110, first actuation assembly 3114a, second actuation assembly 3114b, and one or more anchors 3108 (including paddles 3120 and optional gripping members 3130) of the device 3100. The coupling, base 3110, first actuation assembly 3114a, second actuation assembly 3114b, and the one or more anchors 3108 can take any suitable form, such as, for example, any form described with reference to FIGS. 63-67.

The first actuation assembly 3114a can be configured to move the paddles 3120 of the anchors 3108 between two or more of the closed position, the partially open position, the open position, and the extended position. For example, the first actuation assembly 3114a can include a drive member 3210a, a driven member 3212a, and a rack 3214, each of which can take any suitable form, such as, for example, any form described with reference to the actuation assembly 3114a shown in FIGS. 63-67.

In some implementations, rotation of the drive member 3210a can cause the driven member 3212a to move in directions D1, D2 (FIG. 77), which causes the rack 3214 to move in the directions D1, D2. The engagement and interaction between the first actuation assembly and the one or more paddles 3120 can take any suitable form. For example, movement of the rack 3214 of the first actuation assembly 3114a in the directions D1, D2 can cause the rack 3214 to engage pinion gears 3240 of the paddles 3120 to move the paddles 3120 between two or more of the closed position, the partially open position, the open position, and the extended position.

The second actuation assembly 3114b can take a variety of different forms, such as, for example, any form described with reference to FIGS. 63-67. In some implementations, the second actuation assembly 3114b can include a drive member 3210b and a driven member 3212b (see FIGS. 63-67), each of which can take any suitable form, such as, for example, any form described with reference to the actuation assembly 3114b shown in FIGS. 63-67. Rotation of the drive member 3210b can cause the driven member 3212b to move in directions D1, D2 (FIGS. 75 and 77).

The driven member 3212b of the second actuation assembly 3114b can be operatively connected to one or more components (e.g., gripping members, paddle frame element(s), component(s) of the base, etc.) of the device 3400 such that movement of the driven member 3212b in the directions D1, D2 actuates the connected components. In the illustrated example (FIGS. 74-77), the driven member 3212b is operatively connected to the paddle frame(s) 3424 such that movement of the driven member 3212b in the directions D1, D2 move the paddle frames 3424 between the narrowed and expanded positions.

The paddle frame(s) 3424 can take a variety of different forms. In the illustrated example, each of the paddle frames 3424 include a body portion 3451, a first arm 3453 that is attached to a first side of the body portion 3451, and a second arm 3455 that is attached to a second side of the body portion 3451. The first and second arms 3453, 3455 are pivotable relative to the body portion 3451 such that the paddle frames 3424 can be moved between the narrowed configuration (as shown in FIGS. 76-77) and an expanded configuration (as shown in FIGS. 74-75).

In some implementations, the paddle frames 3424 can be fixedly attached to a corresponding paddle 3120 such that the paddle frames 3424 move with the paddles 3120 as the paddles 3120 moves between the open and closed positions. In the illustrated example, the body portion 3451 is attached to the paddle 3120 by inserting a fastener (e.g., any suitable type of fastener) through openings (e.g., similar to openings 3361 of the paddle frame 3324 shown in FIG. 69) and corresponding openings of the paddle 3120. However, it should be understood that the paddle frame 3424 can be attached to the paddle 3120 by any suitable means. In some examples, the paddle frame includes an opening (e.g., similar to opening 3363 of the paddle frame 3324 shown in FIG. 69) for receiving a portion of the gripping member 3130 (e.g., a fixed arm of the gripping member 3130) such that the gripping member 3130 can be attached to the paddle 3120.

In some implementations, each arm 3453, 3455 of the paddle frames 3424 can be attached to a corresponding frame actuation element 3425, and the frame actuation elements 3425 are movable between an open position (as shown in FIGS. 74-75) and a closed position (as shown in FIGS. 76-77) to cause the arms 3453, 3455 to pivot relative to the body 3451 and move the paddle frame 3424 between the narrowed and expanded configurations. The arms 3453, 3455 can be attached to the actuation element 3325 by any suitable means that allows for the arms 3453, 3455 to pivot relative to the body portion 3451 of the paddle frame 3424 when the frame actuation elements 3425 move between the open and closed positions.

In some implementations, the paddle frames 3424 are movable between the narrowed and expanded configurations via a connection with the second actuation assembly 3114b. For example, the frame actuation elements 3425 can be attached to the driven member 3212b of the second actuation assembly 3114b by one or more actuation lines 3413 (e.g., suture(s), wire(s), etc.) such that movement of the driven member 3212b in the directions D1, D2 causes the frame actuation elements 3325 to move between an open position (as shown in FIGS. 74-75) and a closed position (as shown in FIGS. 76-77). The paddle frame 3424 can be in an expanded configuration when the frame actuation element 3425 is in the open position, and the paddle frame 3424 can be in the narrowed configuration when the frame actuation element 3425 is in the closed position. While the paddle frame(s) 3424 are described as being attached to the second actuation assembly 3114b by the frame actuation element(s) 3425 and the actuation line(s) 3413, it should be understood that the paddle frame(s) 3424 can be attached to the second actuation assembly 3114b by any suitable means. While the paddle frame(s) 3424 are described as being moved between the narrowed and expanded configurations by the second actuation assembly 3114b, it should be understood that the paddle frame(s) 3424 can be moved between the narrowed and expanded configurations by any suitable means.

In the illustrated example, the frame actuation elements 3425 are pivotably connected to a corresponding paddle 3120 by a pivot member 3457 such that the frame actuation elements 3425 can pivot relative to the paddle 3120. For example, the pivot members 3457 can attach the frame actuation elements 3425 to the paddle 3120 by openings 3459 of the paddle 3120. In some implementations, the frame actuation elements 3425 are attached to a proximal portion of the paddles 3120 by pivot members 3457. In some implementations, the frame actuation elements 3425 can have a downward facing opening when in the open position.

In the illustrated example, movement of the driven member 3212b of the second actuation element 3114b in the distal direction D2 causes the frame actuation elements 3425 to pivot inward to the closed position and, consequently, causes the paddle frame 3424 to be in the narrowed configuration, and movement of the driven member 3212b in the proximal direction D1 causes the frame actuation elements 3425 to pivot outward to the open position and, consequently, causes the paddle frame 3424 to be in the expanded configuration.

In the illustrated example, the arms 3453, 3455 of the paddle frame 3424 and/or the frame actuation elements 3425 are attached to the driven member 3212b by actuation line(s) 3413, where the actuation lines 3413 extend through an opening 3465 of the paddle 3120 and attach to the driven member 3212b from an upper portion of the device 3400. In some implementations, movement of the driven member 3212b in the distal direction D2 can cause a force F1 (FIG. 75) on the actuation lines 3413 that causes the frame actuation elements 3425 to move to the closed position. In some implementations, movement of the driven member 3212b in the proximal direction D1 can cause a force F2 (FIG. 75) on the actuation lines 3413 that causes the frame actuation elements 3425 to move to the open position.

In some implementations, the paddle frame 3424 can be biased in the expanded configuration such that movement of the driven member 3212b in the proximal direction D1 removes the force F1 being applied to the actuation lines 3413 and allows the arms 3453, 3455 of the paddle frame 3424 to move back to the normally expanded configuration.

In some implementations, movement of the driven member 3212b of the second actuation element 3114b in the proximal direction D1 can cause the frame actuation elements 3425 to move to the closed position and, consequently, cause the paddle frame 3424 to be in the narrowed configuration. In some implementations, movement of the driven member 3212b in the distal direction D2 can cause the frame actuation elements 3425 to move to the open position and, consequently, cause the paddle frame 3424 to be in the expanded configuration. For example, the arms 3453, 3455 of the paddle frame 3424 and/or the frame actuation elements 3425 can be attached to the driven member 3212b by actuation line(s) 3413, where the actuation lines attach to the driven member 3212b from a lower portion of the device 3400 (e.g., similar to the attachment between the arms 3353, 3355 and the driven member 3212b for the device 3300 shown in FIG. 71).

The paddle frame(s) 3424 can include one or more of the features of any of the paddle frames described in the present disclosure. In some implementations, the paddle frames 3424 are configured to press, coapt, bring together, or bring closer together two native valve leaflets (e.g., leaflets 20, 22 or two of leaflets 30, 32, 34). While the illustrated example shows each of the paddle frames 3424 being made of a single component, it should be understood that the paddle frame can be made of two or more separate components that are attached (e.g., two or more components that are attached via a connection with the corresponding paddle 3120).

The paddle frame 3424 can be made from a variety of different materials and can be shaped in a variety of different ways. For example, the paddle frame 3424 can be made of any material described for a paddle frame in the present disclosure. In some implementations, the paddle frame 3424 can be formed (e.g., cut and/or bent) from sheet material, can be molded, can be cast, 3-D printed, etc.).

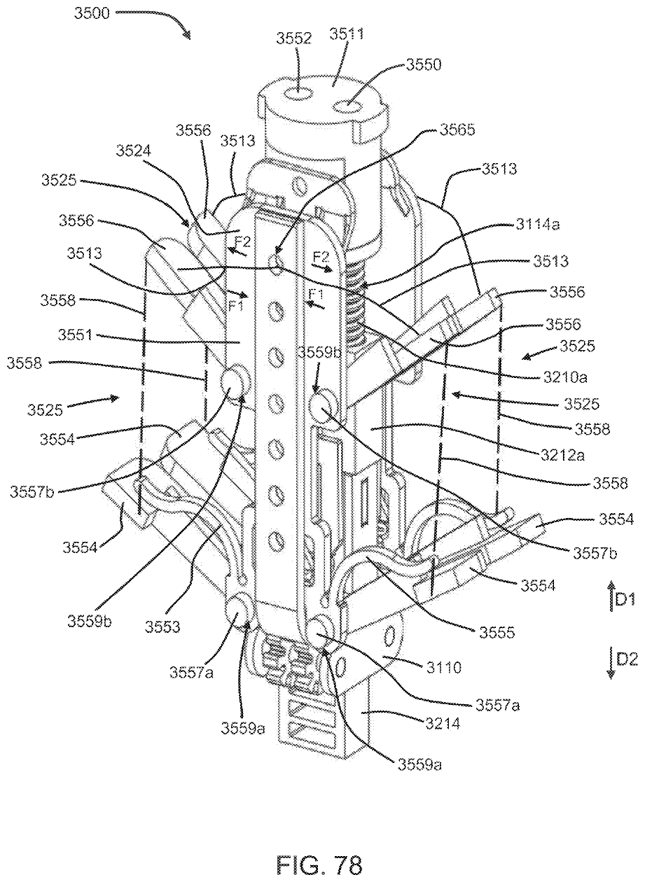

Referring now to FIG. 78, an example of a device 3500 (e.g., a treatment device, a repair device, a prosthetic device, a valve repair device, valve treatment device, implantable device, implant, etc.) is shown. The device 3500 can include any features for a device discussed in the present disclosure, and the device 3500 can be positioned to engage valve tissue 20, 22, 30, 32, 34 as part of any suitable treatment and/or repair system (e.g., any treatment and/or repair system described in the present disclosure).

In the illustrated example, the device 3500 is similar in form to the device 3100 shown in FIGS. 63-67, except that the device 3500 further includes a paddle frame 3524 that is movable between a narrowed configuration (e.g., similar to the configuration shown for the device 3400 shown in FIGS. 76-77) and an expanded configuration (as shown in FIG. 78). In some implementations, the device 3500 can include an optional coupling, base 3110, first actuation assembly 3114a, second actuation assembly 3114b, and one or more anchors 3108 (including paddles 3120 and optional gripping members 3130) of the device 3100. The coupling, base 3110, first actuation assembly 3114a, second actuation assembly 3114b, and the one or more anchors 3108 can take any suitable form, such as, for example, any form described with reference to FIGS. 63-67.

In some implementations, the device 3500 has a coupling 3511. The coupling 3511 can take a variety of different forms. For example, the coupling 3511 can have any of the features of any of the couplings or proximal collars described in the present disclosure and/or can have any of the features of any of the couplings or proximal collars disclosed in PCT application No. PCT/US2018/028189, published as PCT Publication WO2018/195215. In the illustrated example, the coupling 3511 has a first opening 3550 for accepting an actuation element (e.g., a shaft, tube, rod, wire, etc.) for engaging the first actuation assembly 3114a, and the coupling 3511 has a second opening 3552 for accepting an actuation element (e.g., a shaft, tube, rod, wire, etc.) for engaging the second actuation assembly 3114b. The coupling 3511 can have any suitable features, such as, for example, any features for a coupling described in the present disclosure.

In some implementations, the first actuation assembly 3114a can be configured to move the paddles 3120 of the anchors 3108 between two or more of the closed position, the partially open position, the open position, and the extended position. For example, the first actuation assembly 3114a can include a drive member 3210a, a driven member 3212a, and a rack 3214, each of which can take any suitable form, such as, for example, any form described with reference to the actuation assembly 3114a shown in FIGS. 63-67.

In some implementations, rotation of the drive member 3210a can cause the driven member 3212a to move in directions D1, D2, which causes the rack 3214 to move in the directions D1, D2. The engagement and interaction between the first actuation assembly 3114a and the one or more paddles 3120 can take any suitable form. For example, movement of the rack 3214 of the first actuation assembly 3114a in the directions D1, D2 can cause the rack 3214 to engage pinion gears 3240 of the paddles 3120 to move the paddles 3120 between two or more of the closed position, the partially open position, the open position, and the extended position.

The second actuation assembly (not shown) can take a variety of different forms, such as, for example, any form of the second actuation assembly 3114b described with reference to FIGS. 63-67. For example, the second actuation assembly can include a drive member (not shown) and a driven member (not shown), each of which can take any suitable form, such as, for example, any form described with reference to the actuation assembly 3114b shown in FIGS. 63-67. In some implementations, rotation of the drive member can cause the driven member to move in directions D1, D2.

In some implementations, the driven member of the second actuation assembly can be operatively connected to one or more components (e.g., gripping members, paddle frame element(s), component(s) of the base, etc.) of the device 3500 such that movement of the driven member in the directions D1, D2 actuates the connected components. In the illustrated example, the driven member of the second actuation assembly is operatively connected to the paddle frame(s) 3524 such that movement of the driven member in the directions D1, D2 move the paddle frames 3524 between the narrowed and expanded positions.

The paddle frame(s) 3524 can take a variety of different forms. As shown in FIG. 78, each of the paddle frames 3524 include a body portion 3551, a first arm 3553 that is attached to a first side of the body portion 3551, and a second arm 3555 that is attached to a second side of the body portion 3551. The first and second arms 3553, 3555 are pivotable relative to the body portion 3551 such that the paddle frames 3524 can be moved between the narrowed configuration (e.g., similar to the position of the device 3300 shown in FIGS. 72-73) and an expanded configuration (as shown in FIG. 78).

In some implementations, the paddle frames 3524 can be fixedly attached to a corresponding paddle 3120 such that the paddle frames 3524 move with the paddles 3120 as the paddles 3120 moves between the open and closed positions. In some implementations, the body portion 3551 of the paddle frame 3524 is attached to the paddle 3120 by inserting a fastener (e.g., any suitable type of fastener) through openings (e.g., similar to openings 3361 of the paddle frame 3324 shown in FIG. 69) and corresponding openings of the paddle 3120. However, it should be understood that the paddle frame 3524 can be attached to the paddle 3120 by any suitable means. In some implementations, the paddle frame includes an opening (e.g., similar to opening 3363 of the paddle frame 3324 shown in FIG. 69) for receiving a portion of the gripping member 3130 (e.g., a fixed arm of the gripping member 3130) such that the gripping member 3130 can be attached to the paddle 3120.

In some implementations, the device 3500 includes frame actuation elements 3525 that are configured to move the paddle frames 3524 between the narrowed and expanded configurations. The frame actuation elements 3525 can be pivotable relative to the paddles 3120 such that the frame actuation elements 3525 can be moved between an open position (as shown in FIG. 78) and a closed position (e.g., similar to the position of the frame actuation elements 3325 shown in FIGS. 72-73). As shown in FIG. 58, each frame actuation elements 3525 includes a lower actuation element 3554, an upper actuation element 3556, and a connection element 3558 (e.g., a wire, a suture, etc.) that connects the lower actuation element 3554 to the upper actuation element 3556.

In some implementations, each arm 3553, 3555 of the paddle frames 3524 can be attached to a corresponding frame actuation element 3525, and the frame actuation elements 3525 are movable between an open position and a closed position to cause the arms 3553, 3555 to pivot relative to the body 3551 and move the paddle frame 3524 between the narrowed and expanded configurations. In the illustrated example, the arms 3553, 3555 of the paddle frame 3524 are attached to the lower actuation element 3554 of the frame actuation element 3525. The arms 3553, 3555 can be attached to the actuation element 3525 by any suitable means that allows for the arms 3553, 3555 to pivot relative to the body portion 3551 of the paddle frame 3524 when the frame actuation elements 3525 move between the open and closed positions.

In some implementations, the paddle frames 3524 are movable between the narrowed and expanded configurations via a connection with the second actuation assembly. For example, the frame actuation elements 3425 can be attached to the driven member of the second actuation assembly by one or more actuation lines 3513 (e.g., suture(s), wire(s), etc.) such that movement of the driven member of the second actuation assembly in the directions D1, D2 causes the frame actuation elements 3525 to move between the open and closed positions. The paddle frame 3524 can be in an expanded configuration when the frame actuation element 3525 is in the open position, and the paddle frame 3524 can be in the narrowed configuration when the frame actuation element 3525 is in the closed position.

While the paddle frame(s) 3524 are described as being attached to the second actuation assembly by the frame actuation element(s) 3525 and the actuation line(s) 3513, it should be understood that the paddle frame(s) 3524 can be attached to the second actuation assembly by any suitable means. While the paddle frame(s) 3524 are described as being moved between the narrowed and expanded configurations by the second actuation assembly, it should be understood that the paddle frame(s) 3524 can be moved between the narrowed and expanded configurations by any suitable means.

In the illustrated example, the lower actuation element 3554 of the frame actuation element 3525 is pivotably connected to a corresponding paddle 3120 by a pivot member 3557a such that the lower actuation element 3554 can pivot relative to the paddle 3120, and the upper actuation element 3556 of the frame actuation element 3525 is pivotably connected to the corresponding paddle 3120 by a pivot member 3557b such that the upper actuation element 3556 can pivot relative to the paddle 3120. The pivot members 3557a, 3557b can attach the lower and upper actuation elements 3554, 3556 to the paddle 3120 by openings 3559a, 3559b of the paddle 3120. In the illustrated example, the lower and upper actuation elements 3554, 3556 have an upward facing opening when in the open position. In some implementations, the lower and upper actuation elements 3554, 3556 can have a downward facing opening when in the open position.

In some implementations, the actuation lines 3513 connect the driven member of the second actuation assembly to the upper actuation element of the frame actuation elements 3525. In these examples, movement of the driven member of the second actuation assembly in the distal direction D2 causes the upper actuation element 3556 to pivot inward to the closed position. The connection between the upper and lower actuation elements 3554, 3556 by the connection element 3558 causes the lower actuation element 3554 to pivot inward to the closed position when the upper actuation element 3556 pivots inward to the closed position. The movement of the frame actuation element 3525 to the closed position causes the paddle frame 3524 to be in the narrowed configuration. Similarly, movement of the driven member of the second actuation assembly in the proximal direction D1 causes the lower and upper actuation elements 3554, 3556 of the frame actuation elements 3425 to pivot outward to the open position and, consequently, causes the paddle frame 3424 to be in the expanded configuration.

In some implementations, the arms 3553, 3555 of the paddle frame 3524 and/or the frame actuation elements 3425 are attached to the driven member of the second actuation assembly by actuation line(s) 3513, where the actuation lines 3513 extend through an opening 3565 of the paddle 3120 and attach to the driven member from an upper portion of the device 3500. Movement of the driven member in the distal direction D2 can cause a force F1 on the actuation lines 3513 that causes the frame actuation elements 3525 to move to the closed position. Movement of the driven member in the proximal direction D1 can cause a force F2 on the actuation lines 3513 that causes the frame actuation elements 3525 to move to the open position.

In some implementations, the paddle frame 3524 can be biased in the expanded configuration such that movement of the driven member in the proximal direction D1 removes the force F1 being applied to the actuation lines 3513 and allows the arms 3453, 3455 of the paddle frame 3524 to move back to the normally expanded configuration. In some implementations, the paddle frame 3524 being in the normally expanded configuration causes the lower actuation elements 3554 to be in the open position when no force is being applied to the actuation lines 3513, which causes the upper actuation elements 3556 to be in the open position (by the connection with the lower actuation elements via the connection element 3558).

In some implementations, the arms 3553, 3555 of the paddle frame 3524 and/or the frame actuation elements 3425 are attached to the driven member of the second actuation assembly by actuation line(s) 3513, where the actuation lines 3513 attach to the driven member from a lower portion of the device 3500. In these examples, movement of the driven member of the second actuation element in the proximal direction D1 can cause the frame actuation elements 3525 to move to the closed position and, consequently, cause the paddle frame 3524 to be in the narrowed configuration, and movement of the driven member in the distal direction D2 can cause the frame actuation elements 3525 to move to the open position and, consequently, cause the paddle frame 3524 to be in the expanded configuration (e.g., similar to the attachment between the arms 3353, 3355 and the driven member 3212b for the device 3300 shown in FIG. 71).

The paddle frame(s) 3524 can include one or more of the features of any of the paddle frames described in the present disclosure. In some implementations, the paddle frames 3524 are configured to press, coapt, bring together, or bring closer together two native valve leaflets (e.g., leaflets 20, 22 or two of leaflets 30, 32, 34). In some implementations, each of the paddle frames 3524 being made of a single component. In some implementations, the paddle frame can be made of two or more separate components that are attached (e.g., two or more components that are attached via a connection with the corresponding paddle 3120).

The paddle frame 3524 can be made from a variety of different materials and can be shaped in a variety of different ways. For example, the paddle frame 3524 can be made of any material described for a paddle frame in the present disclosure. In some implementations, the paddle frame 3524 can be formed (e.g., cut and/or bent) from sheet material, can be molded, can be cast, 3-D printed, etc.).

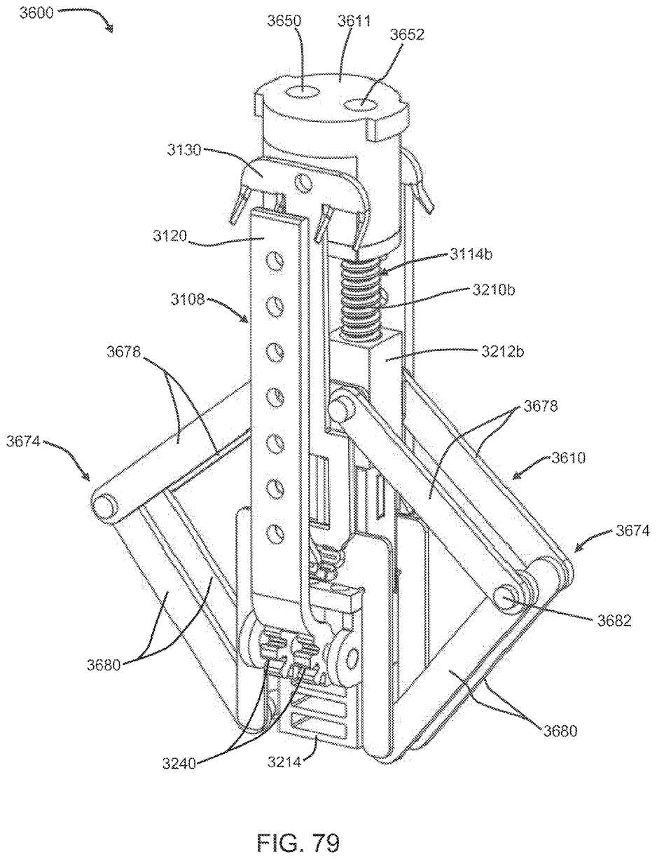

Referring now to FIGS. 79-83, an example of a device 3600 (e.g., a treatment device, a repair device, a prosthetic device, a valve repair device, valve treatment device, implantable device, implant, etc.) is shown. The device 3600 can include any features for a device discussed in the present disclosure, and the device 3600 can be positioned to engage valve tissue 20, 22, 30, 32, 34 as part of any suitable treatment and/or repair system (e.g., any treatment and/or repair system described in the present disclosure).

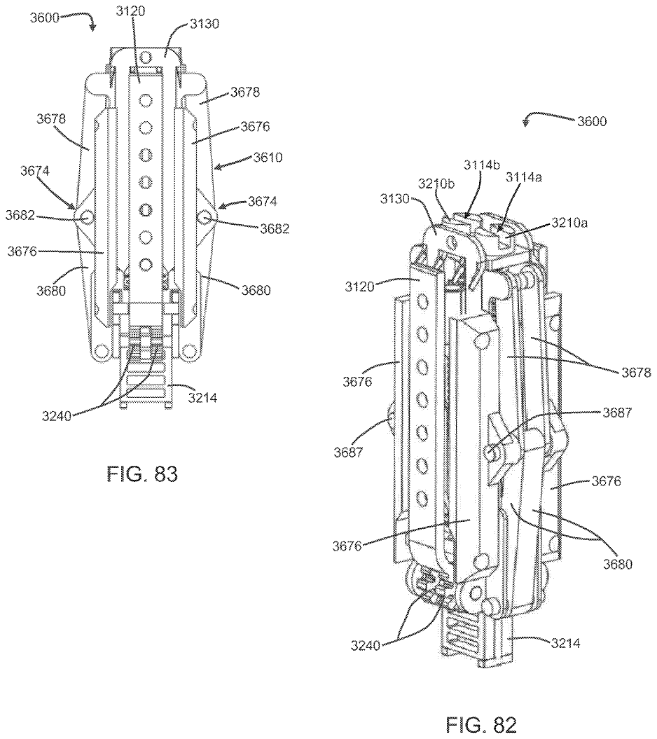

In the illustrated example, the device 3600 takes the form of the device 3100 shown in FIGS. 63-67, except that the device 3600 further includes a base 3610 that is movable between a narrowed configuration (as shown in FIGS. 82-83) and an expanded configuration (as shown in FIGS. 79-81). For example, the device 3600 can include the optional coupling, first actuation assembly 3114a, second actuation assembly 3114b, and one or more anchors 3108 (including paddles 3120 and optional gripping members 3130) of the device 3100. The coupling, first actuation assembly 3114a, second actuation assembly 3114b, and the one or more anchors 3108 can take any suitable form, such as, for example, any form described with reference to FIGS. 63-67. In some implementations, the device 3600 can include paddle frame(s) (not shown) that are attached to the anchors 3108. The paddle frame(s) can take a variety of different forms, such as, for example, any form described in the present disclosure.

In some implementations, the device 3600 has a coupling 3611. The coupling 3611 can take a variety of different forms. For example, the coupling 3611 can have any of the features of any of the couplings or proximal collars described in the present disclosure and/or can have any of the features of any of the couplings or proximal collars disclosed in PCT application No. PCT/US2018/028189, published as PCT Publication WO2018/195215. In the illustrated example, the coupling 3611 has a first opening 3650 for accepting an actuation element (e.g., a shaft, tube, rod, wire, etc.) for engaging the first actuation assembly 3114a, and the coupling 3611 has a second opening 3652 for accepting an actuation element (e.g., a shaft, tube, rod, wire, etc.) for engaging the second actuation assembly 3114b. The coupling 3611 can have any suitable features, such as, for example, any features for a coupling described in the present disclosure.

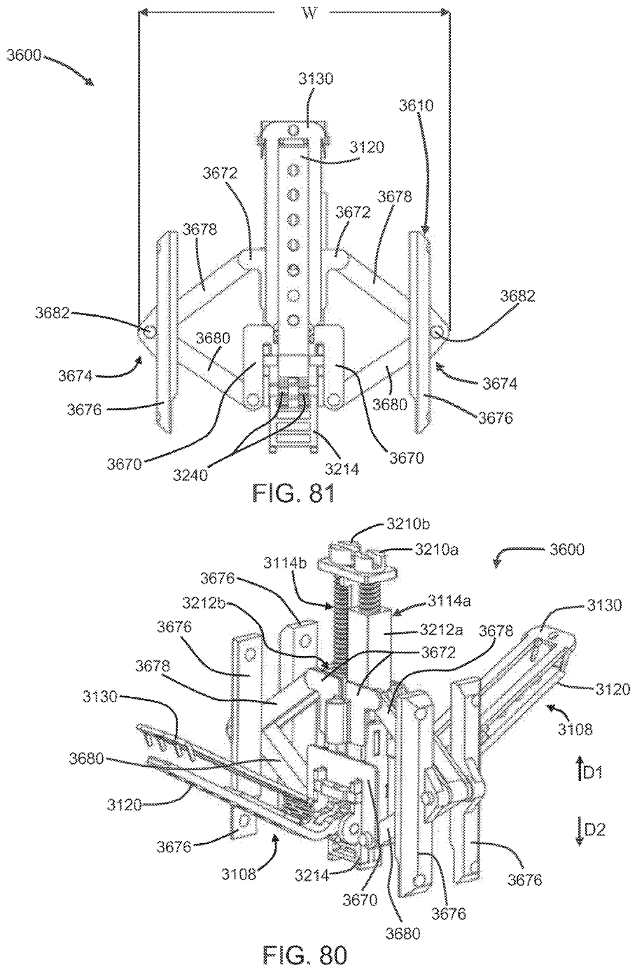

The first actuation assembly 3114a can be configured to move the paddles 3120 of the anchors 3108 between two or more of the closed position, the partially open position, the open position, and the extended position. For example, the first actuation assembly 3114a can include a drive member 3210a, a driven member 3212a, and a rack 3214, each of which can take any suitable form, such as, for example, any form described with reference to the actuation assembly 3114a shown in FIGS. 63-67. Rotation of the drive member 3210a can cause the driven member 3212a to move in directions D1, D2 (FIG. 80), which causes the rack 3214 to move in the directions D1, D2. The engagement and interaction between the first actuation assembly 3114a and the one or more paddles 3120 can take any suitable form. For example, movement of the rack 3214 of the first actuation assembly 3114a in the directions D1, D2 can cause the rack 3214 to engage pinion gears 3240 of the paddles 3120 to move the paddles 3120 between two or more of the closed position, the partially open position, the open position, and the extended position.

The second actuation assembly 3114b can take a variety of different forms, such as, for example, any form of the second actuation assembly 3114b described with reference to FIGS. 63-67. For example, the second actuation assembly can include a drive member 3210b and a driven member 3212b, each of which can take any suitable form, such as, for example, any form described with reference to the actuation assembly 3114b shown in FIGS. 63-67. Rotation of the drive member 3210b can cause the driven member 3212b to move in directions D1, D2.

In some implementations, the driven member 3212b of the second actuation assembly 3114b can be operatively connected to one or more components (e.g., gripping members, paddle frame element(s), component(s) of the base, etc.) of the device 3600 such that movement of the driven member 3212b in the directions D1, D2 actuates the connected components. As shown in FIG. 80, the driven member of the second actuation assembly is operatively connected to the base 3610 such that movement of the driven member 3212b in the directions D1, D2 move the base 3610 between the narrowed and expanded positions.

The base 3610 can take a variety of different forms, such as any form described in the present disclosure. In some implementations, the base can include any of the features described for the base 3110, such as, any features for securing the first and second actuation assemblies 3114a, 3114b within the base 3110 (e.g., longitudinally extending members, laterally extending proximal members, laterally extending distal members, pivot members, etc.), any of the features for securing the paddles 3120 to the base 3110 (e.g. via pivot members).

In some implementations, the base 3610 includes a fixed member 3670, a movable member 3672, one or more link assemblies 3674, and one or more optional longitudinally extending members 3676 (FIGS. 80-83).

In some implementations, each link assembly 3674 includes an upper link 3678 and a lower link 3680 that is pivotably coupled to the upper link 3678 by a pivot member 3682. The lower link 3680 can be pivotally attached to the fixed member 3670, and the upper link 3678 can be pivotally attached to the movable member 3672. The one or more longitudinally extending members 3676 can be attached to the link assembly 3674 (e.g., proximate the pivot member 3682) such that the longitudinally extending members 3676 move with the link assembly 3674.

In some implementations, the movable member 3672 can be attached to the driven member 3212b of the second actuation assembly 3114b such that movement of the driven member 3212b in the directions D1, D2 causes the movable member 3672 to move in the same direction as the driven member 3212b. Movement of the movable member 3672 in the directions D1, D2 causes the upper link 3678 of the link assembly 3674 to move in the same direction as the movable member 3672, which causes the link assembly 3674 to move between an expanded configuration (as shown in FIGS. 79-81) and a narrowed configuration (as shown in FIGS. 82-83).

In some implementations, movement of the movable member 3672 in the distal direction D2 causes the connection between the upper link 3678 and the movable member 3672 to move in the distal direction D2, which causes the pivotal connection between the upper and lower links 3678, 3680 (e.g., via pivot member 3682) to move in an outward direction such that a width W of the base 3610 increases. In some implementations, movement of the movable member 3672 in the proximal direction D1 causes the connection between the upper link 3678 and the movable member 3672 to move in the proximal direction D1, which causes the pivotal connection between the upper and lower links 3678, 3680 (e.g., via pivot member 3682) to move in an inward direction such that a width W of the base 3610 decreases. While the base 3610 is described as being moved between the narrowed and expanded configurations by the second actuation assembly, it should be understood that the base 3610 can be moved between the narrowed and expanded configurations by any suitable means.

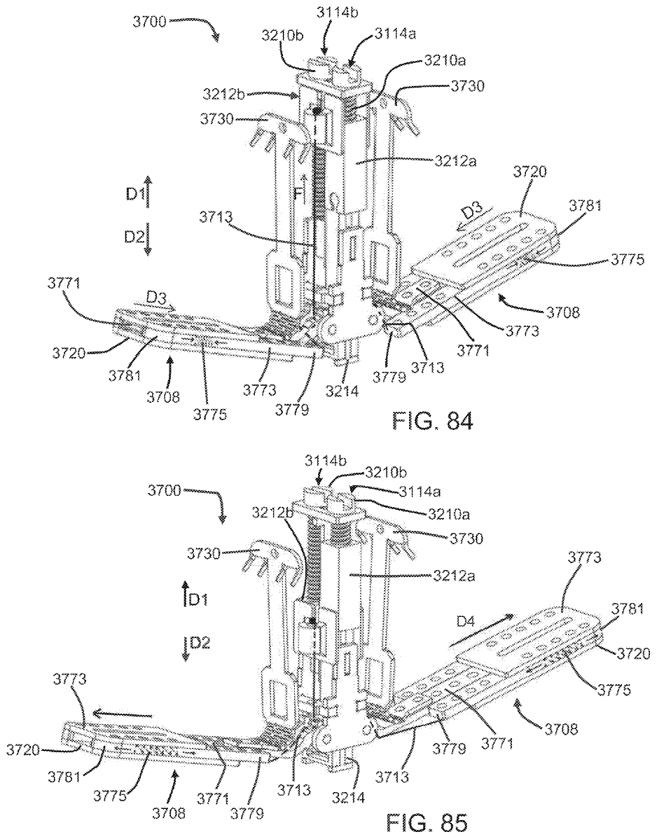

Referring now to FIGS. 84-85, an example of a device 3700 (e.g., a treatment device, a repair device, a prosthetic device, a valve repair device, valve treatment device, implantable device, implant, etc.) is shown. The device 3700 can include any features for a device discussed in the present disclosure, and the device 3700 can be positioned to engage valve tissue 20, 22, 30, 32, 34 as part of any suitable treatment and/or repair system (e.g., any treatment and/or repair system described in the present disclosure).

In the illustrated example, the device 3700 is similar to the device 3100 shown in FIGS. 63-67, except that the device 3700 further includes one or more paddles 3720 of the anchors 3708 that can be moved between a shortened configuration (as shown in FIG. 84) and a lengthened configuration (as shown in FIG. 85).

In some implementations, the device 3700 can include the optional coupling, first actuation assembly 3114a, and second actuation assembly 3114b of the device 3100. The optional coupling, first actuation assembly 3114a, and second actuation assembly 3114b can take any suitable form, such as, for example, any form described with reference to FIGS. 63-67. In some implementations, the device 3700 can include paddle frame(s) (not shown) that are attached to the anchors 3708. The paddle frame(s) can take a variety of different forms, such as, for example, any form described in the present disclosure.

In some implementations, the first actuation assembly 3114a can be configured to move the paddles 3720 of the anchors 3708 between two or more of the closed position, the partially open position, the open position, and the extended position. For example, the first actuation assembly 3114a can include a drive member 3210a, a driven member 3212a, and a rack 3214, each of which can take any suitable form, such as, for example, any form described with reference to the actuation assembly 3114a shown in FIGS. 63-67. Rotation of the drive member 3210a can cause the driven member 3212a to move in directions D1, D2, which causes the rack 3214 to move in the directions D1, D2.

The engagement and interaction between the first actuation assembly 3114a and the one or more paddles 3720 can take any suitable form, such as the engagement and interaction of the first actuation assembly 3114a and the paddles 3120 described with reference to FIGS. 63-67. In some implementations, movement of the rack 3214 of the first actuation assembly 3114a in the directions D1, D2 can cause the rack 3214 to engage pinion gears of the paddles 3720 (e.g., similar to pinion gears 3240 shown in FIGS. 63-67) to move the paddles 3720 between two or more of the closed position, the partially open position, the open position, and the extended position.

The second actuation assembly 3114b can take a variety of different forms, such as, for example, any form of the second actuation assembly 3114b described with reference to FIGS. 63-67. For example, the second actuation assembly can include a drive member 3210b and a driven member 3212b, each of which can take any suitable form, such as, for example, any form described with reference to the actuation assembly 3114b shown in FIGS. 63-67. Rotation of the drive member 3210b can cause the driven member 3212b to move in directions D1, D2.

In some implementations, the driven member 3212b of the second actuation assembly 3114b can be operatively connected to one or more components (e.g., gripping members, paddle frame element(s), component(s) of the base, etc.) of the device 3700 such that movement of the driven member 3212b in the directions D1, D2 actuates the connected components. As shown in FIGS. 84-85, the driven member of the second actuation assembly is operatively connected to the paddles 3720 such that movement of the driven member 3212b in the directions D1, D2 move the paddles 3720 between the shortened and lengthened positions.

The anchors 3708 can take a variety of different forms. For example, the anchors 2708 can include any of the features of any anchors described in the present disclosure. In the illustrated example, each of the anchors 3708 include a paddle 3720 and a gripping member 3730. The gripping members 3730 can take any suitable form, such as, for example, any form described for a gripping member in the present disclosure. In the illustrated example, the paddles 3720 include a fixed member 3771 and a movable member 3773 that is attached to and movable relative to the fixed member 3771. In some implementations, the movable member 3773 can be slidably attached to the fixed member 3771. In some implementations, the paddles 3720 include a biasing member 3775 that is configured to maintain the paddles 3720 in one of the shortened or lengthened position.

In some implementations, the paddles 3720 are movable between the shortened and lengthened configurations via a connection with the second actuation assembly 3114b. For example, the movable member 3773 can be attached to the driven member 3212b by one or more actuation lines 3713 (e.g., suture(s), wire(s), etc.) such that movement of the driven member 3212b in the directions D1, D2 causes the movable member 3773 to move relative to the fixed member 3771, which causes the paddles 3720 to move between the shortened configuration (as shown in FIG. 84) and the lengthened configuration (as shown in FIG. 85).

Referring to FIG. 84, in the illustrated example, movement of the driven member 3212b of the second actuation element 3114b in the proximal direction D1 creates a force F on the actuation line 3713 that causes the movable member 3773 to move in an inward direction D3 to the shortened configuration. Referring to FIG. 85, movement of the driven member 3212b in the distal direction D2 causes the movable member 3773 to move in the outward direction D4 to the lengthened configuration.

In some implementations, the biasing member 3775 is configured such that the paddles 3720 are normally in the lengthened configuration. In some implementations, movement of the driven member 3212b in the distal direction D2 removes the force F (FIG. 84) on the actuation line 3713 such that the paddle 3720 moves to the normally lengthened configuration. In some implementations, the actuation line 3713 can provide a force on the movable member 3773 (when the driven member 3212b is moved in the distal direction D2) that causes the paddle 3720 to be in the lengthened configuration.

In the illustrated example, the actuation line 3713 is attached to a first end 3779 of the movable member 3773, which allows for movement of the driven member 3212b in the proximal direction D1 to cause the paddles 3720 to be in the shortened configuration, and which allows for movement of the driven member 3212b in the distal direction D2 to cause the paddles 3720 to be in the lengthened configuration.

In some implementations, the actuation line 3713 can be attached to a second end 3781 of the movable member 3773 and the driven member 3212b. In these examples, movement of the driven member 3212b of the second actuation element 3114b in the proximal direction D1 can cause the movable member 3773 to move outward to the lengthened configuration, and movement of the driven member 3212b in the distal direction D2 can cause the movable member 3773 to move in the inward direction to the shortened configuration.

In some implementations, the biasing member 3775 is configured such that the paddles 3720 are normally in the shortened configuration. In some implementations, movement of the driven member 3212b in the distal direction D2 removes the force on the actuation line 3713 such that the paddle 3720 moves to the normally shortened configuration. In some implementations, the actuation line 3713 can provide a force on the movable member 3773 (when the driven member 3212b is moved in the distal direction D2) that causes the paddle 3720 to be in the shortened configuration.

Referring now to FIGS. 86-94, an example head 3820 for a drive member of an actuation assembly of a treatment and/or repair device (e.g., any treatment or repair device described in the present disclosure) is shown, along with an example actuation element 3930 of a delivery device (e.g., an actuation element of an implant catheter assembly) for engaging the head 3820 to actuate the actuation assembly of the treatment and/or repair device. The delivery device can take any suitable form, such as, for example, any form described in the present disclosure. The actuation assembly of the treatment and/or repair device can take any suitable form, such as, for example, the form of the actuation assembly 1514 described the reference to FIGS. 31-43, the form of the actuation assembly 3114a described with reference to FIGS. 63-67, the form of the actuation assembly 3114b described with reference to FIGS. 63-67, or any other form of an actuation assembly described in the present disclosure that includes a drive member having a head where the head can take the form of the example head 3820 shown in FIGS. 86-94.

In some implementations, the head 3820 can include a proximal portion 3803 and a distal portion 3805. The proximal portion 3803 includes a connector 3807 (e.g., a proximal connector) for being engaged by and removably connecting to the actuation element 3930 of the delivery device. In the illustrated example, the connector 3807 includes a bar 3813. However, it should be understood that the connector 3807 can take any suitable form that is capable of being engaged by and connecting with the actuation element 3930.

In some implementations, the distal portion 3805 can include a connection feature that is configured to connect to one or more components of a drive member. For example, the distal portion 3805 can be configured to connect to a neck (e.g., neck 1921 of the drive member 1910 described in the present disclosure) and/or threaded portion (e.g., threaded portion 1922 of the drive member 1910 described in the present disclosure). In the illustrated example, the distal portion 3805 includes a connector 3809 (e.g., distal connector) for attaching to a neck, threaded portion, or other component of a drive member of an actuation assembly. The connector 3809 can include an opening 3811 for receiving a component of a drive member. In some implementations, the connector 3809 is integrally formed with one or more components of the drive member (e.g., integrally formed with the neck and/or threaded portion).

In some implementations, the actuation element 3930 can include a drive element 3913 and a securing element 3917. The drive element 3913 can include a drive end 3950 for attaching to the head 3820 such that the drive element 3913 can be used to drive the head 3820. In the illustrated example, the drive end 3950 includes a mating feature 3953 for attaching to the connector 3807 of the head 3820. In some implementations, the mating feature 3953 can be a slot 3955 for receiving the bar 3813 of the head 3820. In some implementations, the slot 3955 can have an arcuate shape that allows for the bar 3813 to be secured within the slot 3955.

In some implementations, the securing element 3917 can be configured to secure the connection between the mating feature 3953 of the actuation element 3930 and the connector 3807 of the head 3820. In some implementations, the drive element 3913 includes a catheter 3919 for delivering the securing element 3917 to the drive end 3950. The drive end 3950 can include a passage 3921 (FIGS. 90-92) that is in communication with the catheter 3919 and the slot 3955 such that the securing element 3917 can be moved to engage the connector 3807 of the head 3820 when the head 3820 is connected to the mating feature 3953 of the drive element 3913.

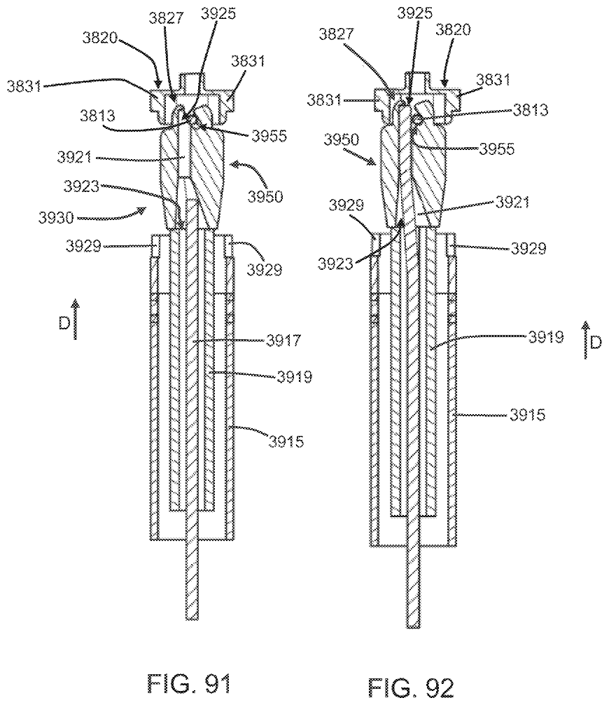

Referring to FIGS. 91 and 92, the head 3820 can be connected to the actuation element 3930 by inserting the bar 3813 of the head 3820 into the slot 3955 of the actuation element 3930. In some examples, the head 3820 can include a recess 3827 for receiving a portion of the drive end 3950 of the actuation element 3930 when the bar 3813 is disposed within the slot 3955. After the bar 3813 is within the slot 3955, the securing element 3917 can be moved in a distal direction D such that the securing element 3917 moves through the passage 3921 of the drive end 3950 and into the slot 3921 such that the securing element 3917 inhibits the bar 3813 of the head 3820 from being removed from the slot 3955.

In some implementations, the passage 3921 can include a proximal opening 3923 that is sized and shaped to align with the catheter 3919 such that the securing element 3917 can be easily moved from the catheter 3919 to the passage 3921. The passage 3921 can include one or more sloped walls that allow for the passage 3921 to narrow from the proximal opening 3923 to the distal opening 3925 to facilitate movement of the securing element 3917 into the slot 3955.

In some implementations, the securing element 3917 can be, for example, a wire, tube, shaft, or any other suitable component that is capable of moving through the passage 3921 and into the slot 3955 to secure the bar 3813 of the head 3820 within the slot 3955. The securing element 3917 can be made, of, for example, metal, plastic, etc. and can be a rod, tube, shaft, etc.

In some implementations, the actuation element 3930 can be connected to the head 3820 prior to the delivery of the treatment and/or repair device to the native valve of a patient, or the actuation element can be connected to the head after the device is delivered to the native valve of the patient.

In some implementations, the above steps are reversed to uncouple the drive end 3950 of the drive element 3913 from the head 3820. For example, the securing element 3917 can be removed from the slot 3955 by moving the securing element in a proximal direction through the passage 3921 and catheter 3919. Once the securing element 3917 is removed from the slot 3955, a force can be applied to the drive end 3950 in a proximal direction such that the bar 3813 is removed from the slot 3955 to detach the actuation element 3930 from the head 3820.

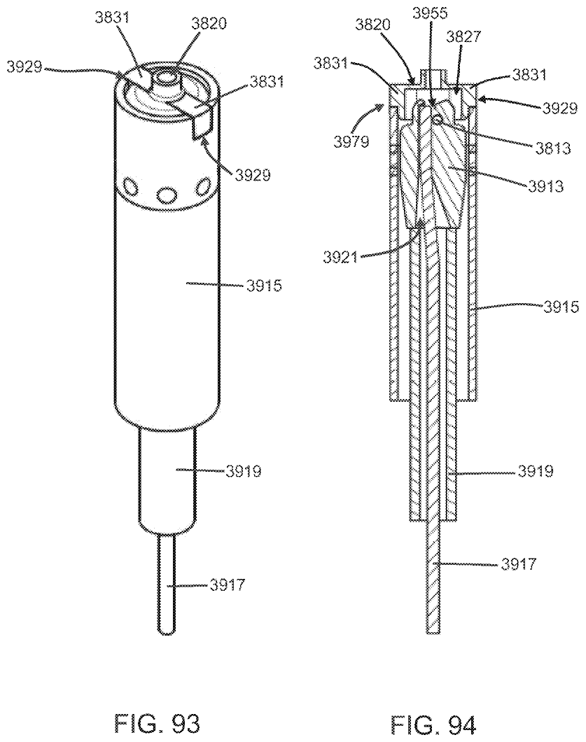

In some implementations, the actuation element 3930 can include and/or be delivered through a catheter 3915. In some implementations, the catheter 3915 can be any component of the delivery device that allows for the drive end of the actuation element 3930 to be delivered to and removed from the head 3820 of the actuation assembly for the treatment and/or repair device. In some implementations, the catheter 3915 can include one or more slots 3929, and the head 3820 can include one or more projections 3831 for extending into the slots 3929 of the catheter 3915 to secure the catheter 3915 to the head 3820 (as shown in FIGS. 93-94).

In some implementations, when the catheter 3915 is secure to the head 3820 by the engagement between the projection(s) 3831 and slot(s) 3929, the catheter 3915 can be used to drive the head 3820 to actuate one or more components of the treatment and/or repair device. The catheter 3915 can be used with the drive element 3913 to drive the head 3820, or the catheter 3915 can be used to independently drive the head 3820.

In some implementations, when the actuation element 3930 is coupled to the head 3820 of an actuation assembly for the treatment and/or repair device, the actuation element 3930 can be used to move the treatment and/or repair device 1500 between various positions, such as, for example, the positions illustrated for the device 1500 by FIGS. 40-43.

In some implementations, the actuation element 3930 can be used to rotate the head 3820 to advance a driven member and a rack such that the rack engages pinion gear(s) of paddle(s) of the device to move the paddles between two or more of a closed position, a partially open position, an open position, and an extended position. In some implementations, the actuation element 3930 can be used to rotate the head 3820 to advance a driven member that is attached or coupled to one or more of gripping member(s), paddle frame(s), a base, and/or any component of the device to actuate the attached components.

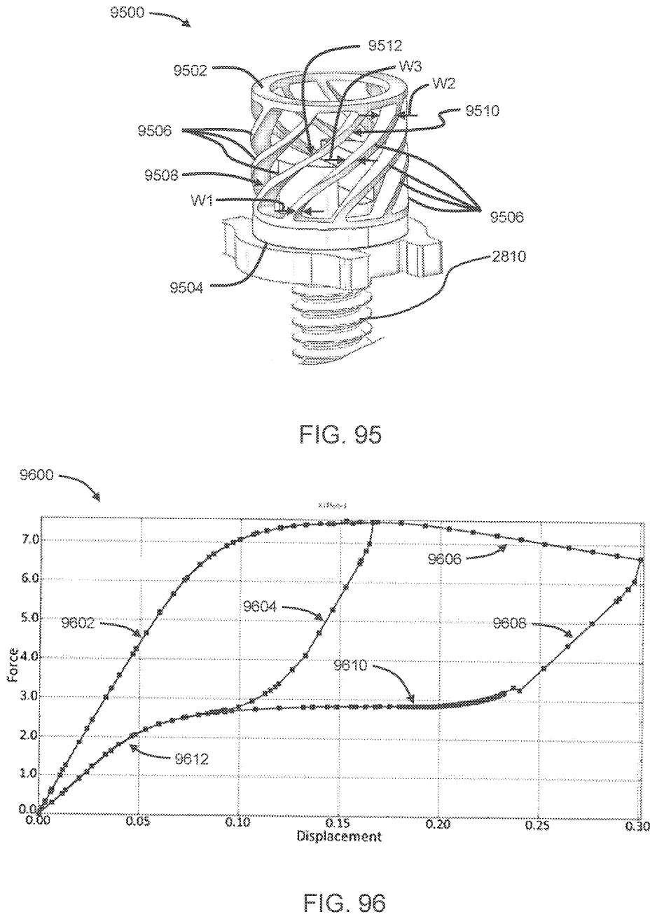

Referring now to FIGS. 95 and 96, an example of a bias member 9500 (e.g., spring, elastic plug, etc.) is illustrated. Biasing device or member 9500 includes a helical-type spring arrangement that includes a top portion or surface 9502, a bottom portion or surface 9504, and a plurality of flexible beams or struts 9506. Each beam or strut 9506 is curved and includes a convex portion 9508, a concave portion 9510, and inflection portion 9512 therebetween. Each strut 9506 is curved and further includes a subtle “S” shape. This creates an arrangement having a plurality of interconnected “S” shaped struts. In some implementations, each beam or strut portion 9506 can include a taper wherein convex portion 9508 and concave portion 9510 taper from a wider width beginning at their connections to top and bottom surfaces 9502 and 9504 (see W1 and W2, respectively) to a smaller width at inflection portion 9512 (see W3). Thus, each beam or strut portion 9506 tapers from wider widths W1 and W2 to narrower width W3. The taper widths W1 and W2 do not need to be the same but can be in some implementations. Bias member 9500, and any bias members disclosed herein, can be made of any of the materials previously disclosed herein including, for example, metal(s) such as nitinol, stainless steel, etc. The spring or biasing member 9500 can be made in a variety of different ways. For example, the spring or biasing member can be cut from a tube, can be cut from a flat sheet and rolled into a tubular shape, can be cast, can be molded, etc.

As shown in FIG. 96, in one example the bias device or member 9500 includes a force displacement behavior 9600 having a plurality of force displacement curves. When force is applied to the bias member 9500, the flexible beams or struts 9506 deform in response. The convex, concave, and inflection portions work together to provide a unique force displacement behavior. This behavior includes multiple force displacement curves that can be tailored for specific applications with specific levels of stiffness, damping, and hysteresis.

In some implementations, measured force and displacement during use of bias member 9500 (e.g., spring, elastic plug, etc.) can be plotted to make a plurality of force displacement curves that include a first loading curve 9602 (or first spring coefficient) and an associated first unloading curve 9604 (or second spring coefficient), second loading curve 9606 (or third spring coefficient) and associated second unloading curves 9608 and 9610 (or fourth spring coefficient(s)), and a common unloading curve 9612 (or fifth spring coefficient). The first loading and unloading curves 9602 and 9604 and common unloading curve 9612 provide a first force displacement characteristic, function or behavior. The second loading and unloading curves 9606, 9608, 9610, and common unloading curve 9612 provide a second force displacement characteristic, function or behavior. In this manner, the bias member 9600 (e.g., spring, elastic plug, etc.) can provide different or a plurality of behaviors (or spring coefficients) with regard to force displacement characteristics. One particular aspect of such an arrangement is that as displacement continues, the change in force is relatively minor. For example, in FIG. 96, the applied force at displacements approximately 0.07-0.30 is substantially unchanging and varies only slightly (i.e., from 6.5 to 7). Hence, displacements beyond approximately 0.07 do not provide increases in bias member spring force and provide a substantially linear or flat loading force displacement characteristic. The force displacement characteristics shown in FIG. 96 are only one example and can be modified or changed by changing the physical shape and material properties of bias member 9500. This includes lengthening or shortening the substantially linear or flat loading force displacement characteristic and/or increasing or decreasing the force(s) at which such characteristics occur. Other force displacement characteristics can also be used including those with more or less force displacement curves than shown and curves having different shapes than those shown.

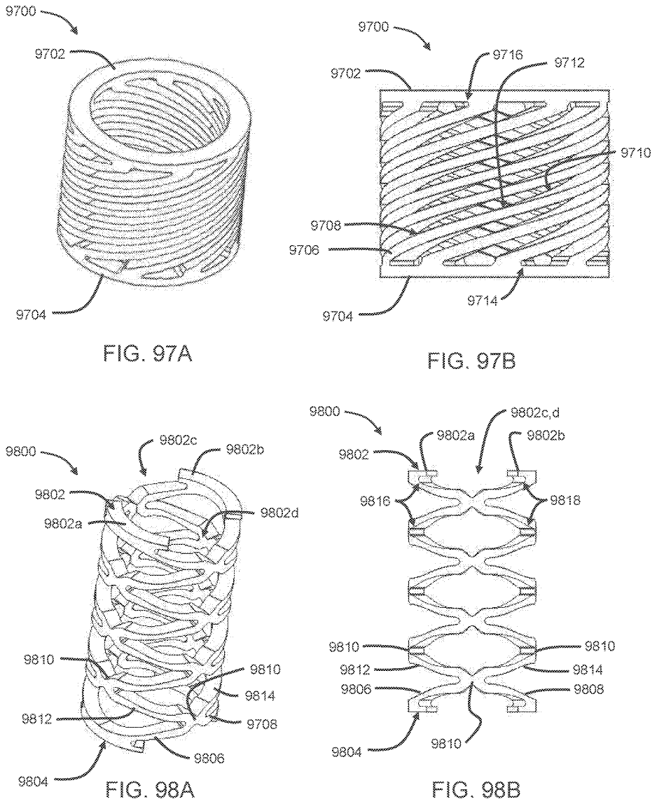

FIGS. 97A and 97B illustrate an example of a bias member 9700 (e.g., spring, elastic plug, etc.) that is similar to bias member 9500 (FIG. 95), except that it includes a different physical arrangement of flexible beams or struts. Similar to bias member 9500, bias member 9700 includes a helical-type spring arrangement that includes a top portion or surface 9702, a bottom portion or surface 9704, and a plurality of flexible beams or struts 9706. Each beam or strut 9706 is curved and includes a convex portion 9708, a concave portion 9710, and inflection portion 9712 therebetween. Each strut 9706 is curved and further includes a subtle “S” shape. This creates an arrangement having a plurality of interconnected “S” shaped struts. In the example of FIGS. 97A and 97B, bias member 9700 includes more struts 9706 and with different angular configurations compared to bias member 9500 of FIG. 95. Bias member 9700 also include stepped or vertical offset portions 9714 and 9716 at their connection to top and bottom surfaces 9702 and 9704. The stepped or vertical offset portions 9714 and 9716 provides a larger fillet between struts 9706 and top and bottom surfaces 9704 that reduces the stress concentrations in those areas. Bias member 9700 can also optionally include the tapered strut configuration described in connection with bias member 9500 of FIG. 95. In operation, bias member 9700 behaves similarly to bias member 9500 and provides a similar plurality of force displacement curves, behaviors or functions (e.g., as shown by of example in FIG. 96), except the magnitude of the force to the corresponding displacement will be different.

Referring to FIGS. 98A and 98B, an example of a bias member 9800 (e.g., spring, elastic plug, etc.) is illustrated. Bias member 9800 has a compound or stacked arrangement that includes a plurality of sections between top and bottom portions 9802 and 9804. Each section can include, for example, a plurality of flexible beams or struts including struts 9806, 9808, 9812, and 9814. In the example shown, curved struts 9806 and 9808 are connected at a central portion 9810, which also connects curved struts 9812 and 9814. Further central portions 9810 connect the remaining curved struts to form the arrangement shown in FIGS. 98A and 98B. Stepped or vertical offset portions 9816 and 9818 can also be included for reducing stress concentrations (as described in connection with FIG. 97). Furthermore, beams or struts 9806, 9808, 9812, and 9814 can also optionally include the tapered arrangement described in connection with FIG. 95). When force is applied to the bias member 9800, the flexible beams or struts 9806, 9808, 9812 and 9814 deform in response by flexing or bending under the applied force. When the force is removed or lessened, the flexible beams or struts return to their original configuration. In some implementations, top and bottom surfaces or portions 9802 and 9804 include a plurality of platform surface portions having a plurality of gaps therebetween (e.g., see top surface portions 9802a and 9802b and top gaps 9802c and 9802d as representative). While bias member 9800 is shown having two cells per revolution, some examples can include more than two cells per revolution including, for example three to five or more.

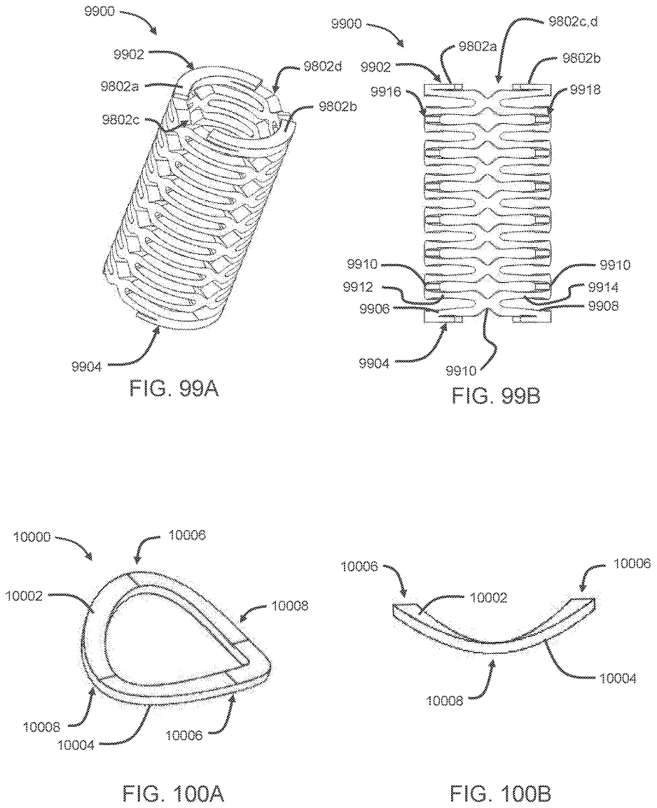

FIGS. 99A and 99B illustrate an example of a bias member (e.g., spring, elastic plug, etc.). Bias member 9900 also has a compound or stacked arrangement that includes a plurality of sections between top and bottom portions 9902 and 9904. Each section can include, for example, a plurality of flexible beams or struts including 9906, 9908, 9912, and 9914. In the example shown, curved struts 9906 and 9908 are connected at a central portion 9910, which also connects curved struts 9912 and 9914. Further central portions 9910 connect the remaining curved struts to form the arrangement shown in FIGS. 99A and 99B. Stepped or vertical offset portions 9916 and 9918 can also be included for reducing stress concentrations (as described in connection with FIG. 97). Furthermore, beams or struts 9906, 9908, 9912, and 9914 can also optionally include the tapered arrangement described in connection with FIG. 95). In the example shown, the beams or struts of bias member 9900 have less of an angular orientation compared to bias member 9800 and, hence, less displacement or compression. When force is applied to the bias member 9900, the flexible beams or struts 9906, 9908, 9912 and 9914 deform in response by flexing or bending under the applied force. When the force is removed or lessened, the flexible beams or struts return to their original configuration. In some implementations, top and bottom surfaces or portions 9902 and 9904 include a plurality of platform surface portions having a plurality of gaps therebetween (e.g., see top surface portions 9802a and 9802b and top gaps 9802c and 9802d as representative). While bias member 9900 is shown having two cells per revolution, in some implementations, can include more than two cells per revolution including, for example three to five or more.

Referring now to FIGS. 100A and 100B, an example of a bias member 10000 (e.g., spring, elastic plug, etc.) is shown. Bias member 10000 can be, for example, of a spring-washer type arrangement that includes top and bottom surfaces or portions 10002 and 10004 with flexible convex beam or strut portions 10006 and concave portions 10008. The thickness and shape of each strut portion can vary. For example, flexible convex beam or strut portions 10006 can have greater thickness or width than concave portions 10008 (or vice versa). When force is applied to the bias member 10000, the flexible beams or struts 10006 and 10008 deform in response by flexing or bending under the applied force and becoming less convex and concave. When the force is removed or lessened, the flexible beams or struts return to their original configuration. Bias member 10000 can be stacked or compounded by affixing flexible convex beam or strut portions 10006 together by, for example, welding or other secure means.

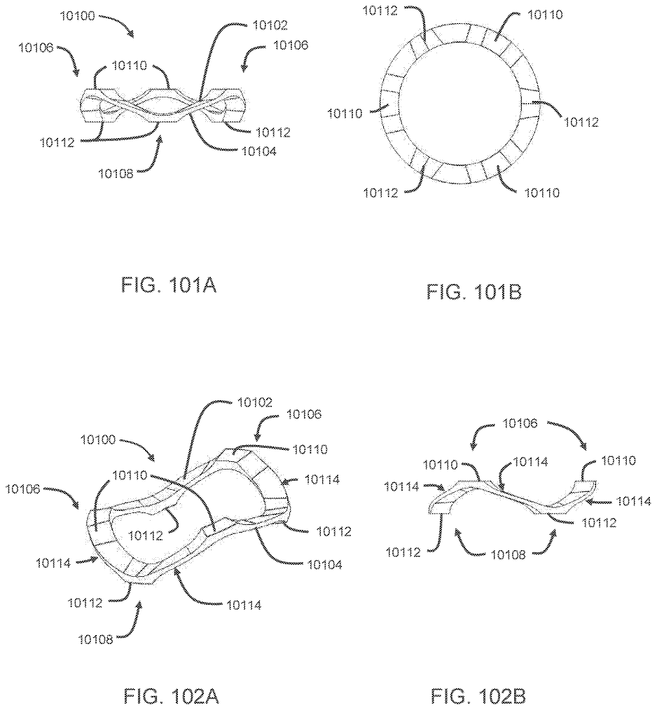

FIGS. 101A, 101B, 102A and 102B illustrate an example of a bias member 10100 (e.g., spring, elastic plug, etc.). Bias member 10100 can also be of a spring washer-type arrangement. Bias member 10100 includes top and bottom surfaces or portions 10102 and 10104 with flexible convex beam or strut portions 10106 and concave portions 10108. In some implementations, bias member 10100 also includes a plurality of flat or plateaued sections 10110 and 10112 distributed around the top and bottom surfaces or portions 10102 and 10104. The plurality of flat or plateaued sections 10110 and 10112 are arranged as bearing portions against which applied forces act and define a plane of stability. While three such sections are shown, more than three can be included such as, for example, four to six or more. The plurality of flat or plateaued sections 10110 and 10112 also provide bias member 10100 with the ability to be stacked or compounded with each bias member contacting or connected to the next via the plurality of flat or plateaued sections 10110 and 10112. In some implementations, a flat washer can be provided between each pair of adjacent bias members 10100 to stabilize the stacking. The beam or strut portions 10114 between the plateaued sections can also include the shape and tapered width arrangement described in connection with the beams or struts of FIG. 95. When force is applied to the bias member 10100, the flexible beams or struts 10106 deform in response by flexing or bending under the applied force and becoming less convex and concave. When the force is removed or lessened, the flexible beams or struts return to their original configuration. Bias member 10100 can be stacked or compounded by affixing plateaued portions 10110 together by, for example, welding or other secure means.

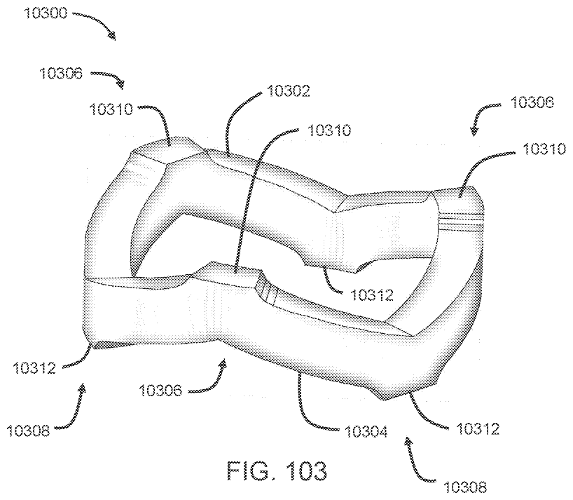

Referring now to FIG. 103, an example of a bias member 10300 (e.g., spring, elastic plug, etc.) is illustrated. Bias member 10300 is similar to bias member 10100 and can also be of a similar spring washer-type arrangement. Bias member 10300 includes top and bottom surfaces or portions 10302 and 10304 also of a generally rectangular configuration compared to bias member 10100 and includes flexible convex beam or strut portions 10306 and concave portions 10308. Bias member 10300 includes a thicker body between top and bottom surfaces or portions 10302 and 10304 compared to bias member 10100. This can provide increased stiffness if desired. Similar to bias member 10100, bias member 10300 also includes a plurality of flat or plateaued sections 10310 and 10312 distributed around the top and bottom surfaces or portions 10302 and 10304. The plurality of flat or plateaued sections 10310 and 10312 are arranged as bearing portions against which applied forces act. The plurality of flat or plateaued sections 10310 and 10312 also provide bias member 10300 with the ability to be stacked or compounded with each bias member contacting or connected to the next via the plurality of flat or plateaued sections 10310 and 10312. The beams or struts between the plateaued sections can include the same shape and tapered width configurations as described in connection with the beams and struts of FIG. 95. When force is applied to the bias member 10300, the flexible beam or strut portions 10306 deform in response by flexing or bending under the applied force and becoming less convex and concave. When the force is removed or lessened, the flexible beams or struts return to their original configuration. Bias member 10300 can be stacked or compounded by affixing plateaued portions 10310 together by, for example, welding or other secure means and/or by providing flat washer(s) between the stacked bias members.

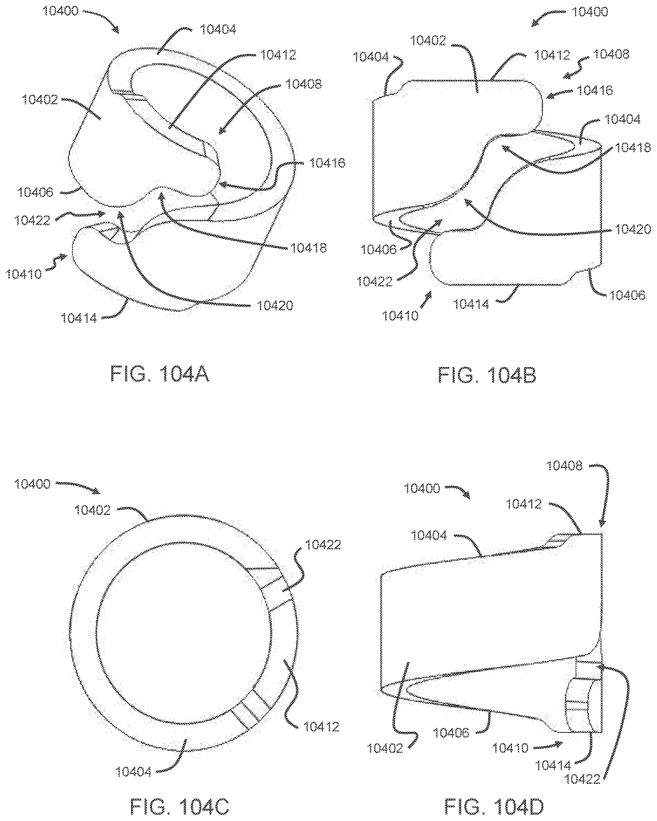

FIGS. 104A, 104B, 104C and 104D illustrate an example of a bias member 10400 arranged as a split spring washer-type or single revolution coil-type. Bias member 10400 includes a flexible body 10402 that is generally circular or cylindrical and has top and bottom surfaces or portions 10404 and 10406. Top and bottom surfaces or portions 10404 and 10406 can each include a projecting bearing portion, shoulder or tab 10412 and 10414. Bias member 10400 is arranged with a split or gap portion 10422 separating first and second end portions 10408 and 10410. Each of the end portions include one or more surface features including, for example, concave portion 10416, convex portion 10418 and concave portion 10420. Other surface features can be used including stepped features and other combinations of extended and/or recessed shapes and geometries. When force is applied to the bias member 10400, flexible body 10402 deforms in response by flexing or bending under the applied force and lessening the size of gap 10422. This shortens the overall height of flexible body 10402 (e.g., the distance between top and bottom surfaces or portions 10404 and 10406). When the force is removed or lessened, flexible body 10402 returns to its original configuration. Due to its configuration and shape, bias member 10400 provides effective fatigue resistance and compliance force output.

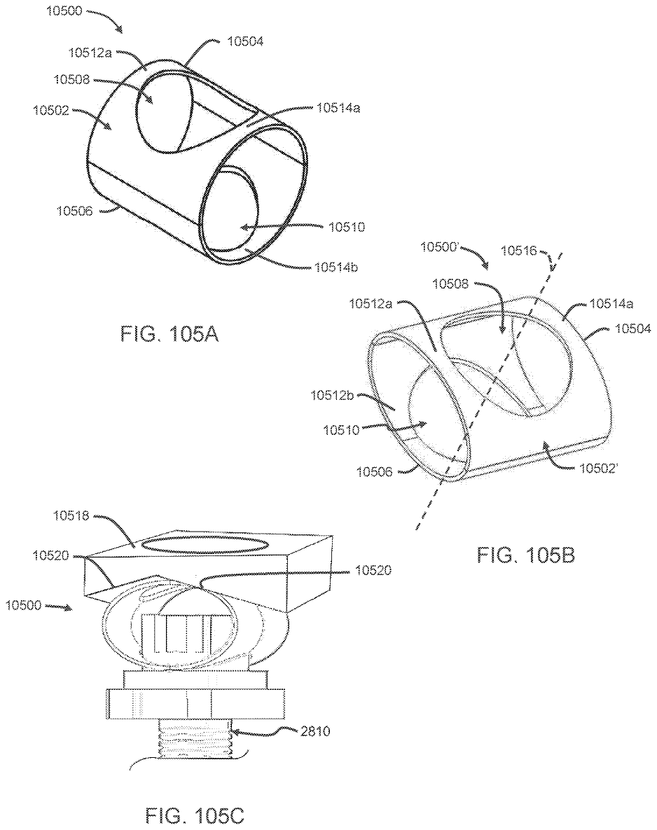

Referring now to FIGS. 105A, 105B and 105C, examples of bias members (e.g., spring, elastic plug, etc.) including bias members 10500 and 10500′ are illustrated. Bias member 10500 includes a generally tubular body 10502, which can be cylindrically shaped. Bias member 10500′ includes a tubular body 10502′ that is oval or elliptical in shape. Generally, tubular bodies 10502 and 10502′ include upper and lower portions 10504 and 10506. Upper portion 10504 includes an opening, aperture or cutout 10508 and lower portion 10506 includes an opening, aperture or cutout 10510. Cutouts 10508 and 10510 can be arranged on a common axis or centerline 10516 and can be disposed equidistant or in the middle generally of tubular body 10502. Cutouts 10508 and 10510 are configured to allow generally tubular body 10502 to receive portions of drive member 2810 therein (e.g., FIG. 105C). Upper and lower portions 10504 and 10506 also include bearing portions or surfaces 10512a, 10512b and 10514a, 10514b that are proximate to cutouts 10508 and cutout 10510. When force is applied to the bias member 10500 or 10500′ and its bearing portions and surfaces, such as by either movable drive member 2810 and/or a housing or position adjustment member 10518, the generally tubular bodies 10502 and 10502′ deform in response by flexing or bending under the applied force and becoming oval/elliptical or more oval/elliptical in shape. Housing or position adjustment member 10518 can include one or more bearing surfaces 10520 which can be angled (or shaped) to bear against wider sections of corresponding bearing portions 10512a and 10514a. When the force is removed or lessened, the flexible bodies 10502 and 10502′ return to their original configuration.

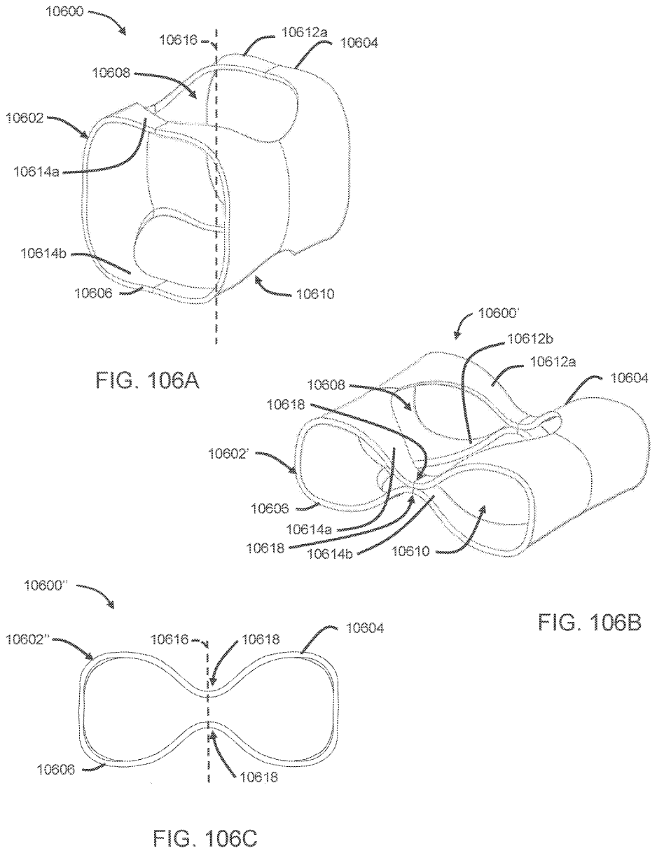

Referring now to FIGS. 106A, 106B and 106C, examples of bias members including bias members 10600, 10600′ and 10600″ (e.g., spring, elastic plug, etc.) are illustrated. Bias member 10600 includes a generally tubular body 10602, which can be generally rectangular in shape. In some implementations, though generally rectangular or square in shape, body 10602 can still include side walls and joints having various amounts of curvature and/or roundness. Bias member 10600′ and 10600″ include bodies 10602′ and 10602″ have a generally looped, figure “8” or infinity “∞” arrangement. Each includes upper and lower portions 10604 and 10606. Upper portion 10604 includes an opening, aperture or cutout 10608 and lower portion 10606 includes an opening, aperture or cutout 10610. Cutouts 10608 and 10610 can also be arranged on a common axis or centerline 10616 and can be disposed equidistant or in the middle of the bodies. Cutouts 10608 and 10610 are similarly configured to allow the bodies to receive portions of drive member 2810 therein (e.g., see FIG. 105C). Upper and lower portions 10604 and 10606 also include bearing portions or surfaces 10612a, 10612b and 10614a, 10614b that are proximate to cutouts 10608 and cutout 10610. When force is applied to the bearing portions and surfaces, such as by either movable drive member 2810 and/or a housing or position adjustment member, the bodies 10602, 10602′ and 10602″ deform in response by flexing or bending under the applied force and becoming more compressed and, in the case of bodies 10602′ and 10602″, wider. When the force is removed or lessened, the flexible body 10602 returns to its original configuration. Bodies 10602″ can also provide multiple force displacement characteristics or curves wherein a first force displacement characteristic is provided until portions 10618 bear against each other and a second is provided thereafter as upper and lower portions 10604 and 10606 continue to bear further forces and displacement. This is in contrast to the configuration of body 10602′ in which portions 10618 already bear against each other.

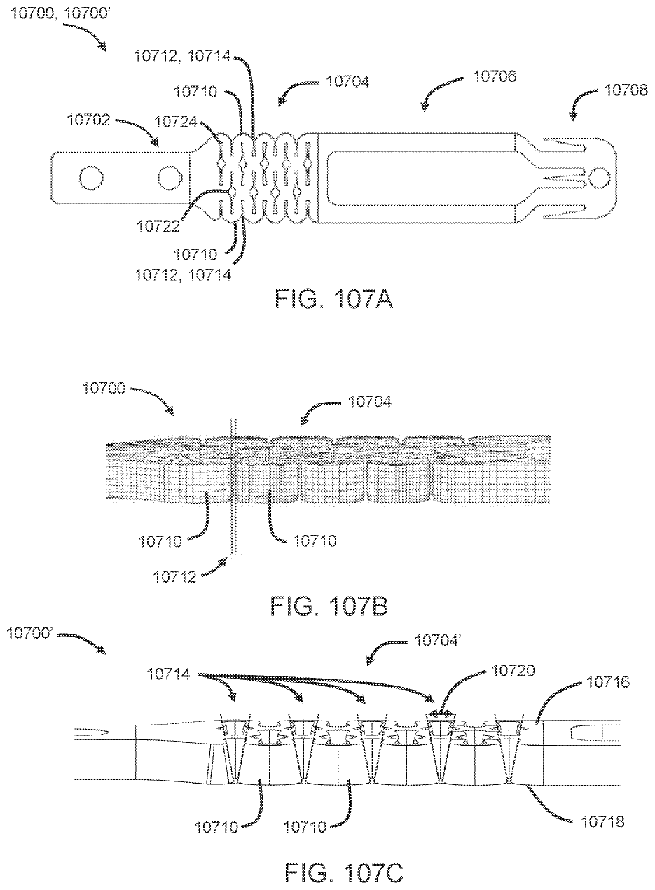

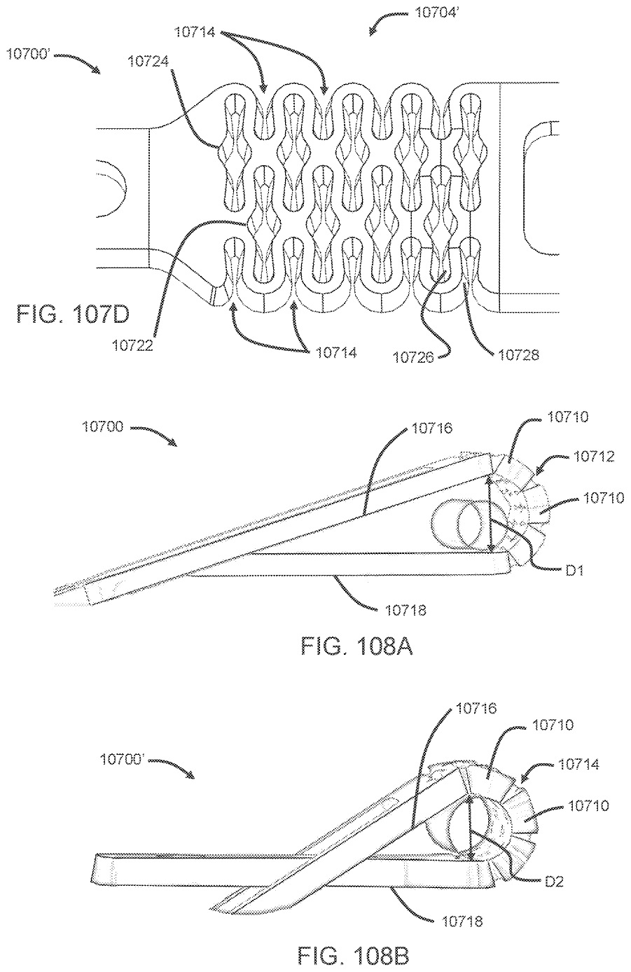

FIGS. 107A, 107B, 107C and 107D illustrate examples of a clasp arrangement for a heart valve repair device. The clasp arrangement can be used in combination with any of the implementations disclosed herein. FIG. 107A illustrates a top view of a flat pattern used to make a clasp 10700 having fixed portion 10702, a joint portion 10704, and moveable arm portion 10706, which can include an optional barb portion 10708. Fixed portion 10702 can be attached to an inner paddle structure. Joint portion 10704 can be formed to include a plurality of spring elements 10710 (e.g., shape set struts) separated by one or more gaps or spaces 10712 (FIGS. 107A, 108A) and/or 10714 (FIGS. 107B, 108B). The plurality of spring elements 10710 and one or more gaps or spaces 10712, 10714 can be made by any suitable means including, for example, being cut or laser cut between the top and bottom sides 10716 and 10718 of the clasp structure. So arranged, joint portion 10704 can flex or bend under spring pressure thereby allowing the clasps to flex or bend under spring pressure.

Referring now to FIG. 107B, one example of joint portion 10704 is illustrated having a plurality of gaps or spaces 10712 that are generally rectangular with parallel side walls. FIGS. 107C and 107D illustrate an example of a joint portion 10704′ having one or more gaps or spaces 10712 that are tapered by side walls of spring elements 10710. The tapering can include a taper angle 10720 between the non-parallel sidewalls that can be in the range of 5 to 45 degrees, or more. In some implementations, the taper angle 10720 includes an angle between 20 to 25 degrees. The tapered gaps, spaces or walls can be linear and/or curved. The tapered gaps or spaces can decrease or increase in size as they transition from top surface 10716 to bottom surface 10718. The tapered gaps or spaces can include end portions with one located on top surface 10716 and one locating on bottom surface 10718 and can be of different sizes thereby gradually forming a taper, as shown by of example by tapered gap, space or walls 10714 in FIG. 107C. The tapered gaps or spaces can also include a plurality of tapers having different arrangements (i.e., the tapered gaps, spaces, or walls do not have be identical). Further, the tapered gaps, spaces or walls 10714 can be positioned on the outer side portions of joint portion 10704′. In some implementations, the tapered gaps or spaces can include all the gaps, spaces, or walls of joint portion 10704′ and not only along the side portions. This includes inner portions as represented by gaps, spaces or walls 10722 and 10724. Thus, all the walls, including inner and outer walls, as represented by inner wall 10726 and outer wall 10728, can include a taper or angle. In the example shown, the taper is such that top surface 10716 has less area than bottom surface 10718 in joint portion 10704′. Therefore, each spring element 10710 has collectively a smaller top surface area than bottom surface area.

FIG. 108A shows clasp 10700 of FIGS. 107A and 107B in its flexed or bent configuration and FIG. 108B shows clasp 10700′ of FIG. 107C in its flexed or bent configuration. In FIG. 108A, it can be seen that clasp 10700 forms a bend having a diameter distance of approximately D1. And, in FIG. 108B, it can be seen that clasp 10700′ forms a bend having a diameter distance of approximately D2. Due to the clasp 10700′ having one or more tapered gaps or spaces 10714, compared to the non-tapered or parallel gaps or spaces 10712 of clasp 10700, clasp 10700′ is able to bend or flex further than clasp 10700. Thus, the bend diameter distance D2 of clasp 10700′ (e.g., tapered) is smaller than the bend diameter distance of D1 of clasp 10700 (non-tapered). Or said another way the bend diameter radius (e.g., ½ of D2) of clasp 10700′ (e.g., tapered) is smaller or tighter than the bend diameter radius (e.g., ½ of D1) of clasp 10700 (non-tapered). This allows the heart valve repair device to be more compact (e.g., FIG. 23) and due to the clasps having a smaller profile between the base, center member, or coaptation element (if included) and the paddles.

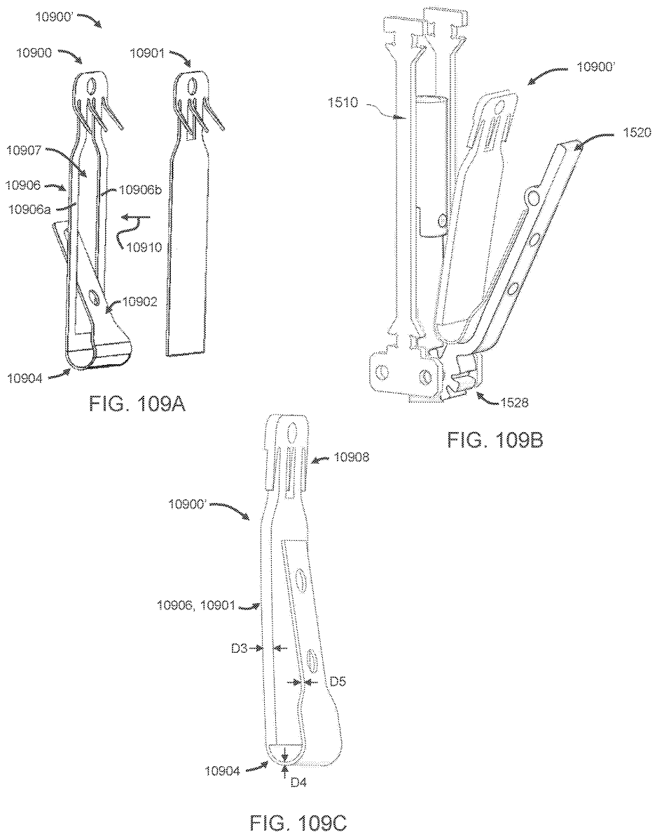

Referring now to FIGS. 109A, 109B, and 109C, clasp examples are shown including, for example, clasps 10900 and 10900′. FIG. 109A illustrates clasp 10900′ in an exploded perspective view. FIG. 109B shows clasp 10900′ in association with base 1510, paddles 1520, and pivotable portion 1528 and in isolation from other components of the heart valve repair device. FIG. 109C shows the clasp 10900 in isolation from all other components.

Clasp 10900′ includes a first clasp portion 10900 that includes a base portion 10902, joint portion 10904 and movable arm portion 10906. The movable arm portion 10906 can include one or more optional barb portions 10908, an opening or aperture 10907, and beam or strut portions 10906a and 10906b. A second clasp portion 10901 is also provided and formed similar to movable arm portion 10906 but without aperture 10907. During the manufacturing process, first clasp 10900 can comprise and/or can be formed from a material such as nitinol (or other similar material) to the shape shown and base portion 10902 is bent to the position shown in FIG. 109A and shape set to that configuration. This shape set configuration provides clasp 10900′ with a pinch force between base portion 10902 and movable arm portion 10906. Reinforcing or second clasp portion 10901 is then attached, as represented by arrow 10910 by welding, gluing, fastening, etc., to movable arm portion 10906 to arrive at the configuration shown in FIG. 109C. This provides beam or strut portions 10906a and 10906b with reinforcement to better handle the stresses that are formed as clasp 10900′ opens and closes. Thus, second clasp portion 10901 acts as a stiffener or thickener for movable arm portion 10906.

So configured, movable arm portion 10906 includes an arm thickness D3 and the joint portion 10904 includes a joint thickness D4. Base portion 10902 further includes a base thickness D5. In the example shown, the arm thickness D3 is greater than the joint thickness D4 and base thickness D5 due to stiffening or thickening clasp portion 10901. This creates different degrees of stiffness or rigidity on clasp 10900′. In the case of arm thickness D3 being greater than joint thickness D4, movable arm portion 10906 is stiffer than joint portion 10904 and, hence, tends to be more resistant to bending or flexing compared to joint portion 10904 (and/or base portion 10902). This assists movable arm portion 10906 in retaining its shape and geometry during the leaflet capture process where the portions of the clasp 10900 are bent and/or flexed. It also reduces the stresses and strains on movable arm portion 10906 caused by such bending and flexing due to the increased stiffness, rigidity, and/or thickness of movable arm portion 10906. Clasp 10900′ can be made of any clasp material disclosed herein including, for example, metals (e.g., nitinol) and formed into shape by any process described herein including, for example, laser cutting.

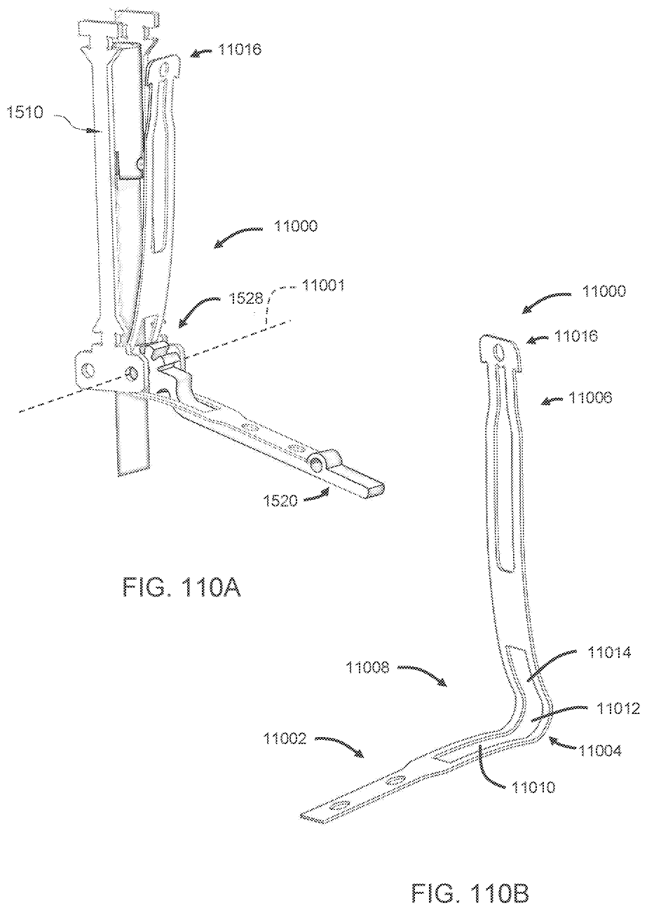

Referring now to FIGS. 110A and 110B, an example of a clasp 11000 is illustrated. Clasp 11000 is suitable for any of the disclosed heart valve repair devices described herein. FIG. 110A shows clasp 11000 in association with base 1510, paddles 1520, and pivotable portion 1528 and in isolation from other components of the heart valve repair device. FIG. 110B shows the clasp 11000 in isolation from all other components.

Clasp 11000 includes a base portion 11002, joint portion 11004 and movable arm portion 11006. The movable arm portion 11006 can include one or more barb portions 11008. The base portion 11002 includes a central cutout, aperture or opening 11012, which can optionally extend via spaces 11010 and 11014 into the base portion 11002 and the movable arm portion 11006. Joint portion 110004 can be curved or arched and include a central axis. As shown in FIG. 110A, opening 11012 receives at least a portion of paddle pivotable portion 1528, which can include a gear portion thereof. In some implementations, the central axis of pivotable portion 1528 and the central axis of joint portion 11004 are coaxial or substantially coaxial as represented by axis 11001. The term “substantially” as used throughout this disclosure further means acceptable for the intended application but not necessarily precise or perfect and allows for a small amount of flexibility or tolerancing due to, for example, minor imperfections that are common and acceptable.

In operation, the coaxial alignment 11001 of pivotable portion 1528 and joint portion 11004 reduces stresses and strains on clasp 11000. For example, the stresses and strains are reduced in joint portion 11004 when paddles 1520 (to which base portion 11002 is attached) are rotated or pivoted outward to a significant degree (e.g., 90 to 180 degrees from their closed position). Opening 11012 allows joint portion 11004 to flex or bend to those high degrees without having to bend over pivotable portion 1528 (e.g., gear portion) and incur high strains. The coaxial arrangement also provides the barb portion 11016 (shown without barbs) with a non-drifting arrangement were the position of barb portion 11016 does not drift up or down relative to the paddles and/or the coaptation element as clasp 11000 is opened and closed. In some implementations, the coaxial arrangement allows the paddles to move from an open position toward a closed position, with the clasp maintained in an open position, without moving or without substantially moving the movable clasp arm 11005 relative to the base 1510. While barb portion 11016 is shown without barbs, barbs can be optionally included according to any of the implementations disclosed herein.

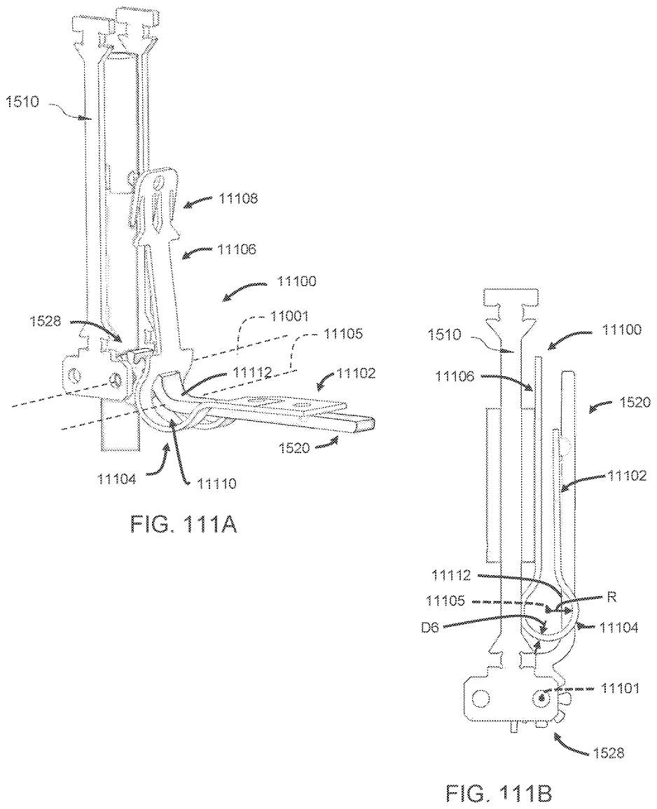

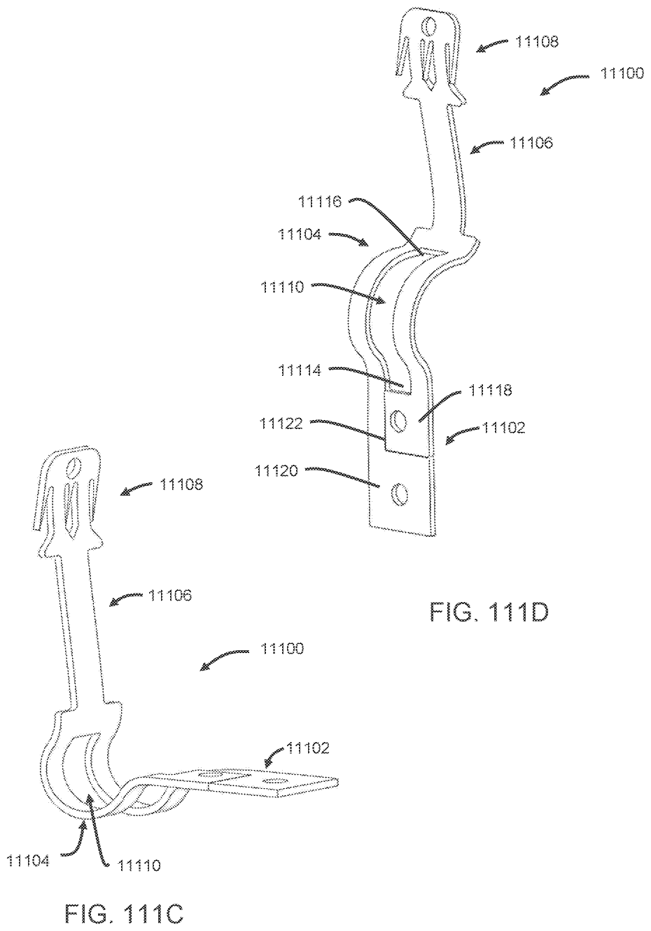

Referring now to FIGS. 111A, 111B, 111C and 111D, an example of a clasp 11100 is illustrated. Clasp 11100 is suitable for any of the disclosed heart valve repair devices described herein. FIGS. 111A and 111B show clasp 11100 in association with base 1510, paddles 1520, and pivotable portion 1528 and in isolation from other components of the heart valve repair device. FIGS. 111C and 111D show the clasp 11100 in isolation from all other components.

Clasp 11100 includes a base portion 11102, joint portion 11104 and movable arm portion 11106. The movable arm portion 11106 can include one or more barb portions 11108. The joint portion 11104 includes a central cutout, aperture or opening 11110, which can optionally extend via spaces 11114 and 11116 into the base portion 11102 (and also into the movable arm portion 11006, if desired). The joint portion 11104 can be curved or arched and include a central axis 11105. As shown in FIG. 111A, opening 11110 receives at least a portion of paddle arm 11112. In some implementations, the central axis 11001 of pivotable portion 1528 and the central axis of opening 11110 are offset by virtue of clasp 11100 being located on pivoting arm portion 11112 of paddle 1520. Positioning clasp 11100 at this general location allows clasp 11100 to optionally be made from a thicker material (e.g., see thickness D6) that can better handle strains created by high bending or flexing movement. The thicker material includes thicker metal sheets (e.g., of nitinol) from which clasp 11100 can be formed or cut. Also, by being positioned offset from axis 11001 of paddle frame member 1529 and straddled on a portion of the arm 11112 of paddle 1520, joint portion 11104 can be formed having a larger bend radius “R” that can also reduce the strains associated with high bending and flexing.

In some implementations, clasp 11100 includes a split base portion 11102 having first and second portions 11118 and 11120 (see FIG. 111D). The split portions 11118 and 11120 can be created by, for example a laser cut 11122 having longitudinal and lateral sub-cuts. The split base portion can also be created by other means including, for example, stamping and other cutting or forming means.

In operation, FIG. 111B shows paddles 1520 and clasp 11100 in their closed or pre-capture ready position. This also shows paddles 1520 and clasp 11100 in their closed or collapse position as used for deployment of the heart valve repair device through a catheter delivery system. FIG. 111A shows paddles 1520 and clasp 11100 in the open position at approximately 90 degrees open. When paddle 1520 (to which base portion 11102 is attached) is rotated or pivoted outward to a significant degree (e.g., 90 to 180 degrees from their closed position), joint portion 11104 moves with paddle arm portion 11112 and also bends or flexes allowing movable arm portion 11106 to maintain its position as shown in FIG. 111A. As paddle 1520 continues to pivot up to about 180 degrees, joint portion 11104 continues to move with paddle 1520 and continues to bend or flex so that movable arm portion 11106 can remain in position (e.g., to be ready for the leaflet capture-ready position) while base portion 11102 moves with paddle 1520. Opening 11110 allows joint portion 11004 flex or bend to those high degrees without having to fully bend over backwards over pivotable portion 1528 (e.g., gear portion) and incur high strains. This is because opening 11110 can receive at least a portion of pivotable portion 1528 (e.g., paddle and/or gears) therein to lessen the bending or flexing by joint portion 11104. Furthermore, the outward position of joint portion 11104 on paddles 1520 allows clasp 11100 to be made of a thicker material (e.g., D6 in FIG. 111B) compared to other examples, which can provide better strain and stress properties to clasp 11100 while also improving or increasing the clasp pinch force under these dynamic circumstances.

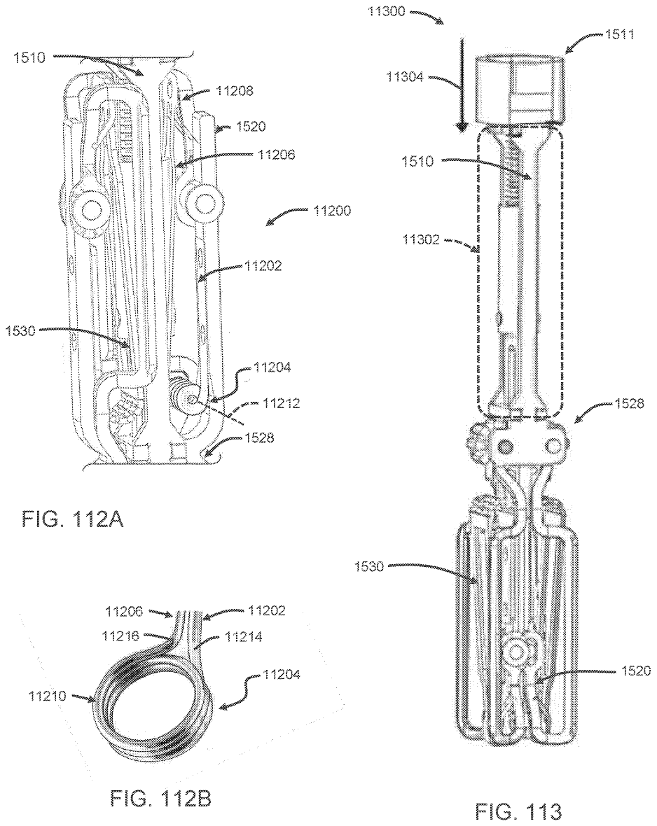

Referring now to FIGS. 112A and 112B, an example of a clasp 11200 is illustrated. Clasp 11200 is suitable for any of the disclosed heart valve repair devices described herein. FIGS. 112A and 112B show clasp 11200 in association with base 1510, paddles 1520, and pivotable portion 1528 along with other components of the heart valve repair device. FIG. 112A also shows for comparison purposes a gripping member or clasp 1530 as previously disclosed herein.

Clasp 11200 includes a base portion 11202, joint portion 11204 and movable arm portion 11206. The movable arm portion 11206 can include one or more barb portions 11208. The joint portion includes a bias element 11210, which can be in the form of a spring coil having one or more coil portions. A first coil (end) portion 11214 can connect to base portion 11202 and a second coil (end) portion 11216 can connect to movable arm portion 11206. Bias element 11210 can include a spring or coil axis 11212 that is offset from paddle pivot axis 11101 of pivotable portion 1528. Bias element 11210 provides a bias force (or pinch force) to clasp 11200 that urges base portion 11202 and movable arm portion 11206 together.

In some implementations, clasp 11200 can be made from a single piece of material. In some implementations, clasp 11200 can be made from multiple pieces of material. For example, base portion 11202 can be made from a first piece of material, the movable arm portion 11206 can be made from a second piece of material, and the coil spring portion 11210 can be made from a third piece of material and wherein the third piece of material is joined to the first and second pieces of material. The joinder or fixation can be by any suitable means including, for example, welding, fastening, glueing, etc.

So arranged, bias element 11210 in the form of one or more spring coils provides clasp 11200 with the ability to bend or flex while under the stresses and strains caused by such movement(s) without significantly impacting the pinch force provided by the bias element.

FIG. 113 illustrates an example of a heart valve repair device 11300 according to any of the examples disclosed herein having a spacer or coaptation element 11302. Spacer or coaptation element 11302 can be according to any of the examples disclosed herein. FIG. 113 shows an example of the valve repair device 11300 in the delivery configuration such as, for example, when moving through a catheter or lumen of a delivery system. Spacer or coaptation element 11302 can be positioned around base 1510, or portions thereof, as the heart valve device travels through the catheter or lumen. Spacer or coaptation element 11302 does significantly increase the overall size of the heart valve device in terms of sizing for the delivery system. In some implementations, the heart valve repair device shown in FIG. 113 can be configured to initially travel through the catheter or lumen without spacer or coaptation element 11302. Spacer or coaptation element 11302 can be subsequently positioned or added through the catheter or lumen, as represented by arrow 11304, to the position shown, which can be sliding or moving spacer or coaptation element 11302 over part of the device until the position shown in FIG. 113 is reached. In either configuration, valve repair device 11300 provides a suitably compact physical arrangement that allows the heart valve repair device to move through the delivery system, while including a coaptation element that enhances sealing between the device and the native heart valve leaflets.

The following includes additional descriptions of examples and implementations. The examples are intended to illustrate examples of combinations of elements and are not intended to limit the scope of any particular example or implementation disclosed herein.

Example 1. A valve treatment device, comprising: a base; an actuation assembly coupled to the base; one or more paddles pivotally coupled to the base; and wherein the actuation assembly is configured to convert a rotational input motion to pivoting movement of the one or more paddles relative to the base.

Example 2. The device of example 1 wherein the base comprises a frame with an open interior.

Example 3. The device of example 2 wherein the actuation assembly is disposed in the open interior of the base.

Example 4. The device of any one of examples 1-3 wherein the actuation assembly comprises one or more of a rack and pinion assemblies, a worm gear assembly, and a planetary gear assembly.

Example 5. The device of any one of examples 1-4 wherein the actuation assembly comprises a threaded drive member, a threaded driven member, and a gear rack.

Example 6. The device of any one of examples 1-5 wherein each of the one or more paddles comprises a pinion gear.

Example 7. The device of example 1 wherein the actuation assembly comprises a rack and each of the one or more paddles comprises a pinion gear that is driven by the rack.

Example 8. The device of any one of examples 1-7 further comprising a gripping member configured to grasp a native valve leaflet.

Example 9. The device of example 8 wherein the gripping member is configured to grasp the native valve leaflet with one of the one or more paddles.

Example 10. The device of any one of examples 1-9 wherein each of the one or more paddles extends from a pivotable portion that is pivotably connected to the base to a free end.

Example 11. The device of any one of examples 1-10 further comprising a paddle frame connected to each of the one or more paddles, wherein the paddle frame is configured to coapt two native valve leaflets together.

Example 12. The device of any one of examples 1-11 wherein each paddle of the one or more paddles can form an angle with the base that is greater than 175 degrees.

Example 13. The device of example 8 wherein the gripping member can form an angle of 180 degrees with one of the one or more paddles.

Example 14. A valve treatment system comprising, comprising: a catheter; a valve treatment device coupled to the catheter, the valve treatment device comprising: a base; an actuation assembly coupled to the base; one or more paddles pivotally coupled to the base; and wherein the actuation assembly is configured to convert a rotational input motion to pivoting movement of the one or more paddles relative to the base.

Example 15. The system of example 14 wherein the base comprises a frame with an open interior.

Example 16. The system of example 15 wherein the actuation assembly is disposed in the open interior of the base.

Example 17. The system of any one of examples 14-16 wherein the actuation assembly comprises one or more of a rack and pinion assembly, a worm gear assembly, and a planetary gear assembly.

Example 18. The system of any one of examples 14-17 wherein the actuation assembly comprises a threaded drive member, a threaded driven member, and a gear rack.

Example 19. The system of any one of examples 14-18 wherein each of the one or more paddles comprises a pinion gear.

Example 20. The system of example 14 wherein the actuation assembly comprises a rack and each of the one or more paddles comprises a pinion gear that is driven by the rack.

Example 21. The system of any one of examples 14-20 further comprising a gripping member configured to grasp a native valve leaflet.

Example 22. The system of example 21 wherein the gripping member is configured to grasp the native valve leaflet with one of the one or more paddles.

Example 23. The system of any one of examples 14-22 wherein each of the one or more paddles extends from a pivotable portion that is pivotably connected to the base to a free end.

Example 24. The system of any one of examples 14-23 further comprising a paddle frame connected to each paddle of the one or more paddles, wherein the paddle frame is configured to coapt two native valve leaflets together.

Example 25. The system of any one of examples 14-24 wherein each paddle of the one or more paddles can form an angle with the base that is greater than 175 degrees.

Example 26. The system of example 21 wherein the gripping member can form an angle of 180 degrees with one of the one or more paddles.

Example 27. A treatment and/or repair system, comprising: a guide sheath; a steerable catheter that extends through the guide sheath; and implant catheter that extends through the steerable catheter; a treatment and/or repair device coupled to the implant catheter; wherein the treatment and/or repair device includes one or more paddles that are movable between an open position and a closed position; wherein the one or more paddles are each configured to be secured to a native valve leaflet by moving the paddle from the open position to the closed position; and wherein a passage of the steerable catheter and a size of the treatment and/or repair device are configured to allow the treatment and/or repair device to slide through the steerable catheter.

Example 28. The treatment and/or repair system of example 27 wherein the treatment and/or repair device is configured to be secured to leaflets of a native mitral valve having a mitral valve area (MVA) in cm² between 2.2 and 6.

Example 29. The treatment and/or repair system of example 27 wherein the treatment and/or repair device is configured to be secured to leaflets of a native mitral valve when a transeptal puncture height in cm is between 2.0 and 4.5.

Example 30. The treatment and/or repair system of example 27 wherein the treatment and/or repair device is configured to be implanted where a mitral valve area is between 2.2 and 6 cm² and a transeptal puncture height is between 2.0 and 4.5 cm.

Example 31. The treatment and/or repair system of any of examples 27-30 wherein a medial-lateral width of the treatment and/or repair device is between 2 mm and 4 mm.

Example 32. The treatment and/or repair system of any one of examples 27-31 wherein an anterior-posterior width of the treatment and/or repair device is between 2 mm and 9 mm.

Example 33. The treatment and/or repair system of any one of examples 27-32 wherein a leaflet capture height is between 5 mm and 10 mm.

Example 34. The treatment and/or repair system of any one of examples 27-33 wherein the treatment and/or repair device includes a base, an actuation assembly coupled to the base, and wherein the actuation assembly is configured to convert a rotational input motion to pivoting movement of the one or more paddles relative to the base.

Example 35. The treatment and/or repair system of example 34 wherein the base comprises a frame with an open interior.

Example 36. The treatment and/or repair system of example 35 wherein the actuation assembly is disposed in the open interior of the base.

Example 37. The treatment and/or repair system of any one of examples 34-36 wherein the actuation assembly comprises one or more of a rack and pinion assembly, a worm gear assembly, and a planetary gear assembly.

Example 38. The treatment and/or repair system of any one of examples 34-37 wherein the actuation assembly comprises a threaded drive member, a threaded driven member, and a gear rack.

Example 39. The treatment and/or repair system of any one of examples 34-38 wherein each of the one or more paddles comprises a pinion gear.

Example 40. The treatment and/or repair system of example 34 wherein the actuation assembly comprises a rack and each of the one or more paddles comprises a pinion gear that is driven by the rack.

Example 41. The treatment and/or repair system of any one of examples 34-40 further comprising a gripping member configured to grasp the native valve leaflet.

Example 42. The treatment and/or repair system of example 41 wherein the gripping member is configured to grasp the native valve leaflet with one of the one or more paddles.

Example 43. The treatment and/or repair system of any one of examples 34-42 wherein each paddle of the one or more paddles extends from a pivotable portion that is pivotably connected to the base to a free end.

Example 44. The treatment and/or repair system of any one of examples 34-43 further comprising a paddle frame connected to each paddle of the one or more paddles, wherein the paddle frame is configured to coapt two native valve leaflets together.

Example 45. The treatment and/or repair system of any one of examples 34-44 wherein each paddle of the one or more paddles can form an angle with the base that is greater than 175 degrees.

Example 46. The treatment and/or repair system of example 41 wherein the gripping member can form an angle of 180 degrees with one of the one or more paddles.

Example 47. A valve treatment device for attaching to a native valve of a patient, the valve treatment device comprising: a base; one or more paddles pivotably coupled to the base such that the paddles are movable between an open position and a closed position; an actuation assembly coupled to the base, wherein the actuation assembly is configured to convert a rotational input motion to pivoting movement of the one or more paddles relative to the base such that the one or more paddles move between the closed position and the open position; wherein a proximal end of the actuation assembly is positioned proximate a gap, such that the actuation assembly can linearly move within the gap; a biasing member that engages the actuation assembly to maintain the actuation assembly in a desired position relative to the gap; and wherein, when the one or more paddles are in the closed position and attached to the native valve of the patient, movement of the leaflets during the diastolic phase causes the actuation assembly to linearly move within the gap.

Example 48. The device of example 47 wherein the base comprises a frame with an open interior.

Example 49. The device of example 48 wherein the actuation assembly is disposed in the open interior of the base.

Example 50. The device of any one of examples 47-49 wherein the actuation assembly comprises one or more of a rack and pinion assembly, a worm gear assembly, and a planetary gear assembly.

Example 51. The device of any one of examples 47-50 wherein the actuation assembly comprises a threaded drive member, a threaded driven member, and a gear rack.

Example 52. The device of example 51 wherein the threaded drive member comprises a head that moves linearly within the gap due to movement of the leaflets during the diastolic phase.

Example 53. The device of any one of examples 47-52 wherein each of the one or more paddles comprises one or more pinion gears.

Example 54. The device of example 47 wherein the actuation assembly comprises a rack and each of the one or more paddles comprises one or more pinion gears that are driven by the rack.

Example 55. The device of any one of examples 47-54 further comprising a gripping member configured to grasp a native valve leaflet.

Example 56. The device of example 55 further comprising a second actuation assembly coupled to the base, wherein the second actuation assembly is configured to move the gripping member between an open position and a closed position, wherein the second actuation assembly comprises a threaded drive member and a threaded driven member, and wherein the gripping member is connected to the threaded driven member such that movement of the threaded driven member by the threaded drive member causes the gripping member to move between the open and closed positions.

Example 57. The device of any one of examples 55-56 wherein the gripping member is configured to grasp the native valve leaflet with one of the one or more paddles.

Example 58. The device of any one of examples 47-57 wherein each of the one or more paddles extends from a pivotable portion, that is pivotably connected to the base, to a free end.

Example 59. The device of any one of examples 47-58 further comprising a paddle frame connected to each of the one or more paddles, wherein the paddle frame is configured to coapt two native valve leaflets together.

Example 60. The device of example 59 further comprising a second actuation assembly coupled to the base, wherein the second actuation assembly is configured to move the paddle frame between a narrowed configuration and an expanded configuration, wherein the second actuation assembly comprises a threaded drive member and a threaded driven member, and wherein the paddle frame is connected to the threaded driven member such that movement of the threaded driven member by the threaded drive member causes the paddle frame to move between the narrowed and expanded configurations.

Example 61. The device of example 60 further comprising frame actuation elements that are pivotably coupled to the one or more paddles, wherein the frame actuation elements are connected to the threaded driven member such that movement of the threaded driven member causes the paddle frame to move between the narrowed and expanded configurations.

Example 62. The device of any one of examples 47-61 wherein each paddle of the one or more paddles can form an angle with the base that is greater than 175 degrees.

Example 63. The device of any one of examples 47-62 further comprising a second actuation assembly that includes a threaded drive member and a threaded driven member, wherein the second actuation assembly is configured to actuate one or more of the base, a gripping member, and a paddle frame of the device.

Example 64. The device of any one of examples 47-63 wherein the biasing member is positioned within the gap and engages a threaded drive member of the actuation assembly to maintain the actuation assembly in a desired position relative to the gap.

Example 65. The device of any one of examples 47-63 wherein the biasing member is positioned outside of the gap and engages a threaded driven member of the actuation assembly to maintain the actuation assembly in a desired position relative to the gap.

Example 66. The device of any one of examples 47-65 further comprising a coupling, wherein the gap is disposed within the coupling.

Example 67. The device of any one of examples 47-66 wherein the biasing member comprises a spring made of Nitinol.

Example 68. A valve treatment system comprising, comprising: a catheter; a valve treatment device for attaching to a native valve of a patient, wherein the valve treatment device is coupled to the catheter, wherein the valve treatment device comprises: a base; one or more paddles pivotably coupled to the base such that the paddles are movable between an open position and a closed position; an actuation assembly coupled to the base, wherein the actuation assembly is configured to convert a rotational input motion to pivoting movement of the one or more paddles relative to the base such that the one or more paddles move between the closed position and the open position; a gap positioned proximate a proximal end of the actuation assembly such that the actuation assembly can linearly move within the gap; a biasing member that engages the actuation assembly to maintain the actuation assembly in a desired position relative to the gap; and wherein, when the one paddles are in the closed position and attached to the native valve of the patient, movement of the leaflets during the diastolic phase causes the actuation assembly to linearly move within the gap.

Example 69. The system of example 68 wherein the base comprises a frame with an open interior.

Example 70. The system of example 69 wherein the actuation assembly is disposed in the open interior of the base.

Example 71. The system of any one of examples 68-70 wherein the actuation assembly comprises one or more of a rack and pinion assembly, a worm gear assembly, and a planetary gear assembly.

Example 72. The system of any one of examples 68-71 wherein the actuation assembly comprises a threaded drive member, a threaded driven member, and a gear rack.

Example 73. The system of example 72 wherein the threaded drive member comprises a head that moves linearly within the gap due to movement of the leaflets during the diastolic phase.

Example 74. The system of any one of examples 68-73 wherein each of the one or more paddles comprises one or more pinion gears.

Example 75. The system of example 68 wherein the actuation assembly comprises a rack and each of the one or more paddles comprises one or more pinion gears that are driven by the rack.

Example 76. The system of any one of examples 68-75 wherein the valve treatment device further comprises a gripping member configured to grasp a native valve leaflet.

Example 77. The system of example 76 wherein the valve treatment device further comprises a second actuation assembly coupled to the base, wherein the second actuation assembly is configured to move the gripping member between an open position and a closed position, wherein the second actuation assembly comprises a threaded drive member and a threaded driven member, and wherein the gripping member is connected to the threaded driven member such that movement of the threaded driven member by the threaded drive member causes the gripping member to move between the open and closed positions.

Example 78. The system of any one of examples 76-77 wherein the gripping member is configured to grasp the native valve leaflet with one of the one or more paddles.

Example 79. The system of any one of examples 68-78 wherein each of the one or more paddles extends from a pivotable portion, that is pivotably connected to the base, to a free end.

Example 80. The system of any one of examples 68-79 wherein the valve treatment device further comprises a paddle frame connected to each of the one or more paddles, wherein the paddle frame is configured to coapt two native valve leaflets together.

Example 81. The system of example 80 wherein the valve treatment device further comprises a second actuation assembly coupled to the base, wherein the second actuation assembly is configured to move the paddle frame between a narrowed configuration and an expanded configuration, wherein the second actuation assembly comprises a threaded drive member and a threaded driven member, and wherein the paddle is connected to the threaded driven member such that movement of the threaded driven member by the threaded drive member causes the paddle frame to move between the narrowed and expanded configurations.

Example 82. The system of example 81 wherein the valve treatment device further comprises frame actuation elements that are pivotably coupled to the one or more paddles, wherein the frame actuation elements are connected to the threaded driven member such that movement of the threaded driven member causes the paddle frame to move between the narrowed and expanded configurations.

Example 83. The system of any one of examples 68-82 wherein each paddle of the one or more paddles can form an angle with the base that is greater than 175 degrees.

Example 84. The system of any one of examples 68-83 wherein the valve treatment device further comprises a second actuation assembly that includes a threaded drive member and a threaded driven member, wherein the second actuation assembly is configured to actuate one or more of the base, a gripping member, and a paddle frame of the device.

Example 85. The system of any one of examples 68-84 wherein the biasing member is positioned within the gap and engages a threaded drive member of the actuation assembly to maintain the actuation assembly in a desired position relative to the gap.

Example 86. The system of any one of examples 68-84 wherein the biasing member is positioned outside of the gap and engages a threaded driven member of the actuation assembly to maintain the actuation assembly in a desired position relative to the gap.

Example 87. The system of any one of examples 68-86 wherein the valve treatment device further comprises a coupling, wherein the gap is disposed within the coupling.

Example 88. The system of any one of examples 68-87 wherein the biasing member comprises a spring made of Nitinol.

Example 89. A valve treatment device for attaching to a native valve of a patient, the valve treatment device comprising: a base; one or more paddles pivotably coupled to the base such that the paddles are movable between an open position and a closed position; a first actuation assembly coupled to the base, wherein the first actuation assembly is configured to convert a rotational input motion to pivoting movement of the one or more paddles relative to the base such that the one or more paddles move between the closed position and the open position; and a second actuation assembly coupled to the base, wherein the second actuation assembly is configured to convert a rotational input motion to movement of one or more components of the valve treatment device relative to the base.

Example 89A. The valve treatment device of Example 89 wherein the second actuation assembly comprises a threaded drive member and a threaded driven member that is operatively attached to the threaded drive member such that rotation of the threaded drive member causes the threaded driven member to move linearly relative to the threaded drive member.

Example 90. The device of any one of examples 89 and 89A wherein the one or more components of the valve treatment device comprises a gripping member that is configured to grasp a native valve leaflet, wherein linear movement of the threaded driven member relative to the threaded drive member causes the gripping member to move between an open position and a closed position.

Example 91. The device of example 90 wherein the gripping member is configured to grasp the native valve leaflet with one of the one or more paddles.

Example 92. The device of any of examples 90-91 wherein the gripping member is attached to the driven member by an actuation line.

Example 93. The device of example 89 wherein the one or more components of the valve treatment device comprises a paddle frame connected to each of the one or more paddles, wherein the paddle frame comprises a body portion, a first arm pivotable relative to the body member, and a second arm pivotable relative to the body portion, and wherein linear movement of the threaded driven member relative to the threaded drive member causes the first and second arms to pivot relative to the body portion to move the paddle frame between a narrowed configuration and an expanded configuration.

Example 94. The device of example 93 further comprising a first frame actuation element and a second frame actuation element, wherein the first and second frame actuation elements are pivotable relative to a paddle of the one or more paddles between an open position and a closed, wherein the first arm of the paddle frame is attached to the first frame actuation element and the second arm of the paddle frame is attached to the second frame actuation element.

Example 95. The device of example 94 wherein the first and second frame actuation elements are attached to the threaded driven member by one or more actuation lines.

Example 96. The device of example 89 wherein the one or more components of the valve treatment device comprises a link assembly of the base, wherein the link assembly comprises a lower link that is pivotably attached to the base and an upper link that is pivotably attached to the threaded driven member, and wherein linear movement of the threaded driven member relative to the threaded drive member causes the upper and lower links to pivot relative to each other and move the base between a narrowed configuration and an expanded configuration.

Example 97. The device of example 89 wherein the one or more components of the valve treatment device comprises a movable member of the one or more paddles, wherein each of the one or more paddles comprises a fixed member and the movable member that is movable relative to the fixed member, and wherein linear movement of the threaded driven member relative to the threaded drive member causes the movable member to move relative to the fixed member such that the one or more paddles move between a shortened configuration and a lengthened configuration.

Example 98. The device of example 97 wherein the movable member is attached to the threaded driven member by one or more actuation lines.

Example 99. A valve treatment system comprising, comprising: a catheter; a valve treatment device for attaching to a native valve of a patient, wherein the valve treatment device is coupled to the catheter, wherein the valve treatment device comprises: a base; one or more paddles pivotably coupled to the base such that the paddles are movable between an open position and a closed position; a first actuation assembly coupled to the base, wherein the first actuation assembly is configured to convert a rotational input motion to pivoting movement of the one or more paddles relative to the base such that the one or more paddles move between the closed position and the open position; and a second actuation assembly coupled to the base, wherein the second actuation assembly is configured to convert a rotational input motion to movement of one or more components of the valve treatment device relative to the base.

Example 99A. The valve treatment system of Example 99, wherein the second actuation assembly comprises a threaded drive member and a threaded driven member that is operatively attached to the threaded drive member such that rotation of the threaded drive member causes the threaded driven member to move linearly relative to the threaded drive member.

Example 100. The device of example 99 or example 99A, wherein the one or more components of the valve treatment device comprises a gripping member that is configured to grasp a native valve leaflet, wherein linear movement of the threaded driven member relative to the threaded drive member causes the gripping member to move between an open position and a closed position.

Example 101. The device of example 100 wherein the gripping member is configured to grasp the native valve leaflet with one of the one or more paddles.

Example 102. The device of any of examples 100-101 wherein the gripping member is attached to the driven member by an actuation line.

Example 103. The device of example 99 wherein the one or more components of the valve treatment device comprises a paddle frame connected to each of the one or more paddles, wherein the paddle frame comprises a body portion, a first arm pivotable relative to the body portion, and a second arm pivotable relative to the body portion, and wherein linear movement of the threaded driven member relative to the threaded drive member causes the first and second arms to pivot relative to the body portion to move the paddle frame between a narrowed configuration and an expanded configuration.

Example 104. The device of example 103 further comprising a first frame actuation element and a second frame actuation element, wherein the first and second frame actuation elements are pivotable relative to a paddle of the one or more paddles between an open position and a closed, wherein the first arm of the paddle frame is attached to the first frame actuation element and the second arm of the paddle frame is attached to the second frame actuation element.

Example 105. The device of example 104 wherein the first and second frame actuation elements are attached to the threaded driven member by one or more actuation lines.

Example 106. The device of example 99 wherein the one or more components of the valve treatment device comprises a link assembly of the base, wherein the link assembly comprises a lower link that is pivotably attached to the base and an upper link that is pivotably attached to the threaded driven member, and wherein linear movement of the threaded driven member relative to the threaded drive member causes the upper and lower links to pivot relative to each other and move the base between a narrowed configuration and an expanded configuration.

Example 107. The device of example 99 wherein the one or more components of the valve treatment device comprises a movable member of the one or more paddles, wherein each of the one or more paddles comprises a fixed member and the movable member that is movable relative to the fixed member, and wherein linear movement of the threaded driven member relative to the threaded drive member causes the movable member to move relative to the fixed member such that the one or more paddles move between a shortened configuration and a lengthened configuration.

Example 108. The device of example 107 wherein the movable member is attached to the threaded driven member by one or more actuation lines.

Example 109. A valve treatment system comprising, comprising: a delivery device that includes an actuation element, the actuation element comprising: a catheter; a drive end connected to the catheter, the drive end having a passage that is in communication with the catheter and a slot at a distal end of the drive end that is in communication with the passage; a securing element that is configured to be moved through the catheter and the passage such that the securing element can be inserted into the slot at the distal end of the drive end; a valve treatment device for attaching to a native valve of a patient, the valve treatment device comprising: a base; an actuation assembly coupled to the base, wherein the actuation assembly comprises a drive member having a head, wherein the head comprises a bar that is configured to be inserted into the slot of the drive end of the actuation element of the delivery device; and wherein the head of the actuation assembly of the valve treatment device is secured to the actuation element of the delivery device when the bar of the head of the drive member is inserted into the slot of the drive end of the actuation element and the securing element of the actuation element is positioned within the slot.

Example 110. The system of example 109 wherein rotation of the drive end of the actuation element causes the drive element of the actuation assembly of the valve treatment device to rotate when the head of the actuation assembly of the valve treatment device is secured to the actuation element.

Example 111. The system of any one of examples 109-110 wherein the delivery device further comprises an outer catheter for housing the actuation element.

Example 112. The system of example 111, wherein the outer catheter comprises one or more slots that are configured to receive one or more projections of the head of the drive member of the valve treatment device such that the outer catheter can be secured to the valve head of the drive member by inserting the projections of the head into the slots of the outer catheter.

Example 113. The system of any one of examples 109-112 wherein the passage of the drive end has a proximal opening that is sized to align with the catheter of the actuation element and one or more sloped walls such that the passage narrows from the proximal opening to a distal opening.

Example 114. A valve treatment device for attaching to a native valve of a patient, the valve treatment device comprising: a base; one or more paddles pivotably coupled to the base such that the one or more paddles are movable between an open position and a closed position; an actuation assembly coupled to the base, wherein the actuation assembly is configured to pivot the one or more paddles relative to the base between the open position and the closed position responsive to the actuation assembly receiving an input; wherein a proximal end of the actuation assembly is positioned proximate a gap such that the actuation assembly can linearly move within the gap; and wherein, when the one or more paddles are in the closed position and attached to the native valve of the patient, movement of the leaflets during the diastolic phase causes the actuation assembly to linearly move within the gap.

Example 115. The device of example 114, further comprising a biasing member that engages the actuation assembly to maintain the actuation assembly in a desired position relative to the gap.

Example 116. The device of example 115, wherein the biasing member is positioned within the gap and engages a threaded drive member of the actuation assembly to maintain the actuation assembly in a desired position relative to the gap.

Example 117. The device of example 115, wherein the biasing member is positioned outside of the gap and engages a threaded driven member of the actuation assembly to maintain the actuation assembly in a desired position relative to the gap.

Example 118. The device of any one of examples 114-117, further comprising a coupling attached to the base, wherein the gap is disposed within the coupling.

Example 119. The device of any one of examples 114-118 wherein the biasing member comprises a spring made of Nitinol.

Example 120. The device of any one of examples 114-119, wherein the base comprises a frame.

Example 120. The device of example 120, wherein the actuation assembly is disposed in an open interior of the frame.

Example 121. The device of any one of examples 114-120, wherein the actuation assembly comprises one or more of a rack and pinion assembly, a worm gear assembly, and a planetary gear assembly.

Example 122. The device of any one of examples 114-121, wherein the actuation assembly comprises a threaded drive member, a threaded driven member, and a gear rack.

Example 123. The device of any of examples 114-122, wherein the threaded drive member comprises a head that moves linearly within the gap due to movement of the leaflets during the diastolic phase.

Example 124. The device of any one of examples 114-123, wherein each of the one or more paddles comprises one or more pinion gears.

Example 125. The device of examples 114-124, wherein the actuation assembly comprises a rack and each of the one or more paddles comprises one or more pinion gears that are driven by the rack.

Example 126. The device of any one of examples 114-125, further comprising a gripping member configured to grasp a native valve leaflet.

Example 127. The device of example 126, further comprising a second actuation assembly coupled to the base, wherein the second actuation assembly is configured to move the gripping member between an open position and a closed position, wherein the second actuation assembly comprises a threaded drive member and a threaded driven member, and wherein the gripping member is connected to the threaded driven member such that movement of the threaded driven member by the threaded drive member causes the gripping member to move between the open and closed positions.

Example 128. The device of any one of examples 126-127, wherein the gripping member is configured to grasp the native valve leaflet with one of the one or more paddles.

Example 129. The device of any one of examples 114-128, wherein each of the one or more paddles extends from a pivotable portion, that is pivotably connected to the base, to a free end.

Example 130. The device of any one of examples 114-129, further comprising a paddle frame connected to each of the one or more paddles, wherein the paddle frame is configured to coapt two native valve leaflets together.

Example 131. The device of example 130, further comprising a second actuation assembly coupled to the base, wherein the second actuation assembly is configured to move the paddle frame between a narrowed configuration and an expanded configuration, wherein the second actuation assembly comprises a threaded drive member and a threaded driven member, and wherein the paddle frame is connected to the threaded driven member such that movement of the threaded driven member by the threaded drive member causes the paddle frame to move between the narrowed and expanded configurations.

Example 132. The device of example 131, further comprising frame actuation elements that are pivotably coupled to the one or more paddles, wherein the frame actuation elements are connected to the threaded driven member such that movement of the threaded driven member causes the paddle frame to move between the narrowed and expanded configurations.

Example 133. The device of any one of examples 114-132, wherein each paddle of the one or more paddles can form an angle with the base that is greater than 175 degrees.

Example 134. The device of any one of examples 114-133, further comprising a second actuation assembly that includes a threaded drive member and a threaded driven member, wherein the second actuation assembly is configured to actuate one or more of the base, a gripping member, and a paddle frame of the device.

Example 135. A valve treatment device for attaching to a native valve of a patient, the valve treatment device comprising: a base; one or more paddles pivotably coupled to the base such that the paddles are movable between an open position and a closed position; an actuation assembly coupled to the base, wherein the actuation assembly is configured to move of the one or more paddles relative to the base such that the one or more paddles move between the closed position and the open position; wherein a proximal end of the actuation assembly is positioned proximate a gap, such that the actuation assembly can linearly move within the gap; a biasing member that engages the actuation assembly to maintain the actuation assembly in a desired position relative to the gap; a bias adjustment assembly configured to position the bias member in any one or more bias positions; and wherein, when the one or more paddles are in the closed position and attached to the native valve of the patient, movement of the leaflets during the diastolic phase causes the actuation assembly to linearly move within the gap.

Example 136. The device of example 135 wherein the bias adjustment assembly comprises a bias member space configured to receive at least a portion of the bias member.

Example 137. The device of any one of examples 135-136 wherein the bias adjustment assembly comprises a positioner space configured to receive at least a portion of a bias member positioner.

Example 138. The device of any one of examples 135-136 wherein the bias adjustment assembly comprises a positioner for changing the position of the bias member.

Example 139. The device of any one of examples 135-136 wherein the bias adjustment assembly comprises a threaded positioner.

Example 140. The device of any one of examples 135-136 wherein the bias adjustment assembly comprises a threaded positioner having a central opening.

Example 141. The device of any one of examples 138-140 wherein the bias adjustment assembly comprises a threaded positioner space configured to receive at least a portion of the threaded positioner.

Example 142. The device of any one of examples 135-141 wherein the bias adjustment assembly comprises at least first and second states and wherein the first state comprises a first bias position and the second state comprises a second bias position different form the first bias position.

Example 143. The device of any one of examples 135-141 wherein the bias adjustment assembly comprises at least first and second states and wherein the first state comprises a first bias position providing a first bias force on the actuation assembly and the second state comprises a second bias position providing a second bias force on the actuation assembly and wherein the second bias force is different from the first bias force.

Example 144. The device of any one of examples 135-141 wherein the bias adjustment assembly comprises at least first and second states and wherein the first state comprises a first bias position providing a first range of bias forces on the actuation assembly and the second state comprises a second bias position providing a second range of bias forces on the actuation assembly and wherein the second range of bias forces is different from the first range of bias forces.

Example 145. The device of any one of examples 47-88 and 135-144 wherein the bias member comprises a first spring coefficient on loading of the bias member and a second spring coefficient on unloading of the bias member.

Example 146. The device of any one of examples 47-88 and 135-144 wherein the bias member comprises a first spring coefficient on loading of the bias member and second and third spring coefficients on unloading of the bias member.

Example 147. The device of any one of examples 47-88 and 135-144 wherein the bias member comprises a first force displacement curve on loading of the bias member and second force displacement curve on unloading of the bias member.

Example 148. The device of any one of examples 47-88 and 135-144 wherein the bias member comprises a first force displacement curve on loading of the bias member and second and third force displacement curves on unloading of the bias member.

Example 149. The device of any one of examples 47-88 and 135-148 wherein the bias member comprises a top and bottom loading surfaces and a plurality of struts connected to the top and bottom surfaces and wherein the struts each comprise a curved shape.

Example 150. The device of any one of examples 47-88 and 135-148 wherein the bias member comprises top and bottom loading surfaces and a plurality of struts connected to the top and bottom surfaces and wherein the struts each comprise a concave portion, a convex portion, and an inflexion portion between the concave and convex portions.

Example 151. The device of any one of examples 47-88 and 135-148 wherein the bias member comprises top and bottom loading surfaces and a plurality of struts connected to the top and bottom surfaces and wherein the struts each comprise a first curved portion, a second curved portion, and an inflexion portion between the first and second curved portion.

Example 152. The device of any one of examples 47-88 and 135-144 wherein the bias member comprises a compound spring member having a plurality of stacked spring portions.

Example 153. The device of any one of examples 47-88 and 135-144 wherein the bias member comprises a spring member having a plurality of arcuate sections with concave and convex portions.

Example 154. The device of any one of examples 47-88 and 135-144 wherein the bias member comprises a plurality of stacked spring members having a plurality of arcuate sections with concave and convex portions.

Example 155. The device of any one of examples 47-88 and 135-144 wherein the bias member comprises a rectangular spring member having a plurality of sections with concave and convex portions.

Example 156. The device of any one of examples 47-88 and 135-144 wherein the bias member comprises a plurality of stacked rectangular spring members having a plurality of sections with concave and convex portions.

Example 157. The device of any one of examples 47-88 and 135-144 wherein the bias member comprises a spring member having a plurality of protruding portions, and each protruding portion comprises a plateaued linear surface.

Example 158. The device of any one of examples 47-88 and 135-144 wherein the bias member comprises a plurality of spring members having a plurality of protruding portions, and each protruding portion comprises a plateaued linear surface.

Example 159. The device of any one of examples 47-88 and 135-144 wherein the bias member comprises curved body having first and second distal end portions and a gap between the distal end portions.

Example 160. The device of any one of examples 47-88 and 135-144 wherein the bias member comprises curved body having first and second distal end portions and a gap between the distal end portions and wherein the first and second distal end portions are offset from each other.

Example 161. The device of any one of examples 47-88 and 135-144 wherein the bias member comprises curved body having first and second distal end portions and a gap between the distal end portions and wherein the first and second distal end portions comprise convex and concave portions.

Example 162. The device of any one of examples 47-88 and 135-144 wherein the bias member comprises cylindrical side wall having first and second openings, the first and second openings having a common center line.

Example 163. The device of any one of examples 47-88 and 135-144 wherein the bias member comprises rectangular side wall having first and second openings, the first and second openings having a common center line.

Example 164. A valve treatment device for attaching to a native valve of a patient, the valve treatment device comprising: a base; one or more paddles pivotably coupled to the base such that the one or more paddles are movable between an open position and a closed position; an actuation assembly coupled to the base, and wherein the actuation assembly is configured to pivot the one or more paddles relative to the base between the open position and the closed position responsive to the actuation assembly receiving an input.

Example 164A. The valve treatment device of example 164 wherein the device further comprises one or more clasps coupled to the one or more paddles and having: a base portion connected to the one or more paddles, a movable arm portion; and a joint portion connected to the base portion and the movable arm portion, the joint portion comprising one or more joint elements and one or more gaps between the joint elements, and wherein the one or more gaps comprise at least one tapered space.

Example 165. The device of example 164 or 164A wherein the at least one tapered space comprises a space that gradually decreases in size.

Example 166. The device of any one of examples 164-165 wherein the at least one tapered space comprises a space having a linear taper.

Example 167. The device of any one of examples 164-166 wherein the at least one tapered space comprises a space having a curvilinear taper.

Example 168. The device of any one of examples 164-167 wherein the at least one tapered space comprises first and second side wall and wherein the first side wall is a side wall of a first joint element, and the second side wall is a side wall of a second joint element and wherein the first and second side walls are non-parallel with respect to each other.

Example 169. The device of any one of examples 164-168 wherein the at least one tapered space comprises first and second end portions and wherein the first and second end portions comprises different sizes.

Example 170. The device of any one of examples 164-169 wherein the at least one tapered space comprises a first end portion having a first opening and a second end portions having a second opening, and wherein the first opening is larger than the second opening.

Example 171. The device of any one of examples 164-170 wherein the joint portion comprises outer side portions and the at least one tapered space is disposed on the outer side portions.

Example 172. The device of any one of examples 164-171 wherein the at least one tapered space comprises a plurality of tapered spaces having a plurality of different tapers.

Example 173. The device of any one of examples 164-172 wherein the at least one tapered space comprises a taper angle in the range of 5 to 45 degrees.

Example 174. A valve treatment device for attaching to a native valve of a patient, the valve treatment device comprising: a base; one or more paddles pivotably coupled to the base such that the one or more paddles are movable between an open position and a closed position; an actuation assembly coupled to the base, wherein the actuation assembly is configured to pivot the one or more paddles relative to the base between the open position and the closed position responsive to the actuation assembly receiving an input,

Example 174A. The valve treatment device of example 174, further comprising one or more clasps coupled to the one or more paddles and having: a base portion connected to the one or more paddles, a movable arm portion; a joint portion connected to the base portion and the movable arm portion; and wherein the movable arm portion comprises an arm thickness and wherein the joint portion comprises a joint thickness and wherein the arm thickness is greater than the joint thickness.

Example 175. The device of example 174 Or example 174A wherein base portion comprises a base thickness and the arm thickness is greater than the base thickness.

Example 176. The device of any of examples 174-175 wherein the movable arm portion comprise a barbed portion having a barb thickness and wherein the base thickness is the same as the arm thickness.

Example 177. The device of any of examples 174-177 wherein the one or more clasps further comprise a transition portion between the movable arm portion and the joint portion, and the transition portion comprises a step transition from the movable arm portion to the joint portion.

Example 178. The device of any of examples 174-177 wherein the one or more clasps further comprise a transition portion between the movable arm portion and the joint portion, and the transition portion comprises a linear slope transition from the movable arm portion to the joint portion.

Example 179. The device of any of examples 174-177 wherein the one or more clasps further comprise a transition portion between the movable arm portion and the joint portion, and the transition portion comprises a curved slope transition from the movable arm portion to the joint portion.

Example 180. The device of any of examples 174-179 wherein the each of the one or more clasps comprise a single piece of material having the base, movable arm and joint portions.

Example 181. The device of any of examples 174-180 wherein the movable arm comprises an arm stiffness and the joint portion comprises a joint stiffness and wherein the arm stiffness is greater than the joint stiffness.

Example 182. A valve treatment device for attaching to a native valve of a patient, the valve treatment device comprising: a base; one or more paddles pivotably coupled to the base such that the one or more paddles are movable between an open position and a closed position; an actuation assembly coupled to the base, wherein the actuation assembly is configured to pivot the one or more paddles relative to the base between the open position and the closed position responsive to the actuation assembly receiving an input.

Example 182A. The valve repair device of example 182, further comprising one or more clasps coupled to the one or more paddles and having: a base portion connected to the one or more paddles, a movable arm portion; a joint portion connected to the base portion and the movable arm portion; and wherein the joint portion comprises an opening for receiving at least a portion of the one or more paddles.

Example 183. The device of example 182 or 182A wherein the portion of the one or more paddles received in the opening comprises a gear portion.

Example 184. The device of example 182 or 182A wherein the portion of the one or more paddles received in the opening comprises a portion of a paddle arm.

Example 185. The device of any one of examples 182-184 wherein the opening comprises a rectangular opening.

Example 186. The device of any one of examples 182-185 wherein the opening comprises a curved opening.

Example 187. The device of any one of examples 182-186 wherein the opening extends into a portion of the base portion.

Example 188. The device of any one of examples 182-187 wherein the base portion comprises first and second base portion separated by a cut.

Example 189. The device of any one of examples 182-188 wherein the one or more paddles comprise a pivot axis and the joint portion of the one or more clasps comprises a central axis and where the central axis is offset from the pivot axis.

Example 190. The device of any one of example 182-188 wherein the one or more paddles comprise a pivot axis and the joint portion of the one or more clasps comprises a central axis and where the central axis is coaxial with the pivot axis.

Example 191. A valve treatment device for attaching to a native valve of a patient, the valve treatment device comprising: a base; one or more paddles pivotably coupled to the base such that the one or more paddles are movable between an open position and a closed position; an actuation assembly coupled to the base, wherein the actuation assembly is configured to pivot the one or more paddles relative to the base between the open position and the closed position responsive to the actuation assembly receiving an input.

Example 191A. The valve treatment device of example 191, further comprising one or more clasps coupled to the one or more paddles and having: a base portion connected to the one or more paddles, a movable arm portion; a joint portion connected to the base portion and the movable arm portion; and wherein the joint portion comprises at least one coil spring portion.

Example 192. The device of example 191 or example 191A wherein the base portion, movable arm portion, joint portion, and coil spring portion are made from a single piece of material.

Example 193. The device of example 191 or example 191A wherein the base portion is made from a first piece of material, the movable arm portion is made from a second piece of material, and the coil spring portion is made from a third piece of material and wherein the third piece of material is joined to the first and second pieces of material.

Example 194. The device of any one of examples 191-193 wherein the coil spring portion comprises a bias force urging the base portion and the movable arm portion together.

Example 195. The device of any one of examples 191-194 wherein the coil spring portion comprises a coil axis that is offset from a paddle pivot axis.

Example 196. A clasp for a valve treatment device for attaching to a native valve of a patient, the clasp comprising a base portion, a movable arm portion, and a joint portion. The joint portion is connected to the base portion and the movable arm portion. The joint portion comprising one or more joint elements and one or more gaps between the joint elements. The one or more gaps comprise at least one tapered space.

Example 197. The clasp of example 196 wherein the at least one tapered space comprises a space that gradually decreases in size.

Example 198. The clasp of any one of examples 196-197 wherein the at least one tapered space comprises a space having a linear taper.

Example 199. The clasp of any one of examples 196-198 wherein the at least one tapered space comprises a space having a curvilinear taper.

Example 200. The clasp of any one of examples 196-198 wherein the at least one tapered space comprises first and second side wall and wherein the first side wall is a side wall of a first joint element, and the second side wall is a side wall of a second joint element and wherein the first and second side walls are non-parallel with respect to each other.

Example 201. The clasp of any one of examples 196-168 wherein the at least one tapered space comprises first and second end portions and wherein the first and second end portions comprises different sizes.

Example 202. The clasp of any one of examples 196-201 wherein the at least one tapered space comprises a first end portion having a first opening and a second end portions having a second opening, and wherein the first opening is larger than the second opening.

Example 203. The clasp of any one of examples 196-202 wherein the joint portion comprises outer side portions and the at least one tapered space is disposed on the outer side portions.

Example 204. The clasp of any one of examples 196-203 wherein the at least one tapered space comprises a plurality of tapered spaces having a plurality of different tapers.

Example 205. The clasp of any one of examples 197-204 wherein the at least one tapered space comprises a taper angle in the range of 5 to 45 degrees.

Example 206. A clasp for a valve treatment device for attaching to a native valve of a patient, the clasp comprising a base portion, a movable arm portion, and a joint portion connected to the base portion and the movable arm portion. The movable arm portion comprises an arm thickness. The joint portion comprises a joint thickness. The arm thickness is greater than the joint thickness.

Example 207. The clasp of example 206 wherein base portion comprises a base thickness and the arm thickness is greater than the base thickness.

Example 208. The clasp of any of examples 206-207 wherein the movable arm portion comprise a barbed portion having a barb thickness and wherein the base thickness is the same as the arm thickness.

Example 209. The clasp of any of examples 206-208 wherein the one or more clasps further comprise a transition portion between the movable arm portion and the joint portion, and the transition portion comprises a step transition from the movable arm portion to the joint portion.

Example 210. The clasp of any of examples 206-209 wherein the one or more clasps further comprise a transition portion between the movable arm portion and the joint portion, and the transition portion comprises a linear slope transition from the movable arm portion to the joint portion.

Example 211. The clasp of any of examples 206-210 wherein the one or more clasps further comprise a transition portion between the movable arm portion and the joint portion, and the transition portion comprises a curved slope transition from the movable arm portion to the joint portion.

Example 212. The clasp of any of examples 206-211 wherein the each of the one or more clasps comprise a single piece of material having the base, movable arm and joint portions.

Example 213. The clasp of any of examples 206-212 wherein the movable arm comprises an arm stiffness and the joint portion comprises a joint stiffness and wherein the arm stiffness is greater than the joint stiffness.

Example 214. A clasp for a valve treatment device for attaching to a native valve of a patient. The clasp includes a base portion, a movable arm portion, and a joint portion. The joint portion is connected to the base portion and the movable arm portion. The joint portion comprises at least one coil spring portion.

Example 215. The clasp of example 214 wherein the base portion, movable arm portion, joint portion, and coil spring portion are made from a single piece of material.

Example 216. The clasp of example 214 wherein the base portion is made from a first piece of material, the movable arm portion is made from a second piece of material, and the coil spring portion is made from a third piece of material and wherein the third piece of material is joined to the first and second pieces of material.

Example 217. The clasp of any one of examples 214-216 wherein the coil spring portion comprises a bias force urging the base portion and the movable arm portion together.

Example 218. The clasp of any one of examples 214-217 wherein the coil spring portion comprises a coil axis that is offset from a paddle pivot axis.

Example 219. A valve repair device comprising: a pair of paddles that are adjustable to a plurality of different implantation positions. The pair of paddles are also able to move in response to movement of the leaflets of the native valve leaflets during the diastolic phase toward an open position from a set implantation position.

Example 220. The valve repair device of example 219 wherein the paddles are allowed to move from the implantation position with the movement of the native valve leaflets by allowing movement of a drive member.

Example 221. The valve repair device of any one of examples 219-220 wherein a degree or distance that the paddles can move from the implantation position is settable or controllable.

Example 222. The valve repair device of any one of examples 219-221 wherein an amount of force and/or a force profile required to move the paddles from the implantation position is settable or controllable.

Example 223. The valve repair device of example 221 wherein the degree or distance that the paddles can move from the implantation position is settable or controllable by adjusting a space or gap that receives a drive member.

Example 223. The valve repair device of example 221 wherein the degree or distance that the paddles can move from the implantation position is settable or controllable by adjusting a stop that engages a drive member.

Example 223. The valve repair device of example 221 wherein the degree or distance that the paddles can move from the implantation position is settable or controllable by adjusting an amount of compression of a spring.

Example 224. The valve repair device of example 223 wherein the spring is configured to bottom out or fully compress to limit movement of the drive member.

Example 225. The valve repair device of any one of examples 219-224 wherein an amount of force and/or a force profile required to move the paddles from the implantation position is settable or controllable by adjusting one or more springs.

Example 226. The valve repair device of any one of examples 219-224 wherein an amount of force and/or a force profile required to move the paddles from the implantation position is settable or controllable compressing or relaxing one or more springs.

Example 226. The valve repair device of any one of examples 219-224 wherein an amount of force and/or a force profile required to move the paddles from the implantation position is settable or controllable compressing or relaxing one or more springs with an adjustment member.

Any of the various systems, assemblies, devices, components, apparatuses, etc. in this disclosure can be sterilized (e.g., with heat, radiation, ethylene oxide, hydrogen peroxide, etc.) to ensure they are safe for use with patients, and the methods herein can comprise (or additional methods comprise or consist of) sterilization of the associated system, device, component, apparatus, etc. (e.g., with heat, radiation, ethylene oxide, hydrogen peroxide, etc.).

While various inventive aspects, concepts and features of the disclosures can be described and illustrated herein as embodied in combination in the examples herein, these various aspects, concepts, and features can be used in many alternative examples, either individually or in various combinations and sub-combinations thereof. Unless expressly excluded herein all such combinations and sub-combinations are intended to be within the scope of the present disclosure. Still further, while various alternative examples as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available alternative examples, whether presently known or later developed. Those skilled in the art can readily adopt one or more of the inventive aspects, concepts, or features into additional examples and uses within the scope of the present disclosure even if such examples are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present disclosure, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. The words used in the claims have their full ordinary meanings and are not limited in any way by the description of the examples in the specification.

What is claimed is:

1. A valve treatment device for attaching to a native valve of a patient, the valve treatment device comprising:
a base;
one or more paddles pivotably coupled to the base such that one or more paddles are movable between an open position and a closed position;
an actuation assembly coupled to the base, wherein the actuation assembly is configured to move of the one or more paddles relative to the base such that the one or more paddles move between the closed position and the open position;
a bias member that engages the actuation assembly to maintain the actuation assembly in a desired position relative to a gap; and
wherein, when the one or more paddles are in the closed position and attached to the native valve of the patient, movement of the valve during a diastolic phase causes the actuation assembly to linearly move within the gap.

2. The device of claim 1, further comprising a bias adjustment assembly, the bias adjustment assembly comprising a positioner for changing the position of the bias member.

3. The device of claim 1, wherein the bias adjustment assembly comprises a threaded positioner having a central opening and the bias adjustment assembly comprises a threaded positioner space configured to receive at least a portion of the threaded positioner.

4. The device of claim 1, wherein the bias adjustment assembly comprises at least first and second states and wherein the first state comprises a first bias position providing a first bias force on the actuation assembly and the second state comprises a second bias position providing a second bias force on the actuation assembly and wherein the second bias force is different from the first bias force.

5. The device of claim 1, wherein the bias member comprises a first spring coefficient on loading of the bias member and a second spring coefficient on unloading of the bias member.

6. The device of claim 1, wherein the bias member comprises a top loading surface and a bottom loading surface and a plurality of struts, a strut of the plurality of struts connected to the top and bottom loading surfaces and the strut comprising a curved shape.

7. The device of claim 1, wherein the bias member comprises a compound spring member having a plurality of stacked spring portions.

8. The device of claim 1, wherein the bias member comprises a spring member having a plurality of arcuate sections with concave and convex portions.

9. The device of claim 1, wherein the bias member comprises a spring member having a plurality of protruding portions, and each protruding portion comprises a plateaued linear surface.

10. The device of claim 1, wherein the bias member comprises curved body having first and second distal end portions and a gap between the first and second distal end portions.

11. The device of claim 1, wherein the bias member comprises a curved body having first and second distal end portions and a gap between the first and second distal end portions and wherein the first and second distal end portions are offset from each other.

12. The device of claim 1, wherein the actuation assembly comprises one or more of a rack and pinion assembly, a worm gear assembly, and a planetary gear assembly.

13. A valve treatment device for attaching to a native valve of a patient, the valve treatment device comprising:
a base;
one or more paddles pivotably coupled to the base such that the one or more paddles are movable between an open position and a closed position;
an actuation assembly coupled to the base, wherein the actuation assembly is configured to pivot the one or more paddles relative to the base between the open position and the closed position responsive to the actuation assembly receiving an input;
one or more clasps coupled to the one or more paddles and having:
a base portion connected to the one or more paddles,
a movable arm portion; and
a joint portion connected to the base portion and the movable arm portion, the joint portion comprising one or more joint elements and one or more gaps between the one or more joint elements, and wherein the one or more gaps comprise at least one tapered space.

14. The device of claim 13, wherein the at least one tapered space comprises first and second side walls and wherein the first side wall is a side wall of a first joint element and the second side wall is a side wall of a second joint element and wherein the first and second side walls are non-parallel with respect to each other.

15. A valve repair system, comprising:
a steerable catheter;
an implant catheter that extends through the steerable catheter;
a valve repair device coupled to the implant catheter;
wherein the valve repair device includes one or more paddles that are movable between an open position and a closed position;
wherein the one or more paddles are each configured to be secured to a native valve leaflet by moving the one or more paddles from the open position to the closed position; and
wherein a passage of the steerable catheter and a size of the valve repair device are configured to allow the valve repair device to slide through the steerable catheter.

16. The valve repair system of claim 15, wherein the valve repair device is configured to be implanted where a mitral valve area is between 2.2 and 6 cm², a transeptal puncture height is between 2.0 and 4.5 cm, a medial-lateral width of the valve repair device is between 2 mm and 4 mm, and an anterior-posterior width of the valve repair device is between 2 mm and 9 mm.

17. The valve repair system of claim 15, further comprising a steerable guide sheath.

18. The valve repair system of claim 17, wherein the steerable catheter extends through the steerable guide sheath.

19. The valve repair system of claim 15, further comprising a pair of gripping members disposed between the pair of paddles.

20. The valve repair system of claim 15, further comprising:
an actuation element comprising:
a catheter;
a drive end connected to the catheter, the drive end having a passage that is in communication with the catheter and a slot at a distal end of the drive end that is in communication with the passage;
a securing element that is configured to be moved through the catheter and the passage such that the securing element can be inserted into the slot at the distal end of the drive end;
wherein the valve repair device comprises an actuation assembly that comprises:
a drive member having a head, wherein the head comprises a bar that is configured to be inserted into the slot of the drive end of the actuation element; and
wherein the head is secured to the actuation element when the bar of the head of the drive member is inserted into the slot of the drive end of the actuation element and the securing element of the actuation element is positioned within the slot.
